(19) United States
(12) Patent Application Publication
(10) Pub. No.: US 2018/0231561 A1
(43) Pub. Date: Aug. 16, 2018

(54) METHODS FOR TREATING SOLID TUMORS AND THE USE OF BIOMARKERS AS A PREDICTOR OF CLINICAL SENSITIVITY TO IMMUNOMODULATORY THERAPIES

(71) Applicant: CELGENE CORPORATION, Summit, NJ (US)

(72) Inventors: Anita GANDHI, Bernardsville, NJ (US); Patrick HAGNER, Sparta, NJ (US); Rajesh CHOPRA, Summit, NJ (US); Anke KLIPPEL, Westfield, NJ (US)

(21) Appl. No.: 15/751,844

(22) PCT Filed: Aug. 11, 2016
(86) PCT No.: PCT/US16/46490

§ 371 Date: Feb. 9, 2018

Related U.S. Application Data
(60) Provisional application No. 62/204,405, filed on Aug. 12, 2015.

(51) Int. Cl. G01N 33/574; C12Q 1/6886

(52) U.S. Cl. CPC G01N 33/57484; C12Q 2600/158; C12Q 2600/106; C12Q 1/6886

(57) ABSTRACT

A method of identifying a subject having a solid tumor who is likely to be responsive to a treatment compound, comprising: administering the treatment compound to a subject having the solid tumor; obtaining a sample from the subject; determining the level of a biomarker in the sample from the subject, and diagnosing the subject as being likely to be responsive to the treatment compound if the level of the biomarker in the sample of the subject changes as compared to a reference level of the biomarker.

### CROSS-REFERENCE TO RELATED APPLICATIONS

The present application claims priority to U.S. Provisional Patent Application No. 62/204,405 filed Aug. 12, 2015, the entirety of which is incorporated herein by reference.

### 1. FIELD

Provided herein are biomarkers for use in predicting the clinical sensitivity of a solid tumor, and a subject's response to treatment with an immunomodulatory agent, such as 3-(5-amino-2-methyl-4-oxo-4H-quinazolin-3-yl)-piperidine-2,6-dione (“Compound A”).

### 2. BACKGROUND

### 2.1 Solid Tumors

A solid tumor is an abnormal mass of tissue that usually does not contain cysts or liquid areas. Solid tumors may be benign or malignant. A solid tumor grows in an anatomical site outside the bloodstream (in contrast, for example, to cancers of hematopoietic origin such as leukemias) and requires the formation of small blood vessels and capillaries to supply nutrients, etc. to the growing tumor mass. Solid tumors are named for the type of cells that form them. Non-limiting examples of solid tumors are sarcomas, carcinomas (epithelial tumors), melanomas, and glioblastomas. Exemplary solid tumors include biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma, glioblastomas; medulloblastoma), cervical cancer (e.g., cervical adenocarcinoma), colorectal cancer (e.g., colon cancer, rectal cancer, colorectal adenocarcinoma), gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), pancreatic cancer (e.g., pancreatic andenocarcinoma, intraductal papillary mucinous neoplasm (IPMN)), prostate cancer (e.g., prostate adenocarcinoma), skin cancer (e.g., squamous cell carcinoma (SCC), keratoacanthoma (A), melanoma, basal cell carcinoma (BCC)) and soft tissue sarcoma (e.g., malignant fibrous histiocytoma (WE), liposarcoma, malignant peripheral nerve sheath tumor (MPNST), chondrosarcoma, fibrosarcoma, myxosarcoma, osteosarcoma).

### 2.2 Hepatocellular Carcinoma (HCC)

HCC, also known as malignant hepatoma, is the most common primary malignancy of the liver and accounts for 80-90% of primary liver tumors. HCC is one of the most common and devastating malignant diseases worldwide, responsible for more than 1 million deaths annually in the world (Parkin et al., CA Cancer J. Clin. 1999, 49, 33-64; Bruix et al., Cancer Cell 2004, 5, 215-219).

The major risk factors for the development of HCC include hepatitis B or C viral infection, and alcoholic liver disease (Rustgi, Gastroenterol. Clin. North Am. 1987, 16, 545-551; Bosch et al., Semin. Liver Dis. 1999, 19, 271-285; Bosch et al., Gastroenterology 2004, 127, S5-S16; Moradpour et al., Eur. J. Gastro & Hepatol. 2005, 17, 477-483; Koike et al., J. Gastroenterol. Hepatol. 2008, 23, S87-S91; de Oliveria Andrade, J. Glob. Infect. Dis. 2009, 1, 33-37). HCC arises most commonly in cirrhotic livers following infection with hepatitis B virus (HBV) or hepatitis C virus (HCV) (Liaw, Semin. Liver Dis. 2005, 25, 40-47; Koike Clin. Gastroenterol. Hepatol. 2005, 3, 132-135). HCC is associated with HBV infection in about 50% of cases (Liaw, Semin. Liver Dis. 2005, 25, 40-47). HCV infection is the cause of 70% of the cases of HCC in Japan (Hasan, et al., Hepatology, 1990, 12:589-591; El-Serag et al., N. Engl. J. Med. 1999, 340, 745-750). The HCC incidence has been increasing in Western countries in recent years due to the spread of hepatitis C virus (HCV) infection (El-Serag, Hepatology 2002, 36, S74-83; Trevisani et al., Carcinogenesis 2008, 29, 1299-1305).

HCC is a disease of worldwide significance, of which there is no truly effective therapy, particularly for advanced disease. Therefore, there is a need for biomarkers to aid a HCC treatment and a method of predicting the responsiveness of a HCC subject to a HCC treatment, e.g., chemotherapy.

### 2.3 Glioblastoma (GBM)

Glioblastomas (GBM) are tumors that arise from astrocytes or supportive tissue of the brain. Glioblastomas are generally found in the cerebral hemispheres of the brain, but can be found anywhere in the brain or spinal cord. These tumors are usually highly malignant (cancerous) because the cells reproduce quickly and they are supported by a large network of blood vessels. Glioblastomas usually contain a mix of cell types. It is common for these tumors to contain cystic mineral, calcium deposits, blood vessels, or a mixed grade of cells. There are two types of glioblastomas. The first type of glioblastoma is primary or de novo. The tumors of the first type are very aggressive, and tend to form and make their presence quickly. The second type of glioblastoma is secondary. These tumors have a longer, somewhat slower growth history, yet aggressive. They may begin as lower-grade tumors which eventually become higher grade. The symptoms of glioblastomas are usually caused by increased pressure in the brain, including headache, nausea, vomiting, and drowsiness. Like many tumor types, the exact cause of glioblastoma is not known. The glioblastomas usually contain many different types of cells—some cells may respond well to certain therapies, while others may not be affected at all, thus they are usually difficult to treat and the treatment usually involve combinations of various approaches.

### 2.4 Cereblon

At least two isoforms of the protein cereblon (CRBN) exist, which are 442 and 441 amino acids long, respectively, and CRBN is conserved from plant to human. In humans, the CRBN gene has been identified as a candidate gene of an autosomal recessive nonsyndromic mental retardation (ARNSMR). See Higgins, J. J. et al., Neurology, 2004, 63:1927-1931. CRBN was initially characterized as an RGS-containing novel protein that interacted with a calcium-activated potassium channel protein (SLO1) in the rat brain, and was later shown to interact with a voltage-gated chloride channel (CIC-2) in the retina with AMPK1 and DDB1. See Jo, S. et al., J. Neurochem, 2005, 94:1212-1224; Hohberger B. et al., FEBS Lett, 2009, 583:633-637; Angers S. et al., Nature, 2006, 443:590-593. DDB1 was originally identified as a nucleotide excision repair protein that associates with damaged DNA binding protein 2 (DDB2). Its defective activity causes the repair defect in the patients with xeroderma pigmentosum complementation group E (XPE). DDB1 also appears to function as a component of numerous distinct DCX (DDB1-CUL4-X-box) E3 ubiquitin-protein ligase complexes which mediate the ubiquitination and subsequent proteasomal degradation of target proteins. CRBN has also been identified as a target for the development of therapeutic agents for diseases of the cerebral cortex. See WO 2010/137547 A1.

CRBN has recently been identified as a key molecular target that binds to thalidomide to cause birth defects. See Ito, T. et al., Science, 2010, 327:1345-1350. DDB1 was found to interact with CRBN and, thus, was indirectly associated with thalidomide. Moreover, thalidomide was able to inhibit auto-ubiquitination of CRBN in vitro, suggesting that thalidomide is an E3 ubiquitin-ligase inhibitor. Id. Importantly, this activity was inhibited by thalidomide in wild-type cells, but not in cells with mutated CRBN binding sites that prevent thalidomide binding. Id. The thalidomide binding site was mapped to a highly conserved C-terminal 104 amino acid region in CRBN. Id. Individual point mutants in CRBN, Y384A and W386A were both defective for thalidomide binding, with the double point mutant having the lowest thalidomide-binding activity. Id. A link between CRBN and the teratogenic effect of thalidomide was confirmed in animal models of zebra-fish and chick embryos. Id.

Whether binding to CRBN, the CRBN E3 ubiquitin-ligase complex, or one or more substrates of CRBN, is required for the beneficial effects of thalidomide and other drugs is yet to be established. Understanding these interactions with thalidomide and other drug targets will allow the definition of the molecular mechanisms of efficacy and/or toxicity and may lead to drugs with improved efficacy and toxicity profiles.

### 2.5 Compounds

A number of studies have been conducted with the aim of providing compounds that can safely and effectively be used to treat diseases associated with abnormal production of TNF-α. See, e.g., Marriott, J. B., et al., Expert Opin. Biol. Ther., 2001, 1(4): 1-8; G. W. Muller, et al., J Med Chem., 1996, 39(17): 3238-3240; and G. W. Muller, et al., Bioorg & Med Chem Lett., 1998, 8: 2669-2674. Some studies have focused on a group of compounds selected for their capacity to potently inhibit TNF-α production by LPS stimulated PBMC. L. G. Corral, et al., Ann. Rheum. Dis., 1999, 58:(Suppl I) 1107-1113. These compounds show not only potent inhibition of TNF-α but also marked inhibition of LPS induced monocyte IL1B and IL12 production. LPS induced IL6 is also inhibited by such compounds, albeit partially. These compounds are potent stimulators of LPS induced IL10. Id.

Compounds for the methods provided herein include, but are not limited to, the substituted 2-(2,6-dioxopiperidin-3-yl) phthalimides and substituted 2-(2,6-dioxopiperidin-3-yl)-1-oxoisoindoles described in U.S. Pat. Nos. 6,281,230 and 6,316,471, both to G. W. Muller, et al. Still other specific compounds disclosed herein belong to a class of isoindole-imides disclosed in U.S. Pat. Nos. 6,395,754, 6,555,554, 7,091,353, U.S. Publication No. 2004/0029832, and International Publication No. WO 98/54170, each of which is incorporated herein by reference.

Thalidomide, lenalidomide and pomalidomide have shown remarkable responses in patients with multiple myeloma, lymphoma and other hematological diseases such as myelodysplastic syndrome. See Galustian C, et al., Expert Opin Pharmacother., 2009, 10:125-133. These treatment compounds display a broad spectrum of activity, including anti-angiogenic properties, modulation of pro-inflammatory cytokines, co-stimulation of T cells, increased NK cell toxicity, direct anti-tumor effects and modulation of stem cell differentiation.

For example, thalidomide and lenalidomide have emerged as important options for the treatment of multiple myeloma in newly diagnosed patients, in patients with advanced disease who have failed chemotherapy or transplantation, and in patients with relapsed or refractory multiple myeloma. Lenalidomide in combination with dexamethasone has been approved for the treatment of patients with multiple myeloma who have received at least one prior therapy. Pomalidomide may also be administered in combination with dexamethasone. U.S. Patent Publication No. 2004/0029832 A1, the disclosure of which is hereby incorporated in its entirety, discloses the treatment of multiple myeloma.

Another compound provided herein is 3-(5-amino-2-methyl-4-oxo-4H-quinazolin-3-yl)-piperidine-2,6-dione (“Compound A”), which has the following structure:

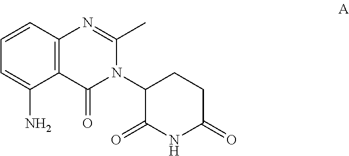 [SMILES: Cc1nc2cccc(N)c2c(=O)n1C1CCC(=O)NC1=O]

or an enantiomer or a mixture of enantiomers thereof; or a pharmaceutically acceptable salt, solvate, hydrate, co-crystal, clathrate, or polymorph thereof.

Compound A can be prepared as described in U.S. Pat. No. 7,635,700, the disclosure of which is incorporated herein by reference in its entirety. The compound can be also synthesized according to other methods apparent to those of skill in the art based upon the teaching herein. In certain embodiments, Compound A is in a crystalline form described in U.S. Provisional Pat. App. No. 61/451,806, filed Mar. 11, 2011, which is incorporated herein by reference in its entirety. In some embodiments, the hydrochloride salt of Compound A is used in the methods provided herein. Methods of treating, preventing and/or managing cancers and other diseases using Compound A are described in U.S. Provisional Pat. App. No. 61/451,995, filed Mar. 11, 2011, which is incorporated herein by reference in its entirety.

Yet another compound provided herein is 3-[4-(4-Morpholin-4-ylmethyl-benzyloxy)-1-oxo-1,3-dihydro-isoindol-2-yl]-piperidine-2,6-dione (Compound B), which has the following structure:

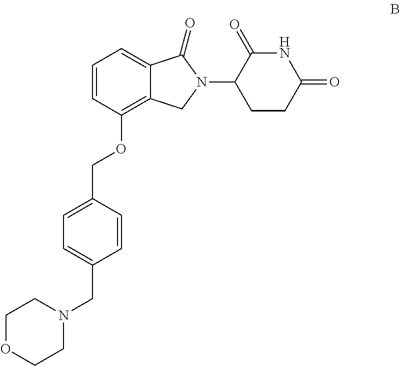 [SMILES: O=C1CCC(N2Cc3c(OCc4ccc(CN5CCOCC5)cc4)cccc3C2=O)C(=O)N1]

or an enantiomer or a mixture of enantiomers thereof; or a pharmaceutically acceptable salt, solvate, hydrate, co-crystal, clathrate, or polymorph thereof.

The conventional methods of assessing the effects of immunomodulatory compounds require live cellular assays or lengthy clinical endpoints. These cellular tests are cumbersome and often require the use of various stimulants (e.g., lipopolysaccharide or anti-CD3 antibody). Indirect endpoints such as cytokine production are evaluated, which can be influenced via multiple pathways. Further, clinical efficacy of these compounds could not be correctly predicted, as it could only be measured in terms of patient response, which usually requires a minimum of several months of treatment. In view of the deficiencies of the conventional methods, there is a need to develop an efficient, sensitive and accurate method to detect, quantify and characterize the pharmacodynamic activity of immunomodulatory compounds.

### 3. SUMMARY

In one aspect, provided herein is a method of identifying a subject having a solid tumor, e.g., HCC or GBM, who is likely to be responsive to a treatment compound, comprising:

(a) administering the treatment compound to a subject having a solid tumor;

(b) obtaining a sample from the subject;

(c) determining the level of a biomarker in the sample from the subject, wherein the biomarker is selected from the group consisting of biomarkers identified in Tables 1 to 6, and combinations thereof; and

(d) diagnosing the subject as being likely to be responsive to the treatment compound if the level of the biomarker in the sample of the subject changes as compared to a reference level of the biomarker. In one embodiment, the solid tumor is HCC. In another embodiment, the solid tumor is GBM.

In another aspect, provided herein is a method of identifying a subject having a solid tumor, e.g., HCC or GBM, who is likely to be responsive to a treatment compound, comprising:

(a) administering the treatment compound to a subject having a solid tumor;

(b) obtaining a sample from the subject;

(c) determining the level of a biomarker in the sample from the subject, wherein the biomarker is selected from the group consisting of biomarkers identified in Table 1, Table 3, and Table 4, and combinations thereof; and

(d) diagnosing the subject as being likely to be responsive to the treatment compound if the level of the biomarker in the sample of the subject is higher than a reference level of the biomarker. In one embodiment, the solid tumor is HCC. In another embodiment, the solid tumor is GBM.

In another aspect, provided herein is a method of identifying a subject having a solid tumor, e.g., HCC or GBM, who is likely to be responsive to a treatment compound, comprising:

(a) administering the treatment compound to a subject having a solid tumor;

(b) obtaining a sample from the subject;

(c) determining the level of a biomarker in the sample from the subject, wherein the biomarker is selected from the group consisting of biomarkers identified in Table 2, Table 5, and Table 6, and combinations thereof; and

(d) diagnosing the subject as being likely to be responsive to the treatment compound if the level of the biomarker in the sample of the subject is lower than a reference level of the biomarker. In one embodiment, the solid tumor is HCC. In another embodiment, the solid tumor is GBM.

In another aspect, provided herein is a method of treating a solid tumor, e.g., HCC or GBM, comprising:

(a) obtaining a sample from the subject;

(b) determining the level of a biomarker in the sample from the subject, wherein the biomarker is selected from the group consisting of biomarkers identified in Tables 1 to 6, and combinations thereof;

(c) diagnosing the subject as being likely to be responsive to a treatment compound if the level of the biomarker in the sample of the subject changes as compared to a reference level of the biomarker; and

(d) administering a therapeutically effective amount of the treatment compound to the subject diagnosed to be likely to be responsive to the treatment compound. In one embodiment, the solid tumor is HCC. In another embodiment, the solid tumor is GBM.

In another aspect, provided herein is a method of treating a solid tumor, e.g., HCC or GBM, comprising:

(a) obtaining a sample from the subject;

(b) determining the level of a biomarker in the sample from the subject, wherein the biomarker is selected from the group consisting of biomarkers identified in Table 1, Table 3, and Table 4, and combinations thereof;

(c) diagnosing the subject as being likely to be responsive to a treatment compound if the level of the biomarker in the sample of the subject is higher as than a reference level of the biomarker; and

(d) administering a therapeutically effective amount of the treatment compound to the subject diagnosed to be likely to be responsive to the treatment compound. In one embodiment, the solid tumor is HCC. In another embodiment, the solid tumor is GBM.

In another aspect, provided herein is a method of treating a solid tumor, e.g., HCC or GBM, comprising:

(a) obtaining a sample from the subject;

(b) determining the level of a biomarker in the sample from the subject, wherein the biomarker is selected from the group consisting of biomarkers identified in Table 2, Table 5, and Table 6, and combinations thereof;

(c) diagnosing the subject as being likely to be responsive to a treatment compound if the level of the biomarker in the sample of the subject is lower as than a reference level of the biomarker; and

(d) administering a therapeutically effective amount of the treatment compound to the subject diagnosed to be likely to be responsive to the treatment compound. In one embodiment, the solid tumor is HCC. In another embodiment, the solid tumor is GBM.

In another aspect, provided herein is a method of predicting the responsiveness of a subject having or suspected of having a solid tumor, e.g., HCC or GBM, to a treatment compound, comprising:

(a) administering the treatment compound to a subject having a solid tumor;

(b) obtaining a sample from the subject;

(c) determining the level of a biomarker in the sample from the subject, wherein the biomarker is selected from the group consisting of biomarkers identified in Tables 1 to 6, and combinations thereof;

(d) diagnosing the subject as being likely to be responsive to a treatment of a solid tumor with the treatment compound if the level of the biomarker in the sample changes as compared to the level of the biomarker obtained from a reference sample. In one embodiment, the solid tumor is HCC. In another embodiment, the solid tumor is GBM.

In another aspect, provided herein is a method of predicting the responsiveness of a subject having or suspected of having a solid tumor, e.g., HCC or GBM, to a treatment compound, comprising:

(a) administering the treatment compound to a subject having a solid tumor;

(b) obtaining a sample from the subject;

(c) determining the level of a biomarker in the sample from the subject, wherein the biomarker is selected from the group consisting of biomarkers identified in Table 1, Table 3, and Table 4, and combinations thereof;

(d) diagnosing the subject as being likely to be responsive to a treatment of a solid tumor with the treatment compound if the level of the biomarker in the sample is higher than the level of the biomarker obtained from a reference sample. In one embodiment, the solid tumor is HCC. In another embodiment, the solid tumor is GBM.

In another aspect, provided herein is a method of predicting the responsiveness of a subject having or suspected of having a solid tumor, e.g., HCC or GBM, to a treatment compound, comprising:

(a) administering the treatment compound to a subject having a solid tumor;

(b) obtaining a sample from the subject;

(c) determining the level of a biomarker in the sample from the subject, wherein the biomarker is selected from the group consisting of biomarkers identified in Table 2, Table 5, and Table 6, and combinations thereof;

(d) diagnosing the subject as being likely to be responsive to a treatment of a solid tumor with the treatment compound if the level of the biomarker in the sample is less than the level of the biomarker obtained from a reference sample. In one embodiment, the solid tumor is HCC. In another embodiment, the solid tumor is GBM.

In another aspect, provided herein is a method of monitoring the efficacy of a treatment of a solid tumor, e.g., HCC or GBM, in a subject with a treatment compound, comprising:

(a) administering the treatment compound to a subject having a solid tumor;

(b) obtaining a sample from the subject;

(c) determining the level of a biomarker in the sample from the subject, wherein the biomarker is selected from the group consisting of biomarkers identified in Tables 1 to 6, and combinations thereof;

(d) comparing the level of the biomarker in the sample with the level of the biomarker obtained from a reference sample, wherein a change in the level as compared to the reference is indicative of the efficacy of the treatment compound in treating the solid tumor in the subject. In one embodiment, the solid tumor is HCC. In another embodiment, the solid tumor is GBM.

In another aspect, provided herein is a method of monitoring the efficacy of a treatment of solid tumor, e.g., HCC or GBM, in a subject with a treatment compound, comprising:

(a) administering the treatment compound to a subject having a solid tumor;

(b) obtaining a sample from the subject;

(c) determining the level of a biomarker in the sample from the subject, wherein the biomarker is selected from the group consisting of biomarkers identified in Table 1, Table 3, and Table 4, and combinations thereof;

(d) comparing the level of the biomarker in the sample with the level of the biomarker obtained from a reference sample, wherein an increased level as compared to the reference is indicative of the efficacy of the treatment compound in treating the solid tumor in the subject. In one embodiment, the solid tumor is HCC. In another embodiment, the solid tumor is GBM.

In another aspect, provided herein is a method of monitoring the efficacy of a treatment of a solid tumor, e.g., HCC or GBM, in a subject with a treatment compound, comprising:

(a) administering the treatment compound to a subject having a solid tumor;

(b) obtaining a sample from the subject;

(c) determining the level of a biomarker in the sample from the subject, wherein the biomarker is selected from the group consisting of biomarkers identified in Table 2, Table 5, and Table 6, and combinations thereof;

(d) comparing the level of the biomarker in the sample with the level of the biomarker obtained from a reference sample, wherein a decreased level as compared to the reference is indicative of the efficacy of the treatment compound in treating the solid tumor in the subject. In one embodiment, the solid tumor is HCC. In another embodiment, the solid tumor is GBM.

In some embodiments of the various methods provided herein, the biomarker is selected from a group consisting of Nestin, KAT1/CCBL1, WIBG, MVP, PARP4, ZFP91, and ZNF198. In some embodiments of the various methods provided herein, the biomarker is selected from a group consisting of Nestin, KAT1/CCBL1, and WIBG. In other embodiments, the biomarker is selected from a group consisting of MVP, PARP4, ZFP91, and ZNF198.

In some embodiments of the various methods provided herein, the biomarker is AHNAK, ALOX5, AMPD3, ANXA4, ANXA6, ARHGAP19, ASNS, ASPM, ATP2B4, B4GALT3, BANK1, BCDIN3D, BLZF1, BMF, BST2, C10orf76, C19orf66, CA2, CA8, CAMSAP3, CCDC69, CCNB1, CD36, CDC7, CDCA3, CENPF, CLN3, CNN3, CORO1B, CPNE2, CRBN, CSNK1A1, CSRP2, CTNND1, CTSH, DAPK2, DDX58, DHPS, DHX58, DLG2, DLGAP5, DOK3, DTX3L, ECT2, EFCAB4B, EHMT1, EHMT2, EIF2AK2, EPB41L1, EPCAM, ESRP1, ETV6, EXTL2, F13A1, FAM195A, FAM65B, FBRSL1, FCGR2B, FES, FHOD1, FIGNL1, FMNL3, GBP1, GMFG, GMPR, GPT2, GRAMD1A, GRAMD1B, GRPEL2, HIP1, HJURP, HLA-B, HLA-DMA, HMCES, HMMR, HOXC4, HPSE, ICAM2, ID3, IF135, IFIH1, IFIT1, IFIT3, IFIT5, IFITM2, IKZF1, IKZF3, IL4I1, IRF7, IRF9, IRS2, ISG15, ISG20, ITGB7, JAK3, KIF18B, KIF22, KIF2C, LAP3, LGALS1, LGALS3BP, LIMD1, LIPG, LPXN, MAN2A2, MARCKS, MFI2, MGARP, MINA, MIS18BP1, MOV10, MPP7, MUC1, MX1, MX2, MYO1G, NCF2, NEIL1, NFKBID, NME3, NMI, NPIPB5, NT5C3A, OAS1, OAS2, OAS3, OMA1, ORC6, PARP14, PARP9, PARVB, PBK, PBXIP1, PDE6D, PKMYT1, PLD4, PLEKHO1, PLK1, PLSCR1, PLXNB2, PODXL, PODXL2, POLE2, POMP, PPFIBP1, PRDM15, PRNP, PTAFR, PTMS, PTTG1, PYROXD1, QPRT, RAB13, RASA4B, RASSF6, RCN1, RGCC, RGS1, RGS2, RNF213, S100A13, SAMD9L, SAMHD1, SEC14L1, SERPINH1, SGOL1, SGOL2, SLCO3A1, SLCO4A1, SLFN11, SLFN13, SLFN5, SP110, SP140, SPN, SPR, STAP1, STAT1, STAT2, TACC3, TAP1, TAX1BP3, THEMIS2, THTPA, TIMM8B, TNFAIP8L2, TNFSF8, TOP2A, TP5313, TPX2, TREX1, TRIB3, TRIM22, TTC39C, TXNIP, UBA7, UBE2L6, USP41, VCL, VNN2, WIZ, WSB1, WWC1, ZBTB38, ZFP91, ZMYM2, ZNF385B, ZNF581 or ZNF644. In certain embodiments, any combination of two or more of the above-identified biomarkers is also contemplated.

In other embodiments of the various methods provided herein, the biomarker is selected from a group consisting of AHNAK, ALOX5, AMPD3, ANXA4, ANXA6, ATP2B4, BMF, BST2, C10orf76, C19orf66, CD36, CLN3, CNN3, CORO1B, CPNE2, CRBN, CSRP2, CTNND1, CTSH, DAPK2, DDX58, DHX58, DLG2, DTX3L, EIF2AK2, EPB41L1, ETV6, EXTL2, F13A1, FAM65B, FCGR2B, FES, FMNL3, GBP1, GMFG, GMPR, HIP1, HLA-B, HLA-DMA, HPSE, ID3, IFI35, IFIH1, IFIT1, IFIT3, IFIT5, IFITM2, IL4I1, IRF7, IRF9, ISG15, ISG20, ITGB7, JAK3, LAP3, LGALS1, LGALS3BP, LIMD1, MAN2A2, MARCKS, MFI2, MGARP, MOV10, MPP7, MUC1, MX1, MX2, MYO1G, NCF2, NME3, NMI, NT5C3A, OAS1, OAS2, OAS3, PARP14, PARP9, PBXIP1, PLD4, PLEKHO1, PLSCR1, PLXNB2, POMP, PPFIBP1, PTMS, QPRT, RAB13, RCN1, RGCC, RNF213, S100A13, SAMD9L, SAMHD1, SERPINH1, SLFN11, SLFN13, SLFN5, SP110, SP140, SPN, SPR, STAP1, STAT1, STAT2, TAP1, TAX1BP3, THEMIS2, THTPA, TNFAIP8L2, TNFSF8, TP53I3, TREX1, TRIM22, TTC39C, TXNIP, UBA7, UBE2L6, USP41, VCL, VNN2 and ZBTB38.

In yet other embodiments of the various methods provided herein, the biomarker is selected from a group consisting of ARHGAP19, ASNS, ASPM, B4GALT3, BANK1, BCDIN3D, BLZF1, CA2, CA8, CAMSAP3, CCDC69, CCNB1, CDC7, CDCA3, CENPF, CSNK1A1DHPS, DLGAP5, DOK3, ECT2, EFCAB4B, EHMT1, EHMT2, EPCAM, ESRP1, FAM195A, FBRSL1, FHOD1, FIGNL1, GPT2, GRAMD1A, GRAMD1B, GRPEL2, HJURP, HMCES, HMMR, HOXC4, ICAM2, IKZF1, IKZF3, IRS2, KIF18B, KIF22, KIF2C, LIPG, LPXN, MINA, MIS18BP1, NEIL1, NFKBID, NPIPB5, OMA1, ORC6, PARVB, PBK, PDE6D, PKMYT1, PLK1, PODXL, PODXL2, POLE2, PRDM15, PRNP, PTAFR, PTTG1, PYROXD1, RASA4B, RASSF6, RGS1, RGS2, SEC14L1, SGOL1, SGOL2, SLCO3A1, SLCO4A1, TACC3, TIMM8B, TOP2A, TPX2, TRIB3, WIZ, WSB1, WWC1, ZFP91, ZMYM2, ZNF385B, ZNF581 and ZNF644.

In yet other embodiments of the various methods provided herein, the biomarker is selected from a group consisting of SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTU1, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770. In certain embodiments, any combination of two or more of the above-identified biomarkers is also contemplated.

In yet other embodiments of the various methods provided herein, the biomarker is selected from a group consisting of MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDC38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLII, FNBP1L, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830. In certain embodiments, any combination of two or more of the above-identified biomarkers is also contemplated.

In yet other embodiments of the various methods provided herein, the biomarker is selected from a group consisting of ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Tap, Igf2r, IGFBP1, IGFBP3, IKBKE, JAG1, KIAA0100, KIAA1462, KIF20B, KIF22, KLC2, KRT9, LDLR, LPXN, MAFF, MELK, MET, MGAT4B, MGLL, MMP7, MST1R, MT2A, MVP, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2. In certain embodiments, any combination of two or more of the above-identified biomarkers is also contemplated.

In yet other embodiments of the various methods provided herein, the biomarker is selected from a group consisting of ACSL6, AHSG, ALB, ARPP19, ATP5EP2, ATP5I, ATP5J, AZGP1, BCKDK, BLOC1S3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1H1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBC1D12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2. In certain embodiments, any combination of two or more of the above-identified biomarkers is also contemplated.

In a specific embodiment of the various methods provided herein, the biomarker is ZFP91. In another embodiment of the various methods provided herein, the biomarker is CRBN. In yet another embodiment of the various methods provided herein, the biomarkers are both ZFP91 and CRBN. In another specific embodiment of the various methods provided herein, the biomarker is Nestin. In another specific embodiment of the various methods provided herein, the biomarker is KAT1/CCBL1. In another specific embodiment of the various methods provided herein, the biomarker is WIBG. In another specific embodiment of the various methods provided herein, the biomarker is MVP. In yet another specific embodiment of the various methods provided herein, the biomarker is PARP4. In yet another specific embodiment of the various methods provided herein, the biomarker is ZNF198.

In some embodiments of the various methods provided herein, the level of only one biomarker is determined. In other embodiments of the various methods provided herein, the levels of two, three, four, five or more biomarkers are determined.

In some embodiments of the various methods provided herein, the reference is prepared by using a control sample obtained from the subject prior to administration of the treatment compound to the subject; and wherein the control sample is from the same source as the sample. In other embodiments of the various methods provided herein, the reference is prepared by using a control sample obtained from a healthy subject not having the solid tumor; and wherein the control sample is from the same source as the sample.

In some embodiments of the various methods provided herein, the levels of one or more of the biomarkers are measured by determining the mRNA levels of the biomarkers.

In other embodiments of the various methods provided herein, the levels of one or more of the biomarkers are measured by determining the cDNA levels of the biomarkers.

In yet other embodiments of the various methods provided herein, the levels of one or more of the biomarkers are measured by determining the protein levels of the biomarkers.

In some embodiments of the various methods provided herein, the method provided herein further comprises contacting proteins within the sample with a first antibody that immunospecifically binds to the biomarker protein.

In one embodiment, the method provided herein further comprises (i) contacting the proteins bound to the first antibody with a second antibody with a detectable label, wherein the second antibody immunospecifically binds to the biomarker protein, and wherein the second antibody immunospecifically binds to a different epitope on the biomarker protein than the first antibody; (ii) detecting the presence of second antibody bound to the biomarker protein; and (iii) determining the amount of the biomarker protein based on the amount of detectable label in the second antibody.

In another embodiment, the method provided herein further comprises (i) contacting the proteins bound to the first antibody with a second antibody with a detectable label, wherein the second antibody immunospecifically binds to the first antibody; (ii) detecting the presence of second antibody bound to the first antibody; and (iii) determining the amount of the biomarker protein based on the amount of detectable label in the second antibody.

In some embodiments of the various methods provided herein, the treatment compound is thalidomide, lenalidomide, pomalidomide, Compound A or Compound B, or a stereoisomer thereof, or a pharmaceutically acceptable salt, solvate, hydrate, co-crystal, clathrate, or a polymorph thereof. In one embodiment, the treatment compound is thalidomide, or a stereoisomer thereof, or a pharmaceutically acceptable salt, solvate, hydrate, co-crystal, clathrate, or a polymorph thereof. In another embodiment, the treatment compound is lenalidomide, or a stereoisomer thereof, or a pharmaceutically acceptable salt, solvate, hydrate, co-crystal, clathrate, or a polymorph thereof. In yet another embodiment, the treatment compound is pomalidomide, or a stereoisomer thereof, or a pharmaceutically acceptable salt, solvate, hydrate, co-crystal, clathrate, or a polymorph thereof. In yet another embodiment, the treatment compound is Compound A, or a stereoisomer thereof, or a pharmaceutically acceptable salt, solvate, hydrate, co-crystal, clathrate, or a polymorph thereof. In yet another embodiment, the treatment compound is Compound B, or a stereoisomer thereof, or a pharmaceutically acceptable salt, solvate, hydrate, co-crystal, clathrate, or a polymorph thereof.

Also provided herein is an array of probes for determining the levels of two or more biomarkers in a sample by hybridizing with one or more of the polynucleotides of the biomarkers under stringent condition, wherein the biomarkers are each independently selected from biomarkers identified in Tables 1 to 6, wherein the levels of the biomarkers are used to select a subject for treatment of a solid tumor with a treatment compound; predict or monitor the responsiveness of a subject to the treatment; or monitor the compliance of a subject with the treatment.

Provided herein is an array of probes for determining the levels of two or more biomarkers in a sample by hybridizing with one or more of mRNAs of the biomarkers under stringent condition, wherein the biomarkers are each independently selected from biomarkers identified in Tables 1 to 6, wherein the levels of the biomarkers are used to select a subject for treatment of a solid tumor with a treatment compound; predict or monitor the responsiveness of a subject to the treatment; or monitor the compliance of a subject with the treatment.

Provided herein is an array of antibodies for determining the levels of two or more biomarkers in a sample, wherein the biomarkers are each independently selected from biomarkers identified in Tables 1 to 6, wherein the levels of the biomarkers are used to select a subject for treatment of a solid tumor with a treatment compound; predict or monitor the responsiveness of a subject to the treatment; or monitor the compliance of a subject with the treatment.

Provided herein is a panel of isolated biomarkers comprising two or more biomarkers, each of which is independently selected from Nestin, KAT1/CCBL1, WIBG, MVP, PARP4, ZFP91, and ZFP198.

Provided herein is a panel of isolated biomarkers comprising two or more biomarkers, each of which is independently selected from AHNAK, ALOX5, AMPD3, ANXA4, ANXA6, ARHGAP19, ASNS, ASPM, ATP2B4, B4GALT3, BANK1, BCDIN3D, BLZF1, BMF, BST2, C10orf76, C19orf66, CA2, CA8, CAMSAP3, CCDC69, CCNB1, CD36, CDC7, CDCA3, CENPF, CLN3, CNN3, CORO1B, CPNE2, CRBN, CSNK1A1, CSRP2, CTNND1, CTSH, DAPK2, DDX58, DHPS, DHX58, DLG2, DLGAP5, DOK3, DTX3L, ECT2, EFCAB4B, EHMT1, EHMT2, EIF2AK2, EPB41L1, EPCAM, ESRP1, ETV6, EXTL2, F13A1, FAM195A, FAM65B, FBRSL1, FCGR2B, FES, FHOD1, FIGNL1, FMNL3, GBP1, GMFG, GMPR, GPT2, GRAMD1A, GRAMD1B, GRPEL2, HIP1, HJURP, HLA-B, HLA-DMA, HMCES, HMMR, HOXC4, HPSE, ICAM2, ID3, IFI35, IFIH1, IFIT1, IFIT3, IFIT5, IFITM2, IKZF1, IKZF3, IL4I1, IRF7, IRF9, IRS2, ISG15, ISG20, ITGB7, JAK3, KIF18B, KIF22, KIF2C, LAP3, LGALS1, LGALS3BP, LIMD1, LIPG, LPXN, MAN2A2, MARCKS, MFI2, MGARP, MINA, MIS18BP1, MOV10, MPP7, MUC1, MX1, MX2, MYO1G, NCF2, NEIL1, NFKBID, NME3, NMI, NPIPB5, NT5C3A, OAS1, OAS2, OAS3, OMA1, ORC6, PARP14, PARP9, PARVB, PBK, PBXIP1, PDE6D, PKMYT1, PLD4, PLEKHO1, PLK1, PLSCR1, PLXNB2, PODXL, PODXL2, POLE2, POMP, PPFIBP1, PRDM15, PRNP, PTAFR, PTMS, PTTG1, PYROXD1, QPRT, RAB13, RASA4B, RASSF6, RCN1, RGCC, RGS1, RGS2, RNF213, S100A13, SAMD9L, SAMHD1, SEC14L1, SERPINH1, SGOL1, SGOL2, SLCO3A1, SLCO4A1, SLFN11, SLFN13, SLFN5, SP110, SP140, SPN, SPR, STAP1, STAT1, STAT2, TACC3, TAP1, TAX1BP3, THEMIS2, THTPA, TIMM8B, TNFAIP8L2, TNFSF8, TOP2A, TP5313, TPX2, TREX1, TRIB3, TRIM22, TTC39C, TXNIP, UBA7, UBE2L6, USP41, VCL, VNN2, WIZ, WSB1, WWC1, ZBTB38, ZFP91, ZMYM2, ZNF385B, ZNF581 and ZNF644.

Provided herein is a panel of isolated biomarkers comprising two or more biomarkers, each of which is independently selected from SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTU1, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770.

Provided herein is a panel of isolated biomarkers comprising two or more biomarkers, each of which is independently selected from MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDC38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLII, FNBP1L, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830.

Provided herein is a panel of isolated biomarkers comprising two or more biomarkers, each of which is independently selected from ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Tap, Igf2r, IGFBP1, IGFBP3, IKBKE, JAG1, KIAA0100, KIAA1462, KIF20B, KIF22, KLC2, KRT9, LDLR, LPXN, MAFF, MELK, MET, MGAT4B, MGLL, MMP7, MST1R, MT2A, MW, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2. In certain embodiments, any combination of two or more of the above-identified biomarkers is also contemplated.

Provided herein is a panel of isolated biomarkers comprising two or more biomarkers, each of which is independently selected from ACSL6, AHSG, ALB, ARPP19, ATP5EP2, ATP5I, ATP5J, AZGP1, BCKDK, BLOC1S3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1H1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBC1D12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2.

In some embodiments, the solid tumors are sarcomas, carcinomas (epithelial tumors), melanomas, and glioblastomas. Exemplary solid tumors include, but not limited to, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma, glioblastoma (GBM); medulloblastoma), cervical cancer (e.g., cervical adenocarcinoma), colorectal cancer (e.g., colon cancer, rectal cancer, colorectal adenocarcinoma), gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), pancreatic cancer (e.g., pancreatic andenocarcinoma, intraductal papillary mucinous neoplasm (IPMN)), prostate cancer (e.g., prostate adenocarcinoma), skin cancer (e.g., squamous cell carcinoma (SCC), keratoacanthoma (A), melanoma, basal cell carcinoma (BCC)) and soft tissue sarcoma (e.g., malignant fibrous histiocytoma (MFH), liposarcoma, malignant peripheral nerve sheath tumor (MPNST), chondrosarcoma, fibrosarcoma, myxosarcoma, osteosarcoma). In some embodiments, the solid tumor is a liver cancer. In one embodiment, the solid tumor is HCC. In other embodiments, the solid tumor is a brain cancer. In one embodiment, the solid tumor is GBM.

Provided herein is a kit for determining the level of a biomarker in a biological sample from a subject, wherein the biomarker is selected from the group consisting of biomarkers identified in Tables 1 to 6, and combinations thereof.

### 4. DETAILED DESCRIPTION

The methods, arrays, probes, and kits provided herein are based, in part, on the discovery that a changed level, e.g., an increased level and/or a decreased level, of certain molecules (e.g., mRNAs, cDNA, or proteins) in a biological sample can be utilized as biomarkers to predict responsiveness of a subject having or suspected to have a solid tumor to a treatment compound, e.g., thalidomide, lenalidomide, pomalidomide, Compound A, or Compound B, or a stereoisomer thereof, or a pharmaceutically acceptable salt, solvate, hydrate, co-crystal, clathrate, or a polymorph thereof.

In certain embodiments, the methods provided herein are based on comparison of the level of one or more biomarkers in a biological sample from a subject having or suspected to have a solid tumor to a reference level of the biomarkers or the level of a control. The biomarker level is used to determine or to predict, for example, the likelihood of the subject's responsiveness to a treatment compound, such as thalidomide, lenalidomide, pomalidomide, Compound A, or Compound B, or a stereoisomer thereof, or a pharmaceutically acceptable salt, solvate, hydrate, co-crystal, clathrate, or a polymorph thereof.

### 4.1 BRIEF DESCRIPTION OF FIGURES

FIGS. 1A-C show the relative protein abundance in the proteomics study in two cell lines, Focus and HAK-1AJ. FIG. 1A shows the relative protein abundance in the proteomics study using Focus cells. FIG. 1B shows the relative protein abundance in the proteomics study using HAK-1AJ. FIG. 1C shows proteins commonly differentially abundant at 24 hours after treatment with Compound A in both Focus and HAK-1AJ cell lines.

FIGS. 2A-B show the proteomics study results using a HCC cell line (Focus). FIG. 2A shows the level of CRBN and ZFP91 in control plate and proteomic preparation (established) in 3D Focus cells treated with Compound A for various times. FIG. 2B shows the colony number and total colony area of control plates.

FIGS. 3A-B show the proteomics study results using a HCC cell line (JHH4). FIG. 3A shows the level of CRBN and ZFP91 in control plate and proteomic preparation (established) in 3D JHH4 cells treated with Compound A for various times. FIG. 3B shows the colony number and total colony area of control plates.

FIGS. 4A-D show western blot analysis results using HCC cell lines (Focus and JHH4). FIG. 4A shows the level of ZFP91 reduces in response to treatment with Compound A or Compound B in Focus cells. FIG. 4B shows the level of ZFP91 reduces in response to treatment with Compound A in Focus and JHH4 cell lines, and the level of CRBN increases in response to treatment with Compound A in Focus and JHH4 cell lines. FIG. 4C shows ZFP91 degradation induced by various doses of Compound A for various time periods in Focus and JHH4 cells. FIG. 4D shows confirmation of ZFP91 being degraded in response to Compound A treatment using siRNAs.

FIGS. 5A-D show results of compound treatment on ZFP91. FIG. 5A shows MG132 and MLN block the reduction of the level of ZFP91 in response to Compound A treatment in HCC cell lines using western blot analysis. FIG. 5B shows Compound A accelerates ZFP91 degradation through proteasome-mediated degradation. FIGS. 5C-D show knocking down of CRBN reverses the effect of the treatment compounds on ZFP91 protein level.

FIGS. 6A-B show results of compound treatment in Hep3B cells. FIG. 6A shows that ZFP91 is down-regulated in response to treatment compounds in a CRBN dependent pathway in Hep3B cell lines. FIG. 6B shows that Hep3B cells are sensitive to Compound B, but not to Compound A.

FIGS. 7A-B show the results of Compound A treatment on ZFP91. FIG. 7A shows that ZFP91 is down-regulated in response to treatment with Compound A or lenalidomide in a CRBN dependent pathway in U87 and SNB19 cells. FIG. 7B shows Compound A inhibits U87 tumor growth, and the result of immunostaining assay demonstrating that Compound A degrades ZFP91 in U87 xenograft tissue.

FIG. 8 shows that the levels of MST1R (RON), PARP4, MVP, ZFP91, IKBKE, CK1-alpha, PDE6D, LGALS2, KAT1, CRBN, and GSPT1 change in response to Compound A treatment in a western blot analysis.

### 4.2 DEFINITIONS

The term “treat,” “treating,” or “treatment” refers to alleviating or abrogating a disease, e.g., HCC or GBM, or one or more of the symptoms associated with the disease; or alleviating or eradicating the cause(s) of the disease itself.

As used herein “solid tumor” refers to an abnormal mass of tissue. Solid tumors may be benign or malignant. A solid tumor grows in an anatomical site outside the bloodstream (in contrast, for example, to cancers of hematopoietic origin such as leukemias) and requires the formation of small blood vessels and capillaries to supply nutrients, etc. to the growing tumor mass. Solid tumors are named for the type of cells that form them. Non-limiting examples of solid tumors are sarcomas, carcinomas (epithelial tumors), melanomas, and glioblastomas. Exemplary solid tumors include, but are not limited to, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma, glioblastomas; medulloblastoma), cervical cancer (e.g., cervical adenocarcinoma), colorectal cancer (e.g., colon cancer, rectal cancer, colorectal adenocarcinoma), gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), pancreatic cancer (e.g., pancreatic andenocarcinoma, intraductal papillary mucinous neoplasm (IPMN)), prostate cancer (e.g., prostate adenocarcinoma), skin cancer (e.g., squamous cell carcinoma (SCC), keratoacanthoma (A), melanoma, basal cell carcinoma (BCC)) and soft tissue sarcoma (e.g., malignant fibrous histiocytoma (MFH), liposarcoma, malignant peripheral nerve sheath tumor (MPNST), chondrosarcoma, fibrosarcoma, myxosarcoma, osteosarcoma).

The term “therapeutically effective amount” of a compound refers to the amount of a compound that, when administered, is sufficient to prevent development of, or alleviate to some extent, one or more of the symptoms of a disease, e.g., HCC or GBM, being treated. The term also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician. Furthermore, a therapeutically effective amount of a compound means an amount of a therapeutic agent, alone or in combination with other therapies, which provides a therapeutic benefit in the treatment or management of a disease, e.g., HCC or GBM. The term encompasses an amount that improves overall therapy, reduces, or avoids symptoms or causes of a disease, e.g., HCC or GBM, or enhances the therapeutic efficacy of another therapeutic agent.

The term “level” refers to the amount, accumulation, or rate of a biomarker molecule. A level can be represented, for example, by the amount or the rate of synthesis of a massager RNA (mRNA) encoded by a gene, the amount or the rate of synthesis of a polypeptide or protein encoded by a gene, or the amount or the rate of synthesis of a biological molecule accumulated in a cell or biological fluid. The term “level” refers to an absolute amount of a molecule in a sample or to a relative amount of the molecule, determined under steady-state or non-steady-state conditions.

The term “responsiveness” or “responsive” when used in reference to a treatment refer to the degree of effectiveness of the treatment in lessening or decreasing the symptoms of a disease, e.g., HCC or GBM, being treated. For example, the term “increased responsiveness” when used in reference to a treatment of a cell or a subject refers to an increase in the effectiveness in lessening or decreasing the symptoms of the disease when measured using any methods known in the art. In certain embodiments, the increase in the effectiveness is at least about 5%, at least about 10%, at least about 20%, at least about 30%, at least about 40%, or at least about 50%.

As used herein, the terms “effective subject response,” “effective patient response,” or “effective patient tumor response” refers to any increase in the therapeutic benefit to the patient. An “effective patient tumor response” can be, for example, a 5%, 10%, 15%, 20%, 25%, 30%, 40%, 50%, 60%, 70%, 80%, 90%, or 100% decrease in the rate of progress of the tumor. An “effective patient tumor response” can be, for example, a 5%, 10%, 15%, 20%, 25%, 30%, 40%, 50%, 60%, 70%, 80%, 90%, or 100% decrease in the physical symptoms of a cancer. An “effective patient tumor response” can be, for example, a 5%, 10%, 15%, 20%, 25%, 30%, 40%, 50%, 60%, 70%, 80%, 90%, or 100% decrease in the size of a tumor. An “effective patient tumor response” can be, for example, a 5%, 10%, 15%, 20%, 25%, 30%, 40%, 50%, 60%, 70%, 80%, 90%, or 100% decrease in the physical symptoms of a cancer or the tumor size. An “effective patient tumor response” can also be, for example, a 5%, 10%, 15%, 20%, 25%, 30%, 40%, 50%, 60%, 70%, 80%, 90%, 100%, 200%, or more increase in the response of the patient, as measured by any suitable means, such as gene expression, cell counts, assay results, etc.

The term “likelihood” refers to an increase in the probability of an event. The term “likelihood” when used in reference to the effectiveness of a subject response to a treatment of a disease, e.g., HCC or GBM, contemplates an increased probability that the symptoms of the disease will be lessened or decreased.

The term “predict” generally means to determine or tell in advance. When used to “predict” the effectiveness of the treatment of a disease (e.g., HCC or GBM), for example, the term “predict” can mean that the likelihood of the outcome of the treatment can be determined at the outset, before the treatment has begun, or before the treatment period has progressed substantially.

The term “polypeptide,” “protein,” or “peptide,” as used herein interchangeably, refers to a polymer of two or more amino acids in a serial array, linked through one or more peptide bond(s). The term encompasses proteins, protein fragments, protein analogues, oligopeptides, and peptides. The amino acids of the polypeptide, protein, or peptide can be naturally occurring amino acids or synthetic amino acids (e.g., mimics of naturally occurring amino acids). The polypeptide, protein, or peptide can be made synthetically or purified from a biological sample. The polypeptide, protein, or peptide also encompasses modified polypeptides, proteins, and peptides, e.g., a glycopolypeptide, glycoprotein, or glycopeptide; or a lipopolypeptide, lipoprotein, or lipopeptide.

The term “antibody” refers to a polypeptide that specifically binds an epitope (e.g., an antigen). The term “antibody” is used herein in the broadest sense and covers fully assembled antibodies, antibody fragments which retain the ability to specifically bind to an antigen (e.g., Fab, F(ab′)₂, Fv, and other fragments), single chain antibodies, diabodies, antibody chimeras, hybrid antibodies, bispecific antibodies, and humanized antibodies. The term “antibody” also covers both polyclonal and monoclonal antibodies.

The term “expressed” or “expression” refers to the transcription from a gene to give an RNA nucleic acid molecule, e.g., mRNA, at least complementary in part to a region of one of the two nucleic acid strands of the gene. The term “expressed” or “expression” as used herein also refers to the translation from an RNA molecule to give a protein, a polypeptide or a portion thereof.

A biological marker or “biomarker” is a substance whose detection indicates a particular biological state, such as, for example, the presence of cancer. In some embodiments, biomarkers can either be determined individually, or several biomarkers can be measured simultaneously.

A “biomarker” can indicate a change in the level of mRNA expression that may correlate with the risk or progression of a disease, or with the susceptibility of the disease to a given treatment. The biomarker is a nucleic acid, such as a mRNA or cDNA.

A “biomarker” can indicate a change in the level of polypeptide or protein expression that may correlate with the risk, susceptibility to treatment, or progression of a disease. In some embodiments, the biomarker can be a polypeptide or protein, or a fragment thereof. The relative level of specific proteins can be determined by methods known in the art. For example, antibody based methods, such as an immunoblot, enzyme-linked immunosorbent assay (ELISA), or other methods can be used.

An mRNA that is “upregulated” is generally increased upon a given treatment or condition. An mRNA that is “downregulated” generally refers to a decrease in the level of expression of the mRNA in response to a given treatment or condition. In some situations, the mRNA level can remain unchanged upon a given treatment or condition. An mRNA from a patient sample can be “upregulated” when treated with a drug, as compared to a non-treated control. This upregulation can be, for example, an increase of about 5%, 10%, 20%, 30%, 40%, 50%, 60%, 70%, 90%, 100%, 200%, 300%, 500%, 600%, 700%, 800%, 900%, 1,000%, 1,500%, 2,000%, 2,500%, 3,00%, 3,500%, 4,000%, 4,500%, 5,000% or more of the comparative control mRNA level. Alternatively, an mRNA can be “downregulated”, or expressed at a lower level, in response to administration of certain compounds or other agents. A downregulated mRNA can be, for example, present at a level of about 99%, 95%, 90%, 85%, 80%, 75%, 70%, 65%, 60%, 55%, 50%, 45%, 40%, 35%, 30%, 25%, 20%, 15%, 10%, 5%, 3%, 1% or less of the comparative control mRNA level.

Similarly, the level of a polypeptide or protein biomarker from a patient sample can be increased when treated with a drug, as compared to a non-treated control. This increase can be about 5%, 10%, 20%, 30%, 40%, 50%, 60%, 70%, 80%, 90%, 100%, 200%, 300%, 400%, 500%, 700%, 1,000%, 1,500%, 2,000%, 2,500%, 3,000%, 3,500%, 4,000%, 4,500%, 5,000% or more of the comparative control protein level. Alternatively, the level of a protein biomarker can be decreased in response to administration of certain compounds or other agents. This decrease can be, for example, present at a level of about 99%, 95%, 90%, 80%, 70%, 60%, 50%, 40%, 30%, 20%, 10%, 5%, 3%, 1% or less of the comparative control protein level.

The terms “determining”, “measuring”, “evaluating”, “assessing” and “assaying” are used interchangeably herein to refer to a form of measurement, including determining if an element is present or not. The measurement can be quantitative and/or qualitative determinations. “Assessing the presence of” can include determining the amount of something present, as well as determining whether it is present or absent.

The terms “nucleic acid” and “polynucleotide” are used interchangeably herein to refer to a polymer of nucleotides, e.g., deoxyribonucleotides or ribonucleotides, or compounds, which can hybridize with a naturally occurring nucleic acid in a sequence specific manner analogous to that of two naturally occurring nucleic acids, e.g., participating in Watson-Crick base pairing interactions. As used herein in the context of a polynucleotide sequence, the term “bases” (or “base”) is synonymous with “nucleotides” (or “nucleotide”), i.e., the monomer subunit of a polynucleotide. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. The term “analogue” of a “nucleic acid” or “polynucleotide” refers to a molecule having a structural feature that is recognized in the literature as being a mimetic, derivative, having an analogous structure, or other like terms, and includes, for example, a polynucleotide incorporating a non-natural nucleotide, a nucleotide mimetic such as a 2′-modified nucleoside, peptide nucleic acid, oligomeric nucleoside phosphonate, and any polynucleotide that has added substituent groups, such as protecting groups or linking moieties.

The term “complementary” refers to specific binding between polynucleotides based on the sequences of the polynucleotides. As used herein, a first polynucleotide and a second polynucleotide are complementary if they bind to each other in a hybridization assay under stringent conditions, e.g., if they produce a given or detectable level of signal in a hybridization assay. Portions of polynucleotides are complementary to each other if they follow conventional base-pairing rules, e.g., A pairs with T (or U) and G pairs with C, although small regions (e.g. less than about 3 bases) of mismatch, insertion, or deleted sequence may be present.

The term “sequence identity” or “identity” in the context of two nucleic acid sequences refers to the residues in the two sequences which are the same when aligned for maximum correspondence over a specified comparison window, and can take into consideration additions, deletions and substitutions.

The term “substantial identity” or “homologous” in their various grammatical forms in the context of polynucleotides generally means that a polynucleotide comprises a sequence that has a desired identity, for example, at least 60%, at least 70%, at least 80%, at least 90% and at least 95% sequence identity, compared to a reference sequence. Another indication that nucleotide sequences are substantially identical is if two molecules hybridize to each other under stringent conditions.

As used herein, the term “bound” can be used herein to indicate direct or indirect attachment. In the context of chemical structures, “bound” (or “bonded”) may refer to the existence of a chemical bond directly joining two moieties or indirectly joining two moieties (e.g., via a linking group or any other intervening portion of the molecule). The chemical bond may be a covalent bond, an ionic bond, a coordination complex, hydrogen bonding, van der Waals interactions, or hydrophobic stacking, or may exhibit characteristics of multiple types of chemical bonds. In certain instances, “bound” includes embodiments where the attachment is direct and also embodiments where the attachment is indirect.

The terms “isolated” and “purified” refer to isolation of a substance (such as mRNA or protein) such that the substance comprises a substantial portion of the sample in which it resides, i.e., greater than the substance is typically found in its natural or un-isolated state. Typically, a substantial portion of the sample comprises, e.g., greater than about 1%, greater than about 2%, greater than about 5%, greater than about 10%, greater than about 20%, greater than about 50%, or more, usually up to about 90% to 100% of the sample. For example, a sample of isolated mRNA can typically comprise at least about 1% total mRNA. Techniques for purifying polynucleotides are well known in the art and include, for example, gel electrophoresis, ion-exchange chromatography, affinity chromatography, flow sorting, and sedimentation according to density.

The term “biological sample” as used herein refers to a sample obtained from a biological subject, including a sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, fractions and cells isolated from a mammal. Exemplary biological samples include, but are not limited to, cell lysate, a cell culture, a cell line, a tissue, oral tissue, gastrointestinal tissue, an organ, an organelle, a biological fluid, a blood sample, a urine sample, a skin sample, and the like. In certain embodiments, biological samples include, but are not limited to, whole blood, partially purified blood, PBMCs, tissue biopsies, and the like.

The term “analyte” as used herein, refers to a known or unknown component of a sample.

The term “capture agent,” as used herein, refers to an agent that binds an mRNA or protein through an interaction that is sufficient to permit the agent to bind and concentrate the mRNA or protein from a homogeneous mixture.

The term “probe” as used herein, refers to a capture agent that is directed to a specific target mRNA biomarker sequence. Accordingly, each probe of a probe set has a respective target mRNA biomarker. A probe/target mRNA duplex is a structure formed by hybridizing a probe to its target mRNA biomarker.

The term “nucleic acid probe” or “oligonucleotide probe” refers to a nucleic acid capable of binding to a target nucleic acid of complementary sequence, such as the mRNA biomarkers provided herein, through one or more types of chemical bonds, usually through complementary base pairing, usually through hydrogen bond formation. As used herein, a probe may include natural (e.g., A, G, C, or T) or modified bases (7-deazaguanosine, inosine, etc.). In addition, the bases in a probe may be joined by a linkage other than a phosphodiester bond, so long as it does not interfere with hybridization. It will be understood by one of skill in the art that probes may bind target sequences lacking complete complementarity with the probe sequence depending upon the stringency of the hybridization conditions. The probes are preferably directly labeled with isotopes, for example, chromophores, lumiphores, chromogens, or indirectly labeled with biotin to which a streptavidin complex may later bind. By assaying for the presence or absence of the probe, one can detect the presence or absence of a target mRNA biomarker of interest.

The term “stringent assay conditions” refers to conditions that are compatible to produce binding pairs of nucleic acids, e.g., probes and target mRNAs, of sufficient complementarity to provide for the desired level of specificity in the assay while being generally incompatible to the formation of binding pairs between binding members of insufficient complementarity to provide for the desired specificity. The term “stringent assay conditions” generally refers to the combination of hybridization and wash conditions.

A “label” or a “detectable moiety” in reference to a nucleic acid, refers to a composition that, when linked with a nucleic acid, renders the nucleic acid detectable, for example, by spectroscopic, photochemical, biochemical, immunochemical, or chemical means. Exemplary labels include, but are not limited to, radioactive isotopes, magnetic beads, metallic beads, colloidal particles, fluorescent dyes, enzymes, biotin, digoxigenin, haptens, and the like. A “labeled nucleic acid or oligonucleotide probe” is generally one that is bound, either covalently, through a linker or a chemical bond, or noncovalently, through ionic bonds, van der Waals forces, electrostatic attractions, hydrophobic interactions, or hydrogen bonds, to a label such that the presence of the nucleic acid or probe can be detected by detecting the presence of the label bound to the nucleic acid or probe.

The terms “polymerase chain reaction” or “PCR,” as used herein generally refers to a procedure wherein small amounts of a nucleic acid, RNA and/or DNA, are amplified as described, for example, in U.S. Pat. No. 4,683,195 to Mullis. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol., 51: 263 (1987); Erlich, ed., PCR Technology, (Stockton Press, N Y, 1989).

The term “cycle number” or “CT” when used herein in reference to PCR methods, refers to the PCR cycle number at which the fluorescence level passes a given set threshold level. The CT measurement can be used, for example, to approximate levels of mRNA in an original sample. The CT measurement is often used in terms of “dCT” or the “difference in the CT” score, when the CT of one nucleic acid is subtracted from the CT of another nucleic acid.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The practice of the embodiments provided herein will employ, unless otherwise indicated, conventional techniques of molecular biology, microbiology, and immunology, which are within the skill of those working in the art. Such techniques are explained fully in the literature. Examples of particularly suitable texts for consultation include the following: Sambrook et al., Molecular Cloning; A Laboratory Manual (2d ed.), 1989; Glover, ed. DNA Cloning, Volumes I and II, 1985; Gait, ed., Oligonucleotide Synthesis, 1984; Hames & Higgins, eds. Nucleic Acid Hybridization, 1984; Hames &. Higgins, eds., Transcription and Translation, 1984; Freshney, ed., Animal Cell Culture, 1986; Immobilized Cells and Enzymes, IRL Press, 1986; Immunochemical Methods in Cell and Molecular Biology (Academic Press, London); Scopes, Protein Purification: Principles and Practice (2d ed.; Springer Verlag, N.Y.), 1987; and Weir and Blackwell, eds. Handbook of Experimental Immunology, Volumes I-IV, 1986.

### 4.3 BIOMARKERS

A biological marker or “biomarker” is a substance, the change and/or the detection of which indicates a particular biological state, such as, for example, the responsiveness of a disease, e.g., HCC or GBM, to a given treatment, e.g., a treatment by a treatment compound, e.g., thalidomide, lenalidomide, pomalidomide, Compound A, or Compound B, or a stereoisomer thereof, or a pharmaceutically acceptable salt, solvate, hydrate, co-crystal, clathrate, or a polymorph thereof.

The present application is based, in part, on the finding that the levels of certain proteins change in response to treatment compound, e.g., as shown in the proteomics study in the Examples. For example, as shown, certain proteins upregulated in response to Compound A treatment include Nestin, KAT1/CCBL1, WIBG, CK1-alpha, GSPT1, and certain proteins downregulated in response to Compound A treatment include MVP, PARP4, ZFP91, and ZNF198. Thus, in some embodiments, the biomarker provided herein is selected from a group consisting of Nestin, KAT1/CCBL1, WIBG, MVP, PARP4, ZFP91, ZNF198, GSPT1, and CK1-alpha. In some embodiments of the various methods provided herein, the biomarker is selected from a group consisting of Nestin, KAT1/CCBL1, WIBG, CK1-alpha, and GSPT1. In other embodiments, the biomarker is selected from a group consisting of MVP, PARP4, ZFP91, and ZNF198.

Other biomarkers provided herein include proteins selected from AHNAK, ALOX5, AMPD3, ANXA4, ANXA6, ARHGAP19, ASNS, ASPM, ATP2B4, B4GALT3, BANK1, BCDIN3D, BLZF1, BMF, BST2, C10orf76, C19orf66, CA2, CA8, CAMSAP3, CCDC69, CCNB1, CD36, CDC7, CDCA3, CENPF, CLN3, CNN3, CORO1B, CPNE2, CRBN, CSNK1A1, CSRP2, CTNND1, CTSH, DAPK2, DDX58, DHPS, DHX58, DLG2, DLGAP5, DOK3, DTX3L, ECT2, EFCAB4B, EHMT1, EHMT2, EIF2AK2, EPB41L1, EPCAM, ESRP1, ETV6, EXTL2, F13A1, FAM195A, FAM65B, FBRSL1, FCGR2B, FES, FHOD1, FIGNL1, FMNL3, GBP1, GMFG, GMPR, GPT2, GRAMD1A, GRAMD1B, GRPEL2, HIP1, HJURP, HLA-B, HLA-DMA, HMCES, HMMR, HOXC4, HPSE, ICAM2, ID3, IFI35, IFIH1, IFIT1, IFIT3, IFIT5, IFITM2, IKZF1, IKZF3, IL4I1, IRF7, IRF9, IRS2, ISG15, ISG20, ITGB7, JAK3, KIF18B, KIF22, KIF2C, LAP3, LGALS1, LGALS3BP, LIMD1, LIPG, LPXN, MAN2A2, MARCKS, MFI2, MGARP, MINA, MIS18BP1, MOV10, MPP7, MUC1, MX1, MX2, MYO1G, NCF2, NEIL1, NFKBID, NME3, NMI, NPIPB5, NT5C3A, OAS1, OAS2, OAS3, OMA1, ORC6, PARP14, PARP9, PARVB, PBK, PBXIP1, PDE6D, PKMYT1, PLD4, PLEKHO1, PLK1, PLSCR1, PLXNB2, PODXL, PODXL2, POLE2, POMP, PPFIBP1, PRDM15, PRNP, PTAFR, PTMS, PTTG1, PYROXD1, QPRT, RAB13, RASA4B, RASSF6, RCN1, RGCC, RGS1, RGS2, RNF213, S100A13, SAMD9L, SAMHD1, SEC14L1, SERPINH1, SGOL1, SGOL2, SLCO3A1, SLCO4A1, SLFN11, SLFN13, SLFN5, SP110, SP140, SPN, SPR, STAP1, STAT1, STAT2, TACC3, TAP1, TAX1BP3, THEMIS2, THTPA, TIMM8B, TNFAIP8L2, TNFSF8, TOP2A, TP5313, TPX2, TREX1, TRIB3, TRIM22, TTC39C, TXNIP, UBA7, UBE2L6, USP41, VCL, VNN2, WIZ, WSB1, WWC1, ZBTB38, ZFP91, ZMYM2, ZNF385B, ZNF581 or ZNF644, or a combination thereof.

In some embodiments, the biomarker is a protein selected from AHNAK, ALOX5, AMPD3, ANXA4, ANXA6, ATP2B4, BMF, BST2, C10orf76, C19orf66, CD36, CLN3, CNN3, CORO1B, CPNE2, CRBN, CSRP2, CTNND1, CTSH, DAPK2, DDX58, DHX58, DLG2, DTX3L, EIF2AK2, EPB41L1, ETV6, EXTL2, F13A1, FAM65B, FCGR2B, FES, FMNL3, GBP1, GMFG, GMPR, HIP1, HLA-B, HLA-DMA, HPSE, ID3, IF135, IFIH1, IFIT1, IFIT3, IFIT5, IFITM2, IL4I1, IRF7, IRF9, ISG15, ISG20, ITGB7, JAK3, LAP3, LGALS1, LGALS3BP, LIMD1, MAN2A2, MARCKS, MFI2, MGARP, MOV10, MPP7, MUC1, MX1, MX2, MYO1G, NCF2, NME3, NMI, NT5C3A, OAS1, OAS2, OAS3, PARP14, PARP9, PBXIP1, PLD4, PLEKHO1, PLSCR1, PLXNB2, POMP, PPFIBP1, PTMS, QPRT, RAB13, RCN1, RGCC, RNF213, S100A13, SAMD9L, SAMHD1, SERPINH1, SLFN11, SLFN13, SLFN5, SP110, SP140, SPN, SPR, STAP1, STAT1, STAT2, TAP1, TAX1BP3, THEMIS2, THTPA, TNFAIP8L2, TNFSF8, TP53I3, TREX1, TRIM22, TTC39C, TXNIP, UBA7, UBE2L6, USP41, VCL, VNN2 and ZBTB38, or a combination thereof.

In other embodiments, the biomarker is a protein selected from ARHGAP19, ASNS, ASPM, B4GALT3, BANK1, BCDIN3D, BLZF1, CA2, CA8, CAMSAP3, CCDC69, CCNB1, CDC7, CDCA3, CENPF, CSNK1A1DHPS, DLGAP5, DOK3, ECT2, EFCAB4B, EHMT1, EHMT2, EPCAM, ESRP1, FAM195A, FBRSL1, FHOD1, FIGNL1, GPT2, GRAMD1A, GRAMD1B, GRPEL2, HJURP, HMCES, HMMR, HOXC4, ICAM2, IKZF1, IKZF3, IRS2, KIF18B, K1F22, KIF2C, LIPG, LPXN, MINA, MIS18BP1, NEIL1, NFKBID, NPIPB5, OMA1, ORC6, PARVB, PBK, PDE6D, PKMYT1, PLK1, PODXL, PODXL2, POLE2, PRDM15, PRNP, PTAFR, PTTG1, PYROXD1, RASA4B, RASSF6, RGS1, RGS2, SEC14L1, SGOL1, SGOL2, SLCO3A1, SLCO4A1, TACC3, TIMM8B, TOP2A, TPX2, TRIB3, WIZ, WSB1, WWC1, ZFP91, ZMYM2, ZNF385B, ZNF581 and ZNF644, or a combination thereof.

In one embodiment, the biomarker is AHNAK. In one embodiment, the biomarker is ALOX5. In one embodiment, the biomarker is AMPD3. In another embodiment, the biomarker is ANXA4. In another embodiment, the biomarker is ANXA6. In another embodiment, the biomarker is ARHGAP19. In another embodiment, the biomarker is ASNS. In another embodiment, the biomarker is ASPM. In another embodiment, the biomarker is ATP2B4. In another embodiment, the biomarker is B4GALT3. In yet another embodiment, the biomarker is BANK1. In yet another embodiment, the biomarker is BCDIN3D. In yet another embodiment, the biomarker is BLZF1. In yet another embodiment, the biomarker is BMF. In yet another embodiment, the biomarker is BST2. In yet another embodiment, the biomarker is C10orf76. In yet another embodiment, the biomarker is C19orf66. In yet another embodiment, the biomarker is CA2. In yet another embodiment, the biomarker is CA8. In yet another embodiment, the biomarker is CAMSAP3. In yet another embodiment, the biomarker is CCDC69. In yet another embodiment, the biomarker is CCNB1. In yet another embodiment, the biomarker is CD36. In yet another embodiment, the biomarker is CDC7. In yet another embodiment, the biomarker is CDCA3. In yet another embodiment, the biomarker is CENPF. In yet another embodiment, the biomarker is CLN3. In yet another embodiment, the biomarker is CNN3. In yet another embodiment, the biomarker is CORO1B. In yet another embodiment, the biomarker is CPNE2. In yet another embodiment, the biomarker is CRBN. In yet another embodiment, the biomarker is CSNK1A1. In yet another embodiment, the biomarker is CSRP2. In yet another embodiment, the biomarker is CTNND1. In yet another embodiment, the biomarker is CTSH. In yet another embodiment, the biomarker is DAPK2. In yet another embodiment, the biomarker is DDX58. In yet another embodiment, the biomarker is DHPS. In yet another embodiment, the biomarker is DHX58. In yet another embodiment, the biomarker is DLG2. In yet another embodiment, the biomarker is DLGAP5. In yet another embodiment, the biomarker is DOK3. In yet another embodiment, the biomarker is DTX3L. In yet another embodiment, the biomarker is ECT2. In yet another embodiment, the biomarker is EFCAB4B. In yet another embodiment, the biomarker is EHMT1. In yet another embodiment, the biomarker is EHMT2. In yet another embodiment, the biomarker is EIF2AK2. In yet another embodiment, the biomarker is EPB41L1. In yet another embodiment, the biomarker is EPCAM. In yet another embodiment, the biomarker is ESRP1. In yet another embodiment, the biomarker is ETV6. In yet another embodiment, the biomarker is EXTL2. In yet another embodiment, the biomarker is F13A1. In yet another embodiment, the biomarker is FAM195A. In yet another embodiment, the biomarker is FAM65B. In yet another embodiment, the biomarker is FBRSL1. In yet another embodiment, the biomarker is FCGR2B. In yet another embodiment, the biomarker is FES. In yet another embodiment, the biomarker is FHOD1. In yet another embodiment, the biomarker is FIGNL1. In yet another embodiment, the biomarker is FMNL3. In yet another embodiment, the biomarker is GBP1. In yet another embodiment, the biomarker is GMFG. In yet another embodiment, the biomarker is GMPR. In yet another embodiment, the biomarker is GPT2. In yet another embodiment, the biomarker is GRAMD1A. In yet another embodiment, the biomarker is GRAMD1B. In yet another embodiment, the biomarker is GRPEL2. In yet another embodiment, the biomarker is HIP1. In yet another embodiment, the biomarker is HJURP. In yet another embodiment, the biomarker is HLA-B. In yet another embodiment, the biomarker is HLA-DMA. In yet another embodiment, the biomarker is HMCES. In yet another embodiment, the biomarker is HMMR. In yet another embodiment, the biomarker is HOXC4. In yet another embodiment, the biomarker is HPSE. In yet another embodiment, the biomarker is ICAM2. In yet another embodiment, the biomarker is ID3. In yet another embodiment, the biomarker is IFI35. In yet another embodiment, the biomarker is IFIH1. In yet another embodiment, the biomarker is IFIT1. In yet another embodiment, the biomarker is IFIT3. In yet another embodiment, the biomarker is IFIT5. In yet another embodiment, the biomarker is IFITM2. In yet another embodiment, the biomarker is IKZF1. In yet another embodiment, the biomarker is IKZF3. In yet another embodiment, the biomarker is IL4I1. In yet another embodiment, the biomarker is IRF7. In yet another embodiment, the biomarker is IRF9. In yet another embodiment, the biomarker is IRS2. In yet another embodiment, the biomarker is ISG15. In yet another embodiment, the biomarker is ISG20. In yet another embodiment, the biomarker is ITGB7. In yet another embodiment, the biomarker is JAK3. In yet another embodiment, the biomarker is KIF18B. In yet another embodiment, the biomarker is KIF22. In yet another embodiment, the biomarker is KIF2C. In yet another embodiment, the biomarker is LAP3. In yet another embodiment, the biomarker is LGALS1. In yet another embodiment, the biomarker is LGALS3BP. In yet another embodiment, the biomarker is LIMD1. In yet another embodiment, the biomarker is LIPG. In yet another embodiment, the biomarker is LPXN. In yet another embodiment, the biomarker is MAN2A2. In yet another embodiment, the biomarker is MARCKS. In yet another embodiment, the biomarker is MFI2. In yet another embodiment, the biomarker is MGARP. In yet another embodiment, the biomarker is MINA. In yet another embodiment, the biomarker is MIS18BP1. In yet another embodiment, the biomarker is MOV10. In yet another embodiment, the biomarker is MPP7. In yet another embodiment, the biomarker is MUC1. In yet another embodiment, the biomarker is MX1. In yet another embodiment, the biomarker is MX2. In yet another embodiment, the biomarker is MYO1G. In yet another embodiment, the biomarker is NCF2. In yet another embodiment, the biomarker is NEIL1. In yet another embodiment, the biomarker is NFKBID. In yet another embodiment, the biomarker is NME3. In yet another embodiment, the biomarker is NMI. In yet another embodiment, the biomarker is NPIPB5. In yet another embodiment, the biomarker is NT5C3A. In yet another embodiment, the biomarker is OAS1. In yet another embodiment, the biomarker is OAS2. In yet another embodiment, the biomarker is OAS3. In yet another embodiment, the biomarker is OMA1. In yet another embodiment, the biomarker is ORC6. In yet another embodiment, the biomarker is PARP14. In yet another embodiment, the biomarker is PARP9. In yet another embodiment, the biomarker is PARVB. In yet another embodiment, the biomarker is PBK. In yet another embodiment, the biomarker is PBXIP1. In yet another embodiment, the biomarker is PDE6D. In yet another embodiment, the biomarker is PKMYT1. In yet another embodiment, the biomarker is PLD4. In yet another embodiment, the biomarker is PLEKHO1. In yet another embodiment, the biomarker is PLK1. In yet another embodiment, the biomarker is PLSCR1. In yet another embodiment, the biomarker is PLXNB2. In yet another embodiment, the biomarker is PODXL. In yet another embodiment, the biomarker is PODXL2. In yet another embodiment, the biomarker is POLE2. In yet another embodiment, the biomarker is POMP. In yet another embodiment, the biomarker is PPFIBP1. In yet another embodiment, the biomarker is PRDM15. In yet another embodiment, the biomarker is PRNP. In yet another embodiment, the biomarker is PTAFR. In yet another embodiment, the biomarker is PTMS. In yet another embodiment, the biomarker is PTTG1. In yet another embodiment, the biomarker is PYROXD1. In yet another embodiment, the biomarker is QPRT. In yet another embodiment, the biomarker is RAB13. In yet another embodiment, the biomarker is RASA4B. In yet another embodiment, the biomarker is RASSF6. In yet another embodiment, the biomarker is RCN1. In yet another embodiment, the biomarker is RGCC. In yet another embodiment, the biomarker is RGS1. In yet another embodiment, the biomarker is RGS2. In yet another embodiment, the biomarker is RNF213. In yet another embodiment, the biomarker is S100A13. In yet another embodiment, the biomarker is SAMD9L. In yet another embodiment, the biomarker is SAMHD1. In yet another embodiment, the biomarker is SEC14L1. In yet another embodiment, the biomarker is SERPINH1. In yet another embodiment, the biomarker is SGOL1. In yet another embodiment, the biomarker is SGOL2. In yet another embodiment, the biomarker is SLCO3A1. In yet another embodiment, the biomarker is SLCO4A1. In yet another embodiment, the biomarker is SLFN11. In yet another embodiment, the biomarker is SLFN13. In yet another embodiment, the biomarker is SLFN5. In yet another embodiment, the biomarker is SP110. In yet another embodiment, the biomarker is SP140. In yet another embodiment, the biomarker is SPN. In yet another embodiment, the biomarker is SPR. In yet another embodiment, the biomarker is STAP1. In yet another embodiment, the biomarker is STAT1. In yet another embodiment, the biomarker is STAT2. In yet another embodiment, the biomarker is TACC3. In yet another embodiment, the biomarker is TAP1. In yet another embodiment, the biomarker is TAX1BP3. In yet another embodiment, the biomarker is THEMIS2. In yet another embodiment, the biomarker is THTPA. In yet another embodiment, the biomarker is TIMM8B. In yet another embodiment, the biomarker is TNFAIP8L2. In yet another embodiment, the biomarker is TNFSF8. In yet another embodiment, the biomarker is TOP2A. In yet another embodiment, the biomarker is TP53I3. In yet another embodiment, the biomarker is TPX2. In yet another embodiment, the biomarker is TREX1. In yet another embodiment, the biomarker is TRIB3. In yet another embodiment, the biomarker is TRIM22. In yet another embodiment, the biomarker is TTC39C. In yet another embodiment, the biomarker is TXNIP. In yet another embodiment, the biomarker is UBA7. In yet another embodiment, the biomarker is UBE2L6. In yet another embodiment, the biomarker is USP41. In yet another embodiment, the biomarker is VCL. In yet another embodiment, the biomarker is VNN2. In yet another embodiment, the biomarker is WIZ. In yet another embodiment, the biomarker is WSB1. In yet another embodiment, the biomarker is WWC1. In yet another embodiment, the biomarker is ZBTB38. In yet another embodiment, the biomarker is ZFP91. In yet another embodiment, the biomarker is ZMYM2. In yet another embodiment, the biomarker is ZNF385B. In yet another embodiment, the biomarker is ZNF581. In yet another embodiment, the biomarker is ZNF644.

In some embodiments, the biomarkers provided herein are upregulated in response to compound treatment. In some embodiments, the biomarker upregulated in response to compound treatment is selected from proteins in Table 1. In some embodiments, the biomarker upregulated in response to compound treatment is selected from a group consisting of Nestin, KAT1/CCBL1, WIBG, CK1-alpha, GSPT1, and CRBN. In a specific embodiment, the biomarker upregulated in response to compound treatment is Nestin. In another specific embodiment, the biomarker upregulated in response to compound treatment is KAT1/CCBL1. In yet another specific embodiment, the biomarker upregulated in response to compound treatment is WIBG. In yet another specific embodiment, the biomarker upregulated in response to compound treatment is CRBN. In yet another specific embodiment, the biomarker upregulated in response to compound treatment is CK1-alpha. In yet another specific embodiment, the biomarker upregulated in response to compound treatment is GSPT1. In some embodiments, the biomarker selected from a group consisting of Nestin, KAT1/CCBL1, WIBG, CRBN, CK1-alpha, and GSPT1 is upregulated in response to Compound A treatment.

In other embodiments, the biomarkers provided herein are downregulated in response to compound treatment. In some embodiments, the biomarker downregulated in response to compound treatment is selected from proteins in Table 2. In some embodiments, the biomarker downregulated in response to compound treatment is selected from a group consisting of MVP, PARP4, ZFP91, and ZNF198. In a specific embodiment, the biomarker downregulated in response to compound treatment is MVP. In another specific embodiment, the biomarker downregulated in response to compound treatment is PARP4. In yet another embodiment, the biomarker downregulated in response to compound treatment is ZFP91. In yet another embodiment, the biomarker downregulated in response to compound treatment is ZNF198. In some embodiments, the biomarker selected from a group consisting of MVP, PARP4, ZFP91, an ZNF198 is downregulated in response to Compound A treatment.

TABLE 1
Biomarkers Upregulated in Response to Compound Treatment

| | | |
|---|---|---|
| KAT1 | Nestin | WIBG |
| CK1-alpha | GSPT1 | CRBN |

TABLE 2
Biomarkers Downregulated in Response to Compound Treatment

| | | |
|---|---|---|
| MVP | PARP4 | ZNF91 |
| ZNF198 | | |

In yet other embodiments, the biomarker is a protein selected from Tables 3-6 below:

TABLE 3
Upregulated Proteins in Response to Compound A Treatment (Set 1)

| | | | | |
|---|---|---|---|---|
| SEPT2 | AMACR | CASR | CTSL | GC |
| A2M | ANPEP | CCBL1 | CTU1 | GPD1 |
| A2mp | Anxa5 | CCDC28A | DAPK2 | GPR39 |
| ACSM2B | ANXA9 | Cct3 | DLG5 | HMBOX1 |
| ADAM22 | APBB2 | CD74 | DUSP23 | HYPK |
| AFP | APCS | CDH16 | ELF3 | ITFG2 |
| AGMAT | AQP1 | CEACAM1 | Eps15 | Itgb1 |
| AGT | ATHL1 | CLN3 | ERBB3 | ITIH1 |
| Ahsg | Atp5c1 | COX5A | FAM83H | ITIH4 |
| AHSG | C11orf52 | CPVL | Fkbp9 | KANSL3 |
| ALB | C3 | CRBN | FLRT3 | KCTD14 |
| Amacr | C4A | CRYM | FUK | KLC4 |
| KPTN | Mrps22 | PRODH2 | SEPHS1 | Trap1 |
| Lamp2 | MYRF | Ptbp3 | SERF2 | TSPAN31 |
| LGALS2 | NCOA3 | RBKS | Serpina10 | TTC13 |
| LGALS9 | NR2C1 | RGS14 | Serpinf1 | Upp1 |
| LIG3 | PAH | RHBDD2 | SLC25A51 | USP30 |
| LMBRD1 | PDZK1 | RNF126 | SLC29A3 | Vcam1 |
| LOC100996516 | Peg10 | RNF7 | SPAG7 | VIL1 |
| LPL | PLEKHO2 | Rpl9 | Suclg2 | Vim |
| LYN | Postn | Rplp1 | SULT1A1 | ZNF770 |
| MAP2K7 | POSTN | Rplp2 | SULT1A3 | |
| MLLT6 | PPP1R13L | RSBN1L | Tollip | |
| Mogs | Prdx3 | SAT2 | Top2a | |

TABLE 4
Upregulated Proteins in Response to Compound A Treatment (Set 2)

| | | | | |
|---|---|---|---|---|
| MARCH7 | Col15a1 | HIRIP3 | MYO5B | SERBP1 |
| SEPT2 | CPN1 | HK1 | N4BP2 | Serpina10 |
| Abcc1 | CSRP2BP | HMGXB3 | NABP2 | Serpinc1 |
| ABCG1 | CXorf67 | HSP90AA4P | NAP1L1 | Serpinf2 |
| ABCG2 | DGAT1 | IGDCC4 | Ndufa10 | Serping1 |
| Adam10 | DHRS2 | IL36B | Ndufa2 | SGCB |
| AFF3 | DLG5 | IMMT | Ndufs2 | SIRT7 |
| Akap9 | Eif2s1 | INA | NFRKB | SLC50A1 |
| AP1AR | ENTPD5 | KCTD18 | NSMCE2 | SMCR8 |
| APOL1 | EPDR1 | KIDINS220 | NTHL1 | SPAG1 |
| ATP11A | Eps8l2 | KIF16B | Nup37 | SPECC1 |
| BANP | Epx | KLC4 | OVOS1 | SUV39H1 |
| BLZF1 | FAM111A | KLHL24 | PDCD2 | Sypl1 |
| C10orf118 | FAM120B | LAMTOR5 | Pgd | TAPBPL |
| C4b | FAM206A | LARP1B | PLA2G4A | Tars |
| C6orf57 | FAM83H | LGALS9 | PLEC | Tarsl2 |
| CAST | Fasn | LMBRD1 | PLXNA3 | TCF3 |
| CCAR2 | FASTKD3 | Lta4h | POLG | TEC |
| CCBL1 | FCGRT | LYRM1 | POLK | TERF1 |
| CCDC38 | FLII | MAT1A | PORCN | TEX9 |
| CCDC71L | FNBP1L | MBIP | PPAPDC2 | TGFBRAP1 |
| CD33 | Gak | MBP | Ppp2r4 | TMC6 |
| CD40 | Gatad2a | METTL21A | RBPMS2 | TMEM120A |
| CEACAM1 | Gm906 | MLLT1 | RNF4 | TMEM179B |
| CHKB | GPBP1L1 | MPV17L2 | SAMD1 | TMEM50A |
| CHURC1 | Gtpbp4 | MTIF3 | SATB2 | TOPORS |
| CLK4 | HABP4 | MTRF1L | SELK | TOR4A |
| TRAPPC9 | TXNDC11 | VRK3 | ZBTB40 | ZNF830 |
| Tspan9 | UGCG | XRRA1 | ZDHHC3 | |
| TSPYL1 | Vps37c | YPEL5 | ZHX1 | |
| TUBB4A | VPS54 | ZBTB1 | ZNF292 | |

TABLE 5
Downregulated Proteins in Response to Compound A Treatment (Set 1)

| | | | | |
|---|---|---|---|---|
| ABHD6 | DEGS1 | KIF20B | PDE6D | SLC4A7 |
| ACVR1 | DHX40 | KIF22 | PHC3 | SMAD3 |
| AGR2 | EHD2 | KLC2 | PHLDA1 | SPANXA1 |
| AHNAK2 | EPHA2 | KRT9 | PLA2G4A | SQSTM1 |
| AKAP12 | EREG | LDLR | PLCD4 | TIMM8A |
| ANLN | ETHE1 | LPXN | PPIL4 | TK1 |
| AP5S1 | FAM160A1 | MAFF | PRC1 | TMEM56 |
| ARL4C | FAM172A | MELK | Prkca | TMSB10 |
| ARL6IP1 | FOSL1 | MET | PRKCDBP | TNFRSF12A |
| ARPIN | GHDC | MGAT4B | PTGES2 | TRIM44 |
| ASH1L | GJA1 | MGLL | PTGS1 | TRIM65 |
| AXL | GLRX | MMP7 | RUNX2 | TSC22D1 |
| C4orf3 | GLS | MST1R | SCD | UBE2C |
| CANX | GNG2 | MT2A | SDSL | UBE2E2 |
| CD44 | GNG5 | MVP | SEL1L3 | UPK1B |
| CD46 | GRB10 | MYOF | SEMA3A | VASP |
| CD59 | GRPEL2 | MYOF | SERPINE1 | VPS13B |
| CDC45 | Iap | NDRG1 | SIRPA | WIZ |
| CENPK | Igf2r | NNMT | SKA1 | YBX3 |
| CEP55 | IGFBP1 | NTN4 | SLC14A1 | ZFP91 |
| COL6A3 | IGFBP3 | OTUB2 | SLC25A29 | ZMYM2 |
| CPA4 | IKBKE | PALMD | SLC2A1 | |
| CTNNAL1 | JAG1 | PANK1 | SLC34A2 | |
| CYP27A1 | KIAA0100 | PARP4 | SLC34A2 | |
| CYR61 | KIAA1462 | PBK | SLC38A2 | |

TABLE 6
Downregulated Proteins in Response to Compound A Treatment (Set 2)

| | | | | | |
|---|---|---|---|---|---|
| ACSL6 | ATP5J | CABLES2 | COG5 | CYC1 | FAM162A |
| AHSG | AZGP1 | CAPN15 | COX17 | DAP3 | FAM84B |
| ALB | BCKDK | CCDC88C | COX7B | DNAJC19 | FAM98A |
| ARPP19 | BLOC1S3 | CDH1 | Cpsf1 | DNM2 | FER |
| ATP5EP2 | BTF3 | CETN2 | CSRP2 | DPY30 | FKBP2 |
| ATP5I | C2orf76 | CLTC | CXXC1 | EHMT2 | FUNDC2 |
| GK5 | MAFG | NOMO2 | RAB8B | RTN4IP1 | TMBIM6 |
| GLRX | MAGEA10 | NT5C2 | RNF166 | S100A11 | TMCO1 |
| Gm14139 | MED11 | Nt5dc2 | ROBO1 | SDHAF2 | TMED9 |
| GOLT1B | MED18 | NT5E | RPL13A | SDSL | TMEM189 |
| GPATCH3 | METTL5 | NUDT3 | Rpl18 | Sec13 | TMEM222 |
| Hbb-b2 | MFSD1 | NUDT5 | RPL18A | SERF2 | TPM1 |
| HIST1H1C | MGAT4B | OASL | RPL19 | SIK2 | TWSG1 |
| Hist1h1e | MGST1 | PARP4 | RPL22 | SLC20A2 | UBE3B |
| HIST1H1E | MGST2 | Parp4 | RPL28 | SLC25A3 | VAPA |
| HIST1H2AH | MRPL9 | PCK1 | RPL35 | SLC25A6 | VHL |
| HIST1H2BC | MRPS18C | PDE6D | RPL36AL | SLC52A2 | VNN1 |
| HMGB2 | MRPS21 | PFN2 | RPL37 | SNRPF | ZDHHC20 |
| HMGN3 | Mt2 | PGPEP1 | Rp17 | SPANXA1 | ZEB1 |
| HSP90B1 | MT2A | PIGK | RPL8 | SPPL2B | ZFP91 |
| HSPB11 | MT-ATP8 | PLOD1 | RPS13 | STIM2 | ZMYM2 |
| IKBKE | MVP | Prdx2 | RPS15 | STK11 | |
| JAGN1 | MYO1F | PTBP2 | RPS17L | SYTL4 | |
| Kxd1 | NDUFB1 | PTDSS1 | RPS19 | TAF15 | |
| LAMA1 | NME7 | RAB28 | RPS25 | TBC1D12 | |
| LPXN | NMES1 | RAB4B | RPS29 | TFAP4 | |

In some embodiments, the biomarker is a protein selected from SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTU1, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770, or a combination thereof.

In one embodiment, the biomarker is RSBN1L. In one embodiment, the biomarker is SULT1A3. In one embodiment, the biomarker is ITFG2. In one embodiment, the biomarker is FUK. In one embodiment, the biomarker is LMBRD1. In one embodiment, the biomarker is AHSG. In one embodiment, the biomarker is MLLT6. In one embodiment, the biomarker is ELF3. In one embodiment, the biomarker is CD74. In one embodiment, the biomarker is AMACR. In one embodiment, the biomarker is Rplpl. In one embodiment, the biomarker is PPP1R13L. In one embodiment, the biomarker is CCDC28A. In one embodiment, the biomarker is ATHL1. In one embodiment, the biomarker is ALB. In one embodiment, the biomarker is ITIH1. In one embodiment, the biomarker is LIG3. In one embodiment, the biomarker is MAP2K7. In one embodiment, the biomarker is NR2C1. In one embodiment, the biomarker is Prdx3. In one embodiment, the biomarker is GC. In one embodiment, the biomarker is COX5A. In one embodiment, the biomarker is GPD1. In one embodiment, the biomarker is RBKS. In one embodiment, the biomarker is Trapl. In one embodiment, the biomarker is MYRF. In one embodiment, the biomarker is ANXA9. In one embodiment, the biomarker is HYPK. In one embodiment, the biomarker is LPL. In one embodiment, the biomarker is ZNF770. In one embodiment, the biomarker is SAT2. In one embodiment, the biomarker is CTU1. In one embodiment, the biomarker is CTSL. In one embodiment, the biomarker is Itgb1. In one embodiment, the biomarker is SERF2. In one embodiment, the biomarker is CRBN. In one embodiment, the biomarker is Peg10. In one embodiment, the biomarker is C4A. In one embodiment, the biomarker is DAPK2. In one embodiment, the biomarker is Lamp2. In one embodiment, the biomarker is SEPHS1. In one embodiment, the biomarker is SLC29A3. In one embodiment, the biomarker is LOC100996516. In one embodiment, the biomarker is KANSL3. In one embodiment, the biomarker is To11ip. In one embodiment, the biomarker is NCOA3. In one embodiment, the biomarker is C11orf52. In one embodiment, the biomarker is LGALS2. In one embodiment, the biomarker is Vim. In one embodiment, the biomarker is RHBDD2. In one embodiment, the biomarker is A2M. In one embodiment, the biomarker is VIL1. In one embodiment, the biomarker is C3. In one embodiment, the biomarker is ACSM2B. In one embodiment, the biomarker is Postn. In one embodiment, the biomarker is Rp19. In one embodiment, the biomarker is USP30. In one embodiment, the biomarker is Anxa5. In one embodiment, the biomarker is TTC13. In one embodiment, the biomarker is APCS. In one embodiment, the biomarker is ADAM22. In one embodiment, the biomarker is DUSP23. In one embodiment, the biomarker is KCTD14. In one embodiment, the biomarker is SEPT2. In one embodiment, the biomarker is Rp1p2. In one embodiment, the biomarker is RNF126. In one embodiment, the biomarker is GPR39. In one embodiment, the biomarker is SLC25A51. In one embodiment, the biomarker is FLRT3. In one embodiment, the biomarker is Ahsg. In one embodiment, the biomarker is AFP. In one embodiment, the biomarker is CLN3. In one embodiment, the biomarker is LYN. In one embodiment, the biomarker is SULT1A1. In one embodiment, the biomarker is Mogs. In one embodiment, the biomarker is Serpina10. In one embodiment, the biomarker is KPTN. In one embodiment, the biomarker is APBB2. In one embodiment, the biomarker is Top2a. In one embodiment, the biomarker is Suclg2. In one embodiment, the biomarker is ITIH4. In one embodiment, the biomarker is PDZK1. In one embodiment, the biomarker is CEACAM1. In one embodiment, the biomarker is AQP1. In one embodiment, the biomarker is ERBB3. In one embodiment, the biomarker is FAM83H. In one embodiment, the biomarker is RGS14. In one embodiment, the biomarker is KLC4. In one embodiment, the biomarker is CDH16. In one embodiment, the biomarker is AGT. In one embodiment, the biomarker is Eps15. In one embodiment, the biomarker is POSTN. In one embodiment, the biomarker is AGMAT. In one embodiment, the biomarker is SPAG7. In one embodiment, the biomarker is DLG5. In one embodiment, the biomarker is CCBL1. In one embodiment, the biomarker is Fkbp9. In one embodiment, the biomarker is Amacr. In one embodiment, the biomarker is CRYM. In one embodiment, the biomarker is TSPAN31. In one embodiment, the biomarker is PRODH2. In one embodiment, the biomarker is LGALS9. In one embodiment, the biomarker is Cct3. In one embodiment, the biomarker is RNF7. In one embodiment, the biomarker is PLEKHO2. In one embodiment, the biomarker is HMBOX1. In one embodiment, the biomarker is Serpinf1. In one embodiment, the biomarker is PAH. In one embodiment, the biomarker is Vcam1. In one embodiment, the biomarker is CPVL. In one embodiment, the biomarker is Ptbp3. In one embodiment, the biomarker is A2mp. In one embodiment, the biomarker is ANPEP. In one embodiment, the biomarker is CASR. In one embodiment, the biomarker is Upp1. In one embodiment, the biomarker is Atp5c1. In one embodiment, the biomarker is Mrps22.

In other embodiments, the biomarker is a protein selected from a group consisting of MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDC38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLIT, FNBP1L, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830, or a combination thereof.

In one embodiment, the biomarker is TGFBRAP1. In one embodiment, the biomarker is TMEM179B. In one embodiment, the biomarker is CCAR2. In one embodiment, the biomarker is Epx. In one embodiment, the biomarker is KCTD18. In one embodiment, the biomarker is SEPT2. In one embodiment, the biomarker is SLC50A1. In one embodiment, the biomarker is MAT1A. In one embodiment, the biomarker is CSRP2BP. In one embodiment, the biomarker is BANP. In one embodiment, the biomarker is FLII. In one embodiment, the biomarker is YPEL5. In one embodiment, the biomarker is Tars. In one embodiment, the biomarker is Adam10. In one embodiment, the biomarker is PLA2G4A. In one embodiment, the biomarker is PPAPDC2. In one embodiment, the biomarker is TCF3. In one embodiment, the biomarker is SERBP1. In one embodiment, the biomarker is TXNDC11. In one embodiment, the biomarker is C6orf57. In one embodiment, the biomarker is Col15a1. In one embodiment, the biomarker is TRAPPC9. In one embodiment, the biomarker is Sypl1. In one embodiment, the biomarker is CCBL1. In one embodiment, the biomarker is PLXNA3. In one embodiment, the biomarker is Nup37. In one embodiment, the biomarker is LGALS9. In one embodiment, the biomarker is ATP11A. In one embodiment, the biomarker is NSMCE2. In one embodiment, the biomarker is SIRT7. In one embodiment, the biomarker is ABCG2. In one embodiment, the biomarker is METTL21A. In one embodiment, the biomarker is FNBP1L. In one embodiment, the biomarker is Pgd. In one embodiment, the biomarker is CLK4. In one embodiment, the biomarker is Eps812. In one embodiment, the biomarker is APOL1. In one embodiment, the biomarker is CHURC1. In one embodiment, the biomarker is LMBRD1. In one embodiment, the biomarker is Eif2s1. In one embodiment, the biomarker is NABP2. In one embodiment, the biomarker is OVOS1. In one embodiment, the biomarker is C10orf118. In one embodiment, the biomarker is Tars12. In one embodiment, the biomarker is SELK. In one embodiment, the biomarker is TMEM50A. In one embodiment, the biomarker is FAM206A. In one embodiment, the biomarker is Ndufs2. In one embodiment, the biomarker is Serpina10. In one embodiment, the biomarker is NTHL1. In one embodiment, the biomarker is HABP4. In one embodiment, the biomarker is POLK. In one embodiment, the biomarker is PLEC. In one embodiment, the biomarker is TEX9. In one embodiment, the biomarker is Lta4h. In one embodiment, the biomarker is CPN1. In one embodiment, the biomarker is FASTKD3. In one embodiment, the biomarker is Serping1. In one embodiment, the biomarker is Akap9. In one embodiment, the biomarker is ZNF292. In one embodiment, the biomarker is SUV39H1. In one embodiment, the biomarker is CCDC71L. In one embodiment, the biomarker is VRK3. In one embodiment, the biomarker is LARP1B. In one embodiment, the biomarker is LYRM1. In one embodiment, the biomarker is CD40. In one embodiment, the biomarker is SPECC1. In one embodiment, the biomarker is TOPORS. In one embodiment, the biomarker is PORCN. In one embodiment, the biomarker is CHKB. In one embodiment, the biomarker is IGDCC4. In one embodiment, the biomarker is FAM111A. In one embodiment, the biomarker is Vps37c. In one embodiment, the biomarker is DHRS2. In one embodiment, the biomarker is ZBTB40. In one embodiment, the biomarker is Serpinf2. In one embodiment, the biomarker is DGAT1. In one embodiment, the biomarker is INA. In one embodiment, the biomarker is NAP1L1. In one embodiment, the biomarker is ZNF830. In one embodiment, the biomarker is CAST. In one embodiment, the biomarker is ZBTB1. In one embodiment, the biomarker is POLG. In one embodiment, the biomarker is TAPBPL. In one embodiment, the biomarker is MYO5B. In one embodiment, the biomarker is HK1. In one embodiment, the biomarker is Gtpbp4. In one embodiment, the biomarker is LAMTOR5. In one embodiment, the biomarker is FAM83H. In one embodiment, the biomarker is TERF1. In one embodiment, the biomarker is AFF3. In one embodiment, the biomarker is TEC. In one embodiment, the biomarker is C4b. In one embodiment, the biomarker is Ndufa2. In one embodiment, the biomarker is Abcc1. In one embodiment, the biomarker is SAMD1. In one embodiment, the biomarker is MPV17L2. In one embodiment, the biomarker is CEACAM1. In one embodiment, the biomarker is TMEM120A. In one embodiment, the biomarker is ENTPD5. In one embodiment, the biomarker is SPAG1. In one embodiment, the biomarker is SGCB. In one embodiment, the biomarker is Gatad2a. In one embodiment, the biomarker is CD33. In one embodiment, the biomarker is EPDR1. In one embodiment, the biomarker is RBPMS2. In one embodiment, the biomarker is GPBP1L1. In one embodiment, the biomarker is RNF4. In one embodiment, the biomarker is KIDINS220. In one embodiment, the biomarker is ABCG1. In one embodiment, the biomarker is KLC4. In one embodiment, the biomarker is BLZF1. In one embodiment, the biomarker is KIF16B. In one embodiment, the biomarker is HMGXB3. In one embodiment, the biomarker is FAM120B. In one embodiment, the biomarker is TMC6. In one embodiment, the biomarker is XRRA1. In one embodiment, the biomarker is Tspan9. In one embodiment, the biomarker is DLG5. In one embodiment, the biomarker is Fasn. In one embodiment, the biomarker is TOR4A. In one embodiment, the biomarker is Serpinc1. In one embodiment, the biomarker is CCDC38. In one embodiment, the biomarker is ZHX1. In one embodiment, the biomarker is Gak. In one embodiment, the biomarker is PDCD2. In one embodiment, the biomarker is MBIP. In one embodiment, the biomarker is VPS54. In one embodiment, the biomarker is AP1AR. In one embodiment, the biomarker is UGCG. In one embodiment, the biomarker is KLHL24. In one embodiment, the biomarker is Gm906. In one embodiment, the biomarker is MTIF3. In one embodiment, the biomarker is CXorf67. In one embodiment, the biomarker is TUBB4A. In one embodiment, the biomarker is HSP90AA4P. In one embodiment, the biomarker is MBP. In one embodiment, the biomarker is Ndufa10. In one embodiment, the biomarker is Ppp2r4. In one embodiment, the biomarker is IMMT. In one embodiment, the biomarker is ZDHHC3. In one embodiment, the biomarker is MTRF1L. In one embodiment, the biomarker is MARCH7. In one embodiment, the biomarker is FCGRT. In one embodiment, the biomarker is SMCR8. In one embodiment, the biomarker is HIRIP3. In one embodiment, the biomarker is N4BP. In one embodiment, the biomarker is NFRKB. In one embodiment, the biomarker is SATB2. In one embodiment, the biomarker is MLLT1. In one embodiment, the biomarker is IL36B. In one embodiment, the biomarker is TSPYL1.

In yet other embodiments, the biomarker is a protein selected from a group consisting of ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Iap, Igf2r, IGFBP1, IGFBP3, IKBKE, JAG1, KIAA0100, KIAA1462, KIF20B, KIF22, KLC2, KRT9, LDLR, LPXN, MAFF, MELK, MET, MGAT4B, MGLL, MMP7, MST1R, MT2A, MVP, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2, or a combination thereof.

In one embodiment, the biomarker is ZFP91. In one embodiment, the biomarker is VPS13B. In one embodiment, the biomarker is SDSL. In one embodiment, the biomarker is CD59. In one embodiment, the biomarker is SEMA3A. In one embodiment, the biomarker is MGAT4B. In one embodiment, the biomarker is MST1R. In one embodiment, the biomarker is Iap. In one embodiment, the biomarker is PANK1. In one embodiment, the biomarker is AGR2. In one embodiment, the biomarker is LDLR. In one embodiment, the biomarker is ACVR1. In one embodiment, the biomarker is ZMYM2. In one embodiment, the biomarker is PLA2G4A. In one embodiment, the biomarker is MT2A. In one embodiment, the biomarker is AP5S1. In one embodiment, the biomarker is EHD2. In one embodiment, the biomarker is PHC3. In one embodiment, the biomarker is PDE6D. In one embodiment, the biomarker is FAM172A. In one embodiment, the biomarker is LPXN. In one embodiment, the biomarker is NTN4. In one embodiment, the biomarker is SLC38A2. In one embodiment, the biomarker is PHLDA1. In one embodiment, the biomarker is ARPIN. In one embodiment, the biomarker is NDRG1. In one embodiment, the biomarker is KIAA1462. In one embodiment, the biomarker is RUNX2. In one embodiment, the biomarker is CEP55. In one embodiment, the biomarker is ETHE1. In one embodiment, the biomarker is EREG. In one embodiment, the biomarker is SPANXA1. In one embodiment, the biomarker is GHDC. In one embodiment, the biomarker is GLS. In one embodiment, the biomarker is KIF20B. In one embodiment, the biomarker is PRC1. In one embodiment, the biomarker is ABHD6. In one embodiment, the biomarker is TRIM65. In one embodiment, the biomarker is SLC25A29. In one embodiment, the biomarker is CDC45. In one embodiment, the biomarker is TMSB10. In one embodiment, the biomarker is SKA1. In one embodiment, the biomarker is MVP. In one embodiment, the biomarker is AXL. In one embodiment, the biomarker is ARL4C. In one embodiment, the biomarker is KIF22. In one embodiment, the biomarker is EPHA2. In one embodiment, the biomarker is PRKCDBP. In one embodiment, the biomarker is CTNNAL1. In one embodiment, the biomarker is GNG5. In one embodiment, the biomarker is MMP7. In one embodiment, the biomarker is NNMT. In one embodiment, the biomarker is DHX40. In one embodiment, the biomarker is TIMM8A. In one embodiment, the biomarker is PLCD4. In one embodiment, the biomarker is ASH1L. In one embodiment, the biomarker is TNFRSF12A. In one embodiment, the biomarker is Igf2r. In one embodiment, the biomarker is JAG1. In one embodiment, the biomarker is SLC34A2. In one embodiment, the biomarker is IGFBP1. In one embodiment, the biomarker is ARL6IP1. In one embodiment, the biomarker is SLC2A1. In one embodiment, the biomarker is MYOF. In one embodiment, the biomarker is UBE2E2. In one embodiment, the biomarker is TK1. In one embodiment, the biomarker is SERPINE1. In one embodiment, the biomarker is TRIM44. In one embodiment, the biomarker is OTUB2. In one embodiment, the biomarker is MYOF. In one embodiment, the biomarker is C4orf3. In one embodiment, the biomarker is CYR61. In one embodiment, the biomarker is KRT9. In one embodiment, the biomarker is TMEM56. In one embodiment, the biomarker is PBK. In one embodiment, the biomarker is SQSTM1. In one embodiment, the biomarker is MGLL. In one embodiment, the biomarker is GRPEL2. In one embodiment, the biomarker is AKAP12. In one embodiment, the biomarker is IGFBP3. In one embodiment, the biomarker is KLC2. In one embodiment, the biomarker is SLC14A1. In one embodiment, the biomarker is PALMD. In one embodiment, the biomarker is PTGES2. In one embodiment, the biomarker is UPK1B. In one embodiment, the biomarker is WIZ. In one embodiment, the biomarker is AHNAK2. In one embodiment, the biomarker is GLRX. In one embodiment, the biomarker is CD46. In one embodiment, the biomarker is SIRPA. In one embodiment, the biomarker is FOSL1. In one embodiment, the biomarker is YBX3. In one embodiment, the biomarker is UBE2C. In one embodiment, the biomarker is SMAD3. In one embodiment, the biomarker is SLC4A7. In one embodiment, the biomarker is GRB10. In one embodiment, the biomarker is GJA1. In one embodiment, the biomarker is CD44. In one embodiment, the biomarker is SLC34A2. In one embodiment, the biomarker is KIAA0100. In one embodiment, the biomarker is ANLN. In one embodiment, the biomarker is CENPK. In one embodiment, the biomarker is PARP4. In one embodiment, the biomarker is Prkca. In one embodiment, the biomarker is SCD. In one embodiment, the biomarker is GNG2. In one embodiment, the biomarker is MET. In one embodiment, the biomarker is SEL1L3. In one embodiment, the biomarker is PPIL4. In one embodiment, the biomarker is COL6A3. In one embodiment, the biomarker is PTGS1. In one embodiment, the biomarker is MAFF. In one embodiment, the biomarker is TSC22D1. In one embodiment, the biomarker is DEGS1. In one embodiment, the biomarker is FAM160A1. In one embodiment, the biomarker is CANX. In one embodiment, the biomarker is CYP27A1. In one embodiment, the biomarker is CPA4. In one embodiment, the biomarker is VASP. In one embodiment, the biomarker is MELK. In one embodiment, the biomarker is IKBKE.

In yet other embodiments, the biomarker is a protein selected from a group consisting of ACSL6, AHSG, ALB, ARPP19, ATP5EP2, ATP5I, ATP5J, AZGP1, BCKDK, BLOC1S3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1H1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD 1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBC1D12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2, or a combination thereof.

In one embodiment, the biomarker is CLTC. In one embodiment, the biomarker is PDE6D. In one embodiment, the biomarker is METTL5. In one embodiment, the biomarker is PARP4. In one embodiment, the biomarker is CSRP2. In one embodiment, the biomarker is RTN4IP1. In one embodiment, the biomarker is LAMA1. In one embodiment, the biomarker is SPPL2B. In one embodiment, the biomarker is SYTL4. In one embodiment, the biomarker is HSPB11. In one embodiment, the biomarker is TPM1. In one embodiment, the biomarker is SDSL. In one embodiment, the biomarker is AZGP1. In one embodiment, the biomarker is Hbb-b2. In one embodiment, the biomarker is HIST1H1E. In one embodiment, the biomarker is ATP5J. In one embodiment, the biomarker is TMEM189. In one embodiment, the biomarker is JAGN1. In one embodiment, the biomarker is MED11. In one embodiment, the biomarker is GOLT1B. In one embodiment, the biomarker is VHL. In one embodiment, the biomarker is MGST1. In one embodiment, the biomarker is ROBO1. In one embodiment, the biomarker is COGS. In one embodiment, the biomarker is ZFP91. In one embodiment, the biomarker is CXXC1. In one embodiment, the biomarker is CDH1. In one embodiment, the biomarker is RPL22. In one embodiment, the biomarker is ATP5EP2. In one embodiment, the biomarker is RPS25. In one embodiment, the biomarker is PCK1. In one embodiment, the biomarker is UBE3B. In one embodiment, the biomarker is GPATCH3. In one embodiment, the biomarker is Sec13. In one embodiment, the biomarker is DNAJC19. In one embodiment, the biomarker is SLC52A2. In one embodiment, the biomarker is Prdx2. In one embodiment, the biomarker is NUDT5. In one embodiment, the biomarker is CETN2. In one embodiment, the biomarker is Cpsf1. In one embodiment, the biomarker is TWSG1. In one embodiment, the biomarker is NOMO2. In one embodiment, the biomarker is TFAP4. In one embodiment, the biomarker is DPY30. In one embodiment, the biomarker is Gm14139. In one embodiment, the biomarker is LPXN. In one embodiment, the biomarker is RAB8B. In one embodiment, the biomarker is Mt2. In one embodiment, the biomarker is RAB28. In one embodiment, the biomarker is HIST1H1C. In one embodiment, the biomarker is MGAT4B. In one embodiment, the biomarker is STIM2. In one embodiment, the biomarker is TMED9. In one embodiment, the biomarker is RPL36AL. In one embodiment, the biomarker is SDHAF2. In one embodiment, the biomarker is C2orf76. In one embodiment, the biomarker is MRPS18C. In one embodiment, the biomarker is VAPA. In one embodiment, the biomarker is MAFG. In one embodiment, the biomarker is VNN1. In one embodiment, the biomarker is SIK2. In one embodiment, the biomarker is HIST1H2AH. In one embodiment, the biomarker is CAPN15. In one embodiment, the biomarker is FER. In one embodiment, the biomarker is RPS19. In one embodiment, the biomarker is PLOD1. In one embodiment, the biomarker is CCDC88C. In one embodiment, the biomarker is PTBP2. In one embodiment, the biomarker is RPL19. In one embodiment, the biomarker is ARPP19. In one embodiment, the biomarker is RPL37. In one embodiment, the biomarker is SLC25A6. In one embodiment, the biomarker is MRPS21. In one embodiment, the biomarker is STK11. In one embodiment, the biomarker is COX7B. In one embodiment, the biomarker is NT5E. In one embodiment, the biomarker is SERF2. In one embodiment, the biomarker is NT5C2. In one embodiment, the biomarker is Nt5dc2. In one embodiment, the biomarker is HIST1H2BC. In one embodiment, the biomarker is NUDT3. In one embodiment, the biomarker is Rp17. In one embodiment, the biomarker is FAM98A. In one embodiment, the biomarker is S100A11. In one embodiment, the biomarker is MFSD1. In one embodiment, the biomarker is FKBP2. In one embodiment, the biomarker is TMBIM6. In one embodiment, the biomarker is PTDSS1. In one embodiment, the biomarker is NMES1. In one embodiment, the biomarker is Parp4. In one embodiment, the biomarker is Hist1h1e. In one embodiment, the biomarker is MAGEA10. In one embodiment, the biomarker is TAF15. In one embodiment, the biomarker is FUNDC2. In one embodiment, the biomarker is SPANXA1. In one embodiment, the biomarker is ACSL6. In one embodiment, the biomarker is HMGN3. In one embodiment, the biomarker is MVP. In one embodiment, the biomarker is BLOC1S3. In one embodiment, the biomarker is FAM84B. In one embodiment, the biomarker is ALB. In one embodiment, the biomarker is RPS17L. In one embodiment, the biomarker is SLC20A2. In one embodiment, the biomarker is SLC25A3. In one embodiment, the biomarker is HMGB2. In one embodiment, the biomarker is RPL8. In one embodiment, the biomarker is PIGK. In one embodiment, the biomarker is RPS13. In one embodiment, the biomarker is RAB4B. In one embodiment, the biomarker is MT2A. In one embodiment, the biomarker is PFN2. In one embodiment, the biomarker is TMCO1. In one embodiment, the biomarker is MT-ATP8. In one embodiment, the biomarker is MYO1F. In one embodiment, the biomarker is CABLES2. In one embodiment, the biomarker is RPL28. In one embodiment, the biomarker is GK5. In one embodiment, the biomarker is RPL18A. In one embodiment, the biomarker is RPS15. In one embodiment, the biomarker is RPL13A. In one embodiment, the biomarker is DNM2. In one embodiment, the biomarker is MGST2. In one embodiment, the biomarker is TMEM222. In one embodiment, the biomarker is AHSG. In one embodiment, the biomarker is BCKDK. In one embodiment, the biomarker is NME7. In one embodiment, the biomarker is HSP90B1. In one embodiment, the biomarker is ATP5I. In one embodiment, the biomarker is BTF3. In one embodiment, the biomarker is SNRPF. In one embodiment, the biomarker is DAP3. In one embodiment, the biomarker is NDUFB1. In one embodiment, the biomarker is ZMYM2. In one embodiment, the biomarker is COX17. In one embodiment, the biomarker is RPS29. In one embodiment, the biomarker is PGPEP1. In one embodiment, the biomarker is MED18. In one embodiment, the biomarker is MRPL9. In one embodiment, the biomarker is ZEB1. In one embodiment, the biomarker is ZDHHC20. In one embodiment, the biomarker is FAM162A. In one embodiment, the biomarker is CYC1. In one embodiment, the biomarker is RPL35. In one embodiment, the biomarker is EHMT2. In one embodiment, the biomarker is RNF166. In one embodiment, the biomarker is Rp118. In one embodiment, the biomarker is TBC1D12. In one embodiment, the biomarker is Kxd1. In one embodiment, the biomarker is GLRX. In one embodiment, the biomarker is OASL. In one embodiment, the biomarker is IKBKE.

The present disclosure is also based, in part, on the discovery that ZFP91 is down-regulated in solid tumor cell lines, e.g., HCC or GBM, in response to a treatment with treatment compounds provided herein. Thus, in a specific embodiment, the biomarker is ZFP91. ZFP91 zinc finger protein (ZFP91) encodes a 63.4 kDa nuclear protein with structural motifs characteristic of transcription factor, and it is expressed ubiquitously in various cell types and is known to be highly conservative. Paschke et al., Pathol Oncol Res., 2013, 20:453-459. ZFP91 is recognized as an atypical E3 ubiquitin-protein ligase, and mediates a K63-linked ubiquitination of MAP3K14. Id. It has also been indicated that ZFP91 has implications in various cancers. For example, it has been shown that ZFP91 expression is upregulated in mononuclear cells from patients with acute myelogenous leukemia (AML) and in many neoplastic blood cell lines. In addition, the function of ZFP91 has been shown to relate to cell apoptosis rate. Id. Furthermore, ZFP91 has been shown to interact with ARF tumor suppressor (cyclin-dependent kinase inhibitor 2A, isoform 4), the von Hippel-Lindau tumor suppressor (pVHL) and the hypoxia inducible factor-1α (HIF-1α). In some embodiments, the biomarker is an isoform of ZFP91 protein.

The present disclosure is also based in part on the discovery that Cereblon (CRBN) is up-regulated in solid tumor cell lines, e.g., HCC or GBM, in response to a treatment with treatment compounds provided herein. Thus, in another specific embodiment, the biomarker is CRBN. CRBN is a 442-amino acid protein conserved from plant to human. In humans, the CRBN gene has been identified as a candidate gene of an autosomal recessive nonsyndromic mental retardation (ARNSMR). See Higgins, J. J. et al., Neurology, 2004, 63:1927-1931. CRBN was initially characterized as an RGS-containing novel protein that interacted with a calcium-activated potassium channel protein (SLO1) in the rat brain, and was later shown to interact with a voltage-gated chloride channel (CIC-2) in the retina with AMPK7 and DDB1. See Jo, S. et al., J. Neurochem, 2005, 94:1212-1224; Hohberger B. et al., FEBS Lett, 2009, 583:633-637; Angers S. et al., Nature, 2006, 443:590-593. DDB1 was originally identified as a nucleotide excision repair protein that associates with damaged DNA binding protein 2 (DDB2). Its defective activity causes the repair defect in the patients with xeroderma pigmentosum complementation group E (XPE). DDB1 also appears to function as a component of numerous distinct DCX (DDB1-CUL4-X-box) E3 ubiquitin-protein ligase complexes which mediate the ubiquitination and subsequent proteasomal degradation of target proteins. CRBN has also been identified as a target for the development of therapeutic agents for diseases of the cerebral cortex. See WO 2010/137547 A1. In some embodiments, the biomarker is an isoform of CRBN.

In certain embodiments, the biomarker is an mRNA of Nestin, KAT1/CCBL1, WIBG, MVP, PARP4, ZFP91, ZNF198 or a combination thereof. In certain embodiments, the biomarker is an mRNA of Nestin, KAT1/CCBL1, and WIBG. In certain embodiments, the biomarker is an mRNA of MVP, PARP4, ZFP91, or ZNF198.

In certain embodiments, the biomarker is an mRNA of AHNAK, ALOX5, AMPD3, ANXA4, ANXA6, ARHGAP19, ASNS, ASPM, ATP2B4, B4GALT3, BANK1, BCDIN3D, BLZF1, BMF, BST2, C10orf76, C19orf66, CA2, CA8, CAMSAP3, CCDC69, CCNB1, CD36, CDC7, CDCA3, CENPF, CLN3, CNN3, CORO1B, CPNE2, CRBN, CSNK1A1, CSRP2, CTNND1, CTSH, DAPK2, DDX58, DHPS, DHX58, DLG2, DLGAP5, DOK3, DTX3L, ECT2, EFCAB4B, EHMT1, EHMT2, EIF2AK2, EPB41L1, EPCAM, ESRP1, ETV6, EXTL2, F13A1, FAM195A, FAM65B, FBRSL1, FCGR2B, FES, FHOD1, FIGNL1, FMNL3, GBP1, GMFG, GMPR, GPT2, GRAMD1A, GRAMD1B, GRPEL2, HIP1, HJURP, HLA-B, HLA-DMA, HMCES, HMMR, HOXC4, HPSE, ICAM2, ID3, IFI35, IFIH1, IFIT1, IFIT3, IFIT5, IFITM2, IKZF1, IKZF3, IL4I1, IRF7, IRF9, IRS2, ISG15, ISG20, ITGB7, JAK3, KIF18B, K1F22, KIF2C, LAP3, LGALS1, LGALS3BP, LIMD1, LIPG, LPXN, MAN2A2, MARCKS, MFI2, MGARP, MINA, MIS18BP1, MOV10, MPP7, MUC1, MX1, MX2, MYO1G, NCF2, NEIL1, NFKBID, NME3, NMI, NPIPB5, NT5C3A, OAS1, OAS2, OAS3, OMA1, ORC6, PARP14, PARP9, PARVB, PBK, PBXIP1, PDE6D, PKMYT1, PLD4, PLEKHO1, PLK1, PLSCR1, PLXNB2, PODXL, PODXL2, POLE2, POMP, PPFIBP1, PRDM15, PRNP, PTAFR, PTMS, PTTG1, PYROXD1, QPRT, RAB13, RASA4B, RASSF6, RCN1, RGCC, RGS1, RGS2, RNF213, S100A13, SAMD9L, SAMHD1, SEC14L1, SERPINH1, SGOL1, SGOL2, SLCO3A1, SLCO4A1, SLFN11, SLFN13, SLFN5, SP110, SP140, SPN, SPR, STAP1, STAT1, STAT2, TACC3, TAP1, TAX1BP3, THEMIS2, THTPA, TIMM8B, TNFAIP8L2, TNFSF8, TOP2A, TP53I3, TPX2, TREX1, TRIB3, TRIM22, TTC39C, TXNIP, UBA7, UBE2L6, USP41, VCL, VNN2, WIZ, WSB1, WWC1, ZBTB38, ZFP91, ZMYM2, ZNF385B, ZNF581 or ZNF644, or a combination thereof.

In other embodiments, the biomarker is an mRNA of AHNAK, ALOX5, AMPD3, ANXA4, ANXA6, ATP2B4, BMF, BST2, C10orf76, C19orf66, CD36, CLN3, CNN3, CORO1B, CPNE2, CRBN, CSRP2, CTNND1, CTSH, DAPK2, DDX58, DHX58, DLG2, DTX3L, EIF2AK2, EPB41L1, ETV6, EXTL2, F13A1, FAM65B, FCGR2B, FES, FMNL3, GBP1, GMFG, GMPR, HIP1, HLA-B, HLA-DMA, HPSE, ID3, IF135, IFIH1, IFIT1, IFIT3, IFIT5, IFITM2, IL4I1, IRF7, IRF9, ISG15, ISG20, ITGB7, JAK3, LAP3, LGALS1, LGALS3BP, LIMD1, MAN2A2, MARCKS, MFI2, MGARP, MOV10, MPP7, MUC1, MX1, MX2, MYO1G, NCF2, NME3, NMI, NT5C3A, OAS1, OAS2, OAS3, PARP14, PARP9, PBXIP1, PLD4, PLEKHO1, PLSCR1, PLXNB2, POMP, PPFIBP1, PTMS, QPRT, RAB13, RCN1, RGCC, RNF213, S100A13, SAMD9L, SAMHD1, SERPINH1, SLFN11, SLFN13, SLFN5, SP110, SP140, SPN, SPR, STAP1, STAT1, STAT2, TAP1, TAX1BP3, THEMIS2, THTPA, TNFAIP8L2, TNFSF8, TP53I3, TREX1, TRIM22, TTC39C, TXNIP, UBA7, UBE2L6, USP41, VCL, VNN2 and ZBTB38, or a combination thereof.

In yet other embodiments, the biomarker is an mRNA of ARHGAP19, ASNS, ASPM, B4GALT3, BANK1, BCDIN3D, BLZF1, CA2, CA8, CAMSAP3, CCDC69, CCNB1, CDC7, CDCA3, CENPF, CSNK1A1DHPS, DLGAP5, DOK3, ECT2, EFCAB4B, EHMT1, EHMT2, EPCAM, ESRP1, FAM195A, FBRSL1, FHOD1, FIGNL1, GPT2, GRAMD1A, GRAMD1B, GRPEL2, HJURP, HMCES, HMMR, HOXC4, ICAM2, IKZF1, IKZF3, IRS2, KIF18B, K1F22, KIF2C, LIPG, LPXN, MINA, MIS18BP1, NEIL1, NFKBID, NPIPB5, OMA1, ORC6, PARVB, PBK, PDE6D, PKMYT1, PLK1, PODXL, PODXL2, POLE2, PRDM15, PRNP, PTAFR, PTTG1, PYROXD1, RASA4B, RASSF6, RGS1, RGS2, SEC14L1, SGOL1, SGOL2, SLCO3A1, SLCO4A1, TACC3, TIMM8B, TOP2A, TPX2, TRIB3, WIZ, WSB1, WWC1, ZFP91, ZMYM2, ZNF385B, ZNF581 and ZNF644, or a combination thereof.

In yet other embodiments, the biomarker is an mRNA of SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTU1, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770, or a combination thereof.

In yet other embodiments, the biomarker is an mRNA of MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDC38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLII, FNBP1L, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830, or a combination thereof.

In yet other embodiments, the biomarker is an mRNA of ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Tap, Igf2r, IGFBP1, IGFBP3, IKBKE, JAG1, KIAA0100, KIAA1462, KIF20B, KIF22, KLC2, KRT9, LDLR, LPXN, MAFF, MELK, MET, MGAT4B, MGLL, MMP7, MST1R, MT2A, MVP, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2, or a combination thereof.

In yet other embodiments, the biomarker is an mRNA of ACSL6, AHSG, ALB, ARPP19, ATP5EP2, ATP5I, ATP5J, AZGP1, BCKDK, BLOC1S3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1H1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBC1D12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2, or a combination thereof.

In a specific embodiment, the biomarker is an mRNA of ZFP91. In another specific embodiment, the biomarker is an mRNA of CRBN. In another specific embodiment, the biomarker is an mRNA of Nestin. In another specific embodiment, the biomarker is an mRNA of KAT1/CCBL1. In another specific embodiment, the biomarker is an mRNA of WIBG. In another specific embodiment, the biomarker is an mRNA of MVP. In another specific embodiment, the biomarker is an mRNA of PARP4. In another specific embodiment, the biomarker is an mRNA of ZNF198. In another specific embodiment, the biomarker is an mRNA of MST1R (RON). In another specific embodiment, the biomarker is an mRNA of ZMYM198. In another specific embodiment, the biomarker is an mRNA of FAM83H. In another specific embodiment, the biomarker is an mRNA of KLC4. In another embodiment, the biomarker is an mRNA of EHMT2. In another embodiment, the biomarker is an mRNA of GLRX. In another embodiment, the biomarker is an mRNA of SDSL. In another specific embodiment, the biomarker is an mRNA of LIG3. In another specific embodiment, the biomarker is an mRNA of IKBKE. In another specific embodiment, the biomarker is an mRNA of PDE6D. In another specific embodiment, the biomarker is an mRNA of LGALS2. In another specific embodiment, the biomarker is an mRNA of LGALS9. In another specific embodiment, the biomarker is an mRNA of GSPT1. In another specific embodiment, the biomarker is an mRNA of CK1-alpha.

In some embodiments, the biomarker is a cDNA of AHNAK, ALOX5, AMPD3, ANXA4, ANXA6, ARHGAP19, ASNS, ASPM, ATP2B4, B4GALT3, BANK1, BCDIN3D, BLZF1, BMF, BST2, C10orf76, C19orf66, CA2, CA8, CAMSAP3, CCDC69, CCNB1, CD36, CDC7, CDCA3, CENPF, CLN3, CNN3, CORO1B, CPNE2, CRBN, CSNK1A1, CSRP2, CTNND1, CTSH, DAPK2, DDX58, DHPS, DHX58, DLG2, DLGAP5, DOK3, DTX3L, ECT2, EFCAB4B, EHMT1, EHMT2, EIF2AK2, EPB41L1, EPCAM, ESRP1, ETV6, EXTL2, F13A1, FAM195A, FAM65B, FBRSL1, FCGR2B, FES, FHOD1, FIGNL1, FMNL3, GBP1, GMFG, GMPR, GPT2, GRAMD1A, GRAMD1B, GRPEL2, HIP1, HJURP, HLA-B, HLA-DMA, HMCES, HMMR, HOXC4, HPSE, ICAM2, ID3, IFI35, IFIH1, IFIT1, IFIT3, IFIT5, IFITM2, IKZF1, IKZF3, IL4I1, IRF7, IRF9, IRS2, ISG15, ISG20, ITGB7, JAK3, KIF18B, K1F22, KIF2C, LAP3, LGALS1, LGALS3BP, LIMD1, LIPG, LPXN, MAN2A2, MARCKS, MFI2, MGARP, MINA, MIS18BP1, MOV10, MPP7, MUC1, MX1, MX2, MYO1G, NCF2, NEIL1, NFKBID, NME3, NMI, NPIPB5, NT5C3A, OAS1, OAS2, OAS3, OMA1, ORC6, PARP14, PARP9, PARVB, PBK, PBXIP1, PDE6D, PKMYT1, PLD4, PLEKHO1, PLK1, PLSCR1, PLXNB2, PODXL, PODXL2, POLE2, POMP, PPFIBP1, PRDM15, PRNP, PTAFR, PTMS, PTTG1, PYROXD1, QPRT, RAB13, RASA4B, RASSF6, RCN1, RGCC, RGS1, RGS2, RNF213, S100A13, SAMD9L, SAMHD1, SEC14L1, SERPINH1, SGOL1, SGOL2, SLCO3A1, SLCO4A1, SLFN11, SLFN13, SLFN5, SP110, SP140, SPN, SPR, STAP1, STAT1, STAT2, TACC3, TAP1, TAX1BP3, THEMIS2, THTPA, TIMM8B, TNFAIP8L2, TNFSF8, TOP2A, TP53I3, TPX2, TREX1, TRIB3, TRIM22, TTC39C, TXNIP, UBA7, UBE2L6, USP41, VCL, VNN2, WIZ, WSB1, WWC1, ZBTB38, ZFP91, ZMYM2, ZNF385B, ZNF581 or ZNF644, or a combination thereof.

In other embodiments, the biomarker is a cDNA of AHNAK, ALOX5, AMPD3, ANXA4, ANXA6, ATP2B4, BMF, BST2, C10orf76, C19orf66, CD36, CLN3, CNN3, CORO1B, CPNE2, CRBN, CSRP2, CTNND1, CTSH, DAPK2, DDX58, DHX58, DLG2, DTX3L, EIF2AK2, EPB41L1, ETV6, EXTL2, F13A1, FAM65B, FCGR2B, FES, FMNL3, GBP1, GMFG, GMPR, HIP1, HLA-B, HLA-DMA, HPSE, ID3, IF135, IFIH1, IFIT1, IFIT3, IFIT5, IFITM2, IL4I1, IRF7, IRF9, ISG15, ISG20, ITGB7, JAK3, LAP3, LGALS1, LGALS3BP, LIMD1, MAN2A2, MARCKS, MFI2, MGARP, MOV10, MPP7, MUC1, MX1, MX2, MYO1G, NCF2, NME3, NMI, NT5C3A, OAS1, OAS2, OAS3, PARP14, PARP9, PBXIP1, PLD4, PLEKHO1, PLSCR1, PLXNB2, POMP, PPFIBP1, PTMS, QPRT, RAB13, RCN1, RGCC, RNF213, S100A13, SAMD9L, SAMHD1, SERPINH1, SLFN11, SLFN13, SLFN5, SP110, SP140, SPN, SPR, STAP1, STAT1, STAT2, TAP1, TAX1BP3, THEMIS2, THTPA, TNFAIP8L2, TNFSF8, TP53I3, TREX1, TRIM22, TTC39C, TXNIP, UBA7, UBE2L6, USP41, VCL, VNN2 and ZBTB38, or a combination thereof

In yet other embodiments, the biomarker is a cDNA of ARHGAP19, ASNS, ASPM, B4GALT3, BANK1, BCDIN3D, BLZF1, CA2, CA8, CAMSAP3, CCDC69, CCNB1, CDC7, CDCA3, CENPF, CSNK1A1DHPS, DLGAP5, DOK3, ECT2, EFCAB4B, EHMT1, EHMT2, EPCAM, ESRP1, FAM195A, FBRSL1, FHOD1, FIGNL1, GPT2, GRAMD1A, GRAMD1B, GRPEL2, HJURP, HMCES, HMMR, HOXC4, ICAM2, IKZF1, IKZF3, IRS2, KIF18B, KIF22, KIF2C, LIPG, LPXN, MINA, MIS18BP1, NEIL1, NFKBID, NPIPB5, OMA1, ORC6, PARVB, PBK, PDE6D, PKMYT1, PLK1, PODXL, PODXL2, POLE2, PRDM15, PRNP, PTAFR, PTTG1, PYROXD1, RASA4B, RASSF6, RGS1, RGS2, SEC14L1, SGOL1, SGOL2, SLCO3A1, SLCO4A1, TACC3, TIMM8B, TOP2A, TPX2, TRIB3, WIZ, WSB1, WWC1, ZFP91, ZMYM2, ZNF385B, ZNF581 and ZNF644, or a combination thereof.

In yet other embodiments, the biomarker is a cDNA of SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTU1, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770, or a combination thereof.

In yet other embodiments, the biomarker is a cDNA of MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDC38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLII, FNBP1L, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830, or a combination thereof.

In yet other embodiments, the biomarker is a cDNA of ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Tap, Igf2r, IGFBP1, IGFBP3, JAG1, IKBKE, KIAA0100, KIAA1462, KIF20B, KIF22, KLC2, KRT9, LDLR, LPXN, MAFF, MELK, MET, MGAT4B, MGLL, MMP7, MST1R, MT2A, MVP, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2, or a combination thereof.

In yet other embodiments, the biomarker is a cDNA of ACSL6, AHSG, ALB, ARPP19, ATP5EP2, ATP5I, ATP5J, AZGP1, BCKDK, BLOC1S3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1H1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBC1D12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2, or a combination thereof.

In a specific embodiment, the biomarker is a cDNA of ZFP91. In another specific embodiment, the biomarker is a cDNA of CRBN. In certain embodiments, the biomarker is a cDNA of Nestin, KAT1/CCBL1, WIBG, MVP, PARP4, ZFP91, ZNF198, or a combination thereof. In certain embodiments, the biomarker is a cDNA of Nestin, KAT1/CCBL1, and WIBG. In certain embodiments, the biomarker is a cDNA of MVP, PARP4, ZFP91, and ZNF198. In another specific embodiment, the biomarker is a cDNA of Nestin. In another specific embodiment, the biomarker is a cDNA of KAT1/CCBL1. In another specific embodiment, the biomarker is a cDNA of WIBG. In another specific embodiment, the biomarker is a cDNA of MVP. In another specific embodiment, the biomarker is a cDNA of PARP4. In another specific embodiment, the biomarker is a cDNA of ZNF198. In another specific embodiment, the biomarker is a cDNA of MST1R (RON). In another specific embodiment, the biomarker is a cDNA of ZMYM198. In another specific embodiment, the biomarker is a cDNA of FAM83H. In another specific embodiment, the biomarker is a cDNA of KLC4. In another embodiment, the biomarker is a cDNA of EHMT2. In another embodiment, the biomarker is a cDNA of GLRX. In another embodiment, the biomarker is a cDNA of SDSL. In another specific embodiment, the biomarker is a cDNA of LIG3. In another specific embodiment, the biomarker is a cDNA of IKBKE. In another specific embodiment, the biomarker is a cDNA of PDE6D. In another specific embodiment, the biomarker is a cDNA of LGALS2. In another specific embodiment, the biomarker is a cDNA of LGALS9. In another specific embodiment, the biomarker is a cDNA of GSPT1. In another specific embodiment, the biomarker is a cDNA of CK1-alpha.

In some embodiments, the level of the biomarker provided herein correlates with or is indicative of the responsiveness of a disease (e.g., HCC or GBM) to a treatment, e.g., a treatment by a treatment compound, e.g., thalidomide, lenalidomide, pomalidomide, Compound A, or Compound B, or a stereoisomer thereof, or a pharmaceutically acceptable salt, solvate, hydrate, co-crystal, clathrate, or a polymorph thereof.

In some embodiments, the biomarker is an mRNA. In other embodiments, the biomarker is a cDNA. The level of the biomarker can be determined using the methods provided herein.

In other embodiments, the biomarker is a protein. When a biomarker is a polypeptide, protein, or peptide, the level of the biomarker can be measured by determining the protein level, the mRNA level, or the enzymatic activity of the biomarker. The level of the biomarker can be determined using the methods provided herein.

The reference level can be determined by a plurality of methods. In some embodiments, the reference level is one that a treatment decision is made based on whether a subject having or suspected of having a solid tumor, e.g., HCC or GBM, has the level of the biomarker above the reference level. Subjects who have a level of the biomarker higher than the reference level have a different probability of responsiveness to the treatment than subjects who have a level of the biomarker lower than the reference level. In certain embodiments, the reference level is measured simultaneously with the biological sample from the subject. In some embodiments, the reference level is predetermined.

In some embodiments, the reference level is determined from a sample from the same subject that contains no solid tumor cells. In other embodiments, the reference level is determined from a sample from a group of subjects that contains no solid tumor cells. In yet other embodiments, the reference level is determined from a sample from a group of subjects who do not have the solid tumor. An increased level or a decreased level of the biomarker correlates positively with increased responsiveness of the subject to a treatment by a treatment compound, e.g., thalidomide, lenalidomide, pomalidomide, Compound A, or Compound B, or a stereoisomer thereof, or a pharmaceutically acceptable salt, solvate, hydrate, co-crystal, clathrate, or a polymorph thereof.

In some embodiments, the control sample is a sample containing no solid tumor cells from the same subject. In other embodiments, the control sample is a sample of liver cells containing no solid tumor cells from the same subject. In other embodiments, the control sample is a sample containing no solid tumor cells from a group of subjects. In other embodiments, the control sample is a sample of liver cells containing no solid tumor cells from a group of subjects. In yet other embodiments, the control sample is a sample from a subject having no solid tumor. In yet other embodiments, the control sample is a sample of liver cells from a subject having no solid tumor. In yet other embodiments, the control sample is a sample from a group of subjects having no solid tumor. In yet other embodiments, the control sample is a sample of liver cells from a group of subjects having no solid tumor. An increased or a decreased level of the one or more biomarkers as compared with the level of the control sample correlates positively with increased responsiveness of the subject to a treatment by a treatment compound, e.g., thalidomide, lenalidomide, pomalidomide, Compound A, or Compound B, or a stereoisomer thereof, or a pharmaceutically acceptable salt, solvate, hydrate, co-crystal, clathrate, or a polymorph thereof.

In some embodiments, the reference is prepared by using a second tumor cell not treated with the compound. In other embodiments, the reference is prepared by using a second sample obtained from the subject prior to administration of the treatment compound to the patient; and wherein the second sample is from the same source as the sample. In yet other embodiments, the reference is prepared by using a second sample obtained from a healthy subject not having the solid tumor; and wherein the second sample is from the same source as the sample.

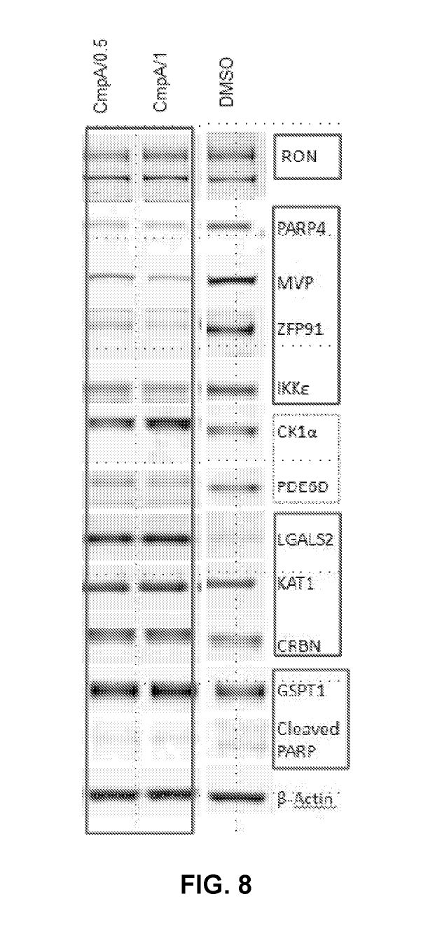

In some embodiments, the biomarkers provided herein are determined individually. In other embodiments, two or more of the biomarkers provided herein are determined simultaneously. For example, as shown in FIG. 8, the levels of MST1R (RON), PARP4, MVP, ZFP91, IKBKE, CK1-alpha, PDE6D, LGALS2, KAT1, CRBN, and GSPT1 change in response to Compound A treatment in one western blot analysis. Thus, in some embodiments, two or more biomarkers independently selected from the group consisting of MST1R (RON), PARP4, MVP, ZFP91, IKBKE, CK1-alpha, PDE6D, LGALS2, KAT1, CRBN, and GSPT1 are determined for selecting patients, predicting responsiveness or monitoring a patient's response.

In some embodiments, the level of a biomarker nucleic acid or polypeptide provided herein is measured in a biological sample from a subject, e.g., a HCC or a GBM cell containing-sample from the subject. In other embodiments, an affinity binding assay is used to measure the level of the biomarker polypeptide. The affinity binding assays that are applicable for use in the methods provided herein include both soluble and solid phase assays.

An example of a soluble phase affinity binding assay is immunoprecipitation using a biomarker binding agent, e.g., an antibody reactive with the biomarker polypeptide. Examples of solid phase affinity binding assays include immunohistochemical binding assays and immunoaffinity binding assays. Examples of immunoaffinity binding assays include, but are not limited to, immunohistochemistry methods, immunoblot methods, ELISA and radioimmunoassay (RIA).

An antibody useful in the methods provided herein includes a polyclonal and monoclonal antibodies. An antibody useful in the methods provided herein includes naturally occurring antibodies as well as non-naturally occurring antibodies, e.g., single chain antibodies, chimeric antibodies, bifunctional antibodies, humanized antibodies, and antigen-binding fragments thereof.

The biological sample can be liver tissue or a fluid such as blood, serum, or urine. In certain embodiments, the sample of cells from a subject is obtained via biopsy. Once a level of a biomarker is determined, this value can be correlated with clinical data on the subject from whom the sample is derived, e.g., the responsiveness of a subject to a given treatment.

In some embodiments, the sample of cells from a subject is obtained via biopsy.

In some embodiments, the level of only one of the biomarkers is monitored. In other embodiments, the levels of two or more of the biomarkers are monitored simultaneously.

4.3.1 Use of Biomarkers for Identifying a Subject for Treatment

Based, in part, on the finding that detectable increase or decrease in certain biomarkers are observed in subjects with a solid tumor, e.g., HCC or GBM, who are responsive to a given treatment (e.g., a treatment by a treatment compound, e.g., thalidomide, lenalidomide, pomalidomide, Compound A, or Compound B, or a stereoisomer thereof, or a pharmaceutically acceptable salt, solvate, hydrate, co-crystal, clathrate, or a polymorph thereof), the levels of these biomarkers may be used for identifying a subject having a solid tumor, e.g., HCC or GBM, for the treatment by a treatment compound provided herein.

In one aspect, provided herein is a method for identifying a subject having a solid tumor, e.g., HCC or GBM, who is likely to be responsive to a treatment compound.

In some embodiments, provided herein is a method of identifying a subject having a solid tumor who is likely to be responsive to a treatment compound, comprising:

(a) administering the treatment compound to a subject having a solid tumor;

(b) obtaining a sample from the subject;

(c) determining the level of a biomarker in the sample from the subject, wherein the biomarker is selected from the group consisting of biomarkers identified in Tables 1 to 6, and combinations thereof; and

(d) diagnosing the subject as being likely to be responsive to the treatment compound if the level of the biomarker in the sample of the subject changes as compared to a reference level of the biomarker. In one embodiment, the solid tumor is HCC. In another embodiment, the solid tumor is GBM.

In another aspect, provided herein is a method of identifying a subject having a solid tumor who is likely to be responsive to a treatment compound, comprising:

(a) administering the treatment compound to a subject having a solid tumor;

(b) obtaining a sample from the subject;

(c) determining the level of a biomarker in the sample from the subject, wherein the biomarker is selected from the group consisting of biomarkers identified in Table 1, Table 3, and Table 4, and combinations thereof; and

(d) diagnosing the subject as being likely to be responsive to the treatment compound if the level of the biomarker in the sample of the subject is higher than a reference level of the biomarker. In one embodiment, the solid tumor is HCC. In another embodiment, the solid tumor is GBM.

In another aspect, provided herein is a method of identifying a subject having a solid tumor, e.g., HCC or GBM, who is likely to be responsive to a treatment compound, comprising:

(a) administering the treatment compound to a subject having a solid tumor;

(b) obtaining a sample from the subject;

(c) determining the level of a biomarker in the sample from the subject, wherein the biomarker is selected from the group consisting of biomarkers identified in Table 2, Table 5, and Table 6, and combinations thereof; and

(d) diagnosing the subject as being likely to be responsive to the treatment compound if the level of the biomarker in the sample of the subject is lower than a reference level of the biomarker. In one embodiment, the solid tumor is HCC. In another embodiment, the solid tumor is GBM.

In certain embodiments, the methods provided herein are coupled with a treatment by a treatment compound provided herein, e.g., thalidomide, lenalidomide, pomalidomide, Compound A, or Compound B, or a stereoisomer thereof, or a pharmaceutically acceptable salt, solvate, hydrate, co-crystal, clathrate, or a polymorph thereof.

Thus, in another aspect, provided herein is a method of treating a solid tumor, e.g., HCC or GBM. In some embodiments, provided herein is a method of treating a solid tumor, comprising:

(a) obtaining a sample from the subject;

(b) determining the level of a biomarker in the sample from the subject, wherein the biomarker is selected from the group consisting of biomarkers identified in Tables 1 to 6, and combinations thereof.

(c) diagnosing the subject as being likely to be responsive to a treatment compound if the level of the biomarker in the sample of the subject changes as compared to a reference level of the biomarker; and

(d) administering a therapeutically effective amount of the treatment compound to the subject diagnosed to be likely to be responsive to the treatment compound. In one embodiment, the solid tumor is HCC. In another embodiment, the solid tumor is GBM.

In another aspect, provided herein is a method of treating a solid tumor, e.g., HCC or GBM, comprising:

(a) obtaining a sample from the subject;

(b) determining the level of a biomarker in the sample from the subject, wherein the biomarker is selected from the group consisting of biomarkers identified in Table 1, Table 3, and Table 4, and combinations thereof.

(c) diagnosing the subject as being likely to be responsive to a treatment compound if the level of the biomarker in the sample of the subject is higher as than a reference level of the biomarker; and

(d) administering a therapeutically effective amount of the treatment compound to the subject diagnosed to be likely to be responsive to the treatment compound. In one embodiment, the solid tumor is HCC. In another embodiment, the solid tumor is GBM.

In another aspect, provided herein is a method of treating a solid tumor, e.g., HCC or GBM, comprising:

(a) obtaining a sample from the subject;

(b) determining the level of a biomarker in the sample from the subject, wherein the biomarker is selected from the group consisting of biomarkers identified in Table 2, Table 5, and Table 6, and combinations thereof;

(c) diagnosing the subject as being likely to be responsive to a treatment compound if the level of the biomarker in the sample of the subject is lower as than a reference level of the biomarker; and

(d) administering a therapeutically effective amount of the treatment compound to the subject diagnosed to be likely to be responsive to the treatment compound. In one embodiment, the solid tumor is HCC. In another embodiment, the solid tumor is GBM.

In some embodiments of the various methods provided herein, the biomarker is selected from a group consisting of CRBN, CK1-alpha, GSPT1, Nestin, KAT1/CCBL1, WIBG, MVP, PARP4, ZFP91, and ZNF198. In some embodiments of the various methods provided herein, the biomarker is selected from a group consisting of CRBN, CK1-alpha, GSPT1, Nestin, KAT1/CCBL1, and WIBG. In other embodiments, the biomarker is selected from a group consisting of MVP, PARP4, ZFP91, and ZNF198.

In some embodiments of the various methods provided herein, the biomarker is selected from a group consisting of AHNAK, ALOX5, AMPD3, ANXA4, ANXA6, ARHGAP19, ASNS, ASPM, ATP2B4, B4GALT3, BANK1, BCDIN3D, BLZF1, BMF, BST2, C10orf76, C19orf66, CA2, CA8, CAMSAP3, CCDC69, CCNB1, CD36, CDC7, CDCA3, CENPF, CLN3, CNN3, CORO1B, CPNE2, CRBN, CSNK1A1, CSRP2, CTNND1, CTSH, DAPK2, DDX58, DHPS, DHX58, DLG2, DLGAP5, DOK3, DTX3L, ECT2, EFCAB4B, EHMT1, EHMT2, EIF2AK2, EPB41L1, EPCAM, ESRP1, ETV6, EXTL2, F13A1, FAM195A, FAM65B, FBRSL1, FCGR2B, FES, FHOD1, FIGNL1, FMNL3, GBP1, GMFG, GMPR, GPT2, GRAMD1A, GRAMD1B, GRPEL2, HIP1, HJURP, HLA-B, HLA-DMA, HMCES, HMMR, HOXC4, HPSE, ICAM2, ID3, IFI35, IFIH1, IFIT1, IFIT3, IFIT5, IFITM2, IKZF1, IKZF3, IL4I1, IRF7, IRF9, IRS2, ISG15, ISG20, ITGB7, JAK3, KIF18B, KIF22, KIF2C, LAP3, LGALS1, LGALS3BP, LIMD1, LIPG, LPXN, MAN2A2, MARCKS, MFI2, MGARP, MINA, MIS18BP1, MOV10, MPP7, MUC1, MX1, MX2, MYO1G, NCF2, NEIL1, NFKBID, NME3, NMI, NPIPB5, NT5C3A, OAS1, OAS2, OAS3, OMA1, ORC6, PARP14, PARP9, PARVB, PBK, PBXIP1, PDE6D, PKMYT1, PLD4, PLEKHO1, PLK1, PLSCR1, PLXNB2, PODXL, PODXL2, POLE2, POMP, PPFIBP1, PRDM15, PRNP, PTAFR, PTMS, PTTG1, PYROXD1, QPRT, RAB13, RASA4B, RASSF6, RCN1, RGCC, RGS1, RGS2, RNF213, S100A13, SAMD9L, SAMHD1, SEC14L1, SERPINH1, SGOL1, SGOL2, SLCO3A1, SLCO4A1, SLFN11, SLFN13, SLFN5, SP110, SP140, SPN, SPR, STAP1, STAT1, STAT2, TACC3, TAP1, TAX1BP3, THEMIS2, THTPA, TIMM8B, TNFAIP8L2, TNFSF8, TOP2A, TP53I3, TPX2, TREX1, TRIB3, TRIM22, TTC39C, TXNIP, UBA7, UBE2L6, USP41, VCL, VNN2, WIZ, WSB1, WWC1, ZBTB38, ZFP91, ZMYM2, ZNF385B, ZNF581 and ZNF644.

In other embodiments of the various methods provided herein, the biomarker is selected from a group consisting of AHNAK, ALOX5, AMPD3, ANXA4, ANXA6, ATP2B4, BMF, BST2, C10orf76, C19orf66, CD36, CLN3, CNN3, CORO1B, CPNE2, CRBN, CSRP2, CTNND1, CTSH, DAPK2, DDX58, DHX58, DLG2, DTX3L, EIF2AK2, EPB41L1, ETV6, EXTL2, F13A1, FAM65B, FCGR2B, FES, FMNL3, GBP1, GMFG, GMPR, HIP1, HLA-B, HLA-DMA, HPSE, ID3, IFI35, IFIH1, IFIT1, IFIT3, IFIT5, IFITM2, IL4I1, IRF7, IRF9, ISG15, ISG20, ITGB7, JAK3, LAP3, LGALS1, LGALS3BP, LIMD1, MAN2A2, MARCKS, MFI2, MGARP, MOV10, MPP7, MUC1, MX1, MX2, MYO1G, NCF2, NME3, NMI, NT5C3A, OAS1, OAS2, OAS3, PARP14, PARP9, PBXIP1, PLD4, PLEKHO1, PLSCR1, PLXNB2, POMP, PPFIBP1, PTMS, QPRT, RAB13, RCN1, RGCC, RNF213, S100A13, SAMD9L, SAMHD1, SERPINH1, SLFN11, SLFN13, SLFN5, SP110, SP140, SPN, SPR, STAP1, STAT1, STAT2, TAP1, TAX1BP3, THEMIS2, THTPA, TNFAIP8L2, TNFSF8, TP53I3, TREX1, TRIM22, TTC39C, TXNIP, UBA7, UBE2L6, USP41, VCL, VNN2 and ZBTB38.

In yet other embodiments of the various methods provided herein, the biomarker is selected from a group consisting of ARHGAP19, ASNS, ASPM, B4GALT3, BANK1, BCDIN3D, BLZF1, CA2, CA8, CAMSAP3, CCDC69, CCNB1, CDC7, CDCA3, CENPF, CSNK1A1DHPS, DLGAP5, DOK3, ECT2, EFCAB4B, EHMT1, EHMT2, EPCAM, ESRP1, FAM195A, FBRSL1, FHOD1, FIGNL1, GPT2, GRAMD1A, GRAMD1B, GRPEL2, HJURP, HMCES, HMMR, HOXC4, ICAM2, IKZF1, IKZF3, IRS2, KIF18B, KIF22, KIF2C, LIPG, LPXN, MINA, MIS18BP1, NEIL1, NFKBID, NPIPB5, OMA1, ORC6, PARVB, PBK, PDE6D, PKMYT1, PLK1, PODXL, PODXL2, POLE2, PRDM15, PRNP, PTAFR, PTTG1, PYROXD1, RASA4B, RASSF6, RGS1, RGS2, SEC14L1, SGOL1, SGOL2, SLCO3A1, SLCO4A1, TACC3, TIMM8B, TOP2A, TPX2, TRIB3, WIZ, WSB1, WWC1, ZFP91, ZMYM2, ZNF385B, ZNF581 and ZNF644.

In yet other embodiments of the various methods provided herein, the biomarker is selected from a group consisting of SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTU1, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770.

In yet other embodiments of the various methods provided herein, the biomarker is selected from a group consisting of MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDC38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLIT, FNBP1L, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830.

In yet other embodiments of the various methods provided herein, the biomarker is selected from a group consisting of ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Tap, Igf2r, IGFBP1, IGFBP3, IKBKE, JAG1, KIAA0100, KIAA1462, KIF20B, KIF22, KLC2, KRT9, LDLR, LPXN, MAFF, MELK, MET, MGAT4B, MGLL, MMP7, MST1R, MT2A, MVP, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2.

In yet other embodiments of the various methods provided herein, the biomarker is selected from a group consisting of ACSL6, AHSG, ALB, ARPP19, ATP5EP2, ATP5I, ATP5J, AZGP1, BCKDK, BLOC1S3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1H1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBC1D12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2.

In certain embodiments, any combination of two or more of the above-identified biomarkers is also contemplated. For example, in some embodiments of the various methods provided herein, two or more biomarkers independently selected from the group consisting of MST1R (RON), PARP4, MVP, ZFP91, IKBKE, CK1-alpha, PDE6D, LGALS2, KAT1, CRBN, and GSPT1 are determined. In some specific embodiments of the various methods provided herein, ZFP91 and one or more additional biomarker selected from the group consisting of MST1R (RON), PARP4, MVP, IKBKE, CK1-alpha, PDE6D, LGALS2, KAT1, and GSPT1 are determined.

In some embodiments of the various methods provided herein, the biomarker is selected from a group consisting of FAM83H, KLC4, LIG3, IKBKE, LGALS2 and LGALS9, and the level of the biomarker increases in response to compound treatment, e.g., by Compound A. In some embodiments of the various methods provided herein, the biomarker is selected from a group consisting of ZMYM198, EHMT2, GLRX, SDSL, and PDE6D, and the level of the biomarker decreases in response to compound treatment, e.g., by Compound A. In a specific embodiment, the biomarker is ZMYM198. In another specific embodiment, the biomarker is FAM83H. In another specific embodiment, the biomarker is KLC4. In another embodiment, the biomarker is EHMT2. In another embodiment, the biomarker is GLRX. In another embodiment, the biomarker is SDSL. In another specific embodiment, the biomarker is LIG3. In another specific embodiment, the biomarker is IKBKE. In another specific embodiment, the biomarker is PDE6D. In another specific embodiment, the biomarker is LGALS2. In another specific embodiment, the biomarker is LGALS9.

In a specific embodiment of the various methods provided herein, the biomarker is ZFP91. In another embodiment of the various methods provided herein, the biomarker is CRBN. In yet another embodiment of the various methods provided herein, the biomarkers are both ZFP91 and CRBN. In another specific embodiment of the various methods provided herein, the biomarker is Nestin. In another specific embodiment of the various methods provided herein, the biomarker is KAT1/CCBL1. In another specific embodiment of the various methods provided herein, the biomarker is WIBG. In another specific embodiment of the various methods provided herein, the biomarker is MVP. In yet another specific embodiment of the various methods provided herein, the biomarker is PARP4. In yet another specific embodiment of the various methods provided herein, the biomarker is ZNF198. In yet another specific embodiment, the biomarker is CK1-alpha. In yet another specific embodiment, the biomarker is GSPT1.

In some embodiments of the various methods provided herein, the level of only one biomarker is determined. In other embodiments of the various methods provided herein, the levels of two, three, four, five or more biomarkers are determined.

In some embodiments of the various methods provided herein, two or more biomarkers identified in Table 1, Table 2, Table 3, Table 4, Table 5, and/or Table 6 are measured and compared with the reference sample. In some embodiments, the two or more biomarkers are identified in Table 1, Table 3, and/or Table 4, and the levels of the biomarkers increase as compared to the levels of the biomarkers in the reference. In other embodiments of the various methods provided herein, the two or more biomarkers are identified in Table 2, Table 5, and/or Table 6, and the levels of the biomarkers decrease as compared to the levels of the biomarkers in the reference. In yet other embodiments of the various methods provided herein, one or more biomarkers are identified in Table 1, Table 3, and/or Table 4, and one or more biomarkers are identified in Table 2, Table 5, and/or Table 6, and the levels of some biomarkers increase as compared to the reference and the level of some biomarkers decrease as compared to the reference.

In some embodiments of the various methods provided herein, the reference is prepared by using a control sample obtained from the subject prior to administration of the treatment compound to the subject; and wherein the control sample is from the same source as the sample. In other embodiments of the various methods provided herein, the reference is prepared by using a control sample obtained from a healthy subject not having the solid tumor; and wherein the control sample is from the same source as the sample.

In some embodiments of the various methods provided herein, the levels of one or more of the biomarkers are measured by determining the mRNA levels of the biomarkers. In some embodiments, the mRNA levels of the biomarkers are determined by reverse transcriptase PCR (RT-PCR). In some embodiments, the mRNA levels of the biomarkers are determined by quantitative RT-PCR (qRT-PCR).

In other embodiments of the various methods provided herein, the levels of one or more of the biomarkers are measured by determining the cDNA levels of the biomarkers. In some embodiments of the various methods provided herein, the cDNA levels of the biomarkers are determined by PCR.

In yet other embodiments of the various methods provided herein, the levels of one or more of the biomarkers are measured by determining the protein levels of the biomarkers.

In some embodiments of the various methods provided herein, the method provided herein further comprises contacting proteins within the sample with a first antibody that immunospecifically binds to the biomarker protein.

In one embodiment, the method provided herein further comprises (i) contacting the proteins bound to the first antibody with a second antibody with a detectable label, wherein the second antibody immunospecifically binds to the biomarker protein, and wherein the second antibody immunospecifically binds to a different epitope on the biomarker protein than the first antibody; (ii) detecting the presence of second antibody bound to the biomarker protein; and (iii) determining the amount of the biomarker protein based on the amount of detectable label in the second antibody.

In another embodiment, the method provided herein further comprises (i) contacting the proteins bound to the first antibody with a second antibody with a detectable label, wherein the second antibody immunospecifically binds to the first antibody; (ii) detecting the presence of second antibody bound to the first antibody; and (iii) determining the amount of the biomarker protein based on the amount of detectable label in the second antibody.

In a specific embodiment of the various methods provided herein, the biomarker is ZFP91. Thus, in some embodiments, the method provided herein comprises selecting a group of subjects having a solid tumor, e.g., HCC or GBM, based on the level of ZFP91, or the levels of ZFP91 expression within the solid tumor, e.g., HCC or GBM, for the purposes of predicting clinical response, monitoring clinical response, or monitoring patient compliance to dosing by a compound. As shown in Examples, ZFP91 protein degrades in response to treatment with various compounds. Thus, a reduced level of ZFP91 can be used to identify subjects who are likely to be responsive to treatment with various compounds and/or to predict if further treatment with compounds will receive responsiveness from the subject.

In some embodiments, provided herein is a method of identifying a patient who is likely to be responsive to a treatment of a solid tumor, e.g., HCC or GBM, with a treatment compound, comprising:

(a) administering a treatment compound to a subject having a solid tumor;

(b) obtaining a sample from the subject;

(c) determining the level of ZFP91 in the sample; and

(d) diagnosing the subject as being likely to be responsive to a treatment of the solid tumor with the treatment compound if the level of ZFP91 in the sample is less than the level of ZFP91 obtained from a reference sample. In one embodiment, the solid tumor is HCC. In another embodiment, the solid tumor is GBM.

In some embodiments, provided herein is a method of treating a solid tumor, comprising:

(a) obtaining a sample from the subject;

(b) determining the level of ZFP91;

(c) diagnosing the subject as being likely to be responsive to a treatment compound if the level of ZFP91 in the sample of the subject is less as than a reference level of ZFP91; and

(d) administering a therapeutically effective amount of the treatment compound to the subject diagnosed to be likely to be responsive to the treatment compound. In one embodiment, the solid tumor is HCC. In another embodiment, the solid tumor is GBM.

In some embodiments, the level of ZFP91 in the sample is less than 90% of the level of ZFP91 of a reference. In some embodiments, the level of ZFP91 in the sample is less than 80% of the level of ZFP91 of a reference. In other embodiments, the level of ZFP91 in the sample is less than 70% of the level of ZFP91 of a reference. In other embodiments, the level of ZFP91 in the sample is less than 60% of the level of ZFP91 of a reference. In other embodiments, the level of ZFP91 in the sample is less than 50% of the level of ZFP91 of a reference. In other embodiments, the level of ZFP91 in the sample is less than 40% of the level of ZFP91 of a reference. In yet other embodiments, the level of ZFP91 in the sample is less than 30% of the level of ZFP91 of a reference. In yet other embodiments, the level of ZFP91 in the sample is less than 20% of the level of ZFP91 of a reference. In yet other embodiments, the level of ZFP91 in the sample is less than 10% of the level of ZFP91 of a reference.

In some embodiments, the protein level of ZFP91 is measured. For example, in some embodiments, the method provided herein comprises contacting proteins within the sample with a first antibody that immunospecifically binds to ZFP91 protein. In some embodiments, the method provided herein further includes (i) contacting the proteins bound to the first antibody with a second antibody with a detectable label, wherein the second antibody immunospecifically binds to ZFP91, and wherein the second antibody immunospecifically binds to a different epitope on ZFP91protein than the first antibody; (ii) detecting the presence of second antibody bound to the proteins; and (iii) determining the amount of ZFP91 protein based on the amount of detectable label in the second antibody. In other embodiments, the method provided herein further comprises (i) contacting the proteins bound to the first antibody with a second antibody with a detectable label, wherein the second antibody immunospecifically binds to the first antibody; (ii) detecting the presence of second antibody bound to the proteins; and (iii) determining the amount of ZFP91 protein based on the amount of detectable label in the second antibody.

In some embodiments, the mRNA level of ZFP91 is measured. For example, in some embodiments, the method provided herein comprises extracting mRNA from the sample. In some embodiments, the method further comprises determining mRNA level of ZFP91 using PCR. In some embodiments, the PCR is a RT-PCR.

In some embodiments, the method provided herein further comprises generating cDNA from the mRNA. In some embodiments, the method provided herein further comprises performing a PCR to quantify the cDNA representing ZFP91.

In some embodiments, the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide. In some embodiments, the solid tumor is HCC. In some embodiments, the solid tumor is GBM. In some embodiments, the treatment compound is Compound A, and the solid tumor is HCC. In some embodiments, the treatment compound is Compound B, and the solid tumor is HCC. In some embodiments, the treatment compound is lenadlidomide, and the solid tumor is HCC. In some embodiments, the treatment compound is Compound A, and the solid tumor is GBM. In some embodiments, the treatment compound is Compound B, and the solid tumor is GBM. In some embodiments, the treatment compound is lenadlidomide, and the solid tumor is GBM.

In a specific embodiment of the various methods provided herein, the biomarker is CRBN. Thus, in some embodiments, the method provided herein comprises selecting a group of subjects having a solid tumor, e.g., HCC or GBM, based on the level of CRBN, or the levels of CRBN expression within the solid tumor, e.g., HCC or GBM, for the purposes of predicting clinical response, monitoring clinical response, or monitoring patient compliance to dosing by a compound. As shown in Examples, CRBN protein level increases in response to treatment with various compounds in HCC or GBM cell lines. Thus, an increased level of CRBN can be used to identify subjects who are likely to be responsive to treatment with various compounds and/or to predict if further treatment with compounds will receive responsiveness from the subjects.

In some embodiments, provided herein is a method of identifying a patient who is likely to be responsive to a treatment of a solid tumor with a treatment compound, comprising:

(a) administering a treatment compound to a subject having a solid tumor;

(b) obtaining a sample from the subject;

(c) determining the level of CRBN in the sample; and

(d) diagnosing the subject as being likely to be responsive to a treatment of the solid tumor with the treatment compound if the level of CRBN in the sample is higher than the level of CRBN obtained from a reference sample. In one embodiment, the solid tumor is HCC. In another embodiment, the solid tumor is GBM.

In some embodiments, provided herein is a method of treating a solid tumor, comprising:

(a) obtaining a sample from the subject;

(b) determining the level of CRBN;

(c) diagnosing the subject as being likely to be responsive to a treatment compound if the level of CRBN in the sample of the subject is higher than a reference level of CRBN; and

(d) administering a therapeutically effective amount of the treatment compound to the subject diagnosed to be likely to be responsive to the treatment compound. In one embodiment, the solid tumor is HCC. In another embodiment, the solid tumor is GBM.

In some embodiments, the level of CRBN in the sample is 10% more than the level of CRBN of a reference. In some embodiments, the level of ZFP91 in the sample is 20% more than the level of CRBN of a reference. In other embodiments, the level of CRBN in the sample is 30% more than the level of CRBN of a reference. In other embodiments, the level of CRBN in the sample is 40% more than the level of CRBN of a reference. In other embodiments, the level of CRBN in the sample is 50% more than the level of CRBN of a reference. In other embodiments, the level of CRBN in the sample is 60% more than the level of CRBN of a reference. In yet other embodiments, the level of CRBN in the sample is 70% more than the level of CRBN of a reference. In yet other embodiments, the level of CRBN in the sample is 80% of the level of CRBN of a reference. In yet other embodiments, the level of CRBN in the sample is 90% more than the level of CRBN of a reference. In yet other embodiments, the level of CRBN in the sample is 2-fold, 3-fold, 4-fold, 5-fold, 6-fold, 7-fold, 8-fold, 9-fold, 10-fold of the CRBN level of a reference.

In some embodiments, the protein level of CRBN is measured. For example, in some embodiments, the method provided herein comprises contacting proteins within the sample with a first antibody that immunospecifically binds to CRBN protein. In some embodiments, the method provided herein further includes (i) contacting the proteins bound to the first antibody with a second antibody with a detectable label, wherein the second antibody immunospecifically binds to CRBN, and wherein the second antibody immunospecifically binds to a different epitope on CRBN protein than the first antibody; (ii) detecting the presence of second antibody bound to the proteins; and (iii) determining the amount of CRBN protein based on the amount of detectable label in the second antibody. In other embodiments, the method provided herein further comprises (i) contacting the proteins bound to the first antibody with a second antibody with a detectable label, wherein the second antibody immunospecifically binds to the first antibody; (ii) detecting the presence of second antibody bound to the proteins; and (iii) determining the amount of CRBN protein based on the amount of detectable label in the second antibody.

In some embodiments, the mRNA level of CRBN is measured. For example, in some embodiments, the method provided herein comprises extracting mRNA from the sample. In some embodiments, the method further comprises determining mRNA level of CRBN using PCR. In some embodiments, the PCR is a RT-PCR.

In some embodiments, the method provided herein further comprises generating cDNA from the mRNA. In some embodiments, the method provided herein further comprises performing a PCR to quantify the cDNA representing CRBN.

In some embodiments, the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide. In some embodiments, the solid tumor is HCC. In some embodiments, the solid tumor is GBM. In some embodiments, the treatment compound is Compound A, and the solid tumor is HCC. In some embodiments, the treatment compound is Compound B, and the solid tumor is HCC. In some embodiments, the treatment compound is lenadlidomide, and the solid tumor is HCC. In some embodiments, the treatment compound is Compound A, and the solid tumor is GBM. In some embodiments, the treatment compound is Compound B, and the solid tumor is GBM. In some embodiments, the treatment compound is lenadlidomide, and the solid tumor is GBM.

In a specific embodiment of the various methods provided herein, the biomarkers are ZFP91 and CRBN. Thus, in some embodiments, the method provided herein comprises selecting a group of subjects having a solid tumor, e.g., HCC or GBM, based on the levels of ZFP91 and CRBN, or the levels of ZFP91 and CRBN expression within the solid tumor, e.g., HCC or GBM, for the purposes of predicting clinical response, monitoring clinical response, or monitoring patient compliance to dosing by a compound. In some embodiments, an increased level of CRBN and a decreased level of ZFP91 can be used to identify subjects who are likely to be responsive to treatment with various compounds and/or to predict if further treatment with compounds will receive responsiveness from the subjects.

In some embodiments, provided herein is a method of identifying a subject who is likely to be responsive to a treatment of a solid tumor, e.g., HCC or GBM, with a treatment compound, comprising:

(a) administering a treatment compound to a subject having a solid tumor;

(b) obtaining a sample from the subject;

(c) determining the levels of ZFP91 and CRBN in the sample; and

(d) diagnosing the subject as being likely to be responsive to a treatment of the solid tumor with the treatment compound if the level of ZFP91 in the sample is lower than the level of ZFP91 obtained from a reference sample and the level of CRBN in the sample is higher than the level of CRBN obtained from the reference sample.

In some embodiments, provided herein is a method of treating a solid tumor, e.g., HCC or GBM, comprising:

(a) obtaining a sample from the subject;

(b) determining the levels of ZFP91 and CRBN in the sample;

(c) diagnosing the subject as being likely to be responsive to a treatment compound if the level of ZFP91 in the sample is lower than the level of ZFP91 obtained from a reference sample and the level of CRBN in the sample is higher than the level of CRBN obtained from the reference sample; and

(d) administering a therapeutically effective amount of the treatment compound to the subject diagnosed to be likely to be responsive to the treatment compound.

In some embodiments, the level of ZFP91 in the sample is less than 90% of the level of ZFP91 of a reference. In some embodiments, the level of ZFP91 in the sample is less than 80% of the level of ZFP91 of a reference. In other embodiments, the level of ZFP91 in the sample is less than 70% of the level of ZFP91 of a reference. In other embodiments, the level of ZFP91 in the sample is less than 60% of the level of ZFP91 of a reference. In other embodiments, the level of ZFP91 in the sample is less than 50% of the level of ZFP91 of a reference. In other embodiments, the level of ZFP91 in the sample is less than 40% of the level of ZFP91 of a reference. In yet other embodiments, the level of ZFP91 in the sample is less than 30% of the level of ZFP91 of a reference. In yet other embodiments, the level of ZFP91 in the sample is less than 20% of the level of ZFP91 of a reference. In yet other embodiments, the level of ZFP91 in the sample is less than 10% of the level of ZFP91 of a reference.

In some embodiments, the level of CRBN in the sample is 10% more than the level of CRBN of a reference. In some embodiments, the level of ZFP91 in the sample is 20% more than the level of CRBN of a reference. In other embodiments, the level of CRBN in the sample is 30% more than the level of CRBN of a reference. In other embodiments, the level of CRBN in the sample is 40% more than the level of CRBN of a reference. In other embodiments, the level of CRBN in the sample is 50% more than the level of CRBN of a reference. In other embodiments, the level of CRBN in the sample is 60% more than the level of CRBN of a reference. In yet other embodiments, the level of CRBN in the sample is 70% more than the level of CRBN of a reference. In yet other embodiments, the level of CRBN in the sample is 80% of the level of CRBN of a reference. In yet other embodiments, the level of CRBN in the sample is 90% more than the level of CRBN of a reference. In yet other embodiments, the level of CRBN in the sample is 2-fold, 3-fold, 4-fold, 5-fold, 6-fold, 7-fold, 8-fold, 9-fold, 10-fold of the CRBN level of a reference.

In some embodiments, the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide. In some embodiments, the solid tumor is HCC. In some embodiments, the solid tumor is GBM. In some embodiments, the treatment compound is Compound A, and the solid tumor is HCC. In some embodiments, the treatment compound is Compound B, and the solid tumor is HCC. In some embodiments, the treatment compound is lenadlidomide, and the solid tumor is HCC. In some embodiments, the treatment compound is Compound A, and the solid tumor is GBM. In some embodiments, the treatment compound is Compound B, and the solid tumor is GBM. In some embodiments, the treatment compound is lenadlidomide, and the solid tumor is GBM.

In some embodiments, the treatment compound is thalidomide, or a stereoisomer thereof, or a pharmaceutically acceptable salt, solvate, hydrate, co-crystal, clathrate, or a polymorph thereof. In other embodiments, the treatment compound is lenalidomide, or a stereoisomer thereof, or a pharmaceutically acceptable salt, solvate, hydrate, co-crystal, clathrate, or a polymorph thereof. In yet other embodiments, the treatment compound is pomalidomide, or a stereoisomer thereof, or a pharmaceutically acceptable salt, solvate, hydrate, co-crystal, clathrate, or a polymorph thereof. In yet other embodiments, the treatment compound is Compound A, or a stereoisomer thereof, or a pharmaceutically acceptable salt, solvate, hydrate, co-crystal, clathrate, or a polymorph thereof. In yet other embodiments, the treatment compound is Compound B, or a stereoisomer thereof, or a pharmaceutically acceptable salt, solvate, hydrate, co-crystal, clathrate, or a polymorph thereof.

In some embodiments, the biomarkers are used to identify a subject having a solid tumor, e.g., HCC or GBM, who is likely to be responsive to treatment by thalidomide, lenalidomide, pomalidomide, Compound A or Compound B, or a stereoisomer thereof, or a pharmaceutically acceptable salt, solvate, hydrate, co-crystal, clathrate, or a polymorph thereof.

In some embodiments, the biomarkers are used to identify a subject having a solid tumor, e.g., HCC or GBM, who is likely to be responsive to treatment by thalidomide. In other embodiments, the biomarkers are used to identify a subject having a solid tumor, e.g., HCC or GBM, who is likely to be responsive to treatment by lenalidomide. In other embodiments, the biomarkers are used to identify a subject having a solid tumor, e.g., HCC or GBM, who is likely to be responsive to treatment by pomalidomide. In other embodiments, the biomarkers are used to identify a subject having a solid tumor, e.g., HCC or GBM, who is likely to be responsive to treatment by Compound A. In yet other embodiments, the biomarkers are used to identify a subject having a solid tumor, e.g., HCC or GBM, who is likely to be responsive to treatment by Compound B.

In some embodiments, provided herein is a method of identifying a subject who is likely to be responsive to a treatment of a solid tumor, e.g., HCC or GBM, with Compound A, comprising:

(a) administering Compound A to a subject having a solid tumor;

(b) obtaining a sample from the subject;

(c) determining the level of a biomarker in the sample from the subject, wherein the biomarker is selected from the group consisting of biomarkers identified in Tables 1 to 6, and combinations thereof; and

(d) diagnosing the subject as being likely to be responsive to Compound A if the level of the biomarker in the sample of the subject changes as compared to a reference level of the biomarker. In one embodiment, the solid tumor is HCC. In another embodiment, the solid tumor is GBM.

In some embodiments, provided herein is a method of treating a solid tumor, e.g., HCC or GBM, comprising:

(a) obtaining a sample from the subject;

(b) determining the level of a biomarker in the sample from the subject, wherein the biomarker is selected from the group consisting of biomarkers identified in Tables 1 to 6, and combinations thereof;

(c) diagnosing the subject as being likely to be responsive to Compound A if the level of the biomarker in the sample of the subject changes as compared to a reference level of the biomarker; and

(d) administering a therapeutically effective amount of Compound A to the subject diagnosed to be likely to be responsive to Compound A. In one embodiment, the solid tumor is HCC. In another embodiment, the solid tumor is GBM.

In some embodiments, the biomarker is selected from a group consisting of CRBN, CK1-alpha, GSPT1, Nestin, KAT1/CCBL1, WIBG, FAM83H, KLC4, LIG3, IKBKE, LGALS2 LGALS9, MVP, PARP4, ZFP91, ZNF198, MST1R (RON), ZMYM198, EHMT2, GLRX, SDSL, and PDE6D. In some embodiments of the various methods provided herein, the biomarker is selected from a group consisting of CRBN, CK1-alpha, GSPT1, Nestin, KAT1/CCBL1, WIBG, FAM83H, KLC4, LIG3, IKBKE, LGALS2 and LGALS9. In other embodiments, the biomarker is selected from a group consisting of MVP, PARP4, ZFP91, ZNF198, MST1R (RON), ZMYM198, EHMT2, GLRX, SDSL, and PDE6D. In a specific embodiment of the various methods provided herein, the biomarker is ZFP91. In another embodiment of the various methods provided herein, the biomarker is CRBN. In yet another embodiment of the various methods provided herein, the biomarkers are both ZFP91 and CRBN. In another specific embodiment of the various methods provided herein, the biomarker is CK1-alpha. In another specific embodiment of the various methods provided herein, the biomarker is GSPT1. In another specific embodiment of the various methods provided herein, the biomarker is MST1R (RON). In another specific embodiment of the various methods provided herein, the biomarker is Nestin. In another specific embodiment of the various methods provided herein, the biomarker is KAT1/CCBL1. In another specific embodiment of the various methods provided herein, the biomarker is WIBG. In another specific embodiment of the various methods provided herein, the biomarker is MVP. In yet another specific embodiment of the various methods provided herein, the biomarker is PARP4. In yet another specific embodiment of the various methods provided herein, the biomarker is ZNF198. In yet another specific embodiment of the various methods provided herein, the biomarker is ZMYM198. In yet another specific embodiment of the various methods provided herein, the biomarker is FAM83H. In yet another specific embodiment of the various methods provided herein, the biomarker is KLC4. In yet another specific embodiment of the various methods provided herein, the biomarker is EHMT2. In yet another specific embodiment of the various methods provided herein, the biomarker is GLRX. In yet another specific embodiment of the various methods provided herein, the biomarker is SDSL. In yet another specific embodiment of the various methods provided herein, the biomarker is LIG3. In yet another specific embodiment of the various methods provided herein, the biomarker is IKBKE. In yet another specific embodiment of the various methods provided herein, the biomarker is PDE6D. In yet another specific embodiment of the various methods provided herein, the biomarker is LGALS2. In yet another specific embodiment of the various methods provided herein, the biomarker is LGALS9. In some embodiments, the solid tumor is HCC. In some embodiments, the solid tumor is GBM.

In a specific embodiment of the various methods provided herein, the biomarker is ZFP91, and the solid tumor is HCC. In another embodiment of the various methods provided herein, the biomarker is CRBN, and the solid tumor is HCC. In yet another embodiment of the various methods provided herein, the biomarkers are both ZFP91 and CRBN, and the solid tumor is HCC. In another specific embodiment of the various methods provided herein, the biomarker is CK1-alpha, and the solid tumor is HCC. In another specific embodiment of the various methods provided herein, the biomarker is GSPT1, and the solid tumor is HCC. In another specific embodiment of the various methods provided herein, the biomarker is MST1R (RON), and the solid tumor is HCC. In another specific embodiment of the various methods provided herein, the biomarker is Nestin, and the solid tumor is HCC. In another specific embodiment of the various methods provided herein, the biomarker is KAT1/CCBL1, and the solid tumor is HCC. In another specific embodiment of the various methods provided herein, the biomarker is WIBG, and the solid tumor is HCC. In another specific embodiment of the various methods provided herein, the biomarker is MVP, and the solid tumor is HCC. In yet another specific embodiment of the various methods provided herein, the biomarker is PARP4, and the solid tumor is HCC. In yet another specific embodiment of the various methods provided herein, the biomarker is ZNF198, and the solid tumor is HCC. In yet another specific embodiment of the various methods provided herein, the biomarker is ZMYM198, and the solid tumor is HCC. In yet another specific embodiment of the various methods provided herein, the biomarker is FAM83H, and the solid tumor is HCC. In yet another specific embodiment of the various methods provided herein, the biomarker is KLC4, and the solid tumor is HCC. In yet another specific embodiment of the various methods provided herein, the biomarker is EHMT2, and the solid tumor is HCC. In yet another specific embodiment of the various methods provided herein, the biomarker is GLRX, and the solid tumor is HCC. In yet another specific embodiment of the various methods provided herein, the biomarker is SDSL, and the solid tumor is HCC. In yet another specific embodiment of the various methods provided herein, the biomarker is LIG3, and the solid tumor is HCC. In yet another specific embodiment of the various methods provided herein, the biomarker is IKBKE, and the solid tumor is HCC. In yet another specific embodiment of the various methods provided herein, the biomarker is PDE6D, and the solid tumor is HCC. In yet another specific embodiment of the various methods provided herein, the biomarker is LGALS2, and the solid tumor is HCC. In yet another specific embodiment of the various methods provided herein, the biomarker is LGALS9, and the solid tumor is HCC.

In a specific embodiment of the various methods provided herein, the biomarker is ZFP91, and the solid tumor is GBM. In another embodiment of the various methods provided herein, the biomarker is CRBN, and the solid tumor is GBM. In yet another embodiment of the various methods provided herein, the biomarkers are both ZFP91 and CRBN, and the solid tumor is GBM. In another specific embodiment of the various methods provided herein, the biomarker is CK1-alpha, and the solid tumor is GBM. In another specific embodiment of the various methods provided herein, the biomarker is GSPT1, and the solid tumor is GBM. In another specific embodiment of the various methods provided herein, the biomarker is MST1R (RON), and the solid tumor is GBM. In another specific embodiment of the various methods provided herein, the biomarker is Nestin, and the solid tumor is GBM. In another specific embodiment of the various methods provided herein, the biomarker is KAT1/CCBL1, and the solid tumor is GBM. In another specific embodiment of the various methods provided herein, the biomarker is WIBG, and the solid tumor is GBM. In another specific embodiment of the various methods provided herein, the biomarker is MVP, and the solid tumor is GBM. In yet another specific embodiment of the various methods provided herein, the biomarker is PARP4, and the solid tumor is GBM. In yet another specific embodiment of the various methods provided herein, the biomarker is ZNF198, and the solid tumor is GBM. In yet another specific embodiment of the various methods provided herein, the biomarker is ZMYM198, and the solid tumor is GBM. In yet another specific embodiment of the various methods provided herein, the biomarker is FAM83H, and the solid tumor is GBM. In yet another specific embodiment of the various methods provided herein, the biomarker is KLC4, and the solid tumor is GBM. In yet another specific embodiment of the various methods provided herein, the biomarker is EHMT2, and the solid tumor is GBM. In yet another specific embodiment of the various methods provided herein, the biomarker is GLRX, and the solid tumor is GBM. In yet another specific embodiment of the various methods provided herein, the biomarker is SDSL, and the solid tumor is GBM. In yet another specific embodiment of the various methods provided herein, the biomarker is LIG3, and the solid tumor is GBM. In yet another specific embodiment of the various methods provided herein, the biomarker is IKBKE, and the solid tumor is GBM. In yet another specific embodiment of the various methods provided herein, the biomarker is PDE6D, and the solid tumor is GBM. In yet another specific embodiment of the various methods provided herein, the biomarker is LGALS2, and the solid tumor is GBM. In yet another specific embodiment of the various methods provided herein, the biomarker is LGALS9, and the solid tumor is GBM.

In yet other embodiments of the various methods provided herein, the biomarker is selected from a group consisting of SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTU1, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770, and the solid tumor is HCC or GBM.

In yet other embodiments of the various methods provided herein, the biomarker is selected from a group consisting of MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDC38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLIT, FNBP1L, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830, and the solid tumor is HCC or GBM.

In yet other embodiments of the various methods provided herein, the biomarker is selected from a group consisting of ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Tap, Igf2r, IGFBP1, IGFBP3, IKBKE, JAG1, KIAA0100, KIAA1462, KIF20B, KIF22, KLC2, KRT9, LDLR, LPXN, MAFF, MELK, MET, MGAT4B, MGLL, MMP7, MST1R, MT2A, MVP, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2, and the solid tumor is HCC or GBM.

In yet other embodiments of the various methods provided herein, the biomarker is selected from a group consisting of ACSL6, AHSG, ALB, ARPP19, ATP5EP2, ATP5I, ATP5J, AZGP1, BCKDK, BLOC1S3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1H1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBC1D12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2, and the solid tumor is HCC or GBM.

In some embodiments, provided herein is a method of identifying a subject who is likely to be responsive to a treatment of a solid tumor, e.g., HCC or GBM, with Compound B, comprising:

(a) administering Compound B to a subject having a solid tumor;

(b) obtaining a sample from the subject;

(c) determining the level of a biomarker in the sample from the subject, wherein the biomarker is selected from the group consisting of biomarkers identified in Tables 1 to 6, and combinations thereof; and

(d) diagnosing the subject as being likely to be responsive to Compound B if the level of the biomarker in the sample of the subject changes as compared to a reference level of the biomarker. In one embodiment, the solid tumor is HCC. In another embodiment, the solid tumor is GBM.

In some embodiments, provided herein is a method of treating a solid tumor, e.g., HCC or GBM, comprising:

(a) obtaining a sample from the subject;

(b) determining the level of a biomarker in the sample from the subject, wherein the biomarker is selected from the group consisting of biomarkers identified in Tables 1 to 6, and combinations thereof;

(c) diagnosing the subject as being likely to be responsive to Compound B if the level of the biomarker in the sample of the subject changes as compared to a reference level of the biomarker; and

(d) administering a therapeutically effective amount of Compound B to the subject diagnosed to be likely to be responsive to Compound B. In one embodiment, the solid tumor is HCC. In another embodiment, the solid tumor is GBM.

In some embodiments, the biomarker is selected from a group consisting of CRBN, CK1-alpha, GSPT1, Nestin, KAT1/CCBL1, WIBG, FAM83H, KLC4, LIG3, IKBKE, LGALS2 LGALS9, MVP, PARP4, ZFP91, ZNF198, MST1R (RON), ZMYM198, EHMT2, GLRX, SDSL, and PDE6D. In some embodiments of the various methods provided herein, the biomarker is selected from a group consisting of CRBN, CK1-alpha, GSPT1, Nestin, KAT1/CCBL1, WIBG, FAM83H, KLC4, LIG3, IKBKE, LGALS2 and LGALS9. In other embodiments, the biomarker is selected from a group consisting of MVP, PARP4, ZFP91, ZNF198, MST1R (RON), ZMYM198, EHMT2, GLRX, SDSL, and PDE6D. In a specific embodiment of the various methods provided herein, the biomarker is ZFP91. In another embodiment of the various methods provided herein, the biomarker is CRBN. In yet another embodiment of the various methods provided herein, the biomarkers are both ZFP91 and CRBN. In another specific embodiment of the various methods provided herein, the biomarker is CK1-alpha. In another specific embodiment of the various methods provided herein, the biomarker is GSPT1. In another specific embodiment of the various methods provided herein, the biomarker is MST1R (RON). In another specific embodiment of the various methods provided herein, the biomarker is Nestin. In another specific embodiment of the various methods provided herein, the biomarker is KAT1/CCBL1. In another specific embodiment of the various methods provided herein, the biomarker is WIBG. In another specific embodiment of the various methods provided herein, the biomarker is MVP. In yet another specific embodiment of the various methods provided herein, the biomarker is PARP4. In yet another specific embodiment of the various methods provided herein, the biomarker is ZNF198. In yet another specific embodiment of the various methods provided herein, the biomarker is ZMYM198. In yet another specific embodiment of the various methods provided herein, the biomarker is FAM83H. In yet another specific embodiment of the various methods provided herein, the biomarker is KLC4. In yet another specific embodiment of the various methods provided herein, the biomarker is EHMT2. In yet another specific embodiment of the various methods provided herein, the biomarker is GLRX. In yet another specific embodiment of the various methods provided herein, the biomarker is SDSL. In yet another specific embodiment of the various methods provided herein, the biomarker is LIG3. In yet another specific embodiment of the various methods provided herein, the biomarker is IKBKE. In yet another specific embodiment of the various methods provided herein, the biomarker is PDE6D. In yet another specific embodiment of the various methods provided herein, the biomarker is LGALS2. In yet another specific embodiment of the various methods provided herein, the biomarker is LGALS9. In some embodiments, the solid tumor is HCC. In some embodiments, the solid tumor is GBM.

In a specific embodiment of the various methods provided herein, the biomarker is ZFP91, and the solid tumor is HCC. In another embodiment of the various methods provided herein, the biomarker is CRBN, and the solid tumor is HCC. In yet another embodiment of the various methods provided herein, the biomarkers are both ZFP91 and CRBN, and the solid tumor is HCC. In another specific embodiment of the various methods provided herein, the biomarker is CK1-alpha, and the solid tumor is HCC. In another specific embodiment of the various methods provided herein, the biomarker is GSPT1, and the solid tumor is HCC. In another specific embodiment of the various methods provided herein, the biomarker is MST1R (RON), and the solid tumor is HCC. In another specific embodiment of the various methods provided herein, the biomarker is Nestin, and the solid tumor is HCC. In another specific embodiment of the various methods provided herein, the biomarker is KAT1/CCBL1, and the solid tumor is HCC. In another specific embodiment of the various methods provided herein, the biomarker is WIBG, and the solid tumor is HCC. In another specific embodiment of the various methods provided herein, the biomarker is MVP, and the solid tumor is HCC. In yet another specific embodiment of the various methods provided herein, the biomarker is PARP4, and the solid tumor is HCC. In yet another specific embodiment of the various methods provided herein, the biomarker is ZNF198, and the solid tumor is HCC. In yet another specific embodiment of the various methods provided herein, the biomarker is ZMYM198, and the solid tumor is HCC. In yet another specific embodiment of the various methods provided herein, the biomarker is FAM83H, and the solid tumor is HCC. In yet another specific embodiment of the various methods provided herein, the biomarker is KLC4, and the solid tumor is HCC. In yet another specific embodiment of the various methods provided herein, the biomarker is EHMT2, and the solid tumor is HCC. In yet another specific embodiment of the various methods provided herein, the biomarker is GLRX, and the solid tumor is HCC. In yet another specific embodiment of the various methods provided herein, the biomarker is SDSL, and the solid tumor is HCC. In yet another specific embodiment of the various methods provided herein, the biomarker is LIG3, and the solid tumor is HCC. In yet another specific embodiment of the various methods provided herein, the biomarker is IKBKE, and the solid tumor is HCC. In yet another specific embodiment of the various methods provided herein, the biomarker is PDE6D, and the solid tumor is HCC. In yet another specific embodiment of the various methods provided herein, the biomarker is LGALS2, and the solid tumor is HCC. In yet another specific embodiment of the various methods provided herein, the biomarker is LGALS9, and the solid tumor is HCC.

In a specific embodiment of the various methods provided herein, the biomarker is ZFP91, and the solid tumor is GBM. In another embodiment of the various methods provided herein, the biomarker is CRBN, and the solid tumor is GBM. In yet another embodiment of the various methods provided herein, the biomarkers are both ZFP91 and CRBN, and the solid tumor is GBM. In another specific embodiment of the various methods provided herein, the biomarker is CK1-alpha, and the solid tumor is GBM. In another specific embodiment of the various methods provided herein, the biomarker is GSPT1, and the solid tumor is GBM. In another specific embodiment of the various methods provided herein, the biomarker is MST1R (RON), and the solid tumor is GBM. In another specific embodiment of the various methods provided herein, the biomarker is Nestin, and the solid tumor is GBM. In another specific embodiment of the various methods provided herein, the biomarker is KAT1/CCBL1, and the solid tumor is GBM. In another specific embodiment of the various methods provided herein, the biomarker is WIBG, and the solid tumor is GBM. In another specific embodiment of the various methods provided herein, the biomarker is MVP, and the solid tumor is GBM. In yet another specific embodiment of the various methods provided herein, the biomarker is PARP4, and the solid tumor is GBM. In yet another specific embodiment of the various methods provided herein, the biomarker is ZNF198, and the solid tumor is GBM. In yet another specific embodiment of the various methods provided herein, the biomarker is ZMYM198, and the solid tumor is GBM. In yet another specific embodiment of the various methods provided herein, the biomarker is FAM83H, and the solid tumor is GBM. In yet another specific embodiment of the various methods provided herein, the biomarker is KLC4, and the solid tumor is GBM. In yet another specific embodiment of the various methods provided herein, the biomarker is EHMT2, and the solid tumor is GBM. In yet another specific embodiment of the various methods provided herein, the biomarker is GLRX, and the solid tumor is GBM. In yet another specific embodiment of the various methods provided herein, the biomarker is SDSL, and the solid tumor is GBM. In yet another specific embodiment of the various methods provided herein, the biomarker is LIG3, and the solid tumor is GBM. In yet another specific embodiment of the various methods provided herein, the biomarker is IKBKE, and the solid tumor is GBM. In yet another specific embodiment of the various methods provided herein, the biomarker is PDE6D, and the solid tumor is GBM. In yet another specific embodiment of the various methods provided herein, the biomarker is LGALS2, and the solid tumor is GBM. In yet another specific embodiment of the various methods provided herein, the biomarker is LGALS9, and the solid tumor is GBM.

In yet other embodiments of the various methods provided herein, the biomarker is selected from a group consisting of SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTU1, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770, and the solid tumor is HCC or GBM.

In yet other embodiments of the various methods provided herein, the biomarker is selected from a group consisting of MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDC38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLIT, FNBP1L, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830, and the solid tumor is HCC or GBM.

In yet other embodiments of the various methods provided herein, the biomarker is selected from a group consisting of ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Tap, Igf2r, IGFBP1, IGFBP3, IKBKE, JAG1, KIAA0100, KIAA1462, KIF20B, KIF22, KLC2, KRT9, LDLR, LPXN, MAFF, MELK, MET, MGAT4B, MGLL, MMP7, MST1R, MT2A, MVP, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2, and the solid tumor is HCC or GBM.

In yet other embodiments of the various methods provided herein, the biomarker is selected from a group consisting of ACSL6, AHSG, ALB, ARPP19, ATP5EP2, ATP5I, ATP5J, AZGP1, BCKDK, BLOC1S3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1H1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBC1D12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2, and the solid tumor is HCC or GBM.

In some embodiments, provided herein is a method of identifying a subject who is likely to be responsive to a treatment of a solid tumor, e.g., HCC or GBM, with lenalidomide, comprising:

(a) administering lenalidomide to a subject having a solid tumor;

(b) obtaining a sample from the subject;

(c) determining the level of a biomarker in the sample from the subject, wherein the biomarker is selected from the group consisting of biomarkers identified in Tables 1 to 6, and combinations thereof; and

(d) diagnosing the subject as being likely to be responsive to lenalidomide if the level of the biomarker in the sample of the subject changes as compared to a reference level of the biomarker. In one embodiment, the solid tumor is HCC. In another embodiment, the solid tumor is GBM.

In some embodiments, provided herein is a method of treating a solid tumor, e.g., HCC or GBM, comprising:

(a) obtaining a sample from the subject;

(b) determining the level of a biomarker in the sample from the subject, wherein the biomarker is selected from the group consisting of biomarkers identified in Tables 1 to 6, and combinations thereof;

(c) diagnosing the subject as being likely to be responsive to lenalidomide if the level of the biomarker in the sample of the subject changes as compared to a reference level of the biomarker; and

(d) administering a therapeutically effective amount of lenalidomide to the subject diagnosed to be likely to be responsive to lenalidomide. In one embodiment, the solid tumor is HCC. In another embodiment, the solid tumor is GBM.

In some embodiments, the biomarker is selected from a group consisting of CRBN, CK1-alpha, GSPT1, Nestin, KAT1/CCBL1, WIBG, FAM83H, KLC4, LIG3, IKBKE, LGALS2 LGALS9, MVP, PARP4, ZFP91, ZNF198, MST1R (RON), ZMYM198, EHMT2, GLRX, SDSL, and PDE6D. In some embodiments of the various methods provided herein, the biomarker is selected from a group consisting of CRBN, CK1-alpha, GSPT1, Nestin, KAT1/CCBL1, WIBG, FAM83H, KLC4, LIG3, IKBKE, LGALS2 and LGALS9. In other embodiments, the biomarker is selected from a group consisting of MVP, PARP4, ZFP91, ZNF198, MST1R (RON), ZMYM198, EHMT2, GLRX, SDSL, and PDE6D. In a specific embodiment of the various methods provided herein, the biomarker is ZFP91. In another embodiment of the various methods provided herein, the biomarker is CRBN. In yet another embodiment of the various methods provided herein, the biomarkers are both ZFP91 and CRBN. In another specific embodiment of the various methods provided herein, the biomarker is CK1-alpha. In another specific embodiment of the various methods provided herein, the biomarker is GSPT1. In another specific embodiment of the various methods provided herein, the biomarker is MST1R (RON). In another specific embodiment of the various methods provided herein, the biomarker is Nestin. In another specific embodiment of the various methods provided herein, the biomarker is KAT1/CCBL1. In another specific embodiment of the various methods provided herein, the biomarker is WIBG. In another specific embodiment of the various methods provided herein, the biomarker is MVP. In yet another specific embodiment of the various methods provided herein, the biomarker is PARP4. In yet another specific embodiment of the various methods provided herein, the biomarker is ZNF198. In yet another specific embodiment of the various methods provided herein, the biomarker is ZMYM198. In yet another specific embodiment of the various methods provided herein, the biomarker is FAM83H. In yet another specific embodiment of the various methods provided herein, the biomarker is KLC4. In yet another specific embodiment of the various methods provided herein, the biomarker is EHMT2. In yet another specific embodiment of the various methods provided herein, the biomarker is GLRX. In yet another specific embodiment of the various methods provided herein, the biomarker is SDSL. In yet another specific embodiment of the various methods provided herein, the biomarker is LIG3. In yet another specific embodiment of the various methods provided herein, the biomarker is IKBKE. In yet another specific embodiment of the various methods provided herein, the biomarker is PDE6D. In yet another specific embodiment of the various methods provided herein, the biomarker is LGALS2. In yet another specific embodiment of the various methods provided herein, the biomarker is LGALS9. In some embodiments, the solid tumor is HCC. In some embodiments, the solid tumor is GBM.

In a specific embodiment of the various methods provided herein, the biomarker is ZFP91, and the solid tumor is HCC. In another embodiment of the various methods provided herein, the biomarker is CRBN, and the solid tumor is HCC. In yet another embodiment of the various methods provided herein, the biomarkers are both ZFP91 and CRBN, and the solid tumor is HCC. In another specific embodiment of the various methods provided herein, the biomarker is CK1-alpha, and the solid tumor is HCC. In another specific embodiment of the various methods provided herein, the biomarker is GSPT1, and the solid tumor is HCC. In another specific embodiment of the various methods provided herein, the biomarker is MST1R (RON), and the solid tumor is HCC. In another specific embodiment of the various methods provided herein, the biomarker is Nestin, and the solid tumor is HCC. In another specific embodiment of the various methods provided herein, the biomarker is KAT1/CCBL1, and the solid tumor is HCC. In another specific embodiment of the various methods provided herein, the biomarker is WIBG, and the solid tumor is HCC. In another specific embodiment of the various methods provided herein, the biomarker is MVP, and the solid tumor is HCC. In yet another specific embodiment of the various methods provided herein, the biomarker is PARP4, and the solid tumor is HCC. In yet another specific embodiment of the various methods provided herein, the biomarker is ZNF198, and the solid tumor is HCC. In yet another specific embodiment of the various methods provided herein, the biomarker is ZMYM198, and the solid tumor is HCC. In yet another specific embodiment of the various methods provided herein, the biomarker is FAM83H, and the solid tumor is HCC. In yet another specific embodiment of the various methods provided herein, the biomarker is KLC4, and the solid tumor is HCC. In yet another specific embodiment of the various methods provided herein, the biomarker is EHMT2, and the solid tumor is HCC. In yet another specific embodiment of the various methods provided herein, the biomarker is GLRX, and the solid tumor is HCC. In yet another specific embodiment of the various methods provided herein, the biomarker is SDSL, and the solid tumor is HCC. In yet another specific embodiment of the various methods provided herein, the biomarker is LIG3, and the solid tumor is HCC. In yet another specific embodiment of the various methods provided herein, the biomarker is IKBKE, and the solid tumor is HCC. In yet another specific embodiment of the various methods provided herein, the biomarker is PDE6D, and the solid tumor is HCC. In yet another specific embodiment of the various methods provided herein, the biomarker is LGALS2, and the solid tumor is HCC. In yet another specific embodiment of the various methods provided herein, the biomarker is LGALS9, and the solid tumor is HCC.

In a specific embodiment of the various methods provided herein, the biomarker is ZFP91, and the solid tumor is GBM. In another embodiment of the various methods provided herein, the biomarker is CRBN, and the solid tumor is GBM. In yet another embodiment of the various methods provided herein, the biomarkers are both ZFP91 and CRBN, and the solid tumor is GBM. In another specific embodiment of the various methods provided herein, the biomarker is CK1-alpha, and the solid tumor is GBM. In another specific embodiment of the various methods provided herein, the biomarker is GSPT1, and the solid tumor is GBM. In another specific embodiment of the various methods provided herein, the biomarker is MST1R (RON), and the solid tumor is GBM. In another specific embodiment of the various methods provided herein, the biomarker is Nestin, and the solid tumor is GBM. In another specific embodiment of the various methods provided herein, the biomarker is KAT1/CCBL1, and the solid tumor is GBM. In another specific embodiment of the various methods provided herein, the biomarker is WIBG, and the solid tumor is GBM. In another specific embodiment of the various methods provided herein, the biomarker is MVP, and the solid tumor is GBM. In yet another specific embodiment of the various methods provided herein, the biomarker is PARP4, and the solid tumor is GBM. In yet another specific embodiment of the various methods provided herein, the biomarker is ZNF198, and the solid tumor is GBM. In yet another specific embodiment of the various methods provided herein, the biomarker is ZMYM198, and the solid tumor is GBM. In yet another specific embodiment of the various methods provided herein, the biomarker is FAM83H, and the solid tumor is GBM. In yet another specific embodiment of the various methods provided herein, the biomarker is KLC4, and the solid tumor is GBM. In yet another specific embodiment of the various methods provided herein, the biomarker is EHMT2, and the solid tumor is GBM. In yet another specific embodiment of the various methods provided herein, the biomarker is GLRX, and the solid tumor is GBM. In yet another specific embodiment of the various methods provided herein, the biomarker is SDSL, and the solid tumor is GBM. In yet another specific embodiment of the various methods provided herein, the biomarker is LIG3, and the solid tumor is GBM. In yet another specific embodiment of the various methods provided herein, the biomarker is IKBKE, and the solid tumor is GBM. In yet another specific embodiment of the various methods provided herein, the biomarker is PDE6D, and the solid tumor is GBM. In yet another specific embodiment of the various methods provided herein, the biomarker is LGALS2, and the solid tumor is GBM. In yet another specific embodiment of the various methods provided herein, the biomarker is LGALS9, and the solid tumor is GBM.

In yet other embodiments of the various methods provided herein, the biomarker is selected from a group consisting of SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTU1, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770, and the solid tumor is HCC or GBM.

In yet other embodiments of the various methods provided herein, the biomarker is selected from a group consisting of MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDC38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLIT, FNBP1L, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830, and the solid tumor is HCC or GBM.

In yet other embodiments of the various methods provided herein, the biomarker is selected from a group consisting of ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Tap, Igf2r, IGFBP1, IGFBP3, IKBKE, JAG1, KIAA0100, KIAA1462, KIF20B, KIF22, KLC2, KRT9, LDLR, LPXN, MAFF, MELK, MET, MGAT4B, MGLL, MMP7, MST1R, MT2A, MVP, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2, and the solid tumor is HCC or GBM.

In yet other embodiments of the various methods provided herein, the biomarker is selected from a group consisting of ACSL6, AHSG, ALB, ARPP19, ATP5EP2, ATP5I, ATP5J, AZGP1, BCKDK, BLOC1S3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1H1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBC1D12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2, and the solid tumor is HCC or GBM.

In some embodiments, the solid tumors are sarcomas, carcinomas (epithelial tumors), melanomas, and glioblastomas. Exemplary solid tumors include, but not limited to, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma, glioblastoma (GBM); medulloblastoma), cervical cancer (e.g., cervical adenocarcinoma), colorectal cancer (e.g., colon cancer, rectal cancer, colorectal adenocarcinoma), gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), pancreatic cancer (e.g., pancreatic andenocarcinoma, intraductal papillary mucinous neoplasm (IPMN)), prostate cancer (e.g., prostate adenocarcinoma), skin cancer (e.g., squamous cell carcinoma (SCC), keratoacanthoma (A), melanoma, basal cell carcinoma (BCC)) and soft tissue sarcoma (e.g., malignant fibrous histiocytoma (MFH), liposarcoma, malignant peripheral nerve sheath tumor (MPNST), chondrosarcoma, fibrosarcoma, myxosarcoma, osteosarcoma). In some embodiments, the solid tumor is a liver cancer. In one embodiment, the solid tumor is HCC. In other embodiments, the solid tumor is a brain cancer. In one embodiment, the solid tumor is GBM.

In some embodiments, provided herein is a method of identifying a subject having HCC who is likely to be responsive to a treatment compound, comprising:

(a) administering the treatment compound to a subject having HCC;

(b) obtaining a sample from the subject;

(c) determining the level of a biomarker in the sample from the subject, wherein the biomarker is selected from the group consisting of biomarkers identified in Tables 1 to 6, and combinations thereof; and

(d) diagnosing the subject as being likely to be responsive to the treatment compound if the level of the biomarker in the sample of the subject changes as compared to a reference level of the biomarker.

In some embodiments, provided herein is a method of treating HCC, comprising:

(a) obtaining a sample from the subject having HCC;

(b) determining the level of a biomarker in the sample from the subject, wherein the biomarker is selected from the group consisting of biomarkers identified in Tables 1 to 6, and combinations thereof;

(c) diagnosing the subject as being likely to be responsive to a treatment compound if the level of the biomarker in the sample of the subject changes as compared to a reference level of the biomarker; and

(d) administering a therapeutically effective amount of the treatment compound to the subject diagnosed to be likely to be responsive to the treatment compound.

In some embodiments, the biomarker is selected from a group consisting of CRBN, CK1-alpha, GSPT1, Nestin, KAT1/CCBL1, WIBG, FAM83H, KLC4, LIG3, IKBKE, LGALS2 LGALS9, MVP, PARP4, ZFP91, ZNF198, MST1R (RON), ZMYM198, EHMT2, GLRX, SDSL, and PDE6D. In some embodiments of the various methods provided herein, the biomarker is selected from a group consisting of CRBN, CK1-alpha, GSPT1, Nestin, KAT1/CCBL1, WIBG, FAM83H, KLC4, LIG3, IKBKE, LGALS2 and LGALS9. In other embodiments, the biomarker is selected from a group consisting of MVP, PARP4, ZFP91, ZNF198, MST1R (RON), ZMYM198, EHMT2, GLRX, SDSL, and PDE6D. In a specific embodiment of the various methods provided herein, the biomarker is ZFP91. In another embodiment of the various methods provided herein, the biomarker is CRBN. In yet another embodiment of the various methods provided herein, the biomarkers are both ZFP91 and CRBN. In another specific embodiment of the various methods provided herein, the biomarker is CK1-alpha. In another specific embodiment of the various methods provided herein, the biomarker is GSPT1. In another specific embodiment of the various methods provided herein, the biomarker is MST1R (RON). In another specific embodiment of the various methods provided herein, the biomarker is Nestin. In another specific embodiment of the various methods provided herein, the biomarker is KAT1/CCBL1. In another specific embodiment of the various methods provided herein, the biomarker is WIBG. In another specific embodiment of the various methods provided herein, the biomarker is MVP. In yet another specific embodiment of the various methods provided herein, the biomarker is PARP4. In yet another specific embodiment of the various methods provided herein, the biomarker is ZNF198. In yet another specific embodiment of the various methods provided herein, the biomarker is ZMYM198. In yet another specific embodiment of the various methods provided herein, the biomarker is FAM83H. In yet another specific embodiment of the various methods provided herein, the biomarker is KLC4. In yet another specific embodiment of the various methods provided herein, the biomarker is EHMT2. In yet another specific embodiment of the various methods provided herein, the biomarker is GLRX. In yet another specific embodiment of the various methods provided herein, the biomarker is SDSL. In yet another specific embodiment of the various methods provided herein, the biomarker is LIG3. In yet another specific embodiment of the various methods provided herein, the biomarker is IKBKE. In yet another specific embodiment of the various methods provided herein, the biomarker is PDE6D. In yet another specific embodiment of the various methods provided herein, the biomarker is LGALS2. In yet another specific embodiment of the various methods provided herein, the biomarker is LGALS9. In some embodiments, the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In a specific embodiment of the various methods provided herein, the biomarker is ZFP91, and the treatment compound is Compound A. In another embodiment of the various methods provided herein, the biomarker is CRBN, and the treatment compound is Compound A. In yet another embodiment of the various methods provided herein, the biomarkers are both ZFP91 and CRBN, and the treatment compound is Compound A. In another specific embodiment of the various methods provided herein, the biomarker is CK1-alpha, and the treatment compound is Compound A. In another specific embodiment of the various methods provided herein, the biomarker is GSPT1, and the treatment compound is Compound A. In another specific embodiment of the various methods provided herein, the biomarker is MST1R (RON), and the treatment compound is Compound A. In another specific embodiment of the various methods provided herein, the biomarker is Nestin, and the treatment compound is Compound A. In another specific embodiment of the various methods provided herein, the biomarker is KAT1/CCBL1, and the treatment compound is Compound A. In another specific embodiment of the various methods provided herein, the biomarker is WIBG, and the treatment compound is Compound A. In another specific embodiment of the various methods provided herein, the biomarker is MVP, and the treatment compound is Compound A. In yet another specific embodiment of the various methods provided herein, the biomarker is PARP4, and the treatment compound is Compound A. In yet another specific embodiment of the various methods provided herein, the biomarker is ZNF198, and the treatment compound is Compound A. In yet another specific embodiment of the various methods provided herein, the biomarker is ZMYM198, and the treatment compound is Compound A. In yet another specific embodiment of the various methods provided herein, the biomarker is FAM83H, and the treatment compound is Compound A. In yet another specific embodiment of the various methods provided herein, the biomarker is KLC4, and the treatment compound is Compound A. In yet another specific embodiment of the various methods provided herein, the biomarker is EHMT2, and the treatment compound is Compound A. In yet another specific embodiment of the various methods provided herein, the biomarker is GLRX, and the treatment compound is Compound A. In yet another specific embodiment of the various methods provided herein, the biomarker is SDSL, and the treatment compound is Compound A. In yet another specific embodiment of the various methods provided herein, the biomarker is LIG3, and the treatment compound is Compound A. In yet another specific embodiment of the various methods provided herein, the biomarker is IKBKE, and the treatment compound is Compound A. In yet another specific embodiment of the various methods provided herein, the biomarker is PDE6D, and the treatment compound is Compound A. In yet another specific embodiment of the various methods provided herein, the biomarker is LGALS2, and the treatment compound is Compound A. In yet another specific embodiment of the various methods provided herein, the biomarker is LGALS9, and the treatment compound is Compound A.

In a specific embodiment of the various methods provided herein, the biomarker is ZFP91, and the treatment compound is Compound B. In another embodiment of the various methods provided herein, the biomarker is CRBN, and the treatment compound is Compound B. In yet another embodiment of the various methods provided herein, the biomarkers are both ZFP91 and CRBN, and the treatment compound is Compound B. In another specific embodiment of the various methods provided herein, the biomarker is CK1-alpha, and the treatment compound is Compound B. In another specific embodiment of the various methods provided herein, the biomarker is GSPT1, and the treatment compound is Compound B. In another specific embodiment of the various methods provided herein, the biomarker is MST1R (RON), and the treatment compound is Compound B. In another specific embodiment of the various methods provided herein, the biomarker is Nestin, and the treatment compound is Compound B. In another specific embodiment of the various methods provided herein, the biomarker is KAT1/CCBL1, and the treatment compound is Compound B. In another specific embodiment of the various methods provided herein, the biomarker is WIBG, and the treatment compound is Compound B. In another specific embodiment of the various methods provided herein, the biomarker is MVP, and the treatment compound is Compound B. In yet another specific embodiment of the various methods provided herein, the biomarker is PARP4, and the treatment compound is Compound B. In yet another specific embodiment of the various methods provided herein, the biomarker is ZNF198, and the treatment compound is Compound B. In yet another specific embodiment of the various methods provided herein, the biomarker is ZMYM198, and the treatment compound is Compound B. In yet another specific embodiment of the various methods provided herein, the biomarker is FAM83H, and the treatment compound is Compound B. In yet another specific embodiment of the various methods provided herein, the biomarker is KLC4, and the treatment compound is Compound B. In yet another specific embodiment of the various methods provided herein, the biomarker is EHMT2, and the treatment compound is Compound B. In yet another specific embodiment of the various methods provided herein, the biomarker is GLRX, and the treatment compound is Compound B. In yet another specific embodiment of the various methods provided herein, the biomarker is SDSL, and the treatment compound is Compound B. In yet another specific embodiment of the various methods provided herein, the biomarker is LIG3, and the treatment compound is Compound B. In yet another specific embodiment of the various methods provided herein, the biomarker is IKBKE, and the treatment compound is Compound B. In yet another specific embodiment of the various methods provided herein, the biomarker is PDE6D, and the treatment compound is Compound B. In yet another specific embodiment of the various methods provided herein, the biomarker is LGALS2, and the treatment compound is Compound B. In yet another specific embodiment of the various methods provided herein, the biomarker is LGALS9, and the treatment compound is Compound B.

In a specific embodiment of the various methods provided herein, the biomarker is ZFP91, and the treatment compound is Lenalidomide. In another embodiment of the various methods provided herein, the biomarker is CRBN, and the treatment compound is Lenalidomide. In yet another embodiment of the various methods provided herein, the biomarkers are both ZFP91 and CRBN, and the treatment compound is Lenalidomide. In another specific embodiment of the various methods provided herein, the biomarker is CK1-alpha, and the treatment compound is Lenalidomide. In another specific embodiment of the various methods provided herein, the biomarker is GSPT1, and the treatment compound is Lenalidomide. In another specific embodiment of the various methods provided herein, the biomarker is MST1R (RON), and the treatment compound is Lenalidomide. In another specific embodiment of the various methods provided herein, the biomarker is Nestin, and the treatment compound is Lenalidomide. In another specific embodiment of the various methods provided herein, the biomarker is KAT1/CCBL1, and the treatment compound is Lenalidomide. In another specific embodiment of the various methods provided herein, the biomarker is WIBG, and the treatment compound is Lenalidomide. In another specific embodiment of the various methods provided herein, the biomarker is MVP, and the treatment compound is Lenalidomide. In yet another specific embodiment of the various methods provided herein, the biomarker is PARP4, and the treatment compound is Lenalidomide. In yet another specific embodiment of the various methods provided herein, the biomarker is ZNF198, and the treatment compound is Lenalidomide. In yet another specific embodiment of the various methods provided herein, the biomarker is ZMYM198, and the treatment compound is Lenalidomide. In yet another specific embodiment of the various methods provided herein, the biomarker is FAM83H, and the treatment compound is Lenalidomide. In yet another specific embodiment of the various methods provided herein, the biomarker is KLC4, and the treatment compound is Lenalidomide. In yet another specific embodiment of the various methods provided herein, the biomarker is EHMT2, and the treatment compound is Lenalidomide. In yet another specific embodiment of the various methods provided herein, the biomarker is GLRX, and the treatment compound is Lenalidomide. In yet another specific embodiment of the various methods provided herein, the biomarker is SDSL, and the treatment compound is Lenalidomide. In yet another specific embodiment of the various methods provided herein, the biomarker is LIG3, and the treatment compound is Lenalidomide. In yet another specific embodiment of the various methods provided herein, the biomarker is IKBKE, and the treatment compound is Lenalidomide. In yet another specific embodiment of the various methods provided herein, the biomarker is PDE6D, and the treatment compound is Lenalidomide. In yet another specific embodiment of the various methods provided herein, the biomarker is LGALS2, and the treatment compound is Lenalidomide. In yet another specific embodiment of the various methods provided herein, the biomarker is LGALS9, and the treatment compound is Lenalidomide.

In yet other embodiments of the various methods provided herein, the biomarker is selected from a group consisting of SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTU1, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770, and the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In yet other embodiments of the various methods provided herein, the biomarker is selected from a group consisting of MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDC38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLII, FNBP1L, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830, and the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In yet other embodiments of the various methods provided herein, the biomarker is selected from a group consisting of ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Tap, Igf2r, IGFBP1, IGFBP3, IKBKE, JAG1, KIAA0100, KIAA1462, KIF20B, KIF22, KLC2, KRT9, LDLR, LPXN, MAFF, MELK, MET, MGAT4B, MGLL, MMP7, MST1R, MT2A, MVP, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2, and the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In yet other embodiments of the various methods provided herein, the biomarker is selected from a group consisting of ACSL6, AHSG, ALB, ARPP19, ATP5EP2, ATP5I, ATP5J, AZGP1, BCKDK, BLOC1S3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1H1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBC1D12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2, and the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In some embodiments, provided herein is a method of identifying a subject having GBM who is likely to be responsive to a treatment compound, comprising:

(a) administering the treatment compound to a subject having GBM;

(b) obtaining a sample from the subject;

(c) determining the level of a biomarker in the sample from the subject, wherein the biomarker is selected from the group consisting of biomarkers identified in Tables 1 to 6, and combinations thereof; and

(d) diagnosing the subject as being likely to be responsive to the treatment compound if the level of the biomarker in the sample of the subject changes as compared to a reference level of the biomarker.

In some embodiments, provided herein is a method of treating GBM, comprising:

(a) obtaining a sample from the subject having GBM;

(b) determining the level of a biomarker in the sample from the subject, wherein the biomarker is selected from the group consisting of biomarkers identified in Tables 1 to 6, and combinations thereof;

(c) diagnosing the subject as being likely to be responsive to a treatment compound if the level of the biomarker in the sample of the subject changes as compared to a reference level of the biomarker; and

(d) administering a therapeutically effective amount of the treatment compound to the subject diagnosed to be likely to be responsive to the treatment compound.

In some embodiments, the biomarker is selected from a group consisting of CRBN, CK1-alpha, GSPT1, Nestin, KAT1/CCBL1, WIBG, FAM83H, KLC4, LIG3, IKBKE, LGALS2 LGALS9, MVP, PARP4, ZFP91, ZNF198, MST1R (RON), ZMYM198, EHMT2, GLRX, SDSL, and PDE6D. In some embodiments of the various methods provided herein, the biomarker is selected from a group consisting of CRBN, CK1-alpha, GSPT1, Nestin, KAT1/CCBL1, WIBG, FAM83H, KLC4, LIG3, IKBKE, LGALS2 and LGALS9. In other embodiments, the biomarker is selected from a group consisting of MVP, PARP4, ZFP91, ZNF198, MST1R (RON), ZMYM198, EHMT2, GLRX, SDSL, and PDE6D. In a specific embodiment of the various methods provided herein, the biomarker is ZFP91. In another embodiment of the various methods provided herein, the biomarker is CRBN. In yet another embodiment of the various methods provided herein, the biomarkers are both ZFP91 and CRBN. In another specific embodiment of the various methods provided herein, the biomarker is CK1-alpha. In another specific embodiment of the various methods provided herein, the biomarker is GSPT1. In another specific embodiment of the various methods provided herein, the biomarker is MST1R (RON). In another specific embodiment of the various methods provided herein, the biomarker is Nestin. In another specific embodiment of the various methods provided herein, the biomarker is KAT1/CCBL1. In another specific embodiment of the various methods provided herein, the biomarker is WIBG. In another specific embodiment of the various methods provided herein, the biomarker is MVP. In yet another specific embodiment of the various methods provided herein, the biomarker is PARP4. In yet another specific embodiment of the various methods provided herein, the biomarker is ZNF198. In yet another specific embodiment of the various methods provided herein, the biomarker is ZMYM198. In yet another specific embodiment of the various methods provided herein, the biomarker is FAM83H. In yet another specific embodiment of the various methods provided herein, the biomarker is KLC4. In yet another specific embodiment of the various methods provided herein, the biomarker is EHMT2. In yet another specific embodiment of the various methods provided herein, the biomarker is GLRX. In yet another specific embodiment of the various methods provided herein, the biomarker is SDSL. In yet another specific embodiment of the various methods provided herein, the biomarker is LIG3. In yet another specific embodiment of the various methods provided herein, the biomarker is IKBKE. In yet another specific embodiment of the various methods provided herein, the biomarker is PDE6D. In yet another specific embodiment of the various methods provided herein, the biomarker is LGALS2. In yet another specific embodiment of the various methods provided herein, the biomarker is LGALS9. In some embodiments, the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In a specific embodiment of the various methods provided herein, the biomarker is ZFP91, and the treatment compound is Compound A. In another embodiment of the various methods provided herein, the biomarker is CRBN, and the treatment compound is Compound A. In yet another embodiment of the various methods provided herein, the biomarkers are both ZFP91 and CRBN, and the treatment compound is Compound A. In another specific embodiment of the various methods provided herein, the biomarker is CK1-alpha, and the treatment compound is Compound A. In another specific embodiment of the various methods provided herein, the biomarker is GSPT1, and the treatment compound is Compound A. In another specific embodiment of the various methods provided herein, the biomarker is MST1R (RON), and the treatment compound is Compound A. In another specific embodiment of the various methods provided herein, the biomarker is Nestin, and the treatment compound is Compound A. In another specific embodiment of the various methods provided herein, the biomarker is KAT1/CCBL1, and the treatment compound is Compound A. In another specific embodiment of the various methods provided herein, the biomarker is WIBG, and the treatment compound is Compound A. In another specific embodiment of the various methods provided herein, the biomarker is MVP, and the treatment compound is Compound A. In yet another specific embodiment of the various methods provided herein, the biomarker is PARP4, and the treatment compound is Compound A. In yet another specific embodiment of the various methods provided herein, the biomarker is ZNF198, and the treatment compound is Compound A. In yet another specific embodiment of the various methods provided herein, the biomarker is ZMYM198, and the treatment compound is Compound A. In yet another specific embodiment of the various methods provided herein, the biomarker is FAM83H, and the treatment compound is Compound A. In yet another specific embodiment of the various methods provided herein, the biomarker is KLC4, and the treatment compound is Compound A. In yet another specific embodiment of the various methods provided herein, the biomarker is EHMT2, and the treatment compound is Compound A. In yet another specific embodiment of the various methods provided herein, the biomarker is GLRX, and the treatment compound is Compound A. In yet another specific embodiment of the various methods provided herein, the biomarker is SDSL, and the treatment compound is Compound A. In yet another specific embodiment of the various methods provided herein, the biomarker is LIG3, and the treatment compound is Compound A. In yet another specific embodiment of the various methods provided herein, the biomarker is IKBKE, and the treatment compound is Compound A. In yet another specific embodiment of the various methods provided herein, the biomarker is PDE6D, and the treatment compound is Compound A. In yet another specific embodiment of the various methods provided herein, the biomarker is LGALS2, and the treatment compound is Compound A. In yet another specific embodiment of the various methods provided herein, the biomarker is LGALS9, and the treatment compound is Compound A.

In a specific embodiment of the various methods provided herein, the biomarker is ZFP91, and the treatment compound is Compound B. In another embodiment of the various methods provided herein, the biomarker is CRBN, and the treatment compound is Compound B. In yet another embodiment of the various methods provided herein, the biomarkers are both ZFP91 and CRBN, and the treatment compound is Compound B. In another specific embodiment of the various methods provided herein, the biomarker is CK1-alpha, and the treatment compound is Compound B. In another specific embodiment of the various methods provided herein, the biomarker is GSPT1, and the treatment compound is Compound B. In another specific embodiment of the various methods provided herein, the biomarker is MST1R (RON), and the treatment compound is Compound B. In another specific embodiment of the various methods provided herein, the biomarker is Nestin, and the treatment compound is Compound B. In another specific embodiment of the various methods provided herein, the biomarker is KAT1/CCBL1, and the treatment compound is Compound B. In another specific embodiment of the various methods provided herein, the biomarker is WIBG, and the treatment compound is Compound B. In another specific embodiment of the various methods provided herein, the biomarker is MVP, and the treatment compound is Compound B. In yet another specific embodiment of the various methods provided herein, the biomarker is PARP4, and the treatment compound is Compound B. In yet another specific embodiment of the various methods provided herein, the biomarker is ZNF198, and the treatment compound is Compound B. In yet another specific embodiment of the various methods provided herein, the biomarker is ZMYM198, and the treatment compound is Compound B. In yet another specific embodiment of the various methods provided herein, the biomarker is FAM83H, and the treatment compound is Compound B. In yet another specific embodiment of the various methods provided herein, the biomarker is KLC4, and the treatment compound is Compound B. In yet another specific embodiment of the various methods provided herein, the biomarker is EHMT2, and the treatment compound is Compound B. In yet another specific embodiment of the various methods provided herein, the biomarker is GLRX, and the treatment compound is Compound B. In yet another specific embodiment of the various methods provided herein, the biomarker is SDSL, and the treatment compound is Compound B. In yet another specific embodiment of the various methods provided herein, the biomarker is LIG3, and the treatment compound is Compound B. In yet another specific embodiment of the various methods provided herein, the biomarker is IKBKE, and the treatment compound is Compound B. In yet another specific embodiment of the various methods provided herein, the biomarker is PDE6D, and the treatment compound is Compound B. In yet another specific embodiment of the various methods provided herein, the biomarker is LGALS2, and the treatment compound is Compound B. In yet another specific embodiment of the various methods provided herein, the biomarker is LGALS9, and the treatment compound is Compound B.

In a specific embodiment of the various methods provided herein, the biomarker is ZFP91, and the treatment compound is Lenalidomide. In another embodiment of the various methods provided herein, the biomarker is CRBN, and the treatment compound is Lenalidomide. In yet another embodiment of the various methods provided herein, the biomarkers are both ZFP91 and CRBN, and the treatment compound is Lenalidomide. In another specific embodiment of the various methods provided herein, the biomarker is CK1-alpha, and the treatment compound is Lenalidomide. In another specific embodiment of the various methods provided herein, the biomarker is GSPT1, and the treatment compound is Lenalidomide. In another specific embodiment of the various methods provided herein, the biomarker is MST1R (RON), and the treatment compound is Lenalidomide. In another specific embodiment of the various methods provided herein, the biomarker is Nestin, and the treatment compound is Lenalidomide. In another specific embodiment of the various methods provided herein, the biomarker is KAT1/CCBL1, and the treatment compound is Lenalidomide. In another specific embodiment of the various methods provided herein, the biomarker is WIBG, and the treatment compound is Lenalidomide. In another specific embodiment of the various methods provided herein, the biomarker is MVP, and the treatment compound is Lenalidomide. In yet another specific embodiment of the various methods provided herein, the biomarker is PARP4, and the treatment compound is Lenalidomide. In yet another specific embodiment of the various methods provided herein, the biomarker is ZNF198, and the treatment compound is Lenalidomide. In yet another specific embodiment of the various methods provided herein, the biomarker is ZMYM198, and the treatment compound is Lenalidomide. In yet another specific embodiment of the various methods provided herein, the biomarker is FAM83H, and the treatment compound is Lenalidomide. In yet another specific embodiment of the various methods provided herein, the biomarker is KLC4, and the treatment compound is Lenalidomide. In yet another specific embodiment of the various methods provided herein, the biomarker is EHMT2, and the treatment compound is Lenalidomide. In yet another specific embodiment of the various methods provided herein, the biomarker is GLRX, and the treatment compound is Lenalidomide. In yet another specific embodiment of the various methods provided herein, the biomarker is SDSL, and the treatment compound is Lenalidomide. In yet another specific embodiment of the various methods provided herein, the biomarker is LIG3, and the treatment compound is Lenalidomide. In yet another specific embodiment of the various methods provided herein, the biomarker is IKBKE, and the treatment compound is Lenalidomide. In yet another specific embodiment of the various methods provided herein, the biomarker is PDE6D, and the treatment compound is Lenalidomide. In yet another specific embodiment of the various methods provided herein, the biomarker is LGALS2, and the treatment compound is Lenalidomide. In yet another specific embodiment of the various methods provided herein, the biomarker is LGALS9, and the treatment compound is Lenalidomide.

In yet other embodiments of the various methods provided herein, the biomarker is selected from a group consisting of SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTU1, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770, and the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In yet other embodiments of the various methods provided herein, the biomarker is selected from a group consisting of MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDC38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLII, FNBP1L, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830, and the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In yet other embodiments of the various methods provided herein, the biomarker is selected from a group consisting of ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Tap, Igf2r, IGFBP1, IGFBP3, IKBKE, JAG1, KIAA0100, KIAA1462, KIF20B, KIF22, KLC2, KRT9, LDLR, LPXN, MAFF, MELK, MET, MGAT4B, MGLL, MMP7, MST1R, MT2A, MVP, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2, and the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In yet other embodiments of the various methods provided herein, the biomarker is selected from a group consisting of ACSL6, AHSG, ALB, ARPP19, ATP5EP2, ATP5I, ATP5J, AZGP1, BCKDK, BLOC1S3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1H1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBC1D12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2, and the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In some embodiments of the various methods provided herein, a treatment compound is administered to a patient likely to be responsive to the treatment compound. In certain embodiments, the compound is administered to a patient as a dose of from about 0.1 mg per day to about 100 mg per day. In other embodiments, the treatment compound is administered a patient as a dose of between about 0.5 mg per day to about 100 mg per day. In other embodiments, the treatment compound is administered a patient as a dose of between about 0.5 mg per day to about 20 mg per day. In other embodiments, the treatment compound is administered a patient as a dose of between about 5 mg per day to about 25 mg per day. In some embodiments, the treatment compound is administered a patient as a dose of between about 0.5 mg per day to about 10 mg per day. In certain embodiments, the treatment compound is administered a patient as a dose of between about 0.5 mg per day to about 100 mg per day.

In other embodiments, the treatment compound is administered at a dose of about 0.1 mg, 0.5 mg, 1 mg, 1.5 mg, 2 mg, 2.5 mg, 3 mg, 3.5 mg, 4.0 mg, 4.5 mg, 5 mg, 5.5 mg, 6 mg, 6.5 mg, 7 mg, 7.5 mg, 8 mg, 8.5 mg, 9 mg, 9.5 mg, 10 mg, 11 mg, 12 mg, 13 mg, 14 mg, 15 mg, 20 mg, 25 mg, 30 mg, 35 mg, 40 mg, 45 mg, 50 mg, 60 mg, 70 mg, 80 mg, 90 mg or 100 mg per day.

In some embodiments, the treatment compound is administered once daily. In some embodiments, the treatment compound is administered twice daily. In certain embodiments, the treatment compound is cyclically administered to a patient. Cycling therapy involves the administration of an active agent for a period of time, followed by a rest for a period of time, and repeating this sequential administration. Accordingly, in some embodiments, about 0.5 mg per day to about 100 mg per day of the treatment compound is administered on days 1-12 of a repeated 28 day cycle. In a specific embodiment, 25 mg of the treatment compound is administered once a day on days 1-12 of a repeated 28 day cycle.

It is understood that specific dose levels of a treatment compound described for any particular subject depends upon a variety of factors including the activity of the specific compound employed, the age, body weight, general health, sex, and diet of the patient, the time of administration, the rate of excretion, the treatment compound combination, and the severity of the tumor being treated and form of administration. In it also understand that one of ordinary skill in the art can readily determine the appropriate dose of the treatment compound based on these factors. Treatment dosages generally may be titrated to optimize safety and efficacy.

A treatment compound can be administered by any route of administration known in the art, such as oral, intravenous, subcutaneous, or intramucosal administration. In one embodiment, lenalidomide or a stereoisomer thereof; or a pharmaceutically acceptable salt, solvate, hydrate, co-crystal, clathrate thereof; or a polymorph thereof, is administered to a patient orally. In one embodiment, Compound A or a stereoisomer thereof; or a pharmaceutically acceptable salt, solvate, hydrate, co-crystal, clathrate thereof; or a polymorph thereof, is administered to a patient orally. In one embodiment, thalidomide or a stereoisomer thereof; or a pharmaceutically acceptable salt, solvate, hydrate, co-crystal, clathrate thereof; or a polymorph thereof, is administered to a patient orally. In one embodiment, pomalidomide or a stereoisomer thereof; or a pharmaceutically acceptable salt, solvate, hydrate, co-crystal, clathrate thereof; or a polymorph thereof, is administered to a patient orally. In one embodiment, Compound B or a stereoisomer thereof; or a pharmaceutically acceptable salt, solvate, hydrate, co-crystal, clathrate thereof; or a polymorph thereof, is administered to a patient orally. The oral dosage form can be a tablet or a capsule. In some embodiments, the dosage form is a tablet. In some other embodiments, the dosage for is a capsule.

In some embodiments of the various methods provided herein, the levels of at least two biomarkers are determined. For example, in some embodiments of the various methods provided herein, two or more biomarkers independently selected from the group consisting of MST1R (RON), PARP4, MVP, ZFP91, IKBKE, CK1-alpha, PDE6D, LGALS2, KAT1, CRBN, and GSPT1 are determined. In other embodiments of the various methods provided herein, the levels of at least two biomarkers are determined, and one biomarker is CRBN and at least another biomarker is selected from Tables 1-6. In some embodiments of the various methods provided herein, the levels of at least two biomarkers are determined, and one biomarker is ZFP91 and at least another biomarker is selected from Tables 1-6. In some specific embodiments of the various methods provided herein, ZFP91 and one or more additional biomarker selected from the group consisting of MST1R (RON), PARP4, MVP, IKBKE, CK1-alpha, PDE6D, LGALS2, KAT1, and GSPT1 are determined. In some embodiments of the various methods provided herein, the levels of at least two biomarkers are determined, and one biomarker is CK1-alpha and at least another biomarker is selected from Tables 1-6. In some embodiments of the various methods provided herein, the levels of at least two biomarkers are determined, and one biomarker is GSPT1 and at least another biomarker is selected from Tables 1-6. In some embodiments of the various methods provided herein, the levels of at least two biomarkers are determined, and one biomarker is KAT1 and at least another biomarker is selected from Tables 1-6. In some embodiments of the various methods provided herein, the levels of at least two biomarkers are determined, and one biomarker is Nestin and at least another biomarker is selected from Tables 1-6. In some embodiments of the various methods provided herein, the levels of at least two biomarkers are determined, and one biomarker is WIBG and at least another biomarker is selected from Tables 1-6. In some embodiments of the various methods provided herein, the levels of at least two biomarkers are determined, and one biomarker is FAM83H and at least another biomarker is selected from Tables 1-6. In some embodiments of the various methods provided herein, the levels of at least two biomarkers are determined, and one biomarker is KLC4 and at least another biomarker is selected from Tables 1-6. In some embodiments of the various methods provided herein, the levels of at least two biomarkers are determined, and one biomarker is LIG3 and at least another biomarker is selected from Tables 1-6. In some embodiments of the various methods provided herein, the levels of at least two biomarkers are determined, and one biomarker is IKBKE and at least another biomarker is selected from Tables 1-6. In some embodiments of the various methods provided herein, the levels of at least two biomarkers are determined, and one biomarker is LGALS2 and at least another biomarker is selected from Tables 1-6. In some embodiments of the various methods provided herein, the levels of at least two biomarkers are determined, and one biomarker is LGALS9 and at least another biomarker is selected from Tables 1-6. In some embodiments of the various methods provided herein, the levels of at least two biomarkers are determined, and one biomarker is MVP and at least another biomarker is selected from Tables 1-6. In some embodiments of the various methods provided herein, the levels of at least two biomarkers are determined, and one biomarker is PARP4 and at least another biomarker is selected from Tables 1-6. In some embodiments of the various methods provided herein, the levels of at least two biomarkers are determined, and one biomarker is ZNF198 and at least another biomarker is selected from Tables 1-6. In some embodiments of the various methods provided herein, the levels of at least two biomarkers are determined, and one biomarker is MST1R (RON) and at least another biomarker is selected from Tables 1-6. In some embodiments of the various methods provided herein, the levels of at least two biomarkers are determined, and one biomarker is ZMYM198 and at least another biomarker is selected from Tables 1-6. In some embodiments of the various methods provided herein, the levels of at least two biomarkers are determined, and one biomarker is EHMT2 and at least another biomarker is selected from Tables 1-6. In some embodiments of the various methods provided herein, the levels of at least two biomarkers are determined, and one biomarker is GLRX and at least another biomarker is selected from Tables 1-6. In some embodiments of the various methods provided herein, the levels of at least two biomarkers are determined, and one biomarker is SDSL and at least another biomarker is selected from Tables 1-6. In some embodiments of the various methods provided herein, the levels of at least two biomarkers are determined, and one biomarker is PDE6D and at least another biomarker is selected from Tables 1-6.

Thus, in some specific embodiments, provided herein is a method of identifying a subject having a solid tumor who is likely to be responsive to a treatment compound, comprising:

(a) administering the treatment compound to a subject having a solid tumor;

(b) obtaining a sample from the subject;

(c) determining the levels of two or more biomarkers independently selected from the group consisting of MST1R (RON), PARP4, MVP, ZFP91, IKBKE, CK1-alpha, PDE6D, LGALS2, KAT1, and GSPT1; and

(d) diagnosing the subject as being likely to be responsive to the treatment compound if the level of the biomarkers in the sample of the subject changes as compared to a reference levels of the biomarkers.

In some embodiments, the method comprises determining the levels of ZFP91 and one or more markers selected from the group consisting of MST1R (RON), PARP4, MVP, IKBKE, CK1-alpha, PDE6D, LGALS2, KAT1, and GSPT1.

In some embodiments, the method further comprises administering a therapeutically effective amount of the treatment compound to the subject diagnosed to be likely to be responsive to the treatment compound.

4.3.2 Use of Biomarkers for Predicting or Monitoring the Efficacy

Based, in part, on the finding that detectable increase or decrease in certain biomarkers are observed in subjects with a solid tumor, e.g., HCC or GBM, who are responsive to a given treatment (e.g., a treatment by a treatment compound, e.g., thalidomide, lenalidomide, pomalidomide, Compound A, or Compound B, or a stereoisomer thereof, or a pharmaceutically acceptable salt, solvate, hydrate, co-crystal, clathrate, or a polymorph thereof), the levels of these biomarkers may be used for predicting the responsiveness of the subjects to the treatment.

In another aspect, provided herein is a method of predicting the responsiveness of a subject having or suspected of having a solid tumor, e.g., HCC or GBM, to a treatment compound.

In some embodiments, provided herein is a method of predicting the responsiveness of a subject having or suspected of having a solid tumor, e.g., HCC or GBM, to a treatment compound, comprising:

(a) administering the treatment compound to a subject having a solid tumor;

(b) obtaining a sample from the subject;

(c) determining the level of a biomarker in the sample from the subject, wherein the biomarker is selected from the group consisting of biomarkers identified in Tables 1 to 6, and combinations thereof;

(d) diagnosing the subject as being likely to be responsive to a treatment of the solid tumor with the treatment compound if the level of the biomarker in the sample changes as compared to the level of the biomarker obtained from a reference sample. In one embodiment, the solid tumor is HCC. In another embodiment, the solid tumor is GBM.

In other embodiments, provided herein is a method of predicting the responsiveness of a subject having or suspected of having a solid tumor, e.g., HCC or GBM, to a treatment compound, comprising:

(a) administering the treatment compound to a subject having a solid tumor;

(b) obtaining a sample from the subject;

(c) determining the level of a biomarker in the sample from the subject, wherein the biomarker is selected from the group consisting of biomarkers identified in Table 1, Table 3, and Table 4, and combinations thereof;

(d) diagnosing the subject as being likely to be responsive to a treatment of the solid tumor with the treatment compound if the level of the biomarker in the sample is higher than the level of the biomarker obtained from a reference sample. In one embodiment, the solid tumor is HCC. In another embodiment, the solid tumor is GBM.

In yet other embodiments, provided herein is a method of predicting the responsiveness of a subject having or suspected of having a solid tumor, e.g., HCC or GBM, to a treatment compound, comprising:

(a) administering the treatment compound to a subject having a solid tumor;

(b) obtaining a sample from the subject;

(c) determining the level of a biomarker in the sample from the subject, wherein the biomarker is selected from the group consisting of biomarkers identified in Table 2, Table 5, and Table 6, and combinations thereof;

(d) diagnosing the subject as being likely to be responsive to a treatment of the solid tumor with the treatment compound if the level of the biomarker in the sample is less than the level of the biomarker obtained from a reference sample. In one embodiment, the solid tumor is HCC. In another embodiment, the solid tumor is GBM.

In yet another aspect, provided herein is a method of monitoring the efficacy of a treatment of a solid tumor, e.g., HCC or GBM, in a subject with a treatment compound, comprising:

(a) administering the treatment compound to a subject having a solid tumor;

(b) obtaining a sample from the subject;

(c) determining the level of a biomarker in the sample from the subject, wherein the biomarker is selected from the group consisting of biomarkers identified in Tables 1 to 6, and combinations thereof;

(d) comparing the level of the biomarker in the sample with the level of the biomarker obtained from a reference sample, wherein a change in the level as compared to the reference is indicative of the efficacy of the treatment compound in treating the solid tumor in the subject. In one embodiment, the solid tumor is HCC. In another embodiment, the solid tumor is GBM.

In other embodiments, provided herein is a method of monitoring the efficacy of a treatment of a solid tumor, e.g., HCC or GBM, in a subject with a treatment compound, comprising:

(a) administering the treatment compound to a subject having a solid tumor;

(b) obtaining a sample from the subject;

(c) determining the level of a biomarker in the sample from the subject, wherein the biomarker is selected from the group consisting of biomarkers identified in Table 1, Table 3, and Table 4, and combinations thereof;

(d) comparing the level of the biomarker in the sample with the level of the biomarker obtained from a reference sample, wherein an increased level as compared to the reference is indicative of the efficacy of the treatment compound in treating the solid tumor in the subject. In one embodiment, the solid tumor is HCC. In another embodiment, the solid tumor is GBM.

In yet other embodiments, provided herein is a method of monitoring the efficacy of a treatment of a solid tumor, e.g., HCC or GBM, in a subject with a treatment compound, comprising:

(a) administering the treatment compound to a subject having a solid tumor;

(b) obtaining a sample from the subject;

(c) determining the level of a biomarker in the sample from the subject, wherein the biomarker is selected from the group consisting of biomarkers identified in Table 2, Table 5, and Table 6, and combinations thereof;

(d) comparing the level of the biomarker in the sample with the level of the biomarker obtained from a reference sample, wherein a decreased level as compared to the reference is indicative of the efficacy of the treatment compound in treating the solid tumor in the subject. In one embodiment, the solid tumor is HCC. In another embodiment, the solid tumor is GBM.

In some embodiments of the various methods provided herein, the biomarker is selected from a group consisting of CRBN, CK1-alpha, GSPT1, Nestin, KAT1/CCBL1, WIBG, MVP, PARP4, ZFP91, ZNF198, and MST1R (RON). In some embodiments of the various methods provided herein, the biomarker is selected from a group consisting of CRBN, CK1-alpha, GSPT1, Nestin, KAT1/CCBL1, and WIBG. In other embodiments, the biomarker is selected from a group consisting of MVP, PARP4, ZFP91, ZNF198, and MST1R (RON).

In some embodiments of the various methods provided herein, the biomarker is selected from a group consisting of AHNAK, ALOX5, AMPD3, ANXA4, ANXA6, ARHGAP19, ASNS, ASPM, ATP2B4, B4GALT3, BANK1, BCDIN3D, BLZF1, BMF, BST2, C10orf76, C19orf66, CA2, CA8, CAMSAP3, CCDC69, CCNB1, CD36, CDC7, CDCA3, CENPF, CLN3, CNN3, CORO1B, CPNE2, CRBN, CSNK1A1, CSRP2, CTNND1, CTSH, DAPK2, DDX58, DHPS, DHX58, DLG2, DLGAP5, DOK3, DTX3L, ECT2, EFCAB4B, EHMT1, EHMT2, EIF2AK2, EPB41L1, EPCAM, ESRP1, ETV6, EXTL2, F13A1, FAM195A, FAM65B, FBRSL1, FCGR2B, FES, FHOD1, FIGNL1, FMNL3, GBP1, GMFG, GMPR, GPT2, GRAMD1A, GRAMD1B, GRPEL2, HIP1, HJURP, HLA-B, HLA-DMA, HMCES, HMMR, HOXC4, HPSE, ICAM2, ID3, IFI35, IFIH1, IFIT1, IFIT3, IFIT5, IFITM2, IKZF1, IKZF3, IL4I1, IRF7, IRF9, IRS2, ISG15, ISG20, ITGB7, JAK3, KIF18B, KIF22, KIF2C, LAP3, LGALS1, LGALS3BP, LIMD1, LIPG, LPXN, MAN2A2, MARCKS, MFI2, MGARP, MINA, MIS18BP1, MOV10, MPP7, MUC1, MX1, MX2, MYO1G, NCF2, NEIL1, NFKBID, NME3, NMI, NPIPB5, NT5C3A, OAS1, OAS2, OAS3, OMA1, ORC6, PARP14, PARP9, PARVB, PBK, PBXIP1, PDE6D, PKMYT1, PLD4, PLEKHO1, PLK1, PLSCR1, PLXNB2, PODXL, PODXL2, POLE2, POMP, PPFIBP1, PRDM15, PRNP, PTAFR, PTMS, PTTG1, PYROXD1, QPRT, RAB13, RASA4B, RASSF6, RCN1, RGCC, RGS1, RGS2, RNF213, S100A13, SAMD9L, SAMHD1, SEC14L1, SERPINH1, SGOL1, SGOL2, SLCO3A1, SLCO4A1, SLFN11, SLFN13, SLFN5, SP110, SP140, SPN, SPR, STAP1, STAT1, STAT2, TACC3, TAP1, TAX1BP3, THEMIS2, THTPA, TIMM8B, TNFAIP8L2, TNFSF8, TOP2A, TP5313, TPX2, TREX1, TRIB3, TRIM22, TTC39C, TXNIP, UBA7, UBE2L6, USP41, VCL, VNN2, WIZ, WSB1, WWC1, ZBTB38, ZFP91, ZMYM2, ZNF385B, ZNF581 and ZNF644.

In other embodiments of the various methods provided herein, the biomarker is selected from a group consisting of AHNAK, ALOX5, AMPD3, ANXA4, ANXA6, ATP2B4, BMF, BST2, C10orf76, C19orf66, CD36, CLN3, CNN3, CORO1B, CPNE2, CRBN, CSRP2, CTNND1, CTSH, DAPK2, DDX58, DHX58, DLG2, DTX3L, EIF2AK2, EPB41L1, ETV6, EXTL2, F13A1, FAM65B, FCGR2B, FES, FMNL3, GBP1, GMFG, GMPR, HIP1, HLA-B, HLA-DMA, HPSE, ID3, IFI35, IFIH1, IFIT1, IFIT3, IFIT5, IFITM2, IL4I1, IRF7, IRF9, ISG15, ISG20, ITGB7, JAK3, LAP3, LGALS1, LGALS3BP, LIMD1, MAN2A2, MARCKS, MFI2, MGARP, MOV10, MPP7, MUC1, MX1, MX2, MYO1G, NCF2, NME3, NMI, NT5C3A, OAS1, OAS2, OAS3, PARP14, PARP9, PBXIP1, PLD4, PLEKHO1, PLSCR1, PLXNB2, POMP, PPFIBP1, PTMS, QPRT, RAB13, RCN1, RGCC, RNF213, S100A13, SAMD9L, SAMHD1, SERPINH1, SLFN11, SLFN13, SLFN5, SP110, SP140, SPN, SPR, STAP1, STAT1, STAT2, TAP1, TAX1BP3, THEMIS2, THTPA, TNFAIP8L2, TNFSF8, TP5313, TREX1, TRIM22, TTC39C, TXNIP, UBA7, UBE2L6, USP41, VCL, VNN2 and ZBTB38.

In yet other embodiments of the various methods provided herein, the biomarker is selected from a group consisting of ARHGAP19, ASNS, ASPM, B4GALT3, BANK1, BCDIN3D, BLZF1, CA2, CA8, CAMSAP3, CCDC69, CCNB1, CDC7, CDCA3, CENPF, CSNK1A1DHPS, DLGAP5, DOK3, ECT2, EFCAB4B, EHMT1, EHMT2, EPCAM, ESRP1, FAM195A, FBRSL1, FHOD1, FIGNL1, GPT2, GRAMD1A, GRAMD1B, GRPEL2, HJURP, HMCES, HMMR, HOXC4, ICAM2, IKZF1, IKZF3, IRS2, KIF18B, KIF22, KIF2C, LIPG, LPXN, MINA, MIS18BP1, NEIL1, NFKBID, NPIPB5, OMA1, ORC6, PARVB, PBK, PDE6D, PKMYT1, PLK1, PODXL, PODXL2, POLE2, PRDM15, PRNP, PTAFR, PTTG1, PYROXD1, RASA4B, RASSF6, RGS1, RGS2, SEC14L1, SGOL1, SGOL2, SLCO3A1, SLCO4A1, TACC3, TIMM8B, TOP2A, TPX2, TRIB3, WIZ, WSB1, WWC1, ZFP91, ZMYM2, ZNF385B, ZNF581 and ZNF644.

In yet other embodiments of the various methods provided herein, the biomarker is selected from a group consisting of SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTU1, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770.

In yet other embodiments of the various methods provided herein, the biomarker is selected from a group consisting of MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDC38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLII, FNBP1L, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830.

In yet other embodiments of the various methods provided herein, the biomarker is selected from a group consisting of ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Tap, Igf2r, IGFBP1, IGFBP3, IKBKE, JAG1, KIAA0100, KIAA1462, KIF20B, KIF22, KLC2, KRT9, LDLR, LPXN, MAFF, MELK, MET, MGAT4B, MGLL, MMP7, MST1R, MT2A, MVP, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2.

In yet other embodiments of the various methods provided herein, the biomarker is selected from a group consisting of ACSL6, AHSG, ALB, ARPP19, ATP5EP2, ATP5I, ATP5J, AZGP1, BCKDK, BLOC1S3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1H1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBC1D12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2.

In certain embodiments, any combination of two or more of the above-identified biomarkers is also contemplated.

In some embodiments of the various methods provided herein, the biomarker is selected from a group consisting of FAM83H, KLC4, LIG3, IKBKE, LGALS2 and LGALS9, and the level of the biomarker increases in response to compound treatment, e.g., by Compound A. In some embodiments of the various methods provided herein, the biomarker is selected from a group consisting of ZMYM198, EHMT2, GLRX, SDSL, and PDE6D, and the level of the biomarker decreases in response to compound treatment, e.g., by Compound A. In a specific embodiment, the biomarker is ZMYM198. In another specific embodiment, the biomarker is FAM83H. In another specific embodiment, the biomarker is KLC4. In another embodiment, the biomarker is EHMT2. In another embodiment, the biomarker is GLRX. In another embodiment, the biomarker is SDSL. In another specific embodiment, the biomarker is LIG3. In another specific embodiment, the biomarker is IKBKE. In another specific embodiment, the biomarker is PDE6D. In another specific embodiment, the biomarker is LGALS2. In another specific embodiment, the biomarker is LGALS9.

In a specific embodiment of the various methods provided herein, the biomarker is ZFP91. In another embodiment of the various methods provided herein, the biomarker is CRBN. In yet another embodiment of the various methods provided herein, the biomarkers are both ZFP91 and CRBN. In another specific embodiment of the various methods provided herein, the biomarker is Nestin. In another specific embodiment of the various methods provided herein, the biomarker is KAT1/CCBL1. In another specific embodiment of the various methods provided herein, the biomarker is WIBG. In another specific embodiment of the various methods provided herein, the biomarker is MVP. In yet another specific embodiment of the various methods provided herein, the biomarker is PARP4. In yet another specific embodiment of the various methods provided herein, the biomarker is ZNF198. In yet another specific embodiment, the biomarker is CK1-alpha. In yet another specific embodiment, the biomarker is GSPT1.

In some embodiments of the various methods provided herein, the level of only one biomarker is determined. In other embodiments of the various methods provided herein, the levels of two, three, four, five or more biomarkers are determined.

In some embodiments of the various methods provided herein, two or more biomarkers identified in Table 1, Table 2, Table 3, Table 4, Table 5, and/or Table 6 are measured and compared with the reference sample. In some embodiments, the two or more biomarkers are identified in Table 1, Table 3, and Table 4, and the levels of the biomarkers increase as compared to the levels of the biomarkers in the reference. In other embodiments of the various methods provided herein, the two or more biomarkers are identified in Table 2, Table 5, and Table 6, and the levels of the biomarkers decrease as compared to the levels of the biomarkers in the reference. In yet other embodiments of the various methods provided herein, one or more biomarkers are identified in Table 1, Table 3, and/or Table 4, and one or more biomarkers are identified in Table 2, Table 5, and/or Table 6, and the levels of some biomarkers increase as compared to the reference and the level of some biomarkers decrease as compared to the reference.

In some embodiments of the various methods provided herein, the reference is prepared by using a control sample obtained from the subject prior to administration of the treatment compound to the subject; and wherein the control sample is from the same source as the sample. In other embodiments of the various methods provided herein, the reference is prepared by using a control sample obtained from a healthy subject not having the solid tumor; and wherein the control sample is from the same source as the sample.

In some embodiments of the various methods provided herein, the levels of one or more of the biomarkers are measured by determining the mRNA levels of the biomarkers. In some embodiments, the mRNA levels of the biomarkers are determined by reverse transcriptase PCR (RT-PCR). In some embodiments, the mRNA levels of the biomarkers are determined by quantitative RT-PCR (qRT-PCR).

In other embodiments of the various methods provided herein, the levels of one or more of the biomarkers are measured by determining the cDNA levels of the biomarkers. In some embodiments of the various methods provided herein, the cDNA levels of the biomarkers are determined by PCR.

In yet other embodiments of the various methods provided herein, the levels of one or more of the biomarkers are measured by determining the protein levels of the biomarkers.

In some embodiments of the various methods provided herein, the method provided herein further comprises contacting proteins within the sample with a first antibody that immunospecifically binds to the biomarker protein.

In one embodiment, the method provided herein further comprises (i) contacting the proteins bound to the first antibody with a second antibody with a detectable label, wherein the second antibody immunospecifically binds to the biomarker protein, and wherein the second antibody immunospecifically binds to a different epitope on the biomarker protein than the first antibody; (ii) detecting the presence of second antibody bound to the biomarker protein; and (iii) determining the amount of the biomarker protein based on the amount of detectable label in the second antibody.

In another embodiment, the method provided herein further comprises (i) contacting the proteins bound to the first antibody with a second antibody with a detectable label, wherein the second antibody immunospecifically binds to the first antibody; (ii) detecting the presence of second antibody bound to the first antibody; and (iii) determining the amount of the biomarker protein based on the amount of detectable label in the second antibody.

In a specific embodiment of the various methods provided herein, the biomarker is ZFP91. Thus, in some embodiments, provided herein is a method of predicting the responsiveness of a subject having or suspected of having a solid tumor, e.g., HCC or GBM, to a treatment compound, comprising:

(a) administering the treatment compound to a subject having a solid tumor;

(b) obtaining a sample from the subject;

(c) determining the level of ZFP91 in the sample from the subject; and

(d) diagnosing the subject as being likely to be responsive to a treatment of the solid tumor with the treatment compound if the level of ZFP91 in the sample is less than the level of ZFP91 obtained from a reference sample. In one embodiment, the solid tumor is HCC. In another embodiment, the solid tumor is GBM.

In some embodiments, provided herein is a method of monitoring the efficacy of a treatment of a solid tumor, e.g., HCC or GBM, in a subject with a treatment compound, comprising:

(a) administering the treatment compound to a subject having a solid tumor;

(b) obtaining a sample from the subject;

(c) determining the level of ZFP91 in the sample from the subject;

(d) comparing the level of ZFP91 in the sample with the level of ZFP91 obtained from a reference sample, wherein a decrease in the level as compared to the reference is indicative of the efficacy of the treatment compound in treating the solid tumor in the subject. In one embodiment, the solid tumor is HCC. In another embodiment, the solid tumor is GBM.

In some embodiments, the level of ZFP91 in the sample is less than 90% of the level of ZFP91 of a reference. In some embodiments, the level of ZFP91 in the sample is less than 80% of the level of ZFP91 of a reference. In other embodiments, the level of ZFP91 in the sample is less than 70% of the level of ZFP91 of a reference. In other embodiments, the level of ZFP91 in the sample is less than 60% of the level of ZFP91 of a reference. In other embodiments, the level of ZFP91 in the sample is less than 50% of the level of ZFP91 of a reference. In other embodiments, the level of ZFP91 in the sample is less than 40% of the level of ZFP91 of a reference. In yet other embodiments, the level of ZFP91 in the sample is less than 30% of the level of ZFP91 of a reference. In yet other embodiments, the level of ZFP91 in the sample is less than 20% of the level of ZFP91 of a reference. In yet other embodiments, the level of ZFP91 in the sample is less than 10% of the level of ZFP91 of a reference.

In some embodiments, the protein level of ZFP91 is measured. For example, in some embodiments, the method provided herein comprises contacting proteins within the sample with a first antibody that immunospecifically binds to ZFP91 protein. In some embodiments, the method provided herein further includes (i) contacting the proteins bound to the first antibody with a second antibody with a detectable label, wherein the second antibody immunospecifically binds to ZFP91, and wherein the second antibody immunospecifically binds to a different epitope on ZFP91protein than the first antibody; (ii) detecting the presence of second antibody bound to the proteins; and (iii) determining the amount of ZFP91 protein based on the amount of detectable label in the second antibody. In other embodiments, the method provided herein further comprises (i) contacting the proteins bound to the first antibody with a second antibody with a detectable label, wherein the second antibody immunospecifically binds to the first antibody; (ii) detecting the presence of second antibody bound to the proteins; and (iii) determining the amount of ZFP91 protein based on the amount of detectable label in the second antibody.

In some embodiments, the mRNA level of ZFP91 is measured. For example, in some embodiments, the method provided herein comprises extracting mRNA from the sample. In some embodiments, the method further comprises determining mRNA level of ZFP91 using PCR. In some embodiments, the PCR is a RT-PCR.

In some embodiments, the method provided herein further comprises generating cDNA from the mRNA. In some embodiments, the method provided herein further comprises performing a PCR to quantify the cDNA representing ZFP91.

In some embodiments, the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide. In some embodiments, the solid tumor is HCC. In some embodiments, the solid tumor is GBM. In some embodiments, the treatment compound is Compound A, and the solid tumor is HCC. In some embodiments, the treatment compound is Compound B, and the solid tumor is HCC. In some embodiments, the treatment compound is lenadlidomide, and the solid tumor is HCC. In some embodiments, the treatment compound is Compound A, and the solid tumor is GBM. In some embodiments, the treatment compound is Compound B, and the solid tumor is GBM. In some embodiments, the treatment compound is lenadlidomide, and the solid tumor is GBM.

In a specific embodiment of the various methods provided herein, the biomarker is CRBN. Thus, in some embodiments, provided herein is a method of predicting the responsiveness of a subject having or suspected of having a solid tumor, e.g., HCC or GBM, to a treatment compound, comprising:

(a) administering the treatment compound to a subject having a solid tumor;

(b) obtaining a sample from the subject;

(c) determining the level of CRBN in the sample from the subject; and

(d) diagnosing the subject as being likely to be responsive to a treatment of the solid tumor with the treatment compound if the level of CRBN in the sample is higher than the level of CRBN obtained from a reference sample. In one embodiment, the solid tumor is HCC. In another embodiment, the solid tumor is GBM.

In some embodiments, provided herein is a method of monitoring the efficacy of a treatment of a solid tumor, e.g., HCC or GBM, in a subject with a treatment compound, comprising:

(a) administering the treatment compound to a subject having a solid tumor;

(b) obtaining a sample from the subject;

(c) determining the level of CRBN in the sample from the subject;

(d) comparing the level of CRBN in the sample with the level of CRBN obtained from a reference sample, wherein an increase in the level as compared to the reference is indicative of the efficacy of the treatment compound in treating the solid tumor in the subject. In one embodiment, the solid tumor is HCC. In another embodiment, the solid tumor is GBM.

In some embodiments, the level of CRBN in the sample is 10% more than the level of CRBN of a reference. In some embodiments, the level of ZFP91 in the sample is 20% more than the level of CRBN of a reference. In other embodiments, the level of CRBN in the sample is 30% more than the level of CRBN of a reference. In other embodiments, the level of CRBN in the sample is 40% more than the level of CRBN of a reference. In other embodiments, the level of CRBN in the sample is 50% more than the level of CRBN of a reference. In other embodiments, the level of CRBN in the sample is 60% more than the level of CRBN of a reference. In yet other embodiments, the level of CRBN in the sample is 70% more than the level of CRBN of a reference. In yet other embodiments, the level of CRBN in the sample is 80% of the level of CRBN of a reference. In yet other embodiments, the level of CRBN in the sample is 90% more than the level of CRBN of a reference. In yet other embodiments, the level of CRBN in the sample is 2-fold, 3-fold, 4-fold, 5-fold, 6-fold, 7-fold, 8-fold, 9-fold, 10-fold of the CRBN level of a reference.

In some embodiments, the protein level of CRBN is measured. For example, in some embodiments, the method provided herein comprises contacting proteins within the sample with a first antibody that immunospecifically binds to CRBN protein. In some embodiments, the method provided herein further includes (i) contacting the proteins bound to the first antibody with a second antibody with a detectable label, wherein the second antibody immunospecifically binds to CRBN, and wherein the second antibody immunospecifically binds to a different epitope on CRBN protein than the first antibody; (ii) detecting the presence of second antibody bound to the proteins; and (iii) determining the amount of CRBN protein based on the amount of detectable label in the second antibody. In other embodiments, the method provided herein further comprises (i) contacting the proteins bound to the first antibody with a second antibody with a detectable label, wherein the second antibody immunospecifically binds to the first antibody; (ii) detecting the presence of second antibody bound to the proteins; and (iii) determining the amount of CRBN protein based on the amount of detectable label in the second antibody.

In some embodiments, the mRNA level of CRBN is measured. For example, in some embodiments, the method provided herein comprises extracting mRNA from the sample. In some embodiments, the method further comprises determining mRNA level of CRBN using PCR. In some embodiments, the PCR is a RT-PCR.

In some embodiments, the method provided herein further comprises generating cDNA from the mRNA. In some embodiments, the method provided herein further comprises performing a PCR to quantify the cDNA representing CRBN.

In some embodiments, the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide. In some embodiments, the solid tumor is HCC. In some embodiments, the solid tumor is GBM. In some embodiments, the treatment compound is Compound A, and the solid tumor is HCC. In some embodiments, the treatment compound is Compound B, and the solid tumor is HCC. In some embodiments, the treatment compound is lenadlidomide, and the solid tumor is HCC. In some embodiments, the treatment compound is Compound A, and the solid tumor is GBM. In some embodiments, the treatment compound is Compound B, and the solid tumor is GBM. In some embodiments, the treatment compound is lenadlidomide, and the solid tumor is GBM.

In a specific embodiment of the various methods provided herein, the biomarkers are ZFP91 and CRBN. Thus, in some embodiments, provided herein is a method of predicting the responsiveness of a subject having or suspected of having a solid tumor, e.g., HCC or GBM, to a treatment compound, comprising:

(a) administering the treatment compound to a subject having a solid tumor;

(b) obtaining a sample from the subject;

(c) determining the levels of ZFP91 and CRBN in the sample from the subject; and

(d) diagnosing the subject as being likely to be responsive to a treatment of the solid tumor with the treatment compound if the level of ZFP91 in the sample is lower than the level of ZFP91 obtained from a reference sample and the level of CRBN in the sample is higher than the level of CRBN obtained from a reference sample. In one embodiment, the solid tumor is HCC. In another embodiment, the solid tumor is GBM.

In some embodiments, provided herein is a method of monitoring the efficacy of a treatment of a solid tumor, e.g., HCC or GBM, in a subject with a treatment compound, comprising:

(a) administering the treatment compound to a subject having a solid tumor;

(b) obtaining a sample from the subject;

(c) determining the levels of ZFP91 and CRBN in the sample from the subject;

(d) comparing the level of ZFP91 and CRBN in the sample with the levels of ZFP91 and CRBN obtained from a reference sample, wherein a decrease in the level of ZFP91 as compared to the reference and an increase in the level of CRBN as compared to the reference is indicative of the efficacy of the treatment compound in treating the solid tumor in the subject.

In some embodiments, the level of ZFP91 in the sample is less than 90% of the level of ZFP91 of a reference. In some embodiments, the level of ZFP91 in the sample is less than 80% of the level of ZFP91 of a reference. In other embodiments, the level of ZFP91 in the sample is less than 70% of the level of ZFP91 of a reference. In other embodiments, the level of ZFP91 in the sample is less than 60% of the level of ZFP91 of a reference. In other embodiments, the level of ZFP91 in the sample is less than 50% of the level of ZFP91 of a reference. In other embodiments, the level of ZFP91 in the sample is less than 40% of the level of ZFP91 of a reference. In yet other embodiments, the level of ZFP91 in the sample is less than 30% of the level of ZFP91 of a reference. In yet other embodiments, the level of ZFP91 in the sample is less than 20% of the level of ZFP91 of a reference. In yet other embodiments, the level of ZFP91 in the sample is less than 10% of the level of ZFP91 of a reference.

In some embodiments, the level of CRBN in the sample is 10% more than the level of CRBN of a reference. In some embodiments, the level of ZFP91 in the sample is 20% more than the level of CRBN of a reference. In other embodiments, the level of CRBN in the sample is 30% more than the level of CRBN of a reference. In other embodiments, the level of CRBN in the sample is 40% more than the level of CRBN of a reference. In other embodiments, the level of CRBN in the sample is 50% more than the level of CRBN of a reference. In other embodiments, the level of CRBN in the sample is 60% more than the level of CRBN of a reference. In yet other embodiments, the level of CRBN in the sample is 70% more than the level of CRBN of a reference. In yet other embodiments, the level of CRBN in the sample is 80% of the level of CRBN of a reference. In yet other embodiments, the level of CRBN in the sample is 90% more than the level of CRBN of a reference. In yet other embodiments, the level of CRBN in the sample is 2-fold, 3-fold, 4-fold, 5-fold, 6-fold, 7-fold, 8-fold, 9-fold, 10-fold of the CRBN level of a reference.

In some embodiments, the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide. In some embodiments, the solid tumor is HCC. In some embodiments, the solid tumor is GBM. In some embodiments, the treatment compound is Compound A, and the solid tumor is HCC. In some embodiments, the treatment compound is Compound B, and the solid tumor is HCC. In some embodiments, the treatment compound is lenadlidomide, and the solid tumor is HCC. In some embodiments, the treatment compound is Compound A, and the solid tumor is GBM. In some embodiments, the treatment compound is Compound B, and the solid tumor is GBM. In some embodiments, the treatment compound is lenadlidomide, and the solid tumor is GBM.

In some embodiments of the various methods provided herein, the treatment compound is thalidomide, lenalidomide, pomalidomide, Compound A or Compound B, or a stereoisomer thereof, or a pharmaceutically acceptable salt, solvate, hydrate, co-crystal, clathrate, or a polymorph thereof. In some embodiments, the treatment compound is thalidomide, or a stereoisomer thereof, or a pharmaceutically acceptable salt, solvate, hydrate, co-crystal, clathrate, or a polymorph thereof. In other embodiments, the treatment compound is lenalidomide, or a stereoisomer thereof, or a pharmaceutically acceptable salt, solvate, hydrate, co-crystal, clathrate, or a polymorph thereof. In yet other embodiments, the treatment compound is pomalidomide, or a stereoisomer thereof, or a pharmaceutically acceptable salt, solvate, hydrate, co-crystal, clathrate, or a polymorph thereof. In yet other embodiments, the treatment compound is Compound A, or a stereoisomer thereof, or a pharmaceutically acceptable salt, solvate, hydrate, co-crystal, clathrate, or a polymorph thereof. In yet other embodiments, the treatment compound is Compound B, or a stereoisomer thereof, or a pharmaceutically acceptable salt, solvate, hydrate, co-crystal, clathrate, or a polymorph thereof.

In some embodiments, the biomarkers are used to predict and/or monitor efficacy of a treatment with thalidomide, lenalidomide, pomalidomide, Compound A or Compound B, or a stereoisomer thereof, or a pharmaceutically acceptable salt, solvate, hydrate, co-crystal, clathrate, or a polymorph thereof.

In one embodiment, the biomarkers are used to predict and/or monitor efficacy of a treatment with thalidomide, or a stereoisomer thereof, or a pharmaceutically acceptable salt, solvate, hydrate, co-crystal, clathrate, or a polymorph thereof. In one embodiment, the biomarkers are used to predict and/or monitor efficacy of a treatment with lenalidomide, or a stereoisomer thereof, or a pharmaceutically acceptable salt, solvate, hydrate, co-crystal, clathrate, or a polymorph thereof. In another embodiment, the biomarkers are used to predict and/or monitor efficacy of a treatment with pomalidomide, or a stereoisomer thereof, or a pharmaceutically acceptable salt, solvate, hydrate, co-crystal, clathrate, or a polymorph thereof. In yet another embodiment, the biomarkers are used to predict and/or monitor efficacy of a treatment with Compound A, or a stereoisomer thereof, or a pharmaceutically acceptable salt, solvate, hydrate, co-crystal, clathrate, or a polymorph thereof. In yet another embodiment, the biomarkers are used to predict and/or monitor efficacy of a treatment with Compound B, or a stereoisomer thereof, or a pharmaceutically acceptable salt, solvate, hydrate, co-crystal, clathrate, or a polymorph thereof.

In some embodiments, provided herein is a method of predicting the responsiveness of a subject having or suspected of having a solid tumor, e.g., HCC or GBM, to Compound A, comprising:

(a) administering Compound A to a subject having a solid tumor;

(b) obtaining a sample from the subject;

(c) determining the level of a biomarker in the sample from the subject, wherein the biomarker is selected from the group consisting of biomarkers identified in Tables 1 to 6, and combinations thereof;

(d) diagnosing the subject as being likely to be responsive to a treatment of the solid tumor with Compound A if the level of the biomarker in the sample changes as compared to the level of the biomarker obtained from a reference sample. In one embodiment, the solid tumor is HCC. In another embodiment, the solid tumor is GBM.

In some embodiments, provided herein is a method of monitoring the efficacy of a treatment of a solid tumor, e.g., HCC or GBM, in a subject with Compound A, comprising:

(a) administering Compound A to a subject having a solid tumor;

(b) obtaining a sample from the subject;

(c) determining the level of a biomarker in the sample from the subject, wherein the biomarker is selected from the group consisting of biomarkers identified in Tables 1 to 6, and combinations thereof;

(d) comparing the level of the biomarker in the sample with the level of the biomarker obtained from a reference sample, wherein a change in the level as compared to the reference is indicative of the efficacy of Compound A in treating the solid tumor in the subject. In one embodiment, the solid tumor is HCC. In another embodiment, the solid tumor is GBM.

In some embodiments, the biomarker is selected from a group consisting of CRBN, CK1-alpha, GSPT1, Nestin, KAT1/CCBL1, WIBG, FAM83H, KLC4, LIG3, IKBKE, LGALS2 LGALS9, MVP, PARP4, ZFP91, ZNF198, MST1R (RON), ZMYM198, EHMT2, GLRX, SDSL, and PDE6D. In some embodiments of the various methods provided herein, the biomarker is selected from a group consisting of CRBN, CK1-alpha, GSPT1, Nestin, KAT1/CCBL1, WIBG, FAM83H, KLC4, LIG3, IKBKE, LGALS2 and LGALS9. In other embodiments, the biomarker is selected from a group consisting of MVP, PARP4, ZFP91, ZNF198, MST1R (RON), ZMYM198, EHMT2, GLRX, SDSL, and PDE6D. In a specific embodiment of the various methods provided herein, the biomarker is ZFP91. In another embodiment of the various methods provided herein, the biomarker is CRBN. In yet another embodiment of the various methods provided herein, the biomarkers are both ZFP91 and CRBN. In another specific embodiment of the various methods provided herein, the biomarker is CK1-alpha. In another specific embodiment of the various methods provided herein, the biomarker is GSPT1. In another specific embodiment of the various methods provided herein, the biomarker is MST1R (RON). In another specific embodiment of the various methods provided herein, the biomarker is Nestin. In another specific embodiment of the various methods provided herein, the biomarker is KAT1/CCBL1. In another specific embodiment of the various methods provided herein, the biomarker is WIBG. In another specific embodiment of the various methods provided herein, the biomarker is MVP. In yet another specific embodiment of the various methods provided herein, the biomarker is PARP4. In yet another specific embodiment of the various methods provided herein, the biomarker is ZNF198. In yet another specific embodiment of the various methods provided herein, the biomarker is ZMYM198. In yet another specific embodiment of the various methods provided herein, the biomarker is FAM83H. In yet another specific embodiment of the various methods provided herein, the biomarker is KLC4. In yet another specific embodiment of the various methods provided herein, the biomarker is EHMT2. In yet another specific embodiment of the various methods provided herein, the biomarker is GLRX. In yet another specific embodiment of the various methods provided herein, the biomarker is SDSL. In yet another specific embodiment of the various methods provided herein, the biomarker is LIG3. In yet another specific embodiment of the various methods provided herein, the biomarker is IKBKE. In yet another specific embodiment of the various methods provided herein, the biomarker is PDE6D. In yet another specific embodiment of the various methods provided herein, the biomarker is LGALS2. In yet another specific embodiment of the various methods provided herein, the biomarker is LGALS9. In some embodiments, the solid tumor is HCC. In some embodiments, the solid tumor is GBM.

In a specific embodiment of the various methods provided herein, the biomarker is ZFP91, and the solid tumor is HCC. In another embodiment of the various methods provided herein, the biomarker is CRBN, and the solid tumor is HCC. In yet another embodiment of the various methods provided herein, the biomarkers are both ZFP91 and CRBN, and the solid tumor is HCC. In another specific embodiment of the various methods provided herein, the biomarker is CK1-alpha, and the solid tumor is HCC. In another specific embodiment of the various methods provided herein, the biomarker is GSPT1, and the solid tumor is HCC. In another specific embodiment of the various methods provided herein, the biomarker is MST1R (RON), and the solid tumor is HCC. In another specific embodiment of the various methods provided herein, the biomarker is Nestin, and the solid tumor is HCC. In another specific embodiment of the various methods provided herein, the biomarker is KAT1/CCBL1, and the solid tumor is HCC. In another specific embodiment of the various methods provided herein, the biomarker is WIBG, and the solid tumor is HCC. In another specific embodiment of the various methods provided herein, the biomarker is MVP, and the solid tumor is HCC. In yet another specific embodiment of the various methods provided herein, the biomarker is PARP4, and the solid tumor is HCC. In yet another specific embodiment of the various methods provided herein, the biomarker is ZNF198, and the solid tumor is HCC. In yet another specific embodiment of the various methods provided herein, the biomarker is ZMYM198, and the solid tumor is HCC. In yet another specific embodiment of the various methods provided herein, the biomarker is FAM83H, and the solid tumor is HCC. In yet another specific embodiment of the various methods provided herein, the biomarker is KLC4, and the solid tumor is HCC. In yet another specific embodiment of the various methods provided herein, the biomarker is EHMT2, and the solid tumor is HCC. In yet another specific embodiment of the various methods provided herein, the biomarker is GLRX, and the solid tumor is HCC. In yet another specific embodiment of the various methods provided herein, the biomarker is SDSL, and the solid tumor is HCC. In yet another specific embodiment of the various methods provided herein, the biomarker is LIG3, and the solid tumor is HCC. In yet another specific embodiment of the various methods provided herein, the biomarker is IKBKE, and the solid tumor is HCC. In yet another specific embodiment of the various methods provided herein, the biomarker is PDE6D, and the solid tumor is HCC. In yet another specific embodiment of the various methods provided herein, the biomarker is LGALS2, and the solid tumor is HCC. In yet another specific embodiment of the various methods provided herein, the biomarker is LGALS9, and the solid tumor is HCC.

In a specific embodiment of the various methods provided herein, the biomarker is ZFP91, and the solid tumor is GBM. In another embodiment of the various methods provided herein, the biomarker is CRBN, and the solid tumor is GBM. In yet another embodiment of the various methods provided herein, the biomarkers are both ZFP91 and CRBN, and the solid tumor is GBM. In another specific embodiment of the various methods provided herein, the biomarker is CK1-alpha, and the solid tumor is GBM. In another specific embodiment of the various methods provided herein, the biomarker is GSPT1, and the solid tumor is GBM. In another specific embodiment of the various methods provided herein, the biomarker is MST1R (RON), and the solid tumor is GBM. In another specific embodiment of the various methods provided herein, the biomarker is Nestin, and the solid tumor is GBM. In another specific embodiment of the various methods provided herein, the biomarker is KAT1/CCBL1, and the solid tumor is GBM. In another specific embodiment of the various methods provided herein, the biomarker is WIBG, and the solid tumor is GBM. In another specific embodiment of the various methods provided herein, the biomarker is MVP, and the solid tumor is GBM. In yet another specific embodiment of the various methods provided herein, the biomarker is PARP4, and the solid tumor is GBM. In yet another specific embodiment of the various methods provided herein, the biomarker is ZNF198, and the solid tumor is GBM. In yet another specific embodiment of the various methods provided herein, the biomarker is ZMYM198, and the solid tumor is GBM. In yet another specific embodiment of the various methods provided herein, the biomarker is FAM83H, and the solid tumor is GBM. In yet another specific embodiment of the various methods provided herein, the biomarker is KLC4, and the solid tumor is GBM. In yet another specific embodiment of the various methods provided herein, the biomarker is EHMT2, and the solid tumor is GBM. In yet another specific embodiment of the various methods provided herein, the biomarker is GLRX, and the solid tumor is GBM. In yet another specific embodiment of the various methods provided herein, the biomarker is SDSL, and the solid tumor is GBM. In yet another specific embodiment of the various methods provided herein, the biomarker is LIG3, and the solid tumor is GBM. In yet another specific embodiment of the various methods provided herein, the biomarker is IKBKE, and the solid tumor is GBM. In yet another specific embodiment of the various methods provided herein, the biomarker is PDE6D, and the solid tumor is GBM. In yet another specific embodiment of the various methods provided herein, the biomarker is LGALS2, and the solid tumor is GBM. In yet another specific embodiment of the various methods provided herein, the biomarker is LGALS9, and the solid tumor is GBM.

In yet other embodiments of the various methods provided herein, the biomarker is selected from a group consisting of SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTU1, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770, and the solid tumor is HCC or GBM.

In yet other embodiments of the various methods provided herein, the biomarker is selected from a group consisting of MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDC38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLIT, FNBP1L, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830, and the solid tumor is HCC or GBM.

In yet other embodiments of the various methods provided herein, the biomarker is selected from a group consisting of ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Tap, Igf2r, IGFBP1, IGFBP3, IKBKE, JAG1, KIAA0100, KIAA1462, KIF20B, KIF22, KLC2, KRT9, LDLR, LPXN, MAFF, MELK, MET, MGAT4B, MGLL, MMP7, MST1R, MT2A, MVP, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2, and the solid tumor is HCC or GBM.

In yet other embodiments of the various methods provided herein, the biomarker is selected from a group consisting of ACSL6, AHSG, ALB, ARPP19, ATP5EP2, ATP5I, ATP5J, AZGP1, BCKDK, BLOC1S3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1H1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBC1D12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2, and the solid tumor is HCC or GBM.

In some embodiments, provided herein is a method of predicting the responsiveness of a subject having or suspected of having a solid tumor, e.g., HCC or GBM, to Compound B, comprising:

(a) administering Compound B to a subject having a solid tumor;

(b) obtaining a sample from the subject;

(c) determining the level of a biomarker in the sample from the subject, wherein the biomarker is selected from the group consisting of biomarkers identified in Tables 1 to 6, and combinations thereof;

(d) diagnosing the subject as being likely to be responsive to a treatment of the solid tumor with Compound B if the level of the biomarker in the sample changes as compared to the level of the biomarker obtained from a reference sample. In one embodiment, the solid tumor is HCC. In another embodiment, the solid tumor is GBM.

In some embodiments, provided herein is a method of monitoring the efficacy of a treatment of a solid tumor, e.g., HCC or GBM, in a subject with Compound B, comprising:

(a) administering Compound B to a subject having a solid tumor;

(b) obtaining a sample from the subject;

(c) determining the level of a biomarker in the sample from the subject, wherein the biomarker is selected from the group consisting of biomarkers identified in Tables 1 to 6, and combinations thereof;

(d) comparing the level of the biomarker in the sample with the level of the biomarker obtained from a reference sample, wherein a change in the level as compared to the reference is indicative of the efficacy of Compound B in treating the solid tumor in the subject. In one embodiment, the solid tumor is HCC. In another embodiment, the solid tumor is GBM.

In some embodiments, the biomarker is selected from a group consisting of CRBN, CK1-alpha, GSPT1, Nestin, KAT1/CCBL1, WIBG, FAM83H, KLC4, LIG3, IKBKE, LGALS2 LGALS9, MVP, PARP4, ZFP91, ZNF198, MST1R (RON), ZMYM198, EHMT2, GLRX, SDSL, and PDE6D. In some embodiments of the various methods provided herein, the biomarker is selected from a group consisting of CRBN, CK1-alpha, GSPT1, Nestin, KAT1/CCBL1, WIBG, FAM83H, KLC4, LIG3, IKBKE, LGALS2 and LGALS9. In other embodiments, the biomarker is selected from a group consisting of MVP, PARP4, ZFP91, ZNF198, MST1R (RON), ZMYM198, EHMT2, GLRX, SDSL, and PDE6D. In a specific embodiment of the various methods provided herein, the biomarker is ZFP91. In another embodiment of the various methods provided herein, the biomarker is CRBN. In yet another embodiment of the various methods provided herein, the biomarkers are both ZFP91 and CRBN. In another specific embodiment of the various methods provided herein, the biomarker is CK1-alpha. In another specific embodiment of the various methods provided herein, the biomarker is GSPT1. In another specific embodiment of the various methods provided herein, the biomarker is MST1R (RON). In another specific embodiment of the various methods provided herein, the biomarker is Nestin. In another specific embodiment of the various methods provided herein, the biomarker is KAT1/CCBL1. In another specific embodiment of the various methods provided herein, the biomarker is WIBG. In another specific embodiment of the various methods provided herein, the biomarker is MVP. In yet another specific embodiment of the various methods provided herein, the biomarker is PARP4. In yet another specific embodiment of the various methods provided herein, the biomarker is ZNF198. In yet another specific embodiment of the various methods provided herein, the biomarker is ZMYM198. In yet another specific embodiment of the various methods provided herein, the biomarker is FAM83H. In yet another specific embodiment of the various methods provided herein, the biomarker is KLC4. In yet another specific embodiment of the various methods provided herein, the biomarker is EHMT2. In yet another specific embodiment of the various methods provided herein, the biomarker is GLRX. In yet another specific embodiment of the various methods provided herein, the biomarker is SDSL. In yet another specific embodiment of the various methods provided herein, the biomarker is LIG3. In yet another specific embodiment of the various methods provided herein, the biomarker is IKBKE. In yet another specific embodiment of the various methods provided herein, the biomarker is PDE6D. In yet another specific embodiment of the various methods provided herein, the biomarker is LGALS2. In yet another specific embodiment of the various methods provided herein, the biomarker is LGALS9. In some embodiments, the solid tumor is HCC. In some embodiments, the solid tumor is GBM.

In a specific embodiment of the various methods provided herein, the biomarker is ZFP91, and the solid tumor is HCC. In another embodiment of the various methods provided herein, the biomarker is CRBN, and the solid tumor is HCC. In yet another embodiment of the various methods provided herein, the biomarkers are both ZFP91 and CRBN, and the solid tumor is HCC. In another specific embodiment of the various methods provided herein, the biomarker is CK1-alpha, and the solid tumor is HCC. In another specific embodiment of the various methods provided herein, the biomarker is GSPT1, and the solid tumor is HCC. In another specific embodiment of the various methods provided herein, the biomarker is MST1R (RON), and the solid tumor is HCC. In another specific embodiment of the various methods provided herein, the biomarker is Nestin, and the solid tumor is HCC. In another specific embodiment of the various methods provided herein, the biomarker is KAT1/CCBL1, and the solid tumor is HCC. In another specific embodiment of the various methods provided herein, the biomarker is WIBG, and the solid tumor is HCC. In another specific embodiment of the various methods provided herein, the biomarker is MVP, and the solid tumor is HCC. In yet another specific embodiment of the various methods provided herein, the biomarker is PARP4, and the solid tumor is HCC. In yet another specific embodiment of the various methods provided herein, the biomarker is ZNF198, and the solid tumor is HCC. In yet another specific embodiment of the various methods provided herein, the biomarker is ZMYM198, and the solid tumor is HCC. In yet another specific embodiment of the various methods provided herein, the biomarker is FAM83H, and the solid tumor is HCC. In yet another specific embodiment of the various methods provided herein, the biomarker is KLC4, and the solid tumor is HCC. In yet another specific embodiment of the various methods provided herein, the biomarker is EHMT2, and the solid tumor is HCC. In yet another specific embodiment of the various methods provided herein, the biomarker is GLRX, and the solid tumor is HCC. In yet another specific embodiment of the various methods provided herein, the biomarker is SDSL, and the solid tumor is HCC. In yet another specific embodiment of the various methods provided herein, the biomarker is LIG3, and the solid tumor is HCC. In yet another specific embodiment of the various methods provided herein, the biomarker is IKBKE, and the solid tumor is HCC. In yet another specific embodiment of the various methods provided herein, the biomarker is PDE6D, and the solid tumor is HCC. In yet another specific embodiment of the various methods provided herein, the biomarker is LGALS2, and the solid tumor is HCC. In yet another specific embodiment of the various methods provided herein, the biomarker is LGALS9, and the solid tumor is HCC.

In a specific embodiment of the various methods provided herein, the biomarker is ZFP91, and the solid tumor is GBM. In another embodiment of the various methods provided herein, the biomarker is CRBN, and the solid tumor is GBM. In yet another embodiment of the various methods provided herein, the biomarkers are both ZFP91 and CRBN, and the solid tumor is GBM. In another specific embodiment of the various methods provided herein, the biomarker is CK1-alpha, and the solid tumor is GBM. In another specific embodiment of the various methods provided herein, the biomarker is GSPT1, and the solid tumor is GBM. In another specific embodiment of the various methods provided herein, the biomarker is MST1R (RON), and the solid tumor is GBM. In another specific embodiment of the various methods provided herein, the biomarker is Nestin, and the solid tumor is GBM. In another specific embodiment of the various methods provided herein, the biomarker is KAT1/CCBL1, and the solid tumor is GBM. In another specific embodiment of the various methods provided herein, the biomarker is WIBG, and the solid tumor is GBM. In another specific embodiment of the various methods provided herein, the biomarker is MVP, and the solid tumor is GBM. In yet another specific embodiment of the various methods provided herein, the biomarker is PARP4, and the solid tumor is GBM. In yet another specific embodiment of the various methods provided herein, the biomarker is ZNF198, and the solid tumor is GBM. In yet another specific embodiment of the various methods provided herein, the biomarker is ZMYM198, and the solid tumor is GBM. In yet another specific embodiment of the various methods provided herein, the biomarker is FAM83H, and the solid tumor is GBM. In yet another specific embodiment of the various methods provided herein, the biomarker is KLC4, and the solid tumor is GBM. In yet another specific embodiment of the various methods provided herein, the biomarker is EHMT2, and the solid tumor is GBM. In yet another specific embodiment of the various methods provided herein, the biomarker is GLRX, and the solid tumor is GBM. In yet another specific embodiment of the various methods provided herein, the biomarker is SDSL, and the solid tumor is GBM. In yet another specific embodiment of the various methods provided herein, the biomarker is LIG3, and the solid tumor is GBM. In yet another specific embodiment of the various methods provided herein, the biomarker is IKBKE, and the solid tumor is GBM. In yet another specific embodiment of the various methods provided herein, the biomarker is PDE6D, and the solid tumor is GBM. In yet another specific embodiment of the various methods provided herein, the biomarker is LGALS2, and the solid tumor is GBM. In yet another specific embodiment of the various methods provided herein, the biomarker is LGALS9, and the solid tumor is GBM.

In yet other embodiments of the various methods provided herein, the biomarker is selected from a group consisting of SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTU1, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770, and the solid tumor is HCC or GBM.

In yet other embodiments of the various methods provided herein, the biomarker is selected from a group consisting of MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDC38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLIT, FNBP1L, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830, and the solid tumor is HCC or GBM.

In yet other embodiments of the various methods provided herein, the biomarker is selected from a group consisting of ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Tap, Igf2r, IGFBP1, IGFBP3, IKBKE, JAG1, KIAA0100, KIAA1462, KIF20B, KIF22, KLC2, KRT9, LDLR, LPXN, MAFF, MELK, MET, MGAT4B, MGLL, MMP7, MST1R, MT2A, MVP, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2, and the solid tumor is HCC or GBM.

In yet other embodiments of the various methods provided herein, the biomarker is selected from a group consisting of ACSL6, AHSG, ALB, ARPP19, ATP5EP2, ATP5I, ATP5J, AZGP1, BCKDK, BLOC1S3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1H1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBC1D12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2, and the solid tumor is HCC or GBM.

In some embodiments, provided herein is a method of predicting the responsiveness of a subject having or suspected of having a solid tumor, e.g., HCC or GBM, to lenalidomide, comprising:

(a) administering lenalidomide to a subject having a solid tumor;

(b) obtaining a sample from the subject;

(c) determining the level of a biomarker in the sample from the subject, wherein the biomarker is selected from the group consisting of biomarkers identified in Tables 1 to 6, and combinations thereof;

(d) diagnosing the subject as being likely to be responsive to a treatment of the solid tumor with lenalidomide if the level of the biomarker in the sample changes as compared to the level of the biomarker obtained from a reference sample. In one embodiment, the solid tumor is HCC. In another embodiment, the solid tumor is GBM.

In some embodiments, provided herein is a method of monitoring the efficacy of a treatment of a solid tumor, e.g., HCC or GBM, in a subject with lenalidomide, comprising:

(a) administering lenalidomide to a subject having a solid tumor;

(b) obtaining a sample from the subject;

(c) determining the level of a biomarker in the sample from the subject, wherein the biomarker is selected from the group consisting of biomarkers identified in Tables 1 to 6, and combinations thereof;

(d) comparing the level of the biomarker in the sample with the level of the biomarker obtained from a reference sample, wherein a change in the level as compared to the reference is indicative of the efficacy of lenalidomide in treating the solid tumor in the subject. In one embodiment, the solid tumor is HCC. In another embodiment, the solid tumor is GBM.

In some embodiments, the biomarker is selected from a group consisting of CRBN, CK1-alpha, GSPT1, Nestin, KAT1/CCBL1, WIBG, FAM83H, KLC4, LIG3, IKBKE, LGALS2 LGALS9, MVP, PARP4, ZFP91, ZNF198, MST1R (RON), ZMYM198, EHMT2, GLRX, SDSL, and PDE6D. In some embodiments of the various methods provided herein, the biomarker is selected from a group consisting of CRBN, CK1-alpha, GSPT1, Nestin, KAT1/CCBL1, WIBG, FAM83H, KLC4, LIG3, IKBKE, LGALS2 and LGALS9. In other embodiments, the biomarker is selected from a group consisting of MVP, PARP4, ZFP91, ZNF198, MST1R (RON), ZMYM198, EHMT2, GLRX, SDSL, and PDE6D. In a specific embodiment of the various methods provided herein, the biomarker is ZFP91. In another embodiment of the various methods provided herein, the biomarker is CRBN. In yet another embodiment of the various methods provided herein, the biomarkers are both ZFP91 and CRBN. In another specific embodiment of the various methods provided herein, the biomarker is CK1-alpha. In another specific embodiment of the various methods provided herein, the biomarker is GSPT1. In another specific embodiment of the various methods provided herein, the biomarker is MST1R (RON). In another specific embodiment of the various methods provided herein, the biomarker is Nestin. In another specific embodiment of the various methods provided herein, the biomarker is KAT1/CCBL1. In another specific embodiment of the various methods provided herein, the biomarker is WIBG. In another specific embodiment of the various methods provided herein, the biomarker is MVP. In yet another specific embodiment of the various methods provided herein, the biomarker is PARP4. In yet another specific embodiment of the various methods provided herein, the biomarker is ZNF198. In yet another specific embodiment of the various methods provided herein, the biomarker is ZMYM198. In yet another specific embodiment of the various methods provided herein, the biomarker is FAM83H. In yet another specific embodiment of the various methods provided herein, the biomarker is KLC4. In yet another specific embodiment of the various methods provided herein, the biomarker is EHMT2. In yet another specific embodiment of the various methods provided herein, the biomarker is GLRX. In yet another specific embodiment of the various methods provided herein, the biomarker is SDSL. In yet another specific embodiment of the various methods provided herein, the biomarker is LIG3. In yet another specific embodiment of the various methods provided herein, the biomarker is IKBKE. In yet another specific embodiment of the various methods provided herein, the biomarker is PDE6D. In yet another specific embodiment of the various methods provided herein, the biomarker is LGALS2. In yet another specific embodiment of the various methods provided herein, the biomarker is LGALS9. In some embodiments, the solid tumor is HCC. In some embodiments, the solid tumor is GBM.

In a specific embodiment of the various methods provided herein, the biomarker is ZFP91, and the solid tumor is HCC. In another embodiment of the various methods provided herein, the biomarker is CRBN, and the solid tumor is HCC. In yet another embodiment of the various methods provided herein, the biomarkers are both ZFP91 and CRBN, and the solid tumor is HCC. In another specific embodiment of the various methods provided herein, the biomarker is CK1-alpha, and the solid tumor is HCC. In another specific embodiment of the various methods provided herein, the biomarker is GSPT1, and the solid tumor is HCC. In another specific embodiment of the various methods provided herein, the biomarker is MST1R (RON), and the solid tumor is HCC. In another specific embodiment of the various methods provided herein, the biomarker is Nestin, and the solid tumor is HCC. In another specific embodiment of the various methods provided herein, the biomarker is KAT1/CCBL1, and the solid tumor is HCC. In another specific embodiment of the various methods provided herein, the biomarker is WIBG, and the solid tumor is HCC. In another specific embodiment of the various methods provided herein, the biomarker is MVP, and the solid tumor is HCC. In yet another specific embodiment of the various methods provided herein, the biomarker is PARP4, and the solid tumor is HCC. In yet another specific embodiment of the various methods provided herein, the biomarker is ZNF198, and the solid tumor is HCC. In yet another specific embodiment of the various methods provided herein, the biomarker is ZMYM198, and the solid tumor is HCC. In yet another specific embodiment of the various methods provided herein, the biomarker is FAM83H, and the solid tumor is HCC. In yet another specific embodiment of the various methods provided herein, the biomarker is KLC4, and the solid tumor is HCC. In yet another specific embodiment of the various methods provided herein, the biomarker is EHMT2, and the solid tumor is HCC. In yet another specific embodiment of the various methods provided herein, the biomarker is GLRX, and the solid tumor is HCC. In yet another specific embodiment of the various methods provided herein, the biomarker is SDSL, and the solid tumor is HCC. In yet another specific embodiment of the various methods provided herein, the biomarker is LIG3, and the solid tumor is HCC. In yet another specific embodiment of the various methods provided herein, the biomarker is IKBKE, and the solid tumor is HCC. In yet another specific embodiment of the various methods provided herein, the biomarker is PDE6D, and the solid tumor is HCC. In yet another specific embodiment of the various methods provided herein, the biomarker is LGALS2, and the solid tumor is HCC. In yet another specific embodiment of the various methods provided herein, the biomarker is LGALS9, and the solid tumor is HCC.

In a specific embodiment of the various methods provided herein, the biomarker is ZFP91, and the solid tumor is GBM. In another embodiment of the various methods provided herein, the biomarker is CRBN, and the solid tumor is GBM. In yet another embodiment of the various methods provided herein, the biomarkers are both ZFP91 and CRBN, and the solid tumor is GBM. In another specific embodiment of the various methods provided herein, the biomarker is CK1-alpha, and the solid tumor is GBM. In another specific embodiment of the various methods provided herein, the biomarker is GSPT1, and the solid tumor is GBM. In another specific embodiment of the various methods provided herein, the biomarker is MST1R (RON), and the solid tumor is GBM. In another specific embodiment of the various methods provided herein, the biomarker is Nestin, and the solid tumor is GBM. In another specific embodiment of the various methods provided herein, the biomarker is KAT1/CCBL1, and the solid tumor is GBM. In another specific embodiment of the various methods provided herein, the biomarker is WIBG, and the solid tumor is GBM. In another specific embodiment of the various methods provided herein, the biomarker is MVP, and the solid tumor is GBM. In yet another specific embodiment of the various methods provided herein, the biomarker is PARP4, and the solid tumor is GBM. In yet another specific embodiment of the various methods provided herein, the biomarker is ZNF198, and the solid tumor is GBM. In yet another specific embodiment of the various methods provided herein, the biomarker is ZMYM198, and the solid tumor is GBM. In yet another specific embodiment of the various methods provided herein, the biomarker is FAM83H, and the solid tumor is GBM. In yet another specific embodiment of the various methods provided herein, the biomarker is KLC4, and the solid tumor is GBM. In yet another specific embodiment of the various methods provided herein, the biomarker is EHMT2, and the solid tumor is GBM. In yet another specific embodiment of the various methods provided herein, the biomarker is GLRX, and the solid tumor is GBM. In yet another specific embodiment of the various methods provided herein, the biomarker is SDSL, and the solid tumor is GBM. In yet another specific embodiment of the various methods provided herein, the biomarker is LIG3, and the solid tumor is GBM. In yet another specific embodiment of the various methods provided herein, the biomarker is IKBKE, and the solid tumor is GBM. In yet another specific embodiment of the various methods provided herein, the biomarker is PDE6D, and the solid tumor is GBM. In yet another specific embodiment of the various methods provided herein, the biomarker is LGALS2, and the solid tumor is GBM. In yet another specific embodiment of the various methods provided herein, the biomarker is LGALS9, and the solid tumor is GBM.

In yet other embodiments of the various methods provided herein, the biomarker is selected from a group consisting of SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTU1, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770, and the solid tumor is HCC or GBM.

In yet other embodiments of the various methods provided herein, the biomarker is selected from a group consisting of MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDC38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLIT, FNBP1L, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830, and the solid tumor is HCC or GBM.

In yet other embodiments of the various methods provided herein, the biomarker is selected from a group consisting of ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Tap, Igf2r, IGFBP1, IGFBP3, IKBKE, JAG1, KIAA0100, KIAA1462, KIF20B, KIF22, KLC2, KRT9, LDLR, LPXN, MAFF, MELK, MET, MGAT4B, MGLL, MMP7, MST1R, MT2A, MVP, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2, and the solid tumor is HCC or GBM.

In yet other embodiments of the various methods provided herein, the biomarker is selected from a group consisting of ACSL6, AHSG, ALB, ARPP19, ATP5EP2, ATP5I, ATP5J, AZGP1, BCKDK, BLOC1S3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1H1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBC1D12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2, and the solid tumor is HCC or GBM.

In some embodiments, the level of the biomarker is measured in an in vitro assay to predict the responsiveness of a subject to a treatment of a solid tumor (e.g., a treatment by a treatment compound provided herein), comprising obtaining a sample of cells from the subject, culturing the cells in the presence or absence of a treatment compound, and testing the cells for the levels of the biomarkers, wherein an increased or a decreased level of the biomarker in the presence of the treatment compound indicates the likelihood of responsiveness of the subject to the treatment compound.

In some embodiments, the solid tumors are sarcomas, carcinomas (epithelial tumors), melanomas, and glioblastomas. Exemplary solid tumors include, but not limited to, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma, glioblastoma (GBM); medulloblastoma), cervical cancer (e.g., cervical adenocarcinoma), colorectal cancer (e.g., colon cancer, rectal cancer, colorectal adenocarcinoma), gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), pancreatic cancer (e.g., pancreatic andenocarcinoma, intraductal papillary mucinous neoplasm (IPMN)), prostate cancer (e.g., prostate adenocarcinoma), skin cancer (e.g., squamous cell carcinoma (SCC), keratoacanthoma (A), melanoma, basal cell carcinoma (BCC)) and soft tissue sarcoma (e.g., malignant fibrous histiocytoma (MFH), liposarcoma, malignant peripheral nerve sheath tumor (MPNST), chondrosarcoma, fibrosarcoma, myxosarcoma, osteosarcoma). In some embodiments, the solid tumor is a liver cancer. In one embodiment, the solid tumor is HCC. In other embodiments, the solid tumor is a brain cancer. In one embodiment, the solid tumor is GBM.

In some embodiments, provided herein is a method of predicting the responsiveness of a subject having or suspected of having HCC, to a treatment compound, comprising:

(a) administering the treatment compound to a subject having HCC;

(b) obtaining a sample from the subject;

(c) determining the level of a biomarker in the sample from the subject, wherein the biomarker is selected from the group consisting of biomarkers identified in Tables 1 to 6, and combinations thereof;

(d) diagnosing the subject as being likely to be responsive to a treatment of HCC with the treatment compound if the level of the biomarker in the sample changes as compared to the level of the biomarker obtained from a reference sample.

In some embodiments, provided herein is a method of monitoring the efficacy of a treatment of HCC, in a subject with a treatment compound, comprising:

(a) administering the treatment compound to a subject having HCC;

(b) obtaining a sample from the subject;

(c) determining the level of a biomarker in the sample from the subject, wherein the biomarker is selected from the group consisting of biomarkers identified in Tables 1 to 6, and combinations thereof;

(d) comparing the level of the biomarker in the sample with the level of the biomarker obtained from a reference sample, wherein a change in the level as compared to the reference is indicative of the efficacy of the treatment compound in treating HCC in the subject.

In some embodiments, the biomarker is selected from a group consisting of CRBN, CK1-alpha, GSPT1, Nestin, KAT1/CCBL1, WIBG, FAM83H, KLC4, LIG3, IKBKE, LGALS2 LGALS9, MVP, PARP4, ZFP91, ZNF198, MST1R (RON), ZMYM198, EHMT2, GLRX, SDSL, and PDE6D. In some embodiments of the various methods provided herein, the biomarker is selected from a group consisting of CRBN, CK1-alpha, GSPT1, Nestin, KAT1/CCBL1, WIBG, FAM83H, KLC4, LIG3, IKBKE, LGALS2 and LGALS9. In other embodiments, the biomarker is selected from a group consisting of MVP, PARP4, ZFP91, ZNF198, MST1R (RON), ZMYM198, EHMT2, GLRX, SDSL, and PDE6D. In a specific embodiment of the various methods provided herein, the biomarker is ZFP91. In another embodiment of the various methods provided herein, the biomarker is CRBN. In yet another embodiment of the various methods provided herein, the biomarkers are both ZFP91 and CRBN. In another specific embodiment of the various methods provided herein, the biomarker is CK1-alpha. In another specific embodiment of the various methods provided herein, the biomarker is GSPT1. In another specific embodiment of the various methods provided herein, the biomarker is MST1R (RON). In another specific embodiment of the various methods provided herein, the biomarker is Nestin. In another specific embodiment of the various methods provided herein, the biomarker is KAT1/CCBL1. In another specific embodiment of the various methods provided herein, the biomarker is WIBG. In another specific embodiment of the various methods provided herein, the biomarker is MVP. In yet another specific embodiment of the various methods provided herein, the biomarker is PARP4. In yet another specific embodiment of the various methods provided herein, the biomarker is ZNF198. In yet another specific embodiment of the various methods provided herein, the biomarker is ZMYM198. In yet another specific embodiment of the various methods provided herein, the biomarker is FAM83H. In yet another specific embodiment of the various methods provided herein, the biomarker is KLC4. In yet another specific embodiment of the various methods provided herein, the biomarker is EHMT2. In yet another specific embodiment of the various methods provided herein, the biomarker is GLRX. In yet another specific embodiment of the various methods provided herein, the biomarker is SDSL. In yet another specific embodiment of the various methods provided herein, the biomarker is LIG3. In yet another specific embodiment of the various methods provided herein, the biomarker is IKBKE. In yet another specific embodiment of the various methods provided herein, the biomarker is PDE6D. In yet another specific embodiment of the various methods provided herein, the biomarker is LGALS2. In yet another specific embodiment of the various methods provided herein, the biomarker is LGALS9. In some embodiments, the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In a specific embodiment of the various methods provided herein, the biomarker is ZFP91, and the treatment compound is Compound A. In another embodiment of the various methods provided herein, the biomarker is CRBN, and the treatment compound is Compound A. In yet another embodiment of the various methods provided herein, the biomarkers are both ZFP91 and CRBN, and the treatment compound is Compound A. In another specific embodiment of the various methods provided herein, the biomarker is CK1-alpha, and the treatment compound is Compound A. In another specific embodiment of the various methods provided herein, the biomarker is GSPT1, and the treatment compound is Compound A. In another specific embodiment of the various methods provided herein, the biomarker is MST1R (RON), and the treatment compound is Compound A. In another specific embodiment of the various methods provided herein, the biomarker is Nestin, and the treatment compound is Compound A. In another specific embodiment of the various methods provided herein, the biomarker is KAT1/CCBL1, and the treatment compound is Compound A. In another specific embodiment of the various methods provided herein, the biomarker is WIBG, and the treatment compound is Compound A. In another specific embodiment of the various methods provided herein, the biomarker is MVP, and the treatment compound is Compound A. In yet another specific embodiment of the various methods provided herein, the biomarker is PARP4, and the treatment compound is Compound A. In yet another specific embodiment of the various methods provided herein, the biomarker is ZNF198, and the treatment compound is Compound A. In yet another specific embodiment of the various methods provided herein, the biomarker is ZMYM198, and the treatment compound is Compound A. In yet another specific embodiment of the various methods provided herein, the biomarker is FAM83H, and the treatment compound is Compound A. In yet another specific embodiment of the various methods provided herein, the biomarker is KLC4, and the treatment compound is Compound A. In yet another specific embodiment of the various methods provided herein, the biomarker is EHMT2, and the treatment compound is Compound A. In yet another specific embodiment of the various methods provided herein, the biomarker is GLRX, and the treatment compound is Compound A. In yet another specific embodiment of the various methods provided herein, the biomarker is SDSL, and the treatment compound is Compound A. In yet another specific embodiment of the various methods provided herein, the biomarker is LIG3, and the treatment compound is Compound A. In yet another specific embodiment of the various methods provided herein, the biomarker is IKBKE, and the treatment compound is Compound A. In yet another specific embodiment of the various methods provided herein, the biomarker is PDE6D, and the treatment compound is Compound A. In yet another specific embodiment of the various methods provided herein, the biomarker is LGALS2, and the treatment compound is Compound A. In yet another specific embodiment of the various methods provided herein, the biomarker is LGALS9, and the treatment compound is Compound A.

In a specific embodiment of the various methods provided herein, the biomarker is ZFP91, and the treatment compound is Compound B. In another embodiment of the various methods provided herein, the biomarker is CRBN, and the treatment compound is Compound B. In yet another embodiment of the various methods provided herein, the biomarkers are both ZFP91 and CRBN, and the treatment compound is Compound B. In another specific embodiment of the various methods provided herein, the biomarker is CK1-alpha, and the treatment compound is Compound B. In another specific embodiment of the various methods provided herein, the biomarker is GSPT1, and the treatment compound is Compound B. In another specific embodiment of the various methods provided herein, the biomarker is MST1R (RON), and the treatment compound is Compound B. In another specific embodiment of the various methods provided herein, the biomarker is Nestin, and the treatment compound is Compound B. In another specific embodiment of the various methods provided herein, the biomarker is KAT1/CCBL1, and the treatment compound is Compound B. In another specific embodiment of the various methods provided herein, the biomarker is WIBG, and the treatment compound is Compound B. In another specific embodiment of the various methods provided herein, the biomarker is MVP, and the treatment compound is Compound B. In yet another specific embodiment of the various methods provided herein, the biomarker is PARP4, and the treatment compound is Compound B. In yet another specific embodiment of the various methods provided herein, the biomarker is ZNF198, and the treatment compound is Compound B. In yet another specific embodiment of the various methods provided herein, the biomarker is ZMYM198, and the treatment compound is Compound B. In yet another specific embodiment of the various methods provided herein, the biomarker is FAM83H, and the treatment compound is Compound B. In yet another specific embodiment of the various methods provided herein, the biomarker is KLC4, and the treatment compound is Compound B. In yet another specific embodiment of the various methods provided herein, the biomarker is EHMT2, and the treatment compound is Compound B. In yet another specific embodiment of the various methods provided herein, the biomarker is GLRX, and the treatment compound is Compound B. In yet another specific embodiment of the various methods provided herein, the biomarker is SDSL, and the treatment compound is Compound B. In yet another specific embodiment of the various methods provided herein, the biomarker is LIG3, and the treatment compound is Compound B. In yet another specific embodiment of the various methods provided herein, the biomarker is IKBKE, and the treatment compound is Compound B. In yet another specific embodiment of the various methods provided herein, the biomarker is PDE6D, and the treatment compound is Compound B. In yet another specific embodiment of the various methods provided herein, the biomarker is LGALS2, and the treatment compound is Compound B. In yet another specific embodiment of the various methods provided herein, the biomarker is LGALS9, and the treatment compound is Compound B.

In a specific embodiment of the various methods provided herein, the biomarker is ZFP91, and the treatment compound is Lenalidomide. In another embodiment of the various methods provided herein, the biomarker is CRBN, and the treatment compound is Lenalidomide. In yet another embodiment of the various methods provided herein, the biomarkers are both ZFP91 and CRBN, and the treatment compound is Lenalidomide. In another specific embodiment of the various methods provided herein, the biomarker is CK1-alpha, and the treatment compound is Lenalidomide. In another specific embodiment of the various methods provided herein, the biomarker is GSPT1, and the treatment compound is Lenalidomide. In another specific embodiment of the various methods provided herein, the biomarker is MST1R (RON), and the treatment compound is Lenalidomide. In another specific embodiment of the various methods provided herein, the biomarker is Nestin, and the treatment compound is Lenalidomide. In another specific embodiment of the various methods provided herein, the biomarker is KAT1/CCBL1, and the treatment compound is Lenalidomide. In another specific embodiment of the various methods provided herein, the biomarker is WIBG, and the treatment compound is Lenalidomide. In another specific embodiment of the various methods provided herein, the biomarker is MVP, and the treatment compound is Lenalidomide. In yet another specific embodiment of the various methods provided herein, the biomarker is PARP4, and the treatment compound is Lenalidomide. In yet another specific embodiment of the various methods provided herein, the biomarker is ZNF198, and the treatment compound is Lenalidomide. In yet another specific embodiment of the various methods provided herein, the biomarker is ZMYM198, and the treatment compound is Lenalidomide. In yet another specific embodiment of the various methods provided herein, the biomarker is FAM83H, and the treatment compound is Lenalidomide. In yet another specific embodiment of the various methods provided herein, the biomarker is KLC4, and the treatment compound is Lenalidomide. In yet another specific embodiment of the various methods provided herein, the biomarker is EHMT2, and the treatment compound is Lenalidomide. In yet another specific embodiment of the various methods provided herein, the biomarker is GLRX, and the treatment compound is Lenalidomide. In yet another specific embodiment of the various methods provided herein, the biomarker is SDSL, and the treatment compound is Lenalidomide. In yet another specific embodiment of the various methods provided herein, the biomarker is LIG3, and the treatment compound is Lenalidomide. In yet another specific embodiment of the various methods provided herein, the biomarker is IKBKE, and the treatment compound is Lenalidomide. In yet another specific embodiment of the various methods provided herein, the biomarker is PDE6D, and the treatment compound is Lenalidomide. In yet another specific embodiment of the various methods provided herein, the biomarker is LGALS2, and the treatment compound is Lenalidomide. In yet another specific embodiment of the various methods provided herein, the biomarker is LGALS9, and the treatment compound is Lenalidomide.

In yet other embodiments of the various methods provided herein, the biomarker is selected from a group consisting of SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTU1, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770, and the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In yet other embodiments of the various methods provided herein, the biomarker is selected from a group consisting of MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDC38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLII, FNBP1L, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830, and the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In yet other embodiments of the various methods provided herein, the biomarker is selected from a group consisting of ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Tap, Igf2r, IGFBP1, IGFBP3, IKBKE, JAG1, KIAA0100, KIAA1462, KIF20B, KIF22, KLC2, KRT9, LDLR, LPXN, MAFF, MELK, MET, MGAT4B, MGLL, MMP7, MST1R, MT2A, MVP, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2, and the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In yet other embodiments of the various methods provided herein, the biomarker is selected from a group consisting of ACSL6, AHSG, ALB, ARPP19, ATP5EP2, ATP5I, ATP5J, AZGP1, BCKDK, BLOC1S3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1H1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBC1D12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2, and the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In some embodiments, provided herein is a method of predicting the responsiveness of a subject having or suspected of having GBM, to a treatment compound, comprising:

(a) administering the treatment compound to a subject having GBM;

(b) obtaining a sample from the subject;

(c) determining the level of a biomarker in the sample from the subject, wherein the biomarker is selected from the group consisting of biomarkers identified in Tables 1 to 6, and combinations thereof;

(d) diagnosing the subject as being likely to be responsive to a treatment of GBM with the treatment compound if the level of the biomarker in the sample changes as compared to the level of the biomarker obtained from a reference sample.

In some embodiments, provided herein is a method of monitoring the efficacy of a treatment of GBM, in a subject with a treatment compound, comprising:

(a) administering the treatment compound to a subject having GBM;

(b) obtaining a sample from the subject;

(c) determining the level of a biomarker in the sample from the subject, wherein the biomarker is selected from the group consisting of biomarkers identified in Tables 1 to 6, and combinations thereof;

(d) comparing the level of the biomarker in the sample with the level of the biomarker obtained from a reference sample, wherein a change in the level as compared to the reference is indicative of the efficacy of the treatment compound in treating GBM in the subject.

In some embodiments, the biomarker is selected from a group consisting of CRBN, CK1-alpha, GSPT1, Nestin, KAT1/CCBL1, WIBG, FAM83H, KLC4, LIG3, IKBKE, LGALS2 LGALS9, MVP, PARP4, ZFP91, ZNF198, MST1R (RON), ZMYM198, EHMT2, GLRX, SDSL, and PDE6D. In some embodiments of the various methods provided herein, the biomarker is selected from a group consisting of CRBN, CK1-alpha, GSPT1, Nestin, KAT1/CCBL1, WIBG, FAM83H, KLC4, LIG3, IKBKE, LGALS2 and LGALS9. In other embodiments, the biomarker is selected from a group consisting of MVP, PARP4, ZFP91, ZNF198, MST1R (RON), ZMYM198, EHMT2, GLRX, SDSL, and PDE6D. In a specific embodiment of the various methods provided herein, the biomarker is ZFP91. In another embodiment of the various methods provided herein, the biomarker is CRBN. In yet another embodiment of the various methods provided herein, the biomarkers are both ZFP91 and CRBN. In another specific embodiment of the various methods provided herein, the biomarker is CK1-alpha. In another specific embodiment of the various methods provided herein, the biomarker is GSPT1. In another specific embodiment of the various methods provided herein, the biomarker is MST1R (RON). In another specific embodiment of the various methods provided herein, the biomarker is Nestin. In another specific embodiment of the various methods provided herein, the biomarker is KAT1/CCBL1. In another specific embodiment of the various methods provided herein, the biomarker is WIBG. In another specific embodiment of the various methods provided herein, the biomarker is MVP. In yet another specific embodiment of the various methods provided herein, the biomarker is PARP4. In yet another specific embodiment of the various methods provided herein, the biomarker is ZNF198. In yet another specific embodiment of the various methods provided herein, the biomarker is ZMYM198. In yet another specific embodiment of the various methods provided herein, the biomarker is FAM83H. In yet another specific embodiment of the various methods provided herein, the biomarker is KLC4. In yet another specific embodiment of the various methods provided herein, the biomarker is EHMT2. In yet another specific embodiment of the various methods provided herein, the biomarker is GLRX. In yet another specific embodiment of the various methods provided herein, the biomarker is SDSL. In yet another specific embodiment of the various methods provided herein, the biomarker is LIG3. In yet another specific embodiment of the various methods provided herein, the biomarker is IKBKE. In yet another specific embodiment of the various methods provided herein, the biomarker is PDE6D. In yet another specific embodiment of the various methods provided herein, the biomarker is LGALS2. In yet another specific embodiment of the various methods provided herein, the biomarker is LGALS9. In some embodiments, the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In a specific embodiment of the various methods provided herein, the biomarker is ZFP91, and the treatment compound is Compound A. In another embodiment of the various methods provided herein, the biomarker is CRBN, and the treatment compound is Compound A. In yet another embodiment of the various methods provided herein, the biomarkers are both ZFP91 and CRBN, and the treatment compound is Compound A. In another specific embodiment of the various methods provided herein, the biomarker is CK1-alpha, and the treatment compound is Compound A. In another specific embodiment of the various methods provided herein, the biomarker is GSPT1, and the treatment compound is Compound A. In another specific embodiment of the various methods provided herein, the biomarker is MST1R (RON), and the treatment compound is Compound A. In another specific embodiment of the various methods provided herein, the biomarker is Nestin, and the treatment compound is Compound A. In another specific embodiment of the various methods provided herein, the biomarker is KAT1/CCBL1, and the treatment compound is Compound A. In another specific embodiment of the various methods provided herein, the biomarker is WIBG, and the treatment compound is Compound A. In another specific embodiment of the various methods provided herein, the biomarker is MVP, and the treatment compound is Compound A. In yet another specific embodiment of the various methods provided herein, the biomarker is PARP4, and the treatment compound is Compound A. In yet another specific embodiment of the various methods provided herein, the biomarker is ZNF198, and the treatment compound is Compound A. In yet another specific embodiment of the various methods provided herein, the biomarker is ZMYM198, and the treatment compound is Compound A. In yet another specific embodiment of the various methods provided herein, the biomarker is FAM83H, and the treatment compound is Compound A. In yet another specific embodiment of the various methods provided herein, the biomarker is KLC4, and the treatment compound is Compound A. In yet another specific embodiment of the various methods provided herein, the biomarker is EHMT2, and the treatment compound is Compound A. In yet another specific embodiment of the various methods provided herein, the biomarker is GLRX, and the treatment compound is Compound A. In yet another specific embodiment of the various methods provided herein, the biomarker is SDSL, and the treatment compound is Compound A. In yet another specific embodiment of the various methods provided herein, the biomarker is LIG3, and the treatment compound is Compound A. In yet another specific embodiment of the various methods provided herein, the biomarker is IKBKE, and the treatment compound is Compound A. In yet another specific embodiment of the various methods provided herein, the biomarker is PDE6D, and the treatment compound is Compound A. In yet another specific embodiment of the various methods provided herein, the biomarker is LGALS2, and the treatment compound is Compound A. In yet another specific embodiment of the various methods provided herein, the biomarker is LGALS9, and the treatment compound is Compound A.

In a specific embodiment of the various methods provided herein, the biomarker is ZFP91, and the treatment compound is Compound B. In another embodiment of the various methods provided herein, the biomarker is CRBN, and the treatment compound is Compound B. In yet another embodiment of the various methods provided herein, the biomarkers are both ZFP91 and CRBN, and the treatment compound is Compound B. In another specific embodiment of the various methods provided herein, the biomarker is CK1-alpha, and the treatment compound is Compound B. In another specific embodiment of the various methods provided herein, the biomarker is GSPT1, and the treatment compound is Compound B. In another specific embodiment of the various methods provided herein, the biomarker is MST1R (RON), and the treatment compound is Compound B. In another specific embodiment of the various methods provided herein, the biomarker is Nestin, and the treatment compound is Compound B. In another specific embodiment of the various methods provided herein, the biomarker is KAT1/CCBL1, and the treatment compound is Compound B. In another specific embodiment of the various methods provided herein, the biomarker is WIBG, and the treatment compound is Compound B. In another specific embodiment of the various methods provided herein, the biomarker is MVP, and the treatment compound is Compound B. In yet another specific embodiment of the various methods provided herein, the biomarker is PARP4, and the treatment compound is Compound B. In yet another specific embodiment of the various methods provided herein, the biomarker is ZNF198, and the treatment compound is Compound B. In yet another specific embodiment of the various methods provided herein, the biomarker is ZMYM198, and the treatment compound is Compound B. In yet another specific embodiment of the various methods provided herein, the biomarker is FAM83H, and the treatment compound is Compound B. In yet another specific embodiment of the various methods provided herein, the biomarker is KLC4, and the treatment compound is Compound B. In yet another specific embodiment of the various methods provided herein, the biomarker is EHMT2, and the treatment compound is Compound B. In yet another specific embodiment of the various methods provided herein, the biomarker is GLRX, and the treatment compound is Compound B. In yet another specific embodiment of the various methods provided herein, the biomarker is SDSL, and the treatment compound is Compound B. In yet another specific embodiment of the various methods provided herein, the biomarker is LIG3, and the treatment compound is Compound B. In yet another specific embodiment of the various methods provided herein, the biomarker is IKBKE, and the treatment compound is Compound B. In yet another specific embodiment of the various methods provided herein, the biomarker is PDE6D, and the treatment compound is Compound B. In yet another specific embodiment of the various methods provided herein, the biomarker is LGALS2, and the treatment compound is Compound B. In yet another specific embodiment of the various methods provided herein, the biomarker is LGALS9, and the treatment compound is Compound B.

In a specific embodiment of the various methods provided herein, the biomarker is ZFP91, and the treatment compound is Lenalidomide. In another embodiment of the various methods provided herein, the biomarker is CRBN, and the treatment compound is Lenalidomide. In yet another embodiment of the various methods provided herein, the biomarkers are both ZFP91 and CRBN, and the treatment compound is Lenalidomide. In another specific embodiment of the various methods provided herein, the biomarker is CK1-alpha, and the treatment compound is Lenalidomide. In another specific embodiment of the various methods provided herein, the biomarker is GSPT1, and the treatment compound is Lenalidomide. In another specific embodiment of the various methods provided herein, the biomarker is MST1R (RON), and the treatment compound is Lenalidomide. In another specific embodiment of the various methods provided herein, the biomarker is Nestin, and the treatment compound is Lenalidomide. In another specific embodiment of the various methods provided herein, the biomarker is KAT1/CCBL1, and the treatment compound is Lenalidomide. In another specific embodiment of the various methods provided herein, the biomarker is WIBG, and the treatment compound is Lenalidomide. In another specific embodiment of the various methods provided herein, the biomarker is MVP, and the treatment compound is Lenalidomide. In yet another specific embodiment of the various methods provided herein, the biomarker is PARP4, and the treatment compound is Lenalidomide. In yet another specific embodiment of the various methods provided herein, the biomarker is ZNF198, and the treatment compound is Lenalidomide. In yet another specific embodiment of the various methods provided herein, the biomarker is ZMYM198, and the treatment compound is Lenalidomide. In yet another specific embodiment of the various methods provided herein, the biomarker is FAM83H, and the treatment compound is Lenalidomide. In yet another specific embodiment of the various methods provided herein, the biomarker is KLC4, and the treatment compound is Lenalidomide. In yet another specific embodiment of the various methods provided herein, the biomarker is EHMT2, and the treatment compound is Lenalidomide. In yet another specific embodiment of the various methods provided herein, the biomarker is GLRX, and the treatment compound is Lenalidomide. In yet another specific embodiment of the various methods provided herein, the biomarker is SDSL, and the treatment compound is Lenalidomide. In yet another specific embodiment of the various methods provided herein, the biomarker is LIG3, and the treatment compound is Lenalidomide. In yet another specific embodiment of the various methods provided herein, the biomarker is IKBKE, and the treatment compound is Lenalidomide. In yet another specific embodiment of the various methods provided herein, the biomarker is PDE6D, and the treatment compound is Lenalidomide. In yet another specific embodiment of the various methods provided herein, the biomarker is LGALS2, and the treatment compound is Lenalidomide. In yet another specific embodiment of the various methods provided herein, the biomarker is LGALS9, and the treatment compound is Lenalidomide.

In yet other embodiments of the various methods provided herein, the biomarker is selected from a group consisting of SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTU1, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770, and the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In yet other embodiments of the various methods provided herein, the biomarker is selected from a group consisting of MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDC38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLII, FNBP1L, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830, and the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In yet other embodiments of the various methods provided herein, the biomarker is selected from a group consisting of ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Tap, Igf2r, IGFBP1, IGFBP3, IKBKE, JAG1, KIAA0100, KIAA1462, KIF20B, KIF22, KLC2, KRT9, LDLR, LPXN, MAFF, MELK, MET, MGAT4B, MGLL, MMP7, MST1R, MT2A, MVP, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2, and the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In yet other embodiments of the various methods provided herein, the biomarker is selected from a group consisting of ACSL6, AHSG, ALB, ARPP19, ATP5EP2, ATP5I, ATP5J, AZGP1, BCKDK, BLOC1S3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1H1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBC1D12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2, and the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof. In some embodiments, the treatment compound is Compound A. In some embodiments, the treatment compound is Compound B. In some embodiments, the treatment compound is lenadlidomide.

In some embodiments of the various methods provided herein, a treatment compound is administered to a patient likely to be responsive to the treatment compound. In certain embodiments, the compound is administered to a patient as a dose of from about 0.1 mg per day to about 100 mg per day. In other embodiments, the treatment compound is administered a patient as a dose of between about 0.5 mg per day to about 100 mg per day. In other embodiments, the treatment compound is administered a patient as a dose of between about 0.5 mg per day to about 20 mg per day. In other embodiments, the treatment compound is administered a patient as a dose of between about 5 mg per day to about 25 mg per day. In some embodiments, the treatment compound is administered a patient as a dose of between about 0.5 mg per day to about 10 mg per day. In certain embodiments, the treatment compound is administered a patient as a dose of between about 0.5 mg per day to about 100 mg per day.

In other embodiments, the treatment compound is administered at a dose of about 0.1 mg, 0.5 mg, 1 mg, 1.5 mg, 2 mg, 2.5 mg, 3 mg, 3.5 mg, 4.0 mg, 4.5 mg, 5 mg, 5.5 mg, 6 mg, 6.5 mg, 7 mg, 7.5 mg, 8 mg, 8.5 mg, 9 mg, 9.5 mg, 10 mg, 11 mg, 12 mg, 13 mg, 14 mg, 15 mg, 20 mg, 25 mg, 30 mg, 35 mg, 40 mg, 45 mg, 50 mg, 60 mg, 70 mg, 80 mg, 90 mg or 100 mg per day.

In some embodiments, the treatment compound is administered once daily. In some embodiments, the treatment compound is administered twice daily. In certain embodiments, the treatment compound is cyclically administered to a patient. Cycling therapy involves the administration of an active agent for a period of time, followed by a rest for a period of time, and repeating this sequential administration. Accordingly, in some embodiments, about 0.5 mg per day to about 100 mg per day of the treatment compound is administered on days 1-12 of a repeated 28 day cycle. In a specific embodiment, 25 mg of the treatment compound is administered once a day on days 1-12 of a repeated 28 day cycle.

It is understood that specific dose levels of a treatment compound described for any particular subject depends upon a variety of factors including the activity of the specific compound employed, the age, body weight, general health, sex, and diet of the patient, the time of administration, the rate of excretion, the treatment compound combination, and the severity of the tumor being treated and form of administration. In it also understand that one of ordinary skill in the art can readily determine the appropriate dose of the treatment compound based on these factors. Treatment dosages generally may be titrated to optimize safety and efficacy.

A treatment compound can be administered by any route of administration known in the art, such as oral, intravenous, subcutaneous, or intramucosal administration. In one embodiment, lenalidomide or a stereoisomer thereof; or a pharmaceutically acceptable salt, solvate, hydrate, co-crystal, clathrate thereof; or a polymorph thereof, is administered to a patient orally. In one embodiment, Compound A or a stereoisomer thereof; or a pharmaceutically acceptable salt, solvate, hydrate, co-crystal, clathrate thereof; or a polymorph thereof, is administered to a patient orally. In one embodiment, thalidomide or a stereoisomer thereof; or a pharmaceutically acceptable salt, solvate, hydrate, co-crystal, clathrate thereof; or a polymorph thereof, is administered to a patient orally. In one embodiment, pomalidomide or a stereoisomer thereof; or a pharmaceutically acceptable salt, solvate, hydrate, co-crystal, clathrate thereof; or a polymorph thereof, is administered to a patient orally. In one embodiment, Compound B or a stereoisomer thereof; or a pharmaceutically acceptable salt, solvate, hydrate, co-crystal, clathrate thereof; or a polymorph thereof, is administered to a patient orally. The oral dosage form can be a tablet or a capsule. In some embodiments, the dosage form is a tablet. In some other embodiments, the dosage for is a capsule.

In some embodiments of the various methods provided herein, the levels of at least two biomarkers are determined. For example, in some embodiments of the various methods provided herein, two or more biomarkers independently selected from the group consisting of MST1R (RON), PARP4, MVP, ZFP91, IKBKE, CK1-alpha, PDE6D, LGALS2, KAT1, CRBN, and GSPT1 are determined. In some embodiments of the various methods provided herein, the levels of at least two biomarkers are determined, and one biomarker is CRBN and at least another biomarker is selected from Tables 1-6. In some embodiments of the various methods provided herein, the levels of at least two biomarkers are determined, and one biomarker is ZFP91 and at least another biomarker is selected from Tables 1-6. In some specific embodiments of the various methods provided herein, ZFP91 and one or more additional biomarker selected from the group consisting of MST1R (RON), PARP4, MVP, IKBKE, CK1-alpha, PDE6D, LGALS2, KAT1, and GSPT1 are determined. In some embodiments of the various methods provided herein, the levels of at least two biomarkers are determined, and one biomarker is CK1-alpha and at least another biomarker is selected from Tables 1-6. In some embodiments of the various methods provided herein, the levels of at least two biomarkers are determined, and one biomarker is GSPT1 and at least another biomarker is selected from Tables 1-6. In some embodiments of the various methods provided herein, the levels of at least two biomarkers are determined, and one biomarker is KAT1 and at least another biomarker is selected from Tables 1-6. In some embodiments of the various methods provided herein, the levels of at least two biomarkers are determined, and one biomarker is Nestin and at least another biomarker is selected from Tables 1-6. In some embodiments of the various methods provided herein, the levels of at least two biomarkers are determined, and one biomarker is WIBG and at least another biomarker is selected from Tables 1-6. In some embodiments of the various methods provided herein, the levels of at least two biomarkers are determined, and one biomarker is FAM83H and at least another biomarker is selected from Tables 1-6. In some embodiments of the various methods provided herein, the levels of at least two biomarkers are determined, and one biomarker is KLC4 and at least another biomarker is selected from Tables 1-6. In some embodiments of the various methods provided herein, the levels of at least two biomarkers are determined, and one biomarker is LIG3 and at least another biomarker is selected from Tables 1-6. In some embodiments of the various methods provided herein, the levels of at least two biomarkers are determined, and one biomarker is IKBKE and at least another biomarker is selected from Tables 1-6. In some embodiments of the various methods provided herein, the levels of at least two biomarkers are determined, and one biomarker is LGALS2 and at least another biomarker is selected from Tables 1-6. In some embodiments of the various methods provided herein, the levels of at least two biomarkers are determined, and one biomarker is LGALS9 and at least another biomarker is selected from Tables 1-6. In some embodiments of the various methods provided herein, the levels of at least two biomarkers are determined, and one biomarker is MVP and at least another biomarker is selected from Tables 1-6. In some embodiments of the various methods provided herein, the levels of at least two biomarkers are determined, and one biomarker is PARP4 and at least another biomarker is selected from Tables 1-6. In some embodiments of the various methods provided herein, the levels of at least two biomarkers are determined, and one biomarker is ZNF198 and at least another biomarker is selected from Tables 1-6. In some embodiments of the various methods provided herein, the levels of at least two biomarkers are determined, and one biomarker is MST1R (RON) and at least another biomarker is selected from Tables 1-6. In some embodiments of the various methods provided herein, the levels of at least two biomarkers are determined, and one biomarker is ZMYM198 and at least another biomarker is selected from Tables 1-6. In some embodiments of the various methods provided herein, the levels of at least two biomarkers are determined, and one biomarker is EHMT2 and at least another biomarker is selected from Tables 1-6. In some embodiments of the various methods provided herein, the levels of at least two biomarkers are determined, and one biomarker is GLRX and at least another biomarker is selected from Tables 1-6. In some embodiments of the various methods provided herein, the levels of at least two biomarkers are determined, and one biomarker is SDSL and at least another biomarker is selected from Tables 1-6. In some embodiments of the various methods provided herein, the levels of at least two biomarkers are determined, and one biomarker is PDE6D and at least another biomarker is selected from Tables 1-6.

Thus, in some specific embodiments, provided herein is a method of predicting the responsiveness of a subject having or suspected of having a solid tumor to a treatment compound, comprising:

(a) administering the treatment compound to a subject having a solid tumor;

(b) obtaining a sample from the subject;

(c) determining the levels of two or more biomarkers independently selected from the group consisting of MST1R (RON), PARP4, MVP, ZFP91, IKBKE, CK1-alpha, PDE6D, LGALS2, KAT1, and GSPT1; and

(d) diagnosing the subject as being likely to be responsive to the treatment compound if the level of the biomarkers in the sample of the subject changes as compared to a reference levels of the biomarkers.

In other embodiments, provided herein is a method of monitoring the efficacy of a treatment of a solid tumor in a subject with a treatment compound, comprising:

(a) administering the treatment compound to a subject having a solid tumor;

(b) obtaining a sample from the subject;

(c) determining the levels of two or more biomarkers independently selected from the group consisting of MST1R (RON), PARP4, MVP, ZFP91, IKBKE, CK1-alpha, PDE6D, LGALS2, KAT1, and GSPT1;

(d) comparing the levels of the biomarkers in the sample with the levels of the biomarkers obtained from a reference sample, wherein changes in the levels as compared to the reference is indicative of the efficacy of the treatment compound in treating the solid tumor in the subject.

In some embodiments, the method comprises determining the levels of ZFP91 and one or more markers selected from the group consisting of MST1R (RON), PARP4, MVP, IKBKE, CK1-alpha, PDE6D, LGALS2, KAT1, and GSPT1.

In some embodiments, the method further comprises administering a therapeutically effective amount of the treatment compound to the subject diagnosed to be likely to be responsive to the treatment compound.

4.3.3 Use of mRNAs as Biomarkers for Identifying a Subject for Treatment

Based, in part, on the finding that detectable increase or decrease in certain mRNAs are observed in subjects with a solid tumor, e.g., HCC or GBM, who are responsive to a given treatment (e.g., a treatment by a treatment compound, e.g., thalidomide, lenalidomide, pomalidomide, Compound A, or Compound B, or a stereoisomer thereof, or a pharmaceutically acceptable salt, solvate, hydrate, co-crystal, clathrate, or a polymorph thereof), the levels of the mRNA biomarkers may be used for identifying a subject having the solid tumor for the treatment by a treatment compound provided herein.

In one embodiment, provided herein is a method of identifying a subject having a solid tumor, e.g., HCC or GBM, who is likely to be responsive to a treatment compound, comprising:

(a) determining the level of an mRNA biomarker in a solid tumor cell-containing sample from the subject; and

(b) comparing the level of the mRNA biomarker in the sample to a reference level of the mRNA biomarker; wherein the subject is likely to be responsive to the treatment if the level of the mRNA biomarker in the sample of the subject is higher than the reference level of the mRNA biomarker. In one embodiment, the solid tumor is HCC. In another embodiment, the solid tumor is GBM.

In another embodiment, provided herein is a method of identifying a subject having a solid tumor, e.g., HCC or GBM, who is likely to be responsive to a treatment compound, comprising:

(a) determining the level of an mRNA biomarker in a solid tumor cell-containing sample from the subject; and

(b) comparing the level of the mRNA biomarker in the sample to a reference level of the mRNA biomarker; wherein the subject is likely to be responsive to the treatment if the level of the mRNA biomarker in the sample of the subject is lower than the reference level of the mRNA biomarker. In one embodiment, the solid tumor is HCC. In another embodiment, the solid tumor is GBM.

In yet another embodiment, provided herein is a method of identifying a subject having a solid tumor, e.g., HCC or GBM, who is likely to be responsive to a treatment compound, comprising:

(a) obtaining a biological sample from the subject;

(b) determining the level of an mRNA biomarker in the sample; and

(c) comparing the level of the mRNA biomarker in the sample to a reference level of the mRNA biomarker; wherein the subject is likely to be responsive to the treatment if the level of the mRNA biomarker in the sample of the subject is higher than the reference level of the mRNA biomarker. In one embodiment, the solid tumor is HCC. In another embodiment, the solid tumor is GBM.

In yet another embodiment, provided herein is a method of identifying a subject having a solid tumor, e.g., HCC or GBM, who is likely to be responsive to a treatment compound, comprising:

(a) obtaining a biological sample from the subject;

(b) determining the level of an mRNA biomarker in the sample; and

(c) comparing the level of the mRNA biomarker in the sample to a reference level of the mRNA biomarker; wherein the subject is likely to be responsive to the treatment if the level of the mRNA biomarker in the sample of the subject is lower than the reference level of the mRNA biomarker. In one embodiment, the solid tumor is HCC. In another embodiment, the solid tumor is GBM.

In certain embodiments, the reference level is determined from a non solid tumor-cell-containing sample from the same subject. In certain embodiments, the reference level is determined from a non solid-tumor-cell-containing sample from a group of subjects.

In one embodiment, provided herein is a method of identifying a subject having a solid tumor, e.g., HCC or GBM, who is likely to be responsive to a treatment compound, comprising:

(a) determining the level of an mRNA biomarker in a solid tumor cell-containing sample from the subject; and

(b) determining the level of the mRNA biomarker in a control sample; wherein the subject is likely to be responsive to the treatment if the level of the mRNA biomarker in the sample of the subject is higher than the level of the mRNA biomarker in the control sample. In one embodiment, the solid tumor is HCC. In another embodiment, the solid tumor is GBM.

In one embodiment, provided herein is a method of identifying a subject having a solid tumor, e.g., HCC or GBM, who is likely to be responsive to a treatment compound, comprising:

(a) determining the level of an mRNA biomarker in a solid tumor cell-containing sample from the subject; and

(b) determining the level of the mRNA biomarker in a control sample; wherein the subject is likely to be responsive to the treatment if the level of the mRNA biomarker in the sample of the subject is lower than the level of the mRNA biomarker in the control sample. In one embodiment, the solid tumor is HCC. In another embodiment, the solid tumor is GBM.

In yet another embodiment, provided herein is a method of identifying a subject having a solid tumor, e.g., HCC or GBM, who is likely to be responsive to a treatment compound, comprising:

(a) obtaining a biological sample from the subject;

(b) determining the level of an mRNA biomarker in the sample; and

(c) determining the level of the mRNA biomarker in a control sample; wherein the subject is likely to be responsive to the treatment if the level of the mRNA biomarker in the sample of the subject is higher than the level of the mRNA biomarker in the control sample. In one embodiment, the solid tumor is HCC. In another embodiment, the solid tumor is GBM.

In yet another embodiment, provided herein is a method of identifying a subject having a solid tumor, e.g., HCC or GBM, who is likely to be responsive to a treatment compound, comprising:

(a) obtaining a biological sample from the subject;

(b) determining the level of an mRNA biomarker in the sample; and

(c) determining the level of the mRNA biomarker in a control sample; wherein the subject is likely to be responsive to the treatment if the level of the mRNA biomarker in the sample of the subject is lower than the level of the mRNA biomarker in the control sample. In one embodiment, the solid tumor is HCC. In another embodiment, the solid tumor is GBM.

In certain embodiments, the control sample is a non solid tumor cell containing sample from the same subject. In certain embodiments, the control sample is a non solid tumor cell containing sample from a group of subjects.

Thus, in one embodiment, provided herein is a method of identifying a subject having a solid tumor, e.g., HCC or GBM, who is likely to be responsive to a treatment compound, comprising:

a) determining the level of an mRNA biomarker in a solid tumor cell-containing sample from the subject; and

(b) determining the level of the mRNA biomarker in a control sample from the subject; wherein the subject is likely to be responsive to the treatment if the level of the mRNA biomarker in the sample of the subject is higher than the level of the mRNA biomarker in the control sample. In one embodiment, the solid tumor is HCC. In another embodiment, the solid tumor is GBM.

In another embodiment, provided herein is a method of identifying a subject having a solid tumor, e.g., HCC or GBM, who is likely to be responsive to a treatment compound, comprising:

(a) determining the level of an mRNA biomarker in a solid tumor cell-containing sample from the subject; and

(b) determining the level of the mRNA biomarker in a control sample from the subject; wherein the subject is likely to be responsive to the treatment if the level of the mRNA biomarker in the sample of the subject is lower than the level of the mRNA biomarker in the control sample. In one embodiment, the solid tumor is HCC. In another embodiment, the solid tumor is GBM.

In yet another embodiment, provided herein is a method of identifying a subject having a solid tumor, e.g., HCC or GBM, who is likely to be responsive to a treatment compound, comprising:

(a) obtaining a biological sample from the subject;

(b) determining the level of an mRNA biomarker in the sample; and

(c) determining the level of the mRNA biomarker in a control sample from the subject; wherein the subject is likely to be responsive to the treatment if the level of the mRNA biomarker in the sample of the subject is higher than the level of the mRNA biomarker in the control sample. In one embodiment, the solid tumor is HCC. In another embodiment, the solid tumor is GBM.

In yet another embodiment, provided herein is a method of identifying a subject having a solid tumor, e.g., HCC or GBM, who is likely to be responsive to a treatment compound, comprising:

(a) obtaining a biological sample from the subject;

(b) determining the level of an mRNA biomarker in the sample; and

(c) determining the level of the mRNA biomarker in a control sample from the subject; wherein the subject is likely to be responsive to the treatment if the level of the mRNA biomarker in the sample of the subject is lower than the level of the mRNA biomarker in the control sample. In one embodiment, the solid tumor is HCC. In another embodiment, the solid tumor is GBM.

In certain embodiments, provided herein is a method of identifying a subject having a solid tumor, e.g., HCC or GBM, who is likely to be responsive to a treatment compound, comprising:

(a) administering the treatment compound to a subject having a solid tumor;

(b) obtaining a sample from the subject;

(c) determining the level of an mRNA biomarker in the sample from the subject; and

(d) diagnosing the subject as being likely to be responsive to the treatment compound if the level of the mRNA biomarker in the sample of the subject changes as compared to a reference level of the biomarker. In one embodiment, the solid tumor is HCC. In another embodiment, the solid tumor is GBM.

In one embodiment, provided herein is a method of identifying a subject having a solid tumor, e.g., HCC or GBM, who is likely to be responsive to a treatment compound, comprising:

(a) administering the treatment compound to a subject having a solid tumor;

(b) obtaining a sample from the subject;

(c) determining the level of an mRNA biomarker in the sample from the subject; and

(d) diagnosing the subject as being likely to be responsive to the treatment compound if the level of the mRNA biomarker in the sample of the subject is higher than a reference level of the mRNA biomarker. In one embodiment, the solid tumor is HCC. In another embodiment, the solid tumor is GBM.

In another embodiment, provided herein is a method of identifying a subject having a solid tumor, e.g., HCC or GBM, who is likely to be responsive to a treatment compound, comprising:

(a) administering the treatment compound to a subject having a solid tumor;

(b) obtaining a sample from the subject;

(c) determining the level of an mRNA biomarker in the sample from the subject; and

(d) diagnosing the subject as being likely to be responsive to the treatment compound if the level of the mRNA biomarker in the sample of the subject is lower than a reference level of the mRNA biomarker. In one embodiment, the solid tumor is HCC. In another embodiment, the solid tumor is GBM.

In certain embodiments, the reference is prepared by using a control sample obtained from the subject prior to administration of the treatment compound to the subject; and wherein the control sample is from the same source as the sample.

Thus, in some embodiments, provided herein is a method of identifying a subject having a solid tumor, e.g., HCC or GBM, who is likely to be responsive to a treatment compound, comprising:

(a) administering the treatment compound to a subject having a solid tumor;

(b) obtaining a sample from the subject;

(c) determining the level of an mRNA biomarker in the sample from the subject; and

(d) diagnosing the subject as being likely to be responsive to the treatment compound if the level of the mRNA biomarker in the sample of the subject changes as compared to the level of the biomarker in control sample obtained from the subject prior to administering the treatment compound. In one embodiment, the solid tumor is HCC. In another embodiment, the solid tumor is GBM.

In one embodiment, provided herein is a method of identifying a subject having a solid tumor, e.g., HCC or GBM, who is likely to be responsive to a treatment compound, comprising:

(a) administering the treatment compound to a subject having a solid tumor;

(b) obtaining a sample from the subject;

(c) determining the level of an mRNA biomarker in the sample from the subject; and

(d) diagnosing the subject as being likely to be responsive to the treatment compound if the level of the mRNA biomarker in the sample of the subject is higher than the level of the biomarker in control sample obtained from the subject prior to administering the treatment compound. In one embodiment, the solid tumor is HCC. In another embodiment, the solid tumor is GBM.

In another embodiment, provided herein is a method of identifying a subject having a solid tumor, e.g., HCC or GBM, who is likely to be responsive to a treatment compound, comprising:

(a) administering the treatment compound to a subject having a solid tumor;

(b) obtaining a sample from the subject;

(c) determining the level of an mRNA biomarker in the sample from the subject; and

(d) diagnosing the subject as being likely to be responsive to the treatment compound if the level of the mRNA biomarker in the sample of the subject is lower than the level of the biomarker in control sample obtained from the subject prior to administering the treatment compound. In one embodiment, the solid tumor is HCC. In another embodiment, the solid tumor is GBM.

In certain embodiments, the reference is prepared by using a control sample obtained from a healthy subject not having a solid tumor, e.g., HCC or GBM; and wherein the control sample is from the same source as the sample.

Thus, in some embodiments, provided herein is a method of identifying a subject having a solid tumor, e.g., HCC or GBM, who is likely to be responsive to a treatment compound, comprising:

(a) administering the treatment compound to a subject having a solid tumor;

(b) obtaining a sample from the subject;

(c) determining the level of an mRNA biomarker in the sample from the subject; and

(d) diagnosing the subject as being likely to be responsive to the treatment compound if the level of the mRNA biomarker in the sample of the subject changes as compared to the level of the biomarker in control sample obtained from a healthy subject not having the solid tumor. In one embodiment, the solid tumor is HCC. In another embodiment, the solid tumor is GBM.

In one embodiment, provided herein is a method of identifying a subject having a solid tumor, e.g., HCC or GBM, who is likely to be responsive to a treatment compound, comprising:

(a) administering the treatment compound to a subject having a solid tumor;

(b) obtaining a sample from the subject;

(c) determining the level of an mRNA biomarker in the sample from the subject; and

(d) diagnosing the subject as being likely to be responsive to the treatment compound if the level of the mRNA biomarker in the sample of the subject is higher than the level of the biomarker in control sample obtained from a healthy subject not having the solid tumor. In one embodiment, the solid tumor is HCC. In another embodiment, the solid tumor is GBM.

In another embodiment, provided herein is a method of identifying a subject having a solid tumor, e.g., HCC or GBM, who is likely to be responsive to a treatment compound, comprising:

(a) administering the treatment compound to a subject having a solid tumor;

(b) obtaining a sample from the subject;

(c) determining the level of an mRNA biomarker in the sample from the subject; and

(d) diagnosing the subject as being likely to be responsive to the treatment compound if the level of the mRNA biomarker in the sample of the subject is lower than the level of the biomarker in control sample obtained from a healthy subject not having the solid tumor. In one embodiment, the solid tumor is HCC. In another embodiment, the solid tumor is GBM.

In certain embodiments, the methods provided herein are coupled with a treatment by a treatment compound provided herein, e.g., thalidomide, lenalidomide, pomalidomide, Compound A, or Compound B, or a stereoisomer thereof, or a pharmaceutically acceptable salt, solvate, hydrate, co-crystal, clathrate, or a polymorph thereof.

Thus, in some embodiments, provided herein is a method of treating a solid tumor, e.g., HCC or GBM, comprising:

(a) obtaining a sample from the subject;

(b) determining the level of an mRNA biomarker in the sample from the subject;

(c) diagnosing the subject as being likely to be responsive to a treatment compound if the level of the mRNA biomarker in the sample of the subject changes as compared to a reference level of the mRNA biomarker; and

(d) administering a therapeutically effective amount of the treatment compound to the subject diagnosed to be likely to be responsive to the treatment compound. In one embodiment, the solid tumor is HCC. In another embodiment, the solid tumor is GBM.

In one embodiment, provided herein is a method of treating a solid tumor, e.g., HCC or GBM, comprising:

(a) obtaining a sample from the subject;

(b) determining the level of an mRNA biomarker in the sample from the subject;

(c) diagnosing the subject as being likely to be responsive to a treatment compound if the level of the mRNA biomarker in the sample of the subject is higher than a reference level of the mRNA biomarker; and

(d) administering a therapeutically effective amount of the treatment compound to the subject diagnosed to be likely to be responsive to the treatment compound. In one embodiment, the solid tumor is HCC. In another embodiment, the solid tumor is GBM.

In another embodiment, provided herein is a method of treating a solid tumor, e.g., HCC or GBM, comprising:

(a) obtaining a sample from the subject;

(b) determining the level of an mRNA biomarker in the sample from the subject;

(c) diagnosing the subject as being likely to be responsive to a treatment compound if the level of the mRNA biomarker in the sample of the subject is lower than a reference level of the mRNA biomarker; and

(d) administering a therapeutically effective amount of the treatment compound to the subject diagnosed to be likely to be responsive to the treatment compound. In one embodiment, the solid tumor is HCC. In another embodiment, the solid tumor is GBM.

In certain embodiments, the reference is prepared by using a control sample obtained from the subject prior to administration of the treatment compound to the subject; and wherein the control sample is from the same source as the sample.

Thus, in some embodiments, provided herein is a method of treating a solid tumor, e.g., HCC or GBM, comprising:

(a) obtaining a sample from the subject;

(b) determining the level of an mRNA biomarker in the sample from the subject; and

(c) diagnosing the subject as being likely to be responsive to the treatment compound if the level of the mRNA biomarker in the sample of the subject changes as compared to the level of the biomarker in control sample obtained from the subject prior to administering the treatment compound; and

(d) administering a therapeutically effective amount of the treatment compound to the subject diagnosed to be likely to be responsive to the treatment compound. In one embodiment, the solid tumor is HCC. In another embodiment, the solid tumor is GBM.

In one embodiment, provided herein is a method of treating a solid tumor, e.g., HCC or GBM, comprising:

(a) obtaining a sample from the subject;

(b) determining the level of an mRNA biomarker in the sample from the subject; and

(c) diagnosing the subject as being likely to be responsive to the treatment compound if the level of the mRNA biomarker in the sample of the subject is higher than the level of the biomarker in control sample obtained from the subject prior to administering the treatment compound; and

(d) administering a therapeutically effective amount of the treatment compound to the subject diagnosed to be likely to be responsive to the treatment compound. In one embodiment, the solid tumor is HCC. In another embodiment, the solid tumor is GBM.

In another embodiment, provided herein is a method of treating a solid tumor, e.g., HCC or GBM, comprising:

(a) obtaining a sample from the subject;

(b) determining the level of an mRNA biomarker in the sample from the subject; and

(c) diagnosing the subject as being likely to be responsive to the treatment compound if the level of the mRNA biomarker in the sample of the subject is lower than the level of the biomarker in control sample obtained from the subject prior to administering the treatment compound; and

(d) administering a therapeutically effective amount of the treatment compound to the subject diagnosed to be likely to be responsive to the treatment compound. In one embodiment, the solid tumor is HCC. In another embodiment, the solid tumor is GBM.

In certain embodiments, the reference is prepared by using a control sample obtained from a healthy subject not having the solid tumor; and wherein the control sample is from the same source as the sample.

Thus, in some embodiments, provided herein is a method of treating a solid tumor, e.g., HCC or GBM, comprising:

(a) obtaining a sample from the subject;

(b) determining the level of an mRNA biomarker in the sample from the subject; and

(c) diagnosing the subject as being likely to be responsive to the treatment compound if the level of the mRNA biomarker in the sample of the subject changes as compared to the level of the biomarker in control sample obtained from a healthy subject not having the solid tumor; and

(d) administering a therapeutically effective amount of the treatment compound to the subject diagnosed to be likely to be responsive to the treatment compound. In one embodiment, the solid tumor is HCC. In another embodiment, the solid tumor is GBM.

In one embodiment, provided herein is a method of treating a solid tumor, e.g., HCC or GBM, comprising:

(a) obtaining a sample from the subject;

(b) determining the level of an mRNA biomarker in the sample from the subject; and

(c) diagnosing the subject as being likely to be responsive to the treatment compound if the level of the mRNA biomarker in the sample of the subject is higher than the level of the biomarker in control sample obtained from a healthy subject not having the solid tumor; and

(d) administering a therapeutically effective amount of the treatment compound to the subject diagnosed to be likely to be responsive to the treatment compound. In one embodiment, the solid tumor is HCC. In another embodiment, the solid tumor is GBM.

In another embodiment, provided herein is a method of treating a solid tumor, e.g., HCC or GBM, comprising:

(a) obtaining a sample from the subject;

(b) determining the level of an mRNA biomarker in the sample from the subject; and

(c) diagnosing the subject as being likely to be responsive to the treatment compound if the level of the mRNA biomarker in the sample of the subject is lower than the level of the biomarker in control sample obtained from a healthy subject not having the solid tumor; and

(d) administering a therapeutically effective amount of the treatment compound to the subject diagnosed to be likely to be responsive to the treatment compound. In one embodiment, the solid tumor is HCC. In another embodiment, the solid tumor is GBM.

In certain embodiments, the reference level is determined simultaneously with the sample. In certain embodiments, the reference level is determined independently from the sample.

In some embodiments, the biomarker is an mRNA of a protein selected from a group consisting of CRBN, CK1-alpha, GSPT1, Nestin, KAT1/CCBL1, WIBG, FAM83H, KLC4, LIG3, IKBKE, LGALS2 LGALS9, MVP, PARP4, ZFP91, ZNF198, MST1R (RON), ZMYM198, EHMT2, GLRX, SDSL, and PDE6D. In some embodiments of the various methods provided herein, the biomarker is an mRNA of a protein selected from a group consisting of CRBN, CK1-alpha, GSPT1, Nestin, KAT1/CCBL1, WIBG, FAM83H, KLC4, LIG3, IKBKE, LGALS2 and LGALS9. In other embodiments, the biomarker is an mRNA of a protein selected from a group consisting of MVP, PARP4, ZFP91, ZNF198, MST1R (RON), ZMYM198, EHMT2, GLRX, SDSL, and PDE6D. In a specific embodiment of the various methods provided herein, the biomarker is an mRAN of ZFP91. In another embodiment of the various methods provided herein, the biomarker is an mRNA of CRBN. In yet another embodiment of the various methods provided herein, the biomarkers are mRNAs of both ZFP91 and CRBN. In another specific embodiment of the various methods provided herein, the biomarker is an mRNA of CK1-alpha. In another specific embodiment of the various methods provided herein, the biomarker is an mRNA of GSPT1. In another specific embodiment of the various methods provided herein, the biomarker is an mRNA of MST1R (RON). In another specific embodiment of the various methods provided herein, the biomarker is an mRNA of Nestin. In another specific embodiment of the various methods provided herein, the biomarker is an mRNA of KAT1/CCBL1. In another specific embodiment of the various methods provided herein, the biomarker is an mRNA of WIBG. In another specific embodiment of the various methods provided herein, the biomarker is an mRNA of MVP. In yet another specific embodiment of the various methods provided herein, the biomarker is an mRNA of PARP4. In yet another specific embodiment of the various methods provided herein, the biomarker is an mRNA of ZNF198. In yet another specific embodiment of the various methods provided herein, the biomarker is an mRNA of ZMYM198. In yet another specific embodiment of the various methods provided herein, the biomarker is an mRNA of FAM83H. In yet another specific embodiment of the various methods provided herein, the biomarker is an mRNA of KLC4. In yet another specific embodiment of the various methods provided herein, the biomarker is an mRNA of EHMT2. In yet another specific embodiment of the various methods provided herein, the biomarker is an mRNA of GLRX. In yet another specific embodiment of the various methods provided herein, the biomarker is an mRNA of SDSL. In yet another specific embodiment of the various methods provided herein, the biomarker is an mRNA of LIG3. In yet another specific embodiment of the various methods provided herein, the biomarker is an mRNA of IKBKE. In yet another specific embodiment of the various methods provided herein, the biomarker is an mRNA of PDE6D. In yet another specific embodiment of the various methods provided herein, the biomarker is an mRNA of LGALS2. In yet another specific embodiment of the various methods provided herein, the biomarker is an mRNA of LGALS9.

In some embodiments of the various methods provided herein, the biomarker is an mRNA of a protein selected from a group consisting of AHNAK, ALOX5, AMPD3, ANXA4, ANXA6, ARHGAP19, ASNS, ASPM, ATP2B4, B4GALT3, BANK1, BCDIN3D, BLZF1, BMF, BST2, C10orf76, C19orf66, CA2, CA8, CAMSAP3, CCDC69, CCNB1, CD36, CDC7, CDCA3, CENPF, CLN3, CNN3, CORO1B, CPNE2, CRBN, CSNK1A1, CSRP2, CTNND1, CTSH, DAPK2, DDX58, DHPS, DHX58, DLG2, DLGAP5, DOK3, DTX3L, ECT2, EFCAB4B, EHMT1, EHMT2, EIF2AK2, EPB41L1, EPCAM, ESRP1, ETV6, EXTL2, F13A1, FAM195A, FAM65B, FBRSL1, FCGR2B, FES, FHOD1, FIGNL1, FMNL3, GBP1, GMFG, GMPR, GPT2, GRAMD1A, GRAMD1B, GRPEL2, HIP1, HJURP, HLA-B, HLA-DMA, HMCES, HMMR, HOXC4, HPSE, ICAM2, ID3, IFI35, IFIH1, IFIT1, IFIT3, IFIT5, IFITM2, IKZF1, IKZF3, IL4I1, IRF7, IRF9, IRS2, ISG15, ISG20, ITGB7, JAK3, KIF18B, KIF22, KIF2C, LAP3, LGALS1, LGALS3BP, LIMD1, LIPG, LPXN, MAN2A2, MARCKS, MFI2, MGARP, MINA, MIS18BP1, MOV10, MPP7, MUC1, MX1, MX2, MYO1G, NCF2, NEIL1, NFKBID, NME3, NMI, NPIPB5, NT5C3A, OAS1, OAS2, OAS3, OMA1, ORC6, PARP14, PARP9, PARVB, PBK, PBXIP1, PDE6D, PKMYT1, PLD4, PLEKHO1, PLK1, PLSCR1, PLXNB2, PODXL, PODXL2, POLE2, POMP, PPFIBP1, PRDM15, PRNP, PTAFR, PTMS, PTTG1, PYROXD1, QPRT, RAB13, RASA4B, RASSF6, RCN1, RGCC, RGS1, RGS2, RNF213, S100A13, SAMD9L, SAMHD1, SEC14L1, SERPINH1, SGOL1, SGOL2, SLCO3A1, SLCO4A1, SLFN11, SLFN13, SLFN5, SP110, SP140, SPN, SPR, STAP1, STAT1, STAT2, TACC3, TAP1, TAX1BP3, THEMIS2, THTPA, TIMM8B, TNFAIP8L2, TNFSF8, TOP2A, TP5313, TPX2, TREX1, TRIB3, TRIM22, TTC39C, TXNIP, UBA7, UBE2L6, USP41, VCL, VNN2, WIZ, WSB1, WWC1, ZBTB38, ZFP91, ZMYM2, ZNF385B, ZNF581 and ZNF644.

In other embodiments of the various methods provided herein, the biomarker is an mRNA of a protein selected from a group consisting of AHNAK, ALOX5, AMPD3, ANXA4, ANXA6, ATP2B4, BMF, BST2, C10orf76, C19orf66, CD36, CLN3, CNN3, CORO1B, CPNE2, CRBN, CSRP2, CTNND1, CTSH, DAPK2, DDX58, DHX58, DLG2, DTX3L, EIF2AK2, EPB41L1, ETV6, EXTL2, F13A1, FAM65B, FCGR2B, FES, FMNL3, GBP1, GMFG, GMPR, HIP1, HLA-B, HLA-DMA, HPSE, ID3, IFI35, IFIH1, IFIT1, IFIT3, IFIT5, IFITM2, IL4I1, IRF7, IRF9, ISG15, ISG20, ITGB7, JAK3, LAP3, LGALS1, LGALS3BP, LIMD1, MAN2A2, MARCKS, MFI2, MGARP, MOV10, MPP7, MUC1, MX1, MX2, MYO1G, NCF2, NME3, NMI, NT5C3A, OAS1, OAS2, OAS3, PARP14, PARP9, PBXIP1, PLD4, PLEKHO1, PLSCR1, PLXNB2, POMP, PPFIBP1, PTMS, QPRT, RAB13, RCN1, RGCC, RNF213, S100A13, SAMD9L, SAMHD1, SERPINH1, SLFN11, SLFN13, SLFN5, SP110, SP140, SPN, SPR, STAP1, STAT1, STAT2, TAP1, TAX1BP3, THEMIS2, THTPA, TNFAIP8L2, TNFSF8, TP5313, TREX1, TRIM22, TTC39C, TXNIP, UBA7, UBE2L6, USP41, VCL, VNN2 and ZBTB38.

In yet other embodiments of the various methods provided herein, the biomarker is an mRNA of a protein selected from a group consisting of ARHGAP19, ASNS, ASPM, B4GALT3, BANK1, BCDIN3D, BLZF1, CA2, CA8, CAMSAP3, CCDC69, CCNB1, CDC7, CDCA3, CENPF, CSNK1A1DHPS, DLGAP5, DOK3, ECT2, EFCAB4B, EHMT1, EHMT2, EPCAM, ESRP1, FAM195A, FBRSL1, FHOD1, FIGNL1, GPT2, GRAMD1A, GRAMD1B, GRPEL2, HJURP, HMCES, HMMR, HOXC4, ICAM2, IKZF1, IKZF3, IRS2, KIF18B, KIF22, KIF2C, LIPG, LPXN, MINA, MIS18BP1, NEIL1, NFKBID, NPIPB5, OMA1, ORC6, PARVB, PBK, PDE6D, PKMYT1, PLK1, PODXL, PODXL2, POLE2, PRDM15, PRNP, PTAFR, PTTG1, PYROXD1, RASA4B, RASSF6, RGS1, RGS2, SEC14L1, SGOL1, SGOL2, SLCO3A1, SLCO4A1, TACC3, TIMM8B, TOP2A, TPX2, TRIB3, WIZ, WSB1, WWC1, ZFP91, ZMYM2, ZNF385B, ZNF581 and ZNF644.

In yet other embodiments of the various methods provided herein, the biomarker is an mRNA of a protein selected from a group consisting of SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTU1, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770.

In yet other embodiments of the various methods provided herein, the biomarker is an mRNA of a protein selected from a group consisting of MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDC38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLIT, FNBP1L, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830.

In yet other embodiments of the various methods provided herein, the biomarker is an mRNA of a protein selected from a group consisting of ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Tap, Igf2r, IGFBP1, IGFBP3, IKBKE, JAG1, KIAA0100, KIAA1462, KIF20B, KIF22, KLC2, KRT9, LDLR, LPXN, MAFF, MELK, MET, MGAT4B, MGLL, MMP7, MST1R, MT2A, MVP, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2.

In yet other embodiments of the various methods provided herein, the biomarker is an mRNA of a protein selected from a group consisting of ACSL6, AHSG, ALB, ARPP19, ATP5EP2, ATP5I, ATP5J, AZGP1, BCKDK, BLOC1S3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1H1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBC1D12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2.

In certain embodiments, the mRNA biomarkers are used to identify a subject having a solid tumor, e.g., HCC or GBM, who is likely to be responsive to treatment by thalidomide, lenalidomide, pomalidomide, Compound A, or Compound B, or a stereoisomer thereof, or a pharmaceutically acceptable salt, solvate, hydrate, co-crystal, clathrate, or a polymorph thereof.

In one embodiment, the mRNA biomarkers are used to identify a subject having a solid tumor, e.g., HCC or GBM, who is likely to be responsive to treatment by thalidomide. In another embodiment, the mRNA biomarkers are used to identify a subject having a solid tumor, e.g., HCC or GBM, who is likely to be responsive to treatment by lenalidomide. In yet another embodiment, the mRNA biomarkers are used to identify a subject having a solid tumor, e.g., HCC or GBM, who is likely to be responsive to treatment by pomalidomide. In yet another embodiment, the mRNA biomarkers are used to identify a subject having a solid tumor, e.g., HCC or GBM, who is likely to be responsive to treatment by Compound A. In yet another embodiment, the mRNA biomarkers are used to identify a subject having a solid tumor, e.g., HCC or GBM, who is likely to be responsive to treatment by Compound B.

In certain embodiments, the level of the mRNA biomarker is measured in a biological sample obtained from the subject.

In certain embodiments, the level of the mRNA biomarker is measured in an in vitro assay to predict the responsiveness of a subject to a treatment of a solid tumor, e.g., HCC or GBM, comprising obtaining a sample of cells from the subject, culturing the cells in the presence or absence of a treatment compound, and testing the cells for the levels of the biomarkers, wherein a decreased level of the biomarker in the presence of the treatment compound indicates the likelihood of responsiveness of the subject to the treatment compound.

In certain embodiments, the level of only one of the mRNA biomarkers is monitored. In certain embodiments, the levels of two or more of the mRNA biomarkers are monitored simultaneously.

In some embodiments, the solid tumors are sarcomas, carcinomas (epithelial tumors), melanomas, and glioblastomas. Exemplary solid tumors include, but not limited to, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma, glioblastoma (GBM); medulloblastoma), cervical cancer (e.g., cervical adenocarcinoma), colorectal cancer (e.g., colon cancer, rectal cancer, colorectal adenocarcinoma), gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), pancreatic cancer (e.g., pancreatic andenocarcinoma, intraductal papillary mucinous neoplasm (IPMN)), prostate cancer (e.g., prostate adenocarcinoma), skin cancer (e.g., squamous cell carcinoma (SCC), keratoacanthoma (A), melanoma, basal cell carcinoma (BCC)) and soft tissue sarcoma (e.g., malignant fibrous histiocytoma (MFH), liposarcoma, malignant peripheral nerve sheath tumor (MPNST), chondrosarcoma, fibrosarcoma, myxosarcoma, osteosarcoma). In some embodiments, the solid tumor is a liver cancer. In one embodiment, the solid tumor is HCC. In other embodiments, the solid tumor is a brain cancer. In one embodiment, the solid tumor is GBM.

4.3.4 Use of mRNA as Biomarkers for Predicting the Efficacy

Based, in part, on the finding that detectable increase or decrease in certain mRNA biomarkers are observed in subjects with a solid tumor, e.g., HCC or GBM, who are responsive to a given treatment (e.g., a treatment by a treatment compound provided herein, e.g., thalidomide, lenalidomide, pomalidomide, Compound A, or Compound B, or a stereoisomer thereof, or a pharmaceutically acceptable salt, solvate, hydrate, co-crystal, clathrate, or a polymorph thereof), the levels of these mRNA biomarkers may be used for predicting the responsiveness of the subjects to the treatment.

In one embodiment, provided herein is a method of predicting the responsiveness of a subject having or suspected of having a solid tumor, e.g., HCC or GBM, to a treatment compound, comprising:

a) determining the level of an mRNA biomarker in a solid tumor cell-containing sample from the subject; and

(b) comparing the level of the mRNA biomarker in the sample to a reference level of the mRNA biomarker, wherein an increased level of the mRNA biomarker in the sample correlates with an increased responsiveness of the subject to the compound treatment. In one embodiment, the solid tumor is HCC. In another embodiment, the solid tumor is GBM.

In another embodiment, provided herein is a method of predicting the responsiveness of a subject having or suspected of having a solid tumor, e.g., HCC or GBM, to a treatment compound, comprising:

(a) determining the level of an mRNA biomarker in a solid tumor cell-containing sample from the subject; and

(b) comparing the level of the mRNA biomarker in the sample to a reference level of the mRNA biomarker, wherein a decreased level of the mRNA biomarker in the sample correlates with an increased responsiveness of the subject to the compound treatment. In one embodiment, the solid tumor is HCC. In another embodiment, the solid tumor is GBM.

In yet another embodiment, provided herein is a method of predicting the responsiveness of a subject having or suspected of having a solid tumor, e.g., HCC or GBM, to a treatment compound, comprising:

(a) obtaining a biological sample from the subject;

(b) determining the level of an mRNA biomarker in the sample; and

(c) comparing the level of the mRNA biomarker in the sample to a reference level of the mRNA biomarker, wherein an increased level of the mRNA biomarker in the sample correlates with an increased responsiveness of the subject to the compound treatment. In one embodiment, the solid tumor is HCC. In another embodiment, the solid tumor is GBM.

In yet another embodiment, provided herein is a method of predicting the responsiveness of a subject having or suspected of having a solid tumor, e.g., HCC or GBM, to a treatment compound, comprising:

(a) obtaining a biological sample from the subject;

(b) determining the level of an mRNA biomarker in the sample; and

(c) comparing the level of the mRNA biomarker in the sample to a reference level of the mRNA biomarker, wherein a decreased level of the mRNA biomarker in the sample correlates with an increased responsiveness of the subject to the compound treatment. In one embodiment, the solid tumor is HCC. In another embodiment, the solid tumor is GBM.

In one embodiment, provided herein is a method of predicting the responsiveness of a subject having or suspected of having a solid tumor, e.g., HCC or GBM, to a treatment compound, comprising:

(a) determining the level of an mRNA biomarker in a solid tumor cell-containing sample from the subject;

(b) determining the level of the mRNA biomarker in a control sample; and

(c) comparing the level of the mRNA biomarker in the sample from the subject to the level of the mRNA biomarker in the control sample, wherein an increased level of the mRNA biomarker in the sample correlates with an increased responsiveness of the subject to the compound treatment. In one embodiment, the solid tumor is HCC. In another embodiment, the solid tumor is GBM.

In one embodiment, provided herein is a method of predicting the responsiveness of a subject having or suspected of having a solid tumor, e.g., HCC or GBM, to a treatment compound, comprising:

(a) determining the level of an mRNA biomarker in a solid tumor cell-containing sample from the subject;

(b) determining the level of the mRNA biomarker in a control sample; and

(c) comparing the level of the mRNA biomarker in the sample from the subject to the level of the mRNA biomarker in the control sample, wherein a decreased level of the mRNA biomarker in the sample correlates with an increased responsiveness of the subject to the compound treatment. In one embodiment, the solid tumor is HCC. In another embodiment, the solid tumor is GBM.

In yet another embodiment, provided herein is a method of predicting the responsiveness of a subject having or suspected of having a solid tumor, e.g., HCC or GBM, to a treatment compound, comprising:

(a) obtaining a biological sample from the subject;

(b) determining the level of an mRNA biomarker in the sample;

(c) determining the level of the mRNA biomarker in a control sample; and

(d) comparing the level of the mRNA biomarker in the sample from the subject to the level of the mRNA biomarker in the control sample, wherein an increased level of the mRNA biomarker in the sample correlates with an increased responsiveness of the subject to the compound treatment. In one embodiment, the solid tumor is HCC. In another embodiment, the solid tumor is GBM.

In yet another embodiment, provided herein is a method of predicting the responsiveness of a subject having or suspected of having a solid tumor, e.g., HCC or GBM, to a treatment compound, comprising:

(a) obtaining a biological sample from the subject;

(b) determining the level of an mRNA biomarker in the sample;

(c) determining the level of the mRNA biomarker in a control sample; and

(d) comparing the level of the mRNA biomarker in the sample from the subject to the level of the mRNA biomarker in the control sample, wherein a decreased level of the mRNA biomarker in the sample correlates with an increased responsiveness of the subject to the compound treatment. In one embodiment, the solid tumor is HCC. In another embodiment, the solid tumor is GBM.

In certain embodiments, provided herein is a method of predicting the responsiveness of a subject having or suspected of having a solid tumor, e.g., HCC or GBM, to a treatment compound, comprising:

(a) administering the treatment compound to a subject having a solid tumor;

(b) obtaining a sample from the subject;

(c) determining the level of an mRNA biomarker in the sample from the subject;

(d) diagnosing the subject as being likely to be responsive to a treatment of the solid tumor with the treatment compound if the level of the mRNA biomarker in the sample changes as compared to the level of the biomarker obtained from a reference sample. In one embodiment, the solid tumor is HCC. In another embodiment, the solid tumor is GBM.

In one embodiment, provided herein is a method of predicting the responsiveness of a subject having or suspected of having a solid tumor, e.g., HCC or GBM, to a treatment compound, comprising:

(a) administering the treatment compound to a subject having a solid tumor;

(b) obtaining a sample from the subject;

(c) determining the level of an mRNA biomarker in the sample from the subject;

(d) diagnosing the subject as being likely to be responsive to a treatment of the solid tumor with the treatment compound if the level of the mRNA biomarker in the sample is higher than the level of the biomarker obtained from a reference sample. In one embodiment, the solid tumor is HCC. In another embodiment, the solid tumor is GBM.

In another embodiment, provided herein is a method of predicting the responsiveness of a subject having or suspected of having a solid tumor, e.g., HCC or GBM, to a treatment compound, comprising:

(a) administering the treatment compound to a subject having the solid tumor;

(b) obtaining a sample from the subject;

(c) determining the level of an mRNA biomarker in the sample from the subject;

(d) diagnosing the subject as being likely to be responsive to a treatment of the solid tumor with the treatment compound if the level of the mRNA biomarker in the sample is lower than the level of the biomarker obtained from a reference sample. In one embodiment, the solid tumor is HCC. In another embodiment, the solid tumor is GBM.

In certain embodiments, the reference is prepared by using a control sample obtained from the subject prior to administration of the treatment compound to the subject; and wherein the control sample is from the same source as the sample.

Thus, in some embodiments, provided herein is a method of predicting the responsiveness of a subject having or suspected of having a solid tumor, e.g., HCC or GBM, to a treatment compound, comprising:

(a) administering the treatment compound to a subject having a solid tumor;

(b) obtaining a sample from the subject;

(c) determining the level of an mRNA biomarker in the sample from the subject;

(d) diagnosing the subject as being likely to be responsive to a treatment of the solid tumor with the treatment compound if the level of the mRNA biomarker in the sample changes as compared to the level of the biomarker in a control sample obtained from the subject prior to administration of the treatment compound. In one embodiment, the solid tumor is HCC. In another embodiment, the solid tumor is GBM.

In one embodiment, provided herein is a method of predicting the responsiveness of a subject having or suspected of having a solid tumor, e.g., HCC or GBM, to a treatment compound, comprising:

(a) administering the treatment compound to a subject having a solid tumor;

(b) obtaining a sample from the subject;

(c) determining the level of an mRNA biomarker in the sample from the subject;

(d) diagnosing the subject as being likely to be responsive to a treatment of the solid tumor with the treatment compound if the level of the mRNA biomarker in the sample is higher than the level of the biomarker in a control sample obtained from the subject prior to administration of the treatment compound. In one embodiment, the solid tumor is HCC. In another embodiment, the solid tumor is GBM.

In another embodiment, provided herein is a method of predicting the responsiveness of a subject having or suspected of having a solid tumor, e.g., HCC or GBM, to a treatment compound, comprising:

(a) administering the treatment compound to a subject having a solid tumor;

(b) obtaining a sample from the subject;

(c) determining the level of an mRNA biomarker in the sample from the subject;

(d) diagnosing the subject as being likely to be responsive to a treatment of the solid tumor with the treatment compound if the level of the mRNA biomarker in the sample is lower than the level of the biomarker in a control sample obtained from the subject prior to administration of the treatment compound. In one embodiment, the solid tumor is HCC. In another embodiment, the solid tumor is GBM.

In certain embodiments, the reference is prepared by using a control sample obtained from a healthy subject not having the solid tumor; and wherein the control sample is from the same source as the sample.

Thus, in some embodiments, provided herein is a method of predicting the responsiveness of a subject having or suspected of having a solid tumor, e.g., HCC or GBM, to a treatment compound, comprising:

(a) administering the treatment compound to a subject having the solid tumor;

(b) obtaining a sample from the subject;

(c) determining the level of an mRNA biomarker in the sample from the subject;

(d) diagnosing the subject as being likely to be responsive to a treatment of the solid tumor with the treatment compound if the level of the mRNA biomarker in the sample changes as compared to the level of the biomarker in a control sample obtained from a healthy subject not having the solid tumor. In one embodiment, the solid tumor is HCC. In another embodiment, the solid tumor is GBM.

In one embodiment, provided herein is a method of predicting the responsiveness of a subject having or suspected of having a solid tumor, e.g., HCC or GBM, to a treatment compound, comprising:

(a) administering the treatment compound to a subject having a solid tumor;

(b) obtaining a sample from the subject;

(c) determining the level of an mRNA biomarker in the sample from the subject;

(d) diagnosing the subject as being likely to be responsive to a treatment of the solid tumor with the treatment compound if the level of the mRNA biomarker in the sample is higher than the level of the biomarker in a control sample obtained from a healthy subject not having the solid tumor. In one embodiment, the solid tumor is HCC. In another embodiment, the solid tumor is GBM.

In another embodiment, provided herein is a method of predicting the responsiveness of a subject having or suspected of having a solid tumor, e.g., HCC or GBM, to a treatment compound, comprising:

(a) administering the treatment compound to a subject having a solid tumor;

(b) obtaining a sample from the subject;

(c) determining the level of an mRNA biomarker in the sample from the subject;

(d) diagnosing the subject as being likely to be responsive to a treatment of the solid tumor with the treatment compound if the level of the mRNA biomarker in the sample is lower than the level of the biomarker in a control sample obtained from a healthy subject not having the solid tumor. In one embodiment, the solid tumor is HCC. In another embodiment, the solid tumor is GBM.

In some embodiments of the various methods provided herein, the control sample is a non-solid tumor cell-containing sample from the same subject. In some embodiments of the various methods provided herein, the control sample is a non-solid tumor cell-containing sample from a group of subjects.

In some embodiments of the various methods provided herein, the level of the mRNA biomarker in a control sample is determined simultaneously with the sample. In other embodiments of the various methods provided herein, the level of the mRNA biomarker in a control sample is determined independently from the sample.

In some embodiments of the various methods provided herein, an increased level of the mRNA biomarker in the sample as compared to the reference level correlates positively with increased responsiveness of the subject to a treatment compound provided herein, e.g., thalidomide, lenalidomide, pomalidomide, Compound A, or Compound B, or a stereoisomer thereof, or a pharmaceutically acceptable salt, solvate, hydrate, co-crystal, clathrate, or a polymorph thereof.

In other embodiments of the various methods provided herein, a decreased level of the mRNA biomarker in the sample as compared to the reference level correlates positively with increased responsiveness of the subject to a treatment compound provided herein, e.g., thalidomide, lenalidomide, pomalidomide, Compound A, or Compound B, or a stereoisomer thereof, or a pharmaceutically acceptable salt, solvate, hydrate, co-crystal, clathrate, or a polymorph thereof.

In another aspect, provided herein is a method of monitoring the efficacy of a treatment of a solid tumor, e.g., HCC or GBM, in a subject with a treatment compound.

In some embodiments, provided herein is a method of monitoring the efficacy of a treatment of a solid tumor, e.g., HCC or GBM, in a subject with a treatment compound, comprising:

(a) administering the treatment compound to a subject having a solid tumor;

(b) obtaining a sample from the subject;

(c) determining the level of an mRNA biomarker in the sample from the subject;

(d) comparing the level of the mRNA biomarker in the sample with the level of the mRNA biomarker obtained from a reference sample, wherein a change in the level as compared to the reference is indicative of the efficacy of the treatment compound in treating the solid tumor in the subject. In one embodiment, the solid tumor is HCC. In another embodiment, the solid tumor is GBM.

In one embodiment, provided herein is a method of monitoring the efficacy of a treatment of a solid tumor, e.g., HCC or GBM, in a subject with a treatment compound, comprising:

(a) administering the treatment compound to a subject having a solid tumor;

(b) obtaining a sample from the subject;

(c) determining the level of an mRNA biomarker in the sample from the subject;

(d) comparing the level of the mRNA biomarker in the sample with the level of the mRNA biomarker obtained from a reference sample, wherein an increased level as compared to the reference is indicative of the efficacy of the treatment compound in treating the solid tumor in the subject. In one embodiment, the solid tumor is HCC. In another embodiment, the solid tumor is GBM.

In one embodiment, provided herein is a method of monitoring the efficacy of a treatment of a solid tumor, e.g., HCC or GBM, in a subject with a treatment compound, comprising:

(a) administering the treatment compound to a subject having a solid tumor;

(b) obtaining a sample from the subject;

(c) determining the level of an mRNA biomarker in the sample from the subject;

(d) comparing the level of the mRNA biomarker in the sample with the level of the mRNA biomarker obtained from a reference sample, wherein a decreased level as compared to the reference is indicative of the efficacy of the treatment compound in treating the solid tumor in the subject. In one embodiment, the solid tumor is HCC. In another embodiment, the solid tumor is GBM.

In certain embodiments, the reference is prepared by using a control sample obtained from the subject prior to administration of the treatment compound to the subject; and wherein the control sample is from the same source as the sample.

Thus, in some embodiments, provided herein is a method of monitoring the efficacy of a treatment of a solid tumor, e.g., HCC or GBM, in a subject with a treatment compound, comprising:

(a) administering the treatment compound to a subject having a solid tumor;

(b) obtaining a sample from the subject;

(c) determining the level of an mRNA biomarker in the sample from the subject;

(d) comparing the level of the mRNA biomarker in the sample with the level of the mRNA biomarker in a control sample obtained from the subject prior to administration of the treatment compound, wherein a change in the level as compared to the control is indicative of the efficacy of the treatment compound in treating the solid tumor in the subject. In one embodiment, the solid tumor is HCC. In another embodiment, the solid tumor is GBM.

In one embodiment, provided herein is a method of monitoring the efficacy of a treatment of a solid tumor, e.g., HCC or GBM, in a subject with a treatment compound, comprising:

(a) administering the treatment compound to a subject having a solid tumor;

(b) obtaining a sample from the subject;

(c) determining the level of an mRNA biomarker in the sample from the subject;

(d) comparing the level of the mRNA biomarker in the sample with the level of the mRNA biomarker in a control sample obtained from the subject prior to administration of the treatment compound, wherein an increased level as compared to the control is indicative of the efficacy of the treatment compound in treating the solid tumor in the subject. In one embodiment, the solid tumor is HCC. In another embodiment, the solid tumor is GBM.

In one embodiment, provided herein is a method of monitoring the efficacy of a treatment of a solid tumor, e.g., HCC or GBM, in a subject with a treatment compound, comprising:

(a) administering the treatment compound to a subject having a solid tumor;

(b) obtaining a sample from the subject;

(c) determining the level of an mRNA biomarker in the sample from the subject;

(d) comparing the level of the mRNA biomarker in the sample with the level of the mRNA biomarker in a control sample obtained from the subject prior to administration of the treatment compound, wherein a decreased level as compared to the control is indicative of the efficacy of the treatment compound in treating the solid tumor in the subject. In one embodiment, the solid tumor is HCC. In another embodiment, the solid tumor is GBM.

In certain embodiments, the reference is prepared by using a control sample obtained from a healthy subject not having a solid tumor; and wherein the control sample is from the same source as the sample.

Thus, in some embodiments, provided herein is a method of monitoring the efficacy of a treatment of a solid tumor, e.g., HCC or GBM, in a subject with a treatment compound, comprising:

(a) administering the treatment compound to a subject having a solid tumor;

(b) obtaining a sample from the subject;

(c) determining the level of an mRNA biomarker in the sample from the subject;

(d) comparing the level of the mRNA biomarker in the sample with the level of the mRNA biomarker in a control sample obtained from a healthy subject not having the solid tumor, wherein a change in the level as compared to the control is indicative of the efficacy of the treatment compound in treating the solid tumor in the subject. In one embodiment, the solid tumor is HCC. In another embodiment, the solid tumor is GBM.

In one embodiment, provided herein is a method of monitoring the efficacy of a treatment of a solid tumor, e.g., HCC or GBM, in a subject with a treatment compound, comprising:

(a) administering the treatment compound to a subject having a solid tumor;

(b) obtaining a sample from the subject;

(c) determining the level of an mRNA biomarker in the sample from the subject;

(d) comparing the level of the mRNA biomarker in the sample with the level of the mRNA biomarker in a control sample obtained from a healthy subject not having the solid tumor, wherein an increased level as compared to the control is indicative of the efficacy of the treatment compound in treating the solid tumor in the subject. In one embodiment, the solid tumor is HCC. In another embodiment, the solid tumor is GBM.

In one embodiment, provided herein is a method of monitoring the efficacy of a treatment of a solid tumor, e.g., HCC or GBM, in a subject with a treatment compound, comprising:

(a) administering the treatment compound to a subject having a solid tumor;

(b) obtaining a sample from the subject;

(c) determining the level of an mRNA biomarker in the sample from the subject;

(d) comparing the level of the mRNA biomarker in the sample with the level of the mRNA biomarker in a control sample obtained from a healthy subject not having the solid tumor, wherein a decreased level as compared to the control is indicative of the efficacy of the treatment compound in treating the solid tumor in the subject. In one embodiment, the solid tumor is HCC. In another embodiment, the solid tumor is GBM.

In some embodiments, the biomarker is an mRNA of a protein selected from a group consisting of CRBN, CK1-alpha, GSPT1, Nestin, KAT1/CCBL1, WIBG, FAM83H, KLC4, LIG3, IKBKE, LGALS2 LGALS9, MVP, PARP4, ZFP91, ZNF198, MST1R (RON), ZMYM198, EHMT2, GLRX, SDSL, and PDE6D. In some embodiments of the various methods provided herein, the biomarker is an mRNA of a protein selected from a group consisting of CRBN, CK1-alpha, GSPT1, Nestin, KAT1/CCBL1, WIBG, FAM83H, KLC4, LIG3, IKBKE, LGALS2 and LGALS9. In other embodiments, the biomarker is an mRNA of a protein selected from a group consisting of MVP, PARP4, ZFP91, ZNF198, MST1R (RON), ZMYM198, EHMT2, GLRX, SDSL, and PDE6D. In a specific embodiment of the various methods provided herein, the biomarker is an mRAN of ZFP91. In another embodiment of the various methods provided herein, the biomarker is an mRNA of CRBN. In yet another embodiment of the various methods provided herein, the biomarkers are mRNAs of both ZFP91 and CRBN. In another specific embodiment of the various methods provided herein, the biomarker is an mRNA of CK1-alpha. In another specific embodiment of the various methods provided herein, the biomarker is an mRNA of GSPT1. In another specific embodiment of the various methods provided herein, the biomarker is an mRNA of MST1R (RON). In another specific embodiment of the various methods provided herein, the biomarker is an mRNA of Nestin. In another specific embodiment of the various methods provided herein, the biomarker is an mRNA of KAT1/CCBL1. In another specific embodiment of the various methods provided herein, the biomarker is an mRNA of WIBG. In another specific embodiment of the various methods provided herein, the biomarker is an mRNA of MVP. In yet another specific embodiment of the various methods provided herein, the biomarker is an mRNA of PARP4. In yet another specific embodiment of the various methods provided herein, the biomarker is an mRNA of ZNF198. In yet another specific embodiment of the various methods provided herein, the biomarker is an mRNA of ZMYM198. In yet another specific embodiment of the various methods provided herein, the biomarker is an mRNA of FAM83H. In yet another specific embodiment of the various methods provided herein, the biomarker is an mRNA of KLC4. In yet another specific embodiment of the various methods provided herein, the biomarker is an mRNA of EHMT2. In yet another specific embodiment of the various methods provided herein, the biomarker is an mRNA of GLRX. In yet another specific embodiment of the various methods provided herein, the biomarker is an mRNA of SDSL. In yet another specific embodiment of the various methods provided herein, the biomarker is an mRNA of LIG3. In yet another specific embodiment of the various methods provided herein, the biomarker is an mRNA of IKBKE. In yet another specific embodiment of the various methods provided herein, the biomarker is an mRNA of PDE6D. In yet another specific embodiment of the various methods provided herein, the biomarker is an mRNA of LGALS2. In yet another specific embodiment of the various methods provided herein, the biomarker is an mRNA of LGALS9.

In some embodiments of the various methods provided herein, the biomarker is an mRNA of a protein selected from a group consisting of AHNAK, ALOX5, AMPD3, ANXA4, ANXA6, ARHGAP19, ASNS, ASPM, ATP2B4, B4GALT3, BANK1, BCDIN3D, BLZF1, BMF, BST2, C10orf76, C19orf66, CA2, CA8, CAMSAP3, CCDC69, CCNB1, CD36, CDC7, CDCA3, CENPF, CLN3, CNN3, CORO1B, CPNE2, CRBN, CSNK1A1, CSRP2, CTNND1, CTSH, DAPK2, DDX58, DHPS, DHX58, DLG2, DLGAP5, DOK3, DTX3L, ECT2, EFCAB4B, EHMT1, EHMT2, EIF2AK2, EPB41L1, EPCAM, ESRP1, ETV6, EXTL2, F13A1, FAM195A, FAM65B, FBRSL1, FCGR2B, FES, FHOD1, FIGNL1, FMNL3, GBP1, GMFG, GMPR, GPT2, GRAMD1A, GRAMD1B, GRPEL2, HIP1, HJURP, HLA-B, HLA-DMA, HMCES, HMMR, HOXC4, HPSE, ICAM2, ID3, IFI35, IFIH1, IFIT1, IFIT3, IFIT5, IFITM2, IKZF1, IKZF3, IL4I1, IRF7, IRF9, IRS2, ISG15, ISG20, ITGB7, JAK3, KIF18B, KIF22, KIF2C, LAP3, LGALS1, LGALS3BP, LIMD1, LIPG, LPXN, MAN2A2, MARCKS, MFI2, MGARP, MINA, MIS18BP1, MOV10, MPP7, MUC1, MX1, MX2, MYO1G, NCF2, NEIL1, NFKBID, NME3, NMI, NPIPB5, NT5C3A, OAS1, OAS2, OAS3, OMA1, ORC6, PARP14, PARP9, PARVB, PBK, PBXIP1, PDE6D, PKMYT1, PLD4, PLEKHO1, PLK1, PLSCR1, PLXNB2, PODXL, PODXL2, POLE2, POMP, PPFIBP1, PRDM15, PRNP, PTAFR, PTMS, PTTG1, PYROXD1, QPRT, RAB13, RASA4B, RASSF6, RCN1, RGCC, RGS1, RGS2, RNF213, S100A13, SAMD9L, SAMHD1, SEC14L1, SERPINH1, SGOL1, SGOL2, SLCO3A1, SLCO4A1, SLFN11, SLFN13, SLFN5, SP110, SP140, SPN, SPR, STAP1, STAT1, STAT2, TACC3, TAP1, TAX1BP3, THEMIS2, THTPA, TIMM8B, TNFAIP8L2, TNFSF8, TOP2A, TP53I3, TPX2, TREX1, TRIB3, TRIM22, TTC39C, TXNIP, UBA7, UBE2L6, USP41, VCL, VNN2, WIZ, WSB1, WWC1, ZBTB38, ZFP91, ZMYM2, ZNF385B, ZNF581 and ZNF644.

In other embodiments of the various methods provided herein, the biomarker is an mRNA of a protein selected from a group consisting of AHNAK, ALOX5, AMPD3, ANXA4, ANXA6, ATP2B4, BMF, BST2, C10orf76, C19orf66, CD36, CLN3, CNN3, CORO1B, CPNE2, CRBN, CSRP2, CTNND1, CTSH, DAPK2, DDX58, DHX58, DLG2, DTX3L, EIF2AK2, EPB41L1, ETV6, EXTL2, F13A1, FAM65B, FCGR2B, FES, FMNL3, GBP1, GMFG, GMPR, HIP1, HLA-B, HLA-DMA, HPSE, ID3, IFI35, IFIH1, IFIT1, IFIT3, IFIT5, IFITM2, IL4I1, IRF7, IRF9, ISG15, ISG20, ITGB7, JAK3, LAP3, LGALS1, LGALS3BP, LIMD1, MAN2A2, MARCKS, MFI2, MGARP, MOV10, MPP7, MUC1, MX1, MX2, MYO1G, NCF2, NME3, NMI, NT5C3A, OAS1, OAS2, OAS3, PARP14, PARP9, PBXIP1, PLD4, PLEKHO1, PLSCR1, PLXNB2, POMP, PPFIBP1, PTMS, QPRT, RAB13, RCN1, RGCC, RNF213, S100A13, SAMD9L, SAMHD1, SERPINH1, SLFN11, SLFN13, SLFN5, SP110, SP140, SPN, SPR, STAP1, STAT1, STAT2, TAP1, TAX1BP3, THEMIS2, THTPA, TNFAIP8L2, TNFSF8, TP5313, TREX1, TRIM22, TTC39C, TXNIP, UBA7, UBE2L6, USP41, VCL, VNN2 and ZBTB38.

In yet other embodiments of the various methods provided herein, the biomarker is an mRNA of a protein selected from a group consisting of ARHGAP19, ASNS, ASPM, B4GALT3, BANK1, BCDIN3D, BLZF1, CA2, CA8, CAMSAP3, CCDC69, CCNB1, CDC7, CDCA3, CENPF, CSNK1A1DHPS, DLGAP5, DOK3, ECT2, EFCAB4B, EHMT1, EHMT2, EPCAM, ESRP1, FAM195A, FBRSL1, FHOD1, FIGNL1, GPT2, GRAMD1A, GRAMD1B, GRPEL2, HJURP, HMCES, HMMR, HOXC4, ICAM2, IKZF1, IKZF3, IRS2, KIF18B, KIF22, KIF2C, LIPG, LPXN, MINA, MIS18BP1, NEIL1, NFKBID, NPIPB5, OMA1, ORC6, PARVB, PBK, PDE6D, PKMYT1, PLK1, PODXL, PODXL2, POLE2, PRDM15, PRNP, PTAFR, PTTG1, PYROXD1, RASA4B, RASSF6, RGS1, RGS2, SEC14L1, SGOL1, SGOL2, SLCO3A1, SLCO4A1, TACC3, TIMM8B, TOP2A, TPX2, TRIB3, WIZ, WSB1, WWC1, ZFP91, ZMYM2, ZNF385B, ZNF581 and ZNF644.

In yet other embodiments of the various methods provided herein, the biomarker is an mRNA of a protein selected from a group consisting of SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTU1, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770.

In yet other embodiments of the various methods provided herein, the biomarker is an mRNA of a protein selected from a group consisting of MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDC38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLII, FNBP1L, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830.

In yet other embodiments of the various methods provided herein, the biomarker is an mRNA of a protein selected from a group consisting of ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Tap, Igf2r, IGFBP1, IGFBP3, IKBKE, JAG1, KIAA0100, KIAA1462, KIF20B, KIF22, KLC2, KRT9, LDLR, LPXN, MAFF, MELK, MET, MGAT4B, MGLL, MMP7, MST1R, MT2A, MVP, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2.

In yet other embodiments of the various methods provided herein, the biomarker is an mRNA of a protein selected from a group consisting of ACSL6, AHSG, ALB, ARPP19, ATP5EP2, ATP5I, ATP5J, AZGP1, BCKDK, BLOC1S3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1H1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBC1D12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2.

In certain embodiments, the mRNA biomarkers are used to predict the responsiveness of a subject having or suspected of having a solid tumor, e.g., HCC or GBM, to a treatment by thalidomide, lenalidomide, pomalidomide, Compound A, or Compound B, or a stereoisomer thereof, or a pharmaceutically acceptable salt, solvate, hydrate, co-crystal, clathrate, or a polymorph thereof.

In some embodiments, the mRNA biomarkers are used to predict the responsiveness of a subject having or suspected of having a solid tumor, e.g., HCC or GBM, to a treatment by thalidomide. In some embodiments, the mRNA biomarkers are used to predict the responsiveness of a subject having or suspected of having a solid tumor, e.g., HCC or GBM, to a treatment by lenalidomide. In other embodiments, the mRNA biomarkers are used to predict the responsiveness of a subject having or suspected of having a solid tumor, e.g., HCC or GBM, to a treatment by pomalidomide. In other embodiments, the mRNA biomarkers are used to predict the responsiveness of a subject having or suspected of having a solid tumor, e.g., HCC or GBM, to a treatment by Compound A. In yet other embodiments, the mRNA biomarkers are used to predict the responsiveness of a subject having or suspected of having a solid tumor, e.g., HCC or GBM, to a treatment by Compound B.

In certain embodiments, the mRNA biomarkers are used to monitor the efficacy of treatment of a solid tumor, e.g., HCC or GBM, by thalidomide, lenalidomide, pomalidomide, Compound A, or Compound B, or a stereoisomer thereof, or a pharmaceutically acceptable salt, solvate, hydrate, co-crystal, clathrate, or a polymorph thereof.

In some embodiments, the mRNA biomarkers are used to monitor the efficacy of treatment of a solid tumor, e.g., HCC or GBM, by thalidomide. In some embodiments, the mRNA biomarkers are used to monitor the efficacy of treatment of a solid tumor, e.g., HCC or GBM, by lenalidomide. In other embodiments, the mRNA biomarkers are used to monitor the efficacy of treatment of a solid tumor, e.g., HCC or GBM, by pomalidomide. In other embodiments, the mRNA biomarkers are used to monitor the efficacy of treatment of a solid tumor, e.g., HCC or GBM, by Compound A. In yet other embodiments, the mRNA biomarkers are used to monitor the efficacy of treatment of a solid tumor, e.g., HCC or GBM, by Compound B.

In certain embodiments, the level of the mRNA biomarker is measured in an in vitro assay, comprising obtaining a sample of cells from the subject, culturing the cells in the presence or absence of a treatment compound, and testing the cells for the levels of the mRNA biomarkers, wherein an increased level of the mRNA biomarker in the presence of the treatment compound indicates the likelihood of responsiveness of the subject to the treatment compound.

In certain embodiments, the level of the mRNA biomarker is measured in an in vitro assay, comprising obtaining a sample of cells from the subject, culturing the cells in the presence or absence of a treatment compound, and testing the cells for the levels of the mRNA biomarkers, wherein a decreased level of the mRNA biomarker in the presence of the treatment compound indicates the likelihood of responsiveness of the subject to the treatment compound.

In certain embodiments, the level of the mRNA biomarker is measured in a biological sample obtained from the subject. In certain embodiments, the level of only one of the mRNA biomarkers is monitored. In certain embodiments, the levels of two or more of the mRNA biomarkers are monitored simultaneously.

In some embodiments, the solid tumors are sarcomas, carcinomas (epithelial tumors), melanomas, and glioblastomas. Exemplary solid tumors include, but not limited to, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma, glioblastoma (GBM); medulloblastoma), cervical cancer (e.g., cervical adenocarcinoma), colorectal cancer (e.g., colon cancer, rectal cancer, colorectal adenocarcinoma), gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), pancreatic cancer (e.g., pancreatic andenocarcinoma, intraductal papillary mucinous neoplasm (IPMN)), prostate cancer (e.g., prostate adenocarcinoma), skin cancer (e.g., squamous cell carcinoma (SCC), keratoacanthoma (A), melanoma, basal cell carcinoma (BCC)) and soft tissue sarcoma (e.g., malignant fibrous histiocytoma (MFH), liposarcoma, malignant peripheral nerve sheath tumor (MPNST), chondrosarcoma, fibrosarcoma, myxosarcoma, osteosarcoma). In some embodiments, the solid tumor is a liver cancer. In one embodiment, the solid tumor is HCC. In other embodiments, the solid tumor is a brain cancer. In one embodiment, the solid tumor is GBM.

### 4.4 TREATMENT COMPOUNDS

In some embodiments, the treatment compound is an immunomodulatory compound. In one embodiment, the treatment compounds encompass those immunomodulatory compounds known as IMiDS® from Celgene Corporation.

As used herein and unless otherwise indicated, the term “immunomodulatory compound” encompasses certain small organic molecules that inhibit LPS induced monocyte TNF-α, IL-1β, IL-12, IL-6, MIP-1α, MCP-1, GM-CSF, G-CSF, and COX-2 production. Specific immunomodulatory compounds are provided herein.

TNF-α is an inflammatory cytokine produced by macrophages and monocytes during acute inflammation. TNF-α is responsible for a diverse range of signaling events within cells. Without being limited by a particular theory, one of the biological effects exerted by the immunomodulatory compounds provided herein is the reduction of myeloid cell TNF-α production. In certain embodiments, the immunomodulatory compounds provided herein enhance the degradation of TNF-α mRNA.

Examples of the immunomodulatory compounds provided herein include, but are not limited to, cyano and carboxy derivatives of substituted styrenes, such as those disclosed in U.S. Pat. No. 5,929,117; 1-oxo-2-(2,6-dioxo-3-fluoropiperidin-3-yl)-isoindolines and 1,3-dioxo-2-(2,6-dioxo-3-fluoropiperidine-3-yl)-isoindolines, such as those described in U.S. Pat. Nos. 5,874,448 and 5,955,476; tetra substituted 2-(2,6-dioxopiperdin-3-yl)-1-oxoisoindolines, such as those described in U.S. Pat. No. 5,798,368; 1-oxo- and 1,3-dioxo-2-(2,6-dioxopiperidin-3-yl)-isoindolines (e.g., 4-methyl derivatives of thalidomide), substituted 2-(2,6-dioxopiperidin-3-yl) phthalimides, and substituted 2-(2,6-dioxopiperidin-3-yl)-1-oxoisoindoles, including, but not limited to, those disclosed in U.S. Pat. Nos. 5,635,517, 6,281,230, 6,316,471, 6,403,613, 6,476,052, and 6,555,554; 1-oxo- and 1,3-dioxoisoindolines substituted in the 4- or 5-position of the indoline ring (e.g., 4-(4-amino-1,3-dioxoisoindoline-2-yl)-4-carbamoylbutanoic acid) described in U.S. Pat. No. 6,380,239; isoindoline-1-one and isoindoline-1,3-dione substituted in the 2-position with 2,6-dioxo-3-hydroxypiperidin-5-yl (e.g., 2-(2,6-dioxo-3-hydroxy-5-fluoropiperidin-5-yl)-4-aminoisoindolin-1-one) described in U.S. Pat. No. 6,458,810; a class of non-polypeptide cyclic amides disclosed in U.S. Pat. Nos. 5,698,579 and 5,877,200; and isoindole-imide compounds, such as those described in U.S. Pat. App. Pub. Nos. 2003/0045552 and 2003/0096841, and International Pub. No. WO 02/059106. The disclosure of each of the patents and patent application publications identified herein is incorporated herein by reference in its entirety.

Various immunomodulatory compounds provided herein contain one or more chiral centers, and can exist as mixtures of enantiomers (e.g., racemic mixtures) or mixtures of diastereomers. The methods provided herein encompass the use of stereomerically pure forms of such compounds as well as mixtures of those forms. For example, mixtures comprising equal or unequal amounts of the enantiomers of a particular immunomodulatory compound may be used in methods provided herein. These isomers may be asymmetrically synthesized or resolved using standard techniques, such as chiral columns or chiral resolving agents. See, Jacques et al., Enantiomers, Racemates and Resolutions (Wiley-Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (Eliel, Ed., Univ. of Notre Dame Press, Notre Dame, Ind., 1972).

In certain embodiments, the immunomodulatory compound is an 1-oxo- or 1,3-dioxo-2-(2,6-dioxopiperidin-3-yl)-isoindoline substituted with amino in the benzo ring, including those described in U.S. Pat. No. 5,635,517, the disclosure of which is incorporated herein by reference in its entirety.

In certain embodiments, the immunomodulatory compound has the structure of Formula I:

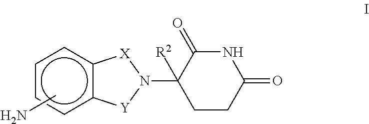

wherein one of X and Y is C═O, the other of X and Y is C═O or CH₂, and R² is hydrogen or lower alkyl, in one embodiment, methyl.

In certain embodiments, the immunomodulatory compound is:

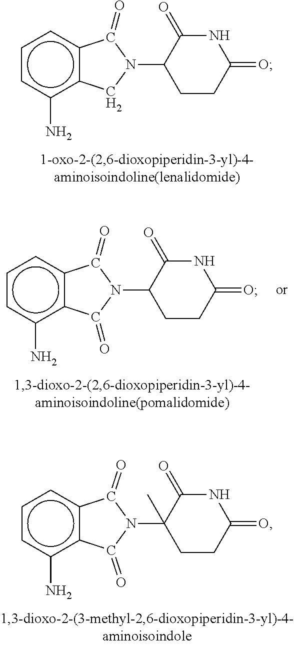

or a optically pure isomer thereof. The immunomodulatory compounds can be obtained via standard, synthetic methods. See U.S. Pat. No. 5,635,517, the disclosure of which is incorporated herein by reference in its entirety. The immunomodulatory compounds are also available from Celgene Corporation, Warren, N.J.

In certain embodiments, the immunomodulatory compound is lenalidomide. In certain embodiments, the immunomodulatory compound is pomalidomide.

In certain embodiments, the immunomodulatory compound is a substituted 2-(2,6-dioxopiperidin-3-yl)-phthalimide or substituted 2-(2,6-dioxopiperidin-3-yl)-1-oxoisoindole, including those described in U.S. Pat. Nos. 6,281,230; 6,316,471; 6,335,349; and 6,476,052, and International Pub. No. WO 98/03502, the disclosure of each of which is incorporated herein by reference in its entirety.

In certain embodiments, the immunomodulatory compound is of formula:

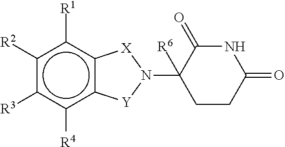

wherein:
one of X and Y is C═O and the other of X and Y is C═O or CH₂;
(i) each of R¹, R², R³, and R⁴, independently of the others, is halo, C₁₋₄ alkyl, or C₁₋₄ alkoxy; or (ii) one of R¹, R², R³, and R⁴ is —NHR⁵ and the remaining of R¹, R², R³, and R⁴ are hydrogen;
R⁵ is hydrogen or C₁₋₈ alkyl;
R⁶ is hydrogen, C₁₋₈ alkyl, benzyl, or halo;
provided that R⁶ is other than hydrogen if X and Y are C═O and (i) each of R¹, R², R³, and R⁴ is fluoro or (ii) one of R¹, R², R³, or R⁴ is amino.

In certain embodiments, the immunomodulatory compound is of formula

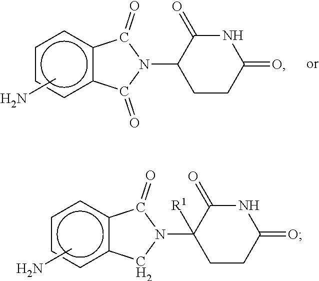

wherein R¹ is hydrogen or methyl.

In certain embodiments, the immunomodulatory compound used in the methods provided herein is enantiomerically pure (e.g. optically pure (R)- or (S)-enantiomers).

In another embodiment, the treatment compound is thalidomide, i.e., 2-(2,6-dioxopiperidin-3-yl)-1H-isoindole-1,3(2H)-dione.

In other embodiments, the treatment compound is a 5-substituted quinazolinone, including those described in U.S. Pat. No. 7,635,700, the disclosure of which is incorporated herein by reference in its entirety.

In certain embodiments, the treatment compound is a compound having the structure of Formula IV:

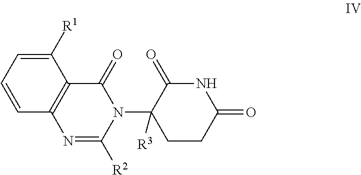

or a pharmaceutically acceptable salt, solvate, or stereoisomer thereof, wherein:
R¹ is:
   hydrogen;
   halo;
   —(CH₂)ₙOH;
   C₁₋₆ alkyl, optionally substituted with one or more halo;
   C₁₋₆ alkoxy, optionally substituted with one or more halo; or
   —(CH₂)ₙNHR^{a}, wherein R^{a} is:
      hydrogen;
      C₁₋₆ alkyl, optionally substituted with one or more halo;
      (CH₂)ₙ-(6 to 10 membered aryl);
      —C(O)—(CH₂)ₙ-(6 to 10 membered aryl) or —C(O)—(CH₂)ₙ-(5 to 10 membered heteroaryl), wherein the aryl or heteroaryl is optionally substituted with one or more of: halo; —SCF₃; C₁₋₆ alkyl, itself optionally substituted with one or more halo; or C₁₋₆ alkoxy, itself optionally substituted with one or more halo;
      —C(O)—C₁₋₈ alkyl, wherein the alkyl is optionally substituted with one or more halo;
      —C(O)—(CH₂)ₙ—(C₃-C₁₀-cycloalkyl);
      —C(O)—(CH₂)ₙ—NR^{b}R^{c}, wherein R^{b} and R^{c} are each independently:
         hydrogen;
         C₁₋₆ alkyl, optionally substituted with one or more halo;
         C₁₋₆ alkoxy, optionally substituted with one or more halo; or
         6 to 10 membered aryl, optionally substituted with one or more of: halo; C₁₋₆ alkyl, itself optionally substituted with one or more halo; or C₁₋₆ alkoxy, itself optionally substituted with one or more halo;
      —C(O)—(CH₂)ₙ—O—C₁₋₆ alkyl; or
      —C(O)—(CH₂)ₙ—O—(CH₂)ₙ-(6 to 10 membered aryl);
R² is: hydrogen; —(CH₂)ₙOH; phenyl; —O—C₁₋₆ alkyl; or C₁₋₆ alkyl, optionally substituted with one or more halo;
R³ is: hydrogen; or C₁₋₆ alkyl, optionally substituted with one or more halo; and
n is 0, 1, or 2.

In certain embodiments, the treatment compound is a compound having the structure of Formula V:

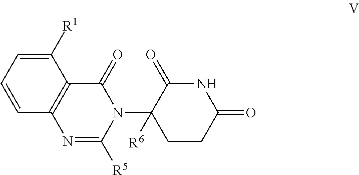

or a pharmaceutically acceptable salt, solvate, or stereoisomer thereof, wherein:

R⁴ is: hydrogen; halo; —(CH₂)—OH; C₁₋₆ alkyl, optionally substituted with one or more halo; or C₁₋₆ alkoxy, optionally substituted with one or more halo

R⁵ is: hydrogen; —(CH₂)—OH; phenyl; —O—C₁₋₆ alkyl; or C₁₋₆ alkyl, optionally substituted with one or more halo;

R⁶ is: hydrogen; or C₁₋₆ alkyl, optionally substituted with one or more halo; and

n is 0, 1, or 2.

In certain embodiments, the treatment compound is:

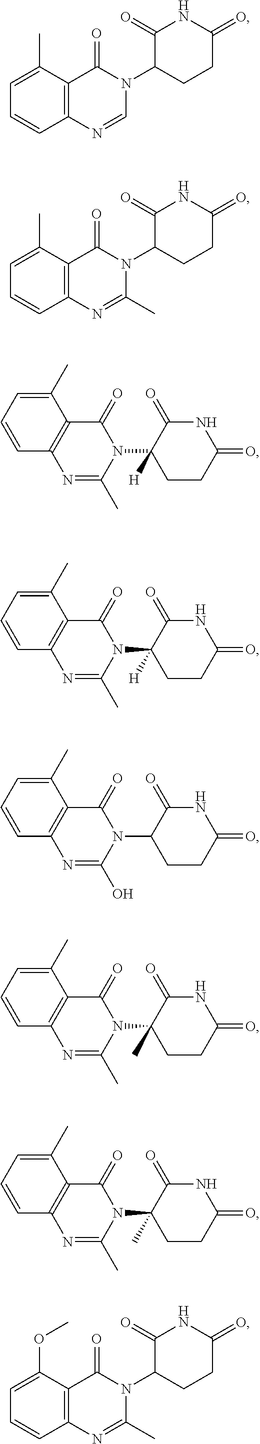

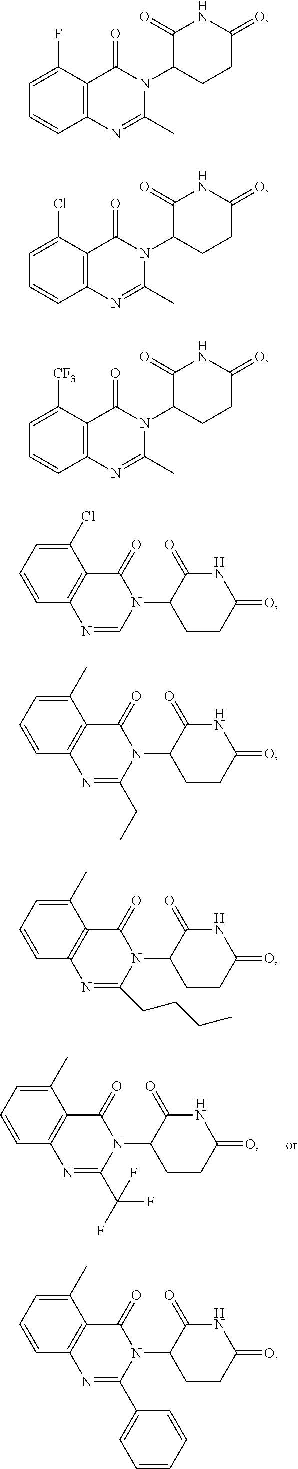

In certain embodiments, the treatment compound is a compound of Formula VI:

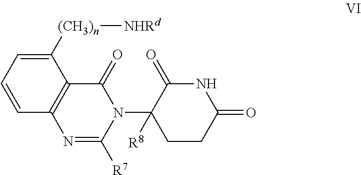

or a pharmaceutically acceptable salt, solvate, or stereoisomer thereof, wherein:
R^{d} is: hydrogen;
   C₁₋₆ alkyl, optionally substituted with one or more halo;
   —C(O)—C₁₋₈ alkyl, wherein the alkyl is optionally substituted with one or more halo;
   —C(O)—(CH₂)ₙ—C₃₋₁₀ cycloalkyl;
   —C(O)—(CH₂)ₙ—NR^{e}R^{f}, wherein R^{e} and R^{f} are each independently:
      hydrogen;
      C₁₋₆ alkyl, optionally substituted with one or more halo; or
      C₁₋₆ alkoxy, optionally substituted with one or more halo; or
—C(O)—(CH₂)ₙ—O—C₁₋₆ alkyl.
R⁷ is: hydrogen; —(CH₂)ₙ—OH; phenyl; —O—C₁₋₆ alkyl; or C₁₋₆ alkyl, optionally substituted with one or more halo;
R⁸ is: hydrogen; or C₁₋₆ alkyl, optionally substituted with one or more halo; and
n is 0, 1, or 2.

In certain embodiments, the treatment compound is:

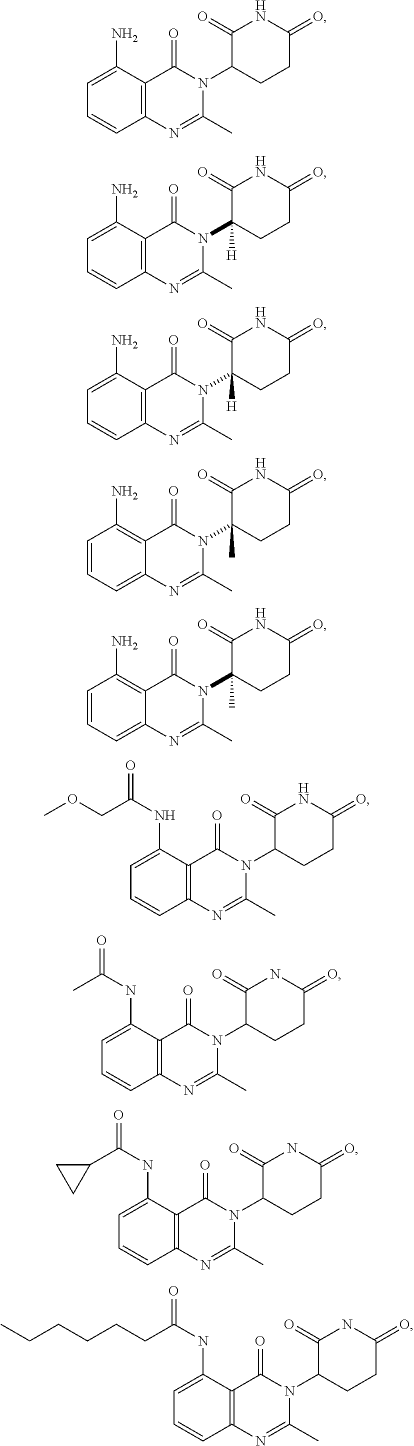

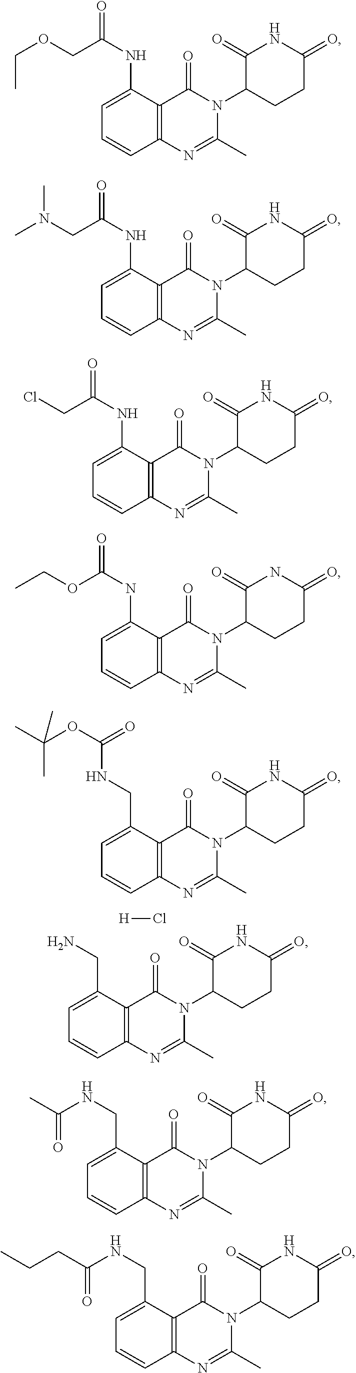

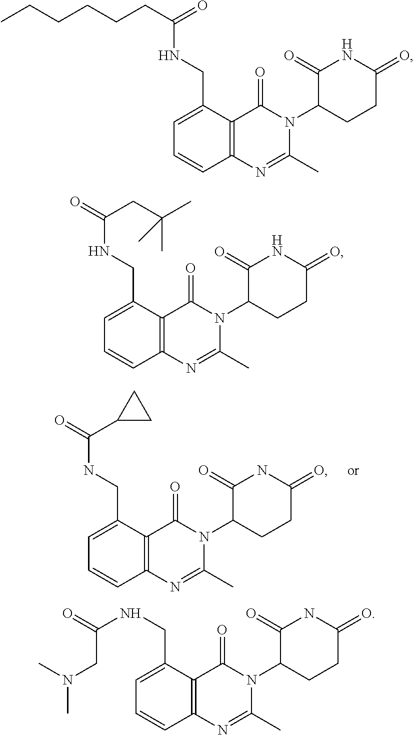

In certain embodiments, the treatment compound is 3-(5-amino-2-methyl-4-oxo-4H-quinazolin-3-yl)-piperidine-2,6-dione, or a stereoisomer thereof, or a pharmaceutically acceptable salt, solvate, hydrate, co-crystal, clathrate, or a polymorph thereof.

In certain embodiments, the treatment compound is a compound of Formula VII:

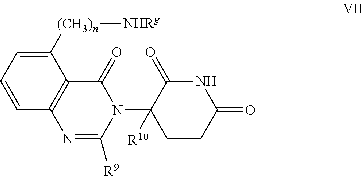

or a pharmaceutically acceptable salt, solvate, or stereoisomers thereof, wherein:
R^{g} is: —(CH₂)ₙ-(6 to 10 membered aryl);
   —C(O)—(CH₂)ₙ-(6 to 10 membered aryl) or —C(O)—(CH₂)ₙ-(5 to 10 membered heteroaryl), wherein the aryl or heteroaryl is optionally substituted with one or more of: halo; —SCF₃; (C₁-C₆)alkyl, itself optionally substituted with one or more halo; or C₁₋₆ alkoxy, itself optionally substituted with one or more halo;
   —C(O)—(CH₂)ₙ—NHR^{h}, wherein R^{h} is: 6 to 10 membered aryl, optionally substituted with one or more of: halo; C₁₋₆ alkyl, itself optionally substituted with one or more halo; or C₁₋₆ alkoxy, itself optionally substituted with one or more halo; or
   —C(O)—(CH₂)ₙ—O—(CH₂)ₙ-(6 to 10 membered aryl);
R⁹ is: hydrogen; —(CH₂)ₙOH; phenyl; —O—C₁₋₆ alkyl; or C₁₋₆ alkyl, optionally substituted with one or more halo;
R¹⁰ is: hydrogen; or C₁₋₆ alkyl, optionally substituted with one or more halo; and
n is 0, 1, or 2.

In certain embodiments, the treatment compound is:

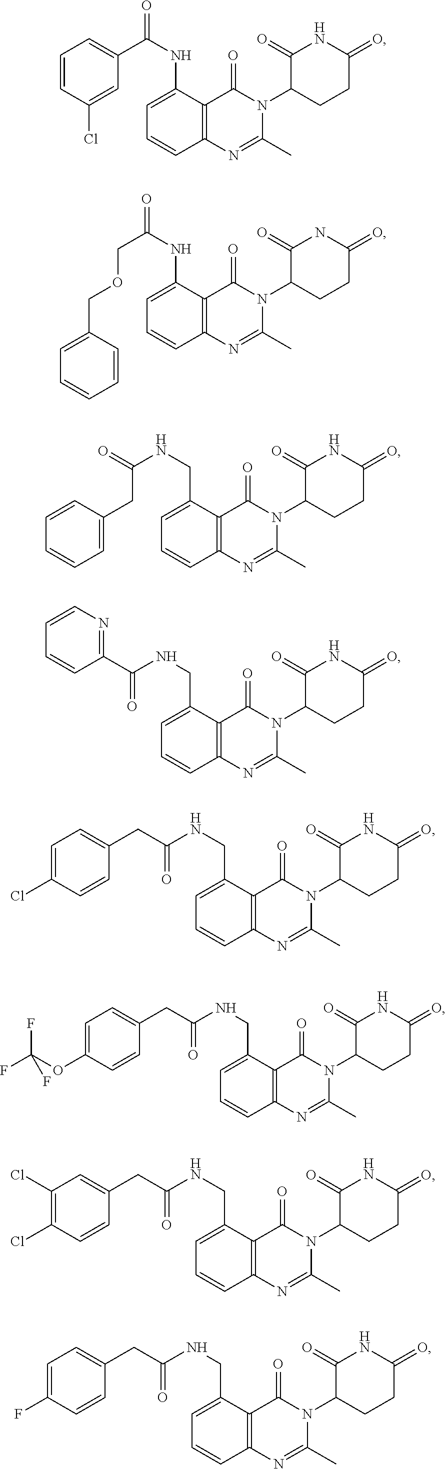

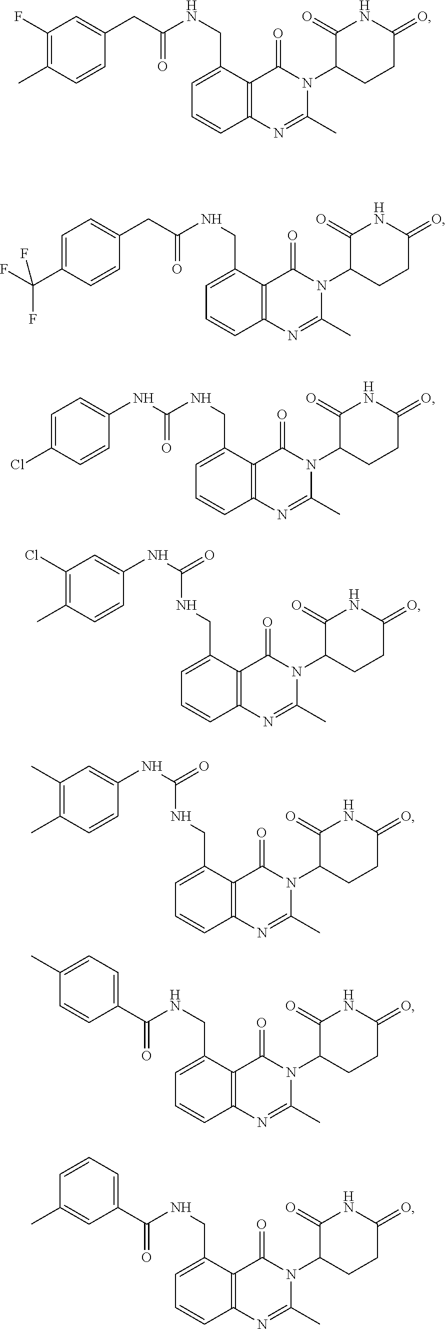

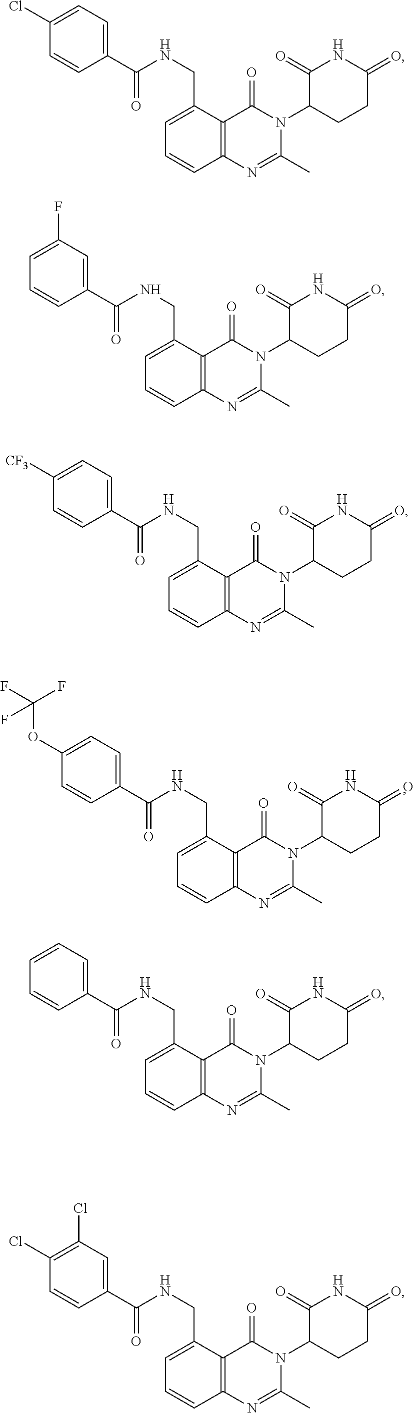 [SMILES: Cc1nc2cccc(CNC(=O)c3ccc(Cl)cc3)c2c(=O)n1C1CCC(=O)NC1=O ; Cc1nc2cccc(CNC(=O)c3cccc(F)c3)c2c(=O)n1C1CCC(=O)NC1=O ; Cc1nc2cccc(CNC(=O)c3ccc(C(F)(F)F)cc3)c2c(=O)n1C1CCC(=O)NC1=O ; Cc1nc2cccc(CNC(=O)c3ccc(OC(F)(F)F)cc3)c2c(=O)n1C1CCC(=O)NC1=O ; Cc1nc2cccc(CNC(=O)c3ccccc3)c2c(=O)n1C1CCC(=O)NC1=O ; Cc1nc2cccc(CNC(=O)c3ccc(Cl)c(Cl)c3)c2c(=O)n1C1CCC(=O)NC1=O]

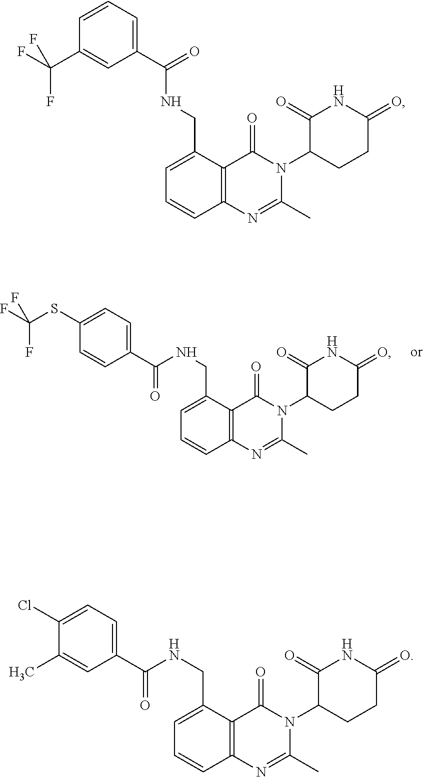 [SMILES: Cc1nc2cccc(CNC(=O)c3cccc(C(F)(F)F)c3)c2c(=O)n1C1CCC(=O)NC1=O ; Cc1nc2cccc(CNC(=O)c3ccc(SC(F)(F)F)cc3)c2c(=O)n1C1CCC(=O)NC1=O ; Cc1cc(C(=O)NCc2cccc3nc(C)n(C4CCC(=O)NC4=O)c(=O)c23)ccc1Cl]

In certain embodiments, the treatment compound is 3-[4-(4-morpholin-4-ylmethyl-benzyloxy)-1-oxo-1,3-dihydro-isoindol-2-yl]-piperidine-2,6-dione (Compound B), which has the following structure:

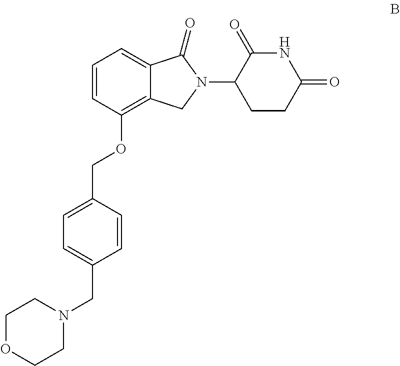 [SMILES: O=C1CCC(N2Cc3c(OCc4ccc(CN5CCOCC5)cc4)cccc3C2=O)C(=O)N1]

or an enantiomer or a mixture of enantiomers thereof; or a pharmaceutically acceptable salt, solvate, hydrate, co-crystal, clathrate, or polymorph thereof.

All of the compounds described herein can either be commercially purchased or prepared according to the methods described in the patents or patent publications disclosed herein. Further, optically pure compounds can be asymmetrically synthesized or resolved using known resolving agents or chiral columns as well as other standard synthetic organic chemistry techniques.

Compounds provided herein may be small organic molecules having a molecular weight less than about 1,000 g/mol, and are not proteins, peptides, oligonucleotides, oligosaccharides or other macromolecules.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 4.5 METHODS OF DETECTING BIOMARKER LEVELS

4.5.1 Methods of Detecting mRNA Levels in a Sample

Several methods of detecting or quantitating mRNA levels are known in the art and are suitable for use in the methods provided herein for measuring the level of the biomarker. Exemplary methods include, but are not limited to, northern blots, ribonuclease protection assays, and PCR-based methods. When the biomarker is an mRNA molecule, the mRNA sequence, or a fragment thereof, can be used to prepare a probe that is at least partially complementary. The probe can then be used to detect the mRNA sequence in a sample, using any suitable assay, such as PCR-based methods, Northern blotting, or a dipstick assay.

The assay method can be varied depending on the type of mRNA information desired. Exemplary methods include, but are not limited to, Northern blots and PCR-based methods (e.g., qRT-PCR). Methods such as qRT-PCR can also accurately quantitate the amount of the mRNA in a sample.

Any suitable assay platform can be used to determine the presence of the mRNA in a sample. For example, an assay may be in the form of a dipstick, a membrane, a chip, a disk, a test strip, a filter, a microsphere, a slide, a multiwell plate, or an optical fiber. An assay system may have a solid support on which a nucleic acid corresponding to the mRNA is attached. The solid support may comprise, for example, a plastic, silicon, a metal, a resin, glass, a membrane, a particle, a precipitate, a gel, a polymer, a sheet, a sphere, a polysaccharide, a capillary, a film a plate, or a slide. The assay components can be prepared and packaged together as a kit for detecting an mRNA.

The nucleic acid can be labeled, if desired, to make a population of labeled mRNAs. In general, a sample can be labeled using methods that are well known in the art (e.g., using DNA ligase, terminal transferase, or by labeling the RNA backbone, etc.; see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). In certain embodiments, the sample is labeled with fluorescent label. Exemplary fluorescent dyes include, but are not limited to, xanthene dyes, fluorescein dyes, rhodamine dyes, fluorescein isothiocyanate (FITC), 6-carboxyfluorescein (FAM), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine 6G (R6G5 or G5), 6-carboxyrhodamine 6G (R6G6 or G6), and rhodamine 110; cyanine dyes, e.g. Cy3, Cy5 and Cy7 dyes; Alexa dyes, e.g. Alexa-fluor-555; coumarin, diethylaminocoumarin, umbelliferone; benzimide dyes, e.g. Hoechst 33258; phenanthridine dyes, e.g., Texas red; ethidium dyes; acridine dyes; carbazole dyes; phenoxazine dyes; porphyrin dyes; polymethine dyes, BODIPY dyes, quinoline dyes, pyrene, fluorescein chlorotriazinyl, R110, Eosin, JOE, R6G, tetramethylrhodamine, lissamine, ROX, and napthofluorescein.

The nucleic acids may be present in specific, addressable locations on a solid support; each corresponding to at least a portion of mRNA sequences of a biomarker.

In certain embodiments, an mRNA assay comprises the steps of 1) obtaining surface-bound probes for one or more biomarkers; 2) hybridizing a population of mRNAs to the surface-bound probes under conditions sufficient to provide for specific binding; (3) removing unbound nucleic acids in the hybridization step; and (4) detecting the hybridized mRNAs.

Hybridization can be carried out under suitable hybridization conditions, which may vary in stringency as desired. Typical conditions are sufficient to produce probe/target complexes on a solid surface between complementary binding members, i.e., between surface-bound probes and complementary mRNAs in a sample.

In certain embodiments, stringent hybridization conditions are used. Standard hybridization techniques (e.g., under conditions sufficient to provide for specific binding of target mRNAs in the sample to the probes) are described in Kallioniemi et al., Science 258:818-821 (1992) and WO 93/18186, the disclosure of each which is incorporated herein by reference in its entirety. Several guides to general techniques are available, e.g., Tijssen, Hybridization with Nucleic Acid Probes, Parts I and II (Elsevier, Amsterdam 1993). For descriptions of techniques suitable for in situ hybridizations, see Gall et al. Meth. Enzymol., 21:470-480 (1981); and Angerer et al. in Genetic Engineering: Principles and Methods (Setlow and Hollaender, Eds.) Vol 7, pages 43-65 (Plenum Press, New York 1985). Selection of appropriate conditions, including temperature, salt concentration, polynucleotide concentration, hybridization time, and stringency of washing conditions, depends on experimental design, including the source of a sample, the identity of capture agents, the degree of complementarity expected, etc., and may be determined as a matter of routine experimentation for those of ordinary skill in the art.

After the mRNA hybridization procedure, the surface bound polynucleotides are washed to remove unbound nucleic acids. Washing may be performed using any convenient washing protocol. In certain embodiments, the washing conditions are stringent. The hybridization of the target mRNAs to the probes is then detected using standard techniques.

4.5.2 PCR-Based Methods of Detecting mRNA Levels in a Sample

In certain embodiments, the mRNA level of a biomarker is determined using a PCR-based method. Examples of PCR assays can be found in U.S. Pat. No. 6,927,024, the disclosure of which is incorporated by reference herein in its entirety. Examples of RT-PCR methods can be found in U.S. Pat. No. 7,122,799, the disclosure of which is incorporated by reference herein in its entirety. Examples of fluorescent in situ PCR methods can be found in U.S. Pat. No. 7,186,507, the disclosure of which is incorporated by reference herein in its entirety.

In certain embodiments, real-time reverse transcription-PCR (qRT-PCR) is used for both the detection and quantification of mRNAs (Bustin, et al., Clin. Sci., 2005, 109, 365-379). Quantitative results obtained by qRT-PCR are generally more informative than qualitative data. Examples of qRT-PCR-based methods can be found in U.S. Pat. No. 7,101,663, the disclosure of which is incorporated by reference herein in its entirety.

In contrast to regular reverse transcriptase-PCR and analysis by agarose gels, real-time PCR gives quantitative results. An additional advantage of real-time PCR is the relative ease and convenience of use. Instruments for real-time PCR, such as Applied Biosystems 7500, are available commercially. The reagents for real-time PCR, such as TaqMan Sequence Detection chemistry, are also commercially available.

To determine the cycle number at which the fluorescence signal associated with a particular amplicon accumulation crosses the threshold (referred to as CT), the data can be analyzed, for example, using a 7500 Real-Time PCR System Sequence Detection software v1.3, using the comparative CT relative quantification calculation method. Using this method, the output is expressed as a fold-change in expression levels. In some embodiments, the threshold level can be selected to be automatically determined by the software. In some embodiments, the threshold level is set to be above the baseline, but sufficiently low to be within the exponential growth region of an amplification curve.

Techniques known to one skilled in the art may be used to measure the amount of an RNA transcript(s). In some embodiments, the amount of one, two, three, four, five or more RNA transcripts is measured using deep sequencing, such as ILLUMINA® RNASeq, ILLUMINA® next generation sequencing (NGS), ION TORRENT™ RNA next generation sequencing, 454™ pyrosequencing, or Sequencing by Oligo Ligation Detection (SOLID™). In other embodiments, the amount of multiple RNA transcripts is measured using a microarray and/or gene chip. In certain embodiments, the amount of one, two, three or more RNA transcripts is determined by RT-PCR. In other embodiments, the amount of one, two, three or more RNA transcripts is measured by RT-qPCR. Techniques for conducting these assays are known to one skilled in the art.

In some embodiments, a statistical analysis or other analysis is performed on data from the assay utilized to measure an RNA transcript or protein. In certain specific embodiments, p value of those RNA transcripts or proteins differentially expressed is 0.1, 0.5, 0.4, 0.3, 0.2, 0.01, 0.05, 0.001, 0.005, or 0.0001. In specific embodiments, a false discovery rate (FDR) of 10%, 9%, 8%, 7%, 6%, 5%, 4%, 3%, 2%, 1% or less is selected.

4.5.3 Methods of Detecting Polypeptide or Protein Biomarkers

When the biomarker is a protein, polypeptide, or peptide, several protein detection and quantitation methods can be used to measure the level of the biomarker. Any suitable protein quantitation method can be used in the methods provided herein. In certain embodiments, antibody-based methods are used. Exemplary methods that can be used include, but are not limited to, immunoblotting (western blot), enzyme-linked immunosorbent assay (ELISA), immunohistochemistry, flow cytometry, cytometric bead array, and mass spectroscopy. In certain embodiments, a biomarker protein is detected using mass spectroscopy. Several types of ELISA are commonly used, including direct ELISA, indirect ELISA, and sandwich ELISA.

### 4.6 KITS FOR DETECTING BIOMARKER LEVELS

In certain embodiments, provided herein is a kit for detecting the mRNA level of one or more biomarkers. In certain embodiments, the kit comprises one or more probes that bind specifically to the mRNAs of the one or more biomarkers. In certain embodiments, the kit further comprises a washing solution. In certain embodiments, the kit further comprises reagents for performing a hybridization assay, mRNA isolation or purification means, detection means, as well as positive and negative controls. In certain embodiments, the kit further comprises an instruction for using the kit. The kit can be tailored for in-home use, clinical use, or research use.

In certain embodiments, provided herein is a kit for detecting the protein level of one or more biomarkers. In certain embodiments, the kits comprises a dipstick coated with an antibody that recognizes the protein biomarker, washing solutions, reagents for performing the assay, protein isolation or purification means, detection means, as well as positive and negative controls. In certain embodiments, the kit further comprises an instruction for using the kit. The kit can be tailored for in-home use, clinical use, or research use.

Such a kit can employ, for example, a dipstick, a membrane, a chip, a disk, a test strip, a filter, a microsphere, a slide, a multiwell plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide. The biological sample can be, for example, a cell culture, a cell line, a tissue, an oral tissue, gastrointestinal tissue, an organ, an organelle, a biological fluid, a blood sample, a urine sample, or a skin sample.

In certain embodiments, provided herein is a kit for detecting the mRNA level of one or more biomarkers. In certain embodiments, the kit comprises one or more probes that bind specifically to the mRNAs of the one or more biomarkers. In certain embodiments, the kit further comprises a washing solution. In certain embodiments, the kit further comprises reagents for performing a hybridization assay, mRNA isolation or purification means, detection means, as well as positive and negative controls. In certain embodiments, the kit further comprises an instruction for using the kit. The kit can be tailored for in-home use, clinical use, or research use.

In certain embodiments, provided herein is a kit for detecting the protein level of one or more biomarkers. In certain embodiments, the kits comprises a dipstick coated with an antibody that recognizes the protein biomarker, washing solutions, reagents for performing the assay, protein isolation or purification means, detection means, as well as positive and negative controls. In certain embodiments, the kit further comprises an instruction for using the kit. The kit can be tailored for in-home use, clinical use, or research use.

Such a kit may employ, for example, a dipstick, a membrane, a chip, a disk, a test strip, a filter, a microsphere, a slide, a multiwell plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide. The biological sample can be, for example, a cell culture, a cell line, a tissue, an oral tissue, gastrointestinal tissue, an organ, an organelle, a biological fluid, a blood sample, a urine sample, or a skin sample. The biological sample can be, for example, a lymph node biopsy, a bone marrow biopsy, or a sample of peripheral blood tumor cells.

In another embodiment, the kit comprises a solid support, nucleic acids contacting the support, where the nucleic acids are complementary to at least 20, 50, 100, 200, 350, or more bases of mRNA, and a means for detecting the expression of the mRNA in a biological sample.

In a specific embodiment, the pharmaceutical or assay kit comprises, in a container, a compound or a pharmaceutical composition thereof, and further comprises, in one or more containers, components for isolating RNA. In another specific embodiment, the pharmaceutical or assay kit comprises, in a container, a compound or a pharmaceutical composition, and further comprises, in one or more containers, components for conducting RT-PCR, RT-qPCR, deep sequencing or a microarray. In some embodiments, the kit comprises a solid support, nucleic acids contacting the support, where the nucleic acids are complementary to at least 20, 50, 100, 200, 350, or more bases of mRNA, and a means for detecting the expression of the mRNA in a biological sample.

In certain embodiments, the kits provided herein employ means for detecting the expression of a biomarker by quantitative real-time PCR (QRT-PCR), microarray, flow cytometry or immunofluorescence. In other embodiments, the expression of the biomarker is measured by ELISA-based methodologies or other similar methods known in the art.

In another specific embodiment, the pharmaceutical or assay kit comprises, in a container, a compound or a pharmaceutical composition thereof, and further comprises, in one or more containers, components for isolating protein. In another specific embodiment, the pharmaceutical or assay kit comprises, in a container, a compound or a pharmaceutical composition, and further comprises, in one or more containers, components for conducting flow cytometry or an ELISA.

In another aspect, provided herein are kits for measuring biomarkers providing the materials necessary to measure the abundance of one or more of the gene products of the genes or a subset of genes (e.g., one, two, three, four, five or more genes) of the biomarkers provided herein. Such kits may comprise materials and reagents required for measuring RNA or protein. In some embodiments, such kits include microarrays, wherein the microarray is comprised of oligonucleotides and/or DNA and/or RNA fragments which hybridize to one or more of the products of one or more of the genes or a subset of genes of the biomarkers provided herein, or any combination thereof. In some embodiments, such kits may include primers for PCR of either the RNA product or the cDNA copy of the RNA product of the genes or subset of genes, or both. In some embodiments, such kits may include primers for PCR as well as probes for Quantitative PCR. In some embodiments, such kits may include multiple primers and multiple probes wherein some of said probes have different fluorophores so as to permit multiplexing of multiple products of a gene product or multiple gene products. In some embodiments, such kits may further include materials and reagents for creating cDNA from RNA. In some embodiments, such kits may include antibodies specific for the protein products of a gene or subset of genes of the biomarkers provided herein. Such kits may additionally comprise materials and reagents for isolating RNA and/or proteins from a biological sample. In addition such kits may include materials and reagents for synthesizing cDNA from RNA isolated from a biological sample. In some embodiments, such kits may include, a computer program product embedded on computer readable media for predicting whether a patient is clinically sensitive to a compound. In some embodiments, the kits may include a computer program product embedded on a computer readable media along with instructions.

In some embodiments, kits for measuring the expression of one or more nucleic acid sequences of a gene or a subset of genes of the biomarkers provided herein. In a specific embodiment, such kits measure the expression of one or more nucleic acid sequences associated with a gene or a subset of genes of the biomarkers provided herein. In accordance with this embodiment, the kits may comprise materials and reagents that are necessary for measuring the expression of particular nucleic acid sequence products of genes or a subset of genes of the biomarkers provided herein. For example, a microarray or RT-PCR kit may be produced for a specific condition and contain only those reagents and materials necessary for measuring the levels of specific RNA transcript products of the genes or a subset of genes of the biomarkers provided herein to predict whether a hematological cancer in a patient is clinically sensitive to a compound. Alternatively, in some embodiments, the kits can comprise materials and reagents that are not limited to those required to measure the expression of particular nucleic acid sequences of any particular gene of the biomarkers provided herein. For example, in certain embodiments, the kits comprise materials and reagents necessary for measuring the levels of expression of 1, 2, 3, 4, 5, 6, 7, 8, 9, 10, 15, 20, 25, 30, 35, 40, 45, 50 or more of the genes of the biomarkers provided herein, in addition to reagents and materials necessary for measuring the levels of the expression of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50 or more genes other than those of the biomarkers provided herein. In other embodiments, the kits contain reagents and materials necessary for measuring the levels of expression of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50 or more of the genes of the biomarkers provided herein, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are genes not of the biomarkers provided herein, or 1-10, 1-100, 1-150, 1-200, 1-300, 1-400, 1-500, 1-1000, 25-100, 25-200, 25-300, 25-400, 25-500, 25-1000, 100-150, 100-200, 100-300, 100-400, 100-500, 100-1000 or 500-1000 genes that are genes not of the biomarkers provided herein.

For nucleic acid microarray kits, the kits generally comprise probes attached to a solid support surface. In one such embodiment, probes can be either oligonucleotides or longer length probes including probes ranging from 150 nucleotides in length to 800 nucleotides in length. The probes may be labeled with a detectable label. In a specific embodiment, the probes are specific for one or more of the gene products of the biomarkers provided herein. The microarray kits may comprise instructions for performing the assay and methods for interpreting and analyzing the data resulting from the performance of the assay. In a specific embodiment, the kits comprise instructions for predicting whether a hematological cancer in a patient is clinically sensitive to a compound. The kits may also comprise hybridization reagents and/or reagents necessary for detecting a signal produced when a probe hybridizes to a target nucleic acid sequence. Generally, the materials and reagents for the microarray kits are in one or more containers. Each component of the kit is generally in its own a suitable container.

In certain embodiments, a nucleic acid microarray kit comprises materials and reagents necessary for measuring the levels of expression of 1, 2, 3, 4, 5, 6, 7, 8, 9, 10, 15, 20, 25, 30, 35, 40, 45, 50 or more of the genes identified of the biomarkers provided herein, or a combination thereof, in addition to reagents and materials necessary for measuring the levels of the expression of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50 or more genes other than those of the biomarkers provided herein. In other embodiments, a nucleic acid microarray kit contains reagents and materials necessary for measuring the levels of expression of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50 or more of the genes of the biomarkers provided herein, or any combination thereof, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are not of the biomarkers provided herein, or 1-10, 1-100, 1-150, 1-200, 1-300, 1-400, 1-500, 1-1000, 25-100, 25-200, 25-300, 25-400, 25-500, 25-1000, 100-150, 100-200, 100-300, 100-400, 100-500, 100-1000 or 500-1000 genes that are not of the biomarkers provided herein.

For Quantitative PCR, the kits generally comprise pre-selected primers specific for particular nucleic acid sequences. The Quantitative PCR kits may also comprise enzymes suitable for amplifying nucleic acids (e.g., polymerases such as Taq), and deoxynucleotides and buffers needed for the reaction mixture for amplification. The Quantitative PCR kits may also comprise probes specific for the nucleic acid sequences associated with or indicative of a condition. The probes may or may not be labeled with a fluorophore. The probes may or may not be labeled with a quencher molecule. In some embodiments the Quantitative PCR kits also comprise components suitable for reverse-transcribing RNA including enzymes (e.g., reverse transcriptases such as AMV, MMLV and the like) and primers for reverse transcription along with deoxynucleotides and buffers needed for the reverse transcription reaction. Each component of the quantitative PCR kit is generally in its own suitable container. Thus, these kits generally comprise distinct containers suitable for each individual reagent, enzyme, primer and probe. Further, the quantitative PCR kits may comprise instructions for performing the assay and methods for interpreting and analyzing the data resulting from the performance of the assay. In a specific embodiment, the kits contain instructions for predicting whether a hematological cancer in a patient is clinically sensitive to a compound.

For antibody based kits, the kit can comprise, for example: (1) a first antibody (which may or may not be attached to a solid support) which binds to a peptide, polypeptide or protein of interest; and, optionally, (2) a second, different antibody which binds to either the peptide, polypeptide or protein, or the first antibody and is conjugated to a detectable label (e.g., a fluorescent label, radioactive isotope or enzyme). In a specific embodiment, the peptide, polypeptide or protein of interest is associated with or indicative of a condition (e.g., a disease). The antibody-based kits may also comprise beads for conducting an immunoprecipitation. Each component of the antibody-based kits is generally in its own suitable container. Thus, these kits generally comprise distinct containers suitable for each antibody. Further, the antibody-based kits may comprise instructions for performing the assay and methods for interpreting and analyzing the data resulting from the performance of the assay. In a specific embodiment, the kits contain instructions for predicting whether a hematological cancer in a patient is clinically sensitive to a compound.

In one embodiment a kit provided herein comprises a compound provided herein, or a pharmaceutically acceptable salt, solvate or hydrate thereof. Kits may further comprise additional active agents, including but not limited to those disclosed herein.

Kits provided herein may further comprise devices that are used to administer the active ingredients. Examples of such devices include, but are not limited to, syringes, drip bags, patches, and inhalers.

Kits may further comprise cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

In certain embodiments of the methods and kits provided herein, solid phase supports are used for purifying proteins, labeling samples or carrying out the solid phase assays. Examples of solid phases suitable for carrying out the methods disclosed herein include beads, particles, colloids, single surfaces, tubes, multiwell plates, microtiter plates, slides, membranes, gels and electrodes. When the solid phase is a particulate material (e.g., beads), it is, in one embodiment, distributed in the wells of multi-well plates to allow for parallel processing of the solid phase supports.

It is noted that any combination of the above-listed embodiments, for example, with respect to one or more reagents, such as, without limitation, nucleic acid primers, solid support and the like, are also contemplated in relation to any of the various methods and/or kits provided and the like, are also contemplated in relation to any of the various methods and/or kits provided herein.

Certain embodiments of the invention are illustrated by the following non-limiting examples.

### 5. EXAMPLES

The examples below are carried out using standard techniques, which are well known and routine to those of skill in the art, except where otherwise described in detail. The examples are intended to be merely illustrative.

### 5.1 Preparation of 3-(4-amino-1-oxo-1,3-dihydro-isoindol-2-yl)-piperidine-2,6-dione (Lenalidomide)

### Methyl 2-bromomethyl-3-nitrobenzoate

A stirred mixture of methyl 2-methyl-3-nitrobenzoate (14.0 g, 71.7 mmol) and N-bromosuccinimide (15.3 g, 86.1 mmol) in carbon tetrachloride (200 mL) was heated under gentle reflux for 15 hours while a 100W bulb situated 2 cm away was shining on the flask. The mixture was filtered and the solid was washed with methylene chloride (50 mL). The filtrate was washed with water (2×100 mL), brine (100 mL) and dried. The solvent was removed in vacuo and the residue was purified by flash chromatography (hexane/ethyl acetate, 8/2) to afford 19 g (96%) of the product as a yellow solid: mp 70.0-71.5° C.; 1H NMR (CDCl₃) δ 8.12-8.09 (dd, J=1.3 and 7.8 Hz, 1H), 7.97-7.94 (dd, J=1.3 and 8.2 Hz, 1H), 7.54 (t, J=8.0 Hz, 1H). 5.15 (s, 2H), 4.00 (s, 3H); ¹³C NMR (CDCl₃) δ 165.85, 150.58, 134.68, 132.38, 129.08, 127.80, 53.06, 22.69; HPLC, Water Nove-Pak/C18, 3.9×150 mm, 4 micron, 1 mL/min, 240 nm, 40/60 CH₃CN/0.1% H₃PO₄(aq) 7.27 min(98.92%); Anal. Calcd for C₉H₈NO₄Br:C, 39.44; H, 2.94; N, 5.11; Br, 29.15. Found: C, 39.46; H, 3.00; N, 5.00; Br, 29.11.

### t-Butyl N-(1-oxo-4-nitroisoindolin-2-yl)-L-glutamine

Triethylamine (2.9 g, 28.6 mmol) was added dropwise to a stirred mixture of methyl 2-bromomethyl-3-nitrobenzoate (3.5 g, 13.0 mmol) and L-glutamine t-butyl ester hydrochloride (3.1 g, 13.0 mmol) in tetrahydrofuran (90 mL). The mixture was heated to reflux for 24 hours. To the cooled mixture was added methylene chloride (150 mL) and the mixture was washed with water (2×40 mL), brine (40 mL) and dried. The solvent was removed in vacuo and the residue was purified by flash chromatography (3% CH₃OH in methylene chloride) to afford 2.84 g (60%) of crude product which was used directly in the next reaction: 1H NMR (CDCl₃) δ 8.40 (d, J=8.1 Hz, 1H), 8.15 (d, J=7.5 Hz, 1H), 7.71 (t, J=7.8 Hz, 1H), 5.83 (s, 1H), 5.61 (s, 1H), 5.12 (d, J=19.4 Hz, 1H), 5.04-4.98 (m, 1H), 4.92 (d, J=19.4 Hz, 1H), 2.49-2.22 (m, 4H). 1.46 (s, 9H); HPLC, Waters Nova-Pak C18, 3.9×150 mm, 4 micron, 1 mL/min, 240 nm, 25/75 CH₃CN/0.1% H₃PO₄(aq) 6.75 min(99.94%).

### N-(1-oxo-4-nitroisoindolin-2-yl)-L-glutamine

Hydrogen chloride gas was bubbled into a stirred 5° C. solution of t-butyl N-(1-oxo-4-nitro-isoindolin-2-yl)-L-glutamine (3.6 g, 9.9 mmol) in methylene chloride (60 mL) for 1 hour. The mixture was then stirred at room temperature for another hour. Ether (40 mL) was added and the resulting mixture was stirred for 30 minutes. The slurry was filtered, washed with ether and dried to afford 3.3 g of the product: 1H NMR (DMSO-d₆) δ 8.45 (d, J=8.1 Hz, 1H), 8.15 (d, J=7.5 Hz, 1H), 7.83 (t, J=7.9 Hz. 1H), 7.24 (s, 1H), 6.76 (s, 1H), 4.93 (s, 2H), 4.84-4.78 (dd, J=4.8amd 10.4 Hz, 1H), 2.34-2.10 (m, 4H); ¹³C NMR (DMSO-d₆) δ 173.03, 171.88, 165.96, 143.35, 137.49, 134.77, 130.10, 129.61, 126.95, 53.65, 48.13, 31.50, 24.69; Anal. Calcd for C₁₃H₁₃N₃O₆: C, 50.82; H, 4.26; N, 13.68. Found: C, 50.53; H. 4.37; N, 13.22.

### (S)-3-(1-oxo-4-nitroisoindolin-2-yl)piperidine-2,6-dione

A stirred suspension mixture of N-(1-oxo-4-nitroisoindolin-2-yl)-L-glutamine (3.2 g, 10.5 mmol) in anhydrous methylene chloride (150 mL) was cooled to −40° C. with isopropanol/dry ice bath. Thionyl chloride (0.82 mL, 11.3 mmol) was added dropwise to the cooled mixture followed by pyridine (0.9 g. 1.1.3 mmol). After 30 min, triethylamine (1.2 g, 11.5 mmol) was added and the mixture was stirred at −30 to −40° C. for 3 hours. The mixture was poured into ice water (200 mL) and the aqueous layer was extracted with methylene chloride (40 mL). The methylene chloride solution was washed with water (2×60 mL), brine (60 mL) and dried. The solvent was removed in vacuo and the solid residue was slurried with ethyl acetate (20 mL) to give 2.2 g (75%) of the product as a white solid: mp 285° C.; 1H NMR (DMSO-d₆) δ: 1.04 (s, 1H), 8.49-8.45 (dd, J=0.8 and 8.2 Hz, 1H), 8.21-8.17 (dd, J=7.3 Hz, 1H), 7.84 (t, J=7.6 Hz, 1H), 5.23-5.15 (dd, J=4.9 and 13.0 Hz, 1H), 4.96 (dd, J=19.3 and 32.4 Hz, 2H), 3.00-2.85 (m, 1H), 2.64-2.49 (m, 2H), 2.08-1.98 (m, 1H); ¹³C NMR (DMSO-d₆) δ 172.79, 170.69, 165.93, 143.33, 137.40, 134.68, 130.15, 129.60, 127.02, 51.82, 48.43, 31.16. 22.23; HPLC, Waters Nove-Pak/C18, 3.9×150 mm, 4 micron, 1 mL/min, 240 nm, 20/80 CH₃CN/0.1% H₃PO₄(aq) 3.67 min(100%); Anal. Calcd for C₁₃HₙN₃O₅: C, 53.98; H, 3.83; N, 14.53. Found: C, 53.92; H, 3.70; N, 14.10.

### 3-(4-amino-1-oxo-1,3-dihydro-isoindol-2-yl)-piperidine-2,6-dione

A mixture of (S)-3-(1-oxo-4-nitroisoindolin-2-yl)piperidine-2,6-dione (1.0 g, 3.5 mmol) and 10% Pd/C (0.3 g) in methanol (600 mL) was hydrogenated in a Parr-Shaker apparatus at 50 psi of hydrogen for 5 hours. The mixture was filtered through Celite and the filtrate was concentrated in vacuo. The solid was slurried in hot ethyl acetate for 30 min, filtered and dried to afford 0.46 g (51%) of the product as a white solid: mp 235.5-239° C.; ¹H NMR (DMSO-d₆) δ 11.01 (s, 1H). 7.19 (t, J=7.6 Hz, 1H). 6.90 (d. J=7.3 Hz, 1H), 6.78 (d, J=7.8 Hz, 1H), 5.42 (s, 2H). 5.12 (dd. J=5.1 and 13.1 Hz, 1H), 4.17 (dd, J=17.0 and 28.8 Hz, 2H), 2.92-2.85 (m, 1H). 2.64-2.49 (m, 1H). 2.34-2.27 (m, 1H), 2.06-1.99 (m, 1H); ¹³C NMR (DMSO-d₆) δ 172.85, 171.19, 168.84, 143.58, 132.22. 128.79, 125.56, 116.37, 110.39, 51.48, 45.49, 31.20, 22.74; HPLC. Waters Nova-Pak/C18, 3.9×150 mm, 4 micron, 1 mL/min, 240 nm, 10/90 CH₃CN/0.1% H₃PO₄(aq) 0.96 min(100%); Chiral analysis, Daicel Chiral Pak AD, 40/60 Hexane/IPA, 6.60 min(99.42%); Anal. Calcd for C₁₃H₁₃N₃O₃: C, 60.23; H, 5.05; N, 16.21. Found: C, 59.96; H. 4.98; N, 15.84.

3-(4-Amino-1-oxo-1,3-dihydro-isoindol-2-yl)-piperidine-2,6-dione may also be prepared by methods known in the art, for example, as provided in Treatment compounds of the Future, 2003, 28(5): 425-431, the entirety of which is incorporated by reference.

### 5.2 Preparation of 3-(5-amino-2-methyl-4-oxo-4H-quinazolin-3-yl)-piperidine-2,6-dione (Compound A)

To a solution of potassium hydroxide (16.1 g, 286 mmol) in water (500 mL), was added 3-nitrophthalimide (25.0 g, 130 mmol) in portion at 0° C. The suspension was stirred at 0° C. for 3 hrs, and then heated to 30° C. for 3 hrs. To the solution, was added HCl (100 mL, 6N). The resulting suspension was cooled to 0° C. for 1 hr. The suspension was filtered and washed with cold water (2×10 mL) to give 3-nitro-phthalamic acid as a white solid (24.6 g, 90% yield): ¹H NMR (DMSO-d₆) δ 7.69 (brs, 1H, NHH), 7.74 (t, J=8 Hz, 1H, Ar), 7.92 (dd, J=1, 8 Hz, 1H, Ar), 8.13 (dd, J=1, 8 Hz, 1H, Ar), 8.15 (brs, 1H, NHH), 13.59 (s, 1H, OH); ¹³C NMR (DMSO-d₆) δ 125.33, 129.15, 130.25, 132.54, 136.72, 147.03, 165.90, 167.31.

To a mixture of 3-nitro-phthalamic acid (24.6 g, 117 mmol) and potassium hydroxide (6.56 g, 117 mmol) in water (118 mL), was added a mixture of bromine (6 mL), potassium hydroxide (13.2 g, 234 mmol) in water (240 mL) at 0° C., followed by addition of a solution of potassium hydroxide (19.8 g, 351 mmol) in water (350 mL). After 5 minutes at 0° C., the mixture was heated in a 100° C. oil bath for 1 hr. The reaction solution was cooled to room temperature, and then, in an ice-water bath for 30 minutes. To the mixture, a solution of HCl (240 mL, 2N) was added dropwise at 0° C., and the resulting mixture was kept for 1 hr. The suspension was filtered and washed with water (5 mL) to give 2-amino-6-nitro-benzoic acid as yellow solid (15.6 g, 73% yield): HPLC: Waters Symmetry C₁₈, 5 μm, 3.9×150 mm, 1 mL/min, 240 nm, CH₃CN/0.1% H₃PO₄, 5% grad to 95% over 5 min, 5.83 min (85%); ¹H NMR (DMSO-d₆) δ 6.90 (dd, J=1, 8 Hz, 1H, Ar), 7.01 (dd, J=1, 9 Hz, 1H, Ar), 7.31 (t, J=8 Hz, 1H, Ar), 8.5-9.5 (brs, 3H, OH, NH₂); ¹³C NMR (DMSO-d₆) δ 105.58, 110.14, 120.07, 131.74, 149.80, 151.36, 166.30; LCMS: MH=183.

A mixture of 2-amino-6-nitro-benzoic acid (1.5 g, 8.2 mmol) in acetic anhydride (15 mL) was heated at 200° C. for 30 minutes in a microwave oven. The mixture was filtered and washed with ethyl acetate (20 mL). The filtrate was concentrated in vacuo. The solid was stirred in ether (20 mL) for 2 hrs. The suspension was filtered and washed with ether (20 mL) to give 2-methyl-5-nitro-benzo[d][1,3]oxazin-4-one as a light brown solid (1.4 g, 85% yield): HPLC: Waters Symmetry C₁₈, 5 μm, 3.9×150 mm, 1 mL/min, 240 nm, CH₃CN/0.1% H₃PO₄, 5% grad 95% in 5 min, 5.36 min (92%); ¹H NMR (DMSO-d₆) δ 2.42 (s, 3H, CH₃), 7.79 (dd, J=1, 8 Hz, 1H, Ar), 7.93 (dd, J=1, 8 Hz, 1H, Ar), 8.06 (t, J=8 Hz, 1H, Ar); ¹³C NMR (DMSO-d₆) 520.87, 107.79, 121.54, 128.87, 137.19, 147.12, 148.46, 155.18, 161.78; LCMS: MH=207.

Two vials each with a suspension of 5-nitro-2-methyl-benzo[d][1,3]oxazin-4-one (0.60 g, 2.91 mmol) and 3-amino-piperidine-2,6-dione hydrogen chloride (0.48 g, 2.91 mmol) in pyridine (15 mL) were heated at 170° C. for 10 minutes in a microwave oven. The suspension was filtered and washed with pyridine (5 mL). The filtrate was concentrated in vacuo. The resulting mixture was stirred in HCl (30 mL, 1N), ethyl acetate (15 mL) and ether (15 mL) for 2 hrs. The suspension was filtered and washed with water (30 mL) and ethyl acetate (30 mL) to give a dark brown solid, which was stirred with methanol (50 mL) at room temperature overnight. The suspension was filtered and washed with methanol to give 3-(2-methyl-5-nitro-4-oxo-4H-quinazolin-3-yl)-piperidine-2,6-dione as a black solid (490 mg, 27% yield). The solid was used in the next step without further purification.

A mixture of 3-(2-methyl-5-nitro-4-oxo-4H-quinazolin-3-yl)-piperidine-2,6-dione (250 mg) and Pd(OH)₂ on carbon (110 mg) in DMF (40 mL) was shaken under hydrogen (50 psi) for 12 hrs. The suspension was filtered through a pad of Celite and washed with DMF (10 mL). The filtrate was concentrated in vacuo and the resulting oil was purified by flash column chromatography (silica gel, methanol/methylene chloride) to give 3-(5-amino-2-methyl-4-oxo-4H-quinazolin-3-yl)-piperidine-2,6-dione as a white solid (156 mg, 69% yield): HPLC: Waters Symmetry C₁₈, 5 μm, 3.9×150 mm, 1 mL/min, 240 nm, 10/90 CH₃CN/0.1% H₃PO₄, 3.52 min (99.9%); mp: 293-295° C.; ¹H NMR (DMSO-d₆) δ 2.10-2.17 (m, 1H, CHH), 2.53 (s, 3H, CH₃), 2.59-2.69 (m, 2H, CH₂), 2.76-2.89 (m, 1H, CHH), 5.14 (dd, J=6, 11 Hz, 1H, NCH), 6.56 (d, J=8 Hz, 1H, Ar), 6.59 (d, J=8 Hz, 1H, Ar), 7.02 (s, 2H, NH₂), 7.36 (t, J=8 Hz, 1H, Ar), 10.98 (s, 1H, NH); ¹³C NMR (DMSO-d₆) δ 20.98, 23.14, 30.52, 55.92, 104.15, 110.48, 111.37, 134.92, 148.17, 150.55, 153.62, 162.59, 169.65, 172.57; LCMS: MH=287; Anal. Calcd. for C14H14N4O3+0.3 H2O: C, 57.65; H, 5.05; N, 19.21. Found: C, 57.50; H, 4.73; N, 19.00.

### 5.3 Preparation of 3-[4-(4-morpholin-4-ylmethyl-benzyloxy)-1-oxo-1,3-dihydro-isoindol-2-yl]-piperidine-2,6-dione (Compound B)

Procedure 1:

Step 1: To the solution of 3-(4-hydroxy-1-oxo-1,3-dihydro-isoindol-2-yl)-piperidine-2,6-dione (2.5 g, 8.56 mmol) in THF (60 mL) was added triphenyl phosphine (polymer supported 1.6 mmol/g, 12 g, 18.8 mmol). The mixture was stirred at room temperature for 15 minutes. Diisopropyl azodicarboxylate (3.96 mL, 18.8 mmol) was added at 0° C., and the mixture was stirred at 0° C. for 30 minutes. (4-Morpholin-4-ylmethyl-phenyl)-methanol (2.62 g, 12.4 mmol) was added at 0° C., and the mixture was allowed to warm to room temperature and stirred at room temperature overnight. The reaction mixture was filtered, and the filtrate was concentrated. The resulting oil was purified on silica gel column eluted with methylene chloride and methanol (gradient, product came out at 6% methanol) to give 4-carbamoyl-4-[4-(4-morpholin-4-ylmethyl-benzyloxy)-1-oxo-1,3-dihydro-isoindol-2-yl]-butyric acid methyl ester (2.2 g, 54% yield). The product was used in the next step without further purification.

Step 2: To the THF solution (50 mL) of 4-carbamoyl-4-[4-(4-morpholin-4-ylmethyl-benzyloxy)-1-oxo-1,3-dihydro-isoindol-2-yl]-butyric acid methyl ester (2.2 g, 4.57 mmol) was added potassium tert-butoxide (0.51 g, 4.57 mmol) at 0° C. The mixture was stirred at 0° C. for 10 minutes and was quenched with 1N HCl (5 mL, 5 mmol) followed by saturated NaHCO₃ (25 mL). The mixture was extracted with EtOAc (2×50 mL). The organic layer was washed with water (30 mL), brine (30 mL), dried over MgSO₄ and concentrated. To the resulting solid was added EtOAc (10 mL) followed by hexane (10 mL) under stirring. The suspension was filtered to give 3-[4-(4-morpholin-4-ylmethyl-benzyloxy)-1-oxo-1,3-dihydro-isoindol-2-yl]-piperidine-2,6-dione as white solid (1.5 g, 73% yield). HPLC: Waters Symmetry C₁₈, 5 μm, 3.9×150 mm, 1 mL/min, 240 nm, gradient to 95/5 acetonitrile/0.1% H₃PO₄ in 5 min, t_{R}=4.78 min (97.5%); mp: 210-212° C.; ¹H NMR (DMSO-d₆) 1.86-2.09 (m, 1H, CHH), 2.29-2.38 (m, 4H, CH₂, CH₂), 2.44 (dd, J=4.3, 13.0 Hz, 1H, CHH), 2.53-2.64 (m, 1H, CHH), 2.82-2.99 (m, 1H, CHH), 3.46 (s, 2H, CH₂), 3.52-3.61 (m, 4H, CH₂, CH₂), 4.18-4.51 (m, 2H, CH₂), 5.11 (dd, J=5.0, 13.3 Hz, 1H, NCH), 5.22 (s, 2H, CH₂), 7.27-7.38 (m, 5H, Ar), 7.40-7.53 (m, 3H, Ar), 10.98 (s, 1H, NH) ¹³C NMR (DMSO-d₆) □22.36, 31.2 127.64, 128.99, 129.81, 129.95, 133.31, 135.29, 137.68, 153.50, 168.01, 170.98, 172.83; LCMS: 465; Anal Calcd for C₂₅H₂₇N₃O₅+0.86 H₂O: C, 64.58; H, 6.23; N, 9.04. Found: C, 64.77; H, 6.24; N, 8.88.

Procedure 2:

Step 1: To a 2-L round bottom flask, were charged methyl 5-amino-4-(4-hydroxy-1-oxoisoindolin-2-yl)-5-oxopentanoate (30 g, 103 mmol), 1,4-bis(bromomethyl)benzene (81 g, 308 mmol) and potassium carbonate (14.19 g, 103 mmol) and acetonitrile (1.2 L). The mixture was stirred at room temperature for 10 minutes and heated to 50° C. for 12 hours. The reaction mixture was allowed to cool to room temperature. The mixture was filtered and the filtrate was concentrated on rota-vap. The resulting solid was dissolved in CH₂Cl₂ and loaded on 2 silica gel columns (330 g each) and eluted using CH₂Cl₂/MeOH to give 4-[4-(4-bromomethyl-benzyloxy)-1-oxo-1,3-dihydro-isoindol-2-yl]-4-carbamoyl-butyric acid methyl ester as white solid (40 g, 82% yield): ¹H NMR (DMSO-d₆) 1.98-2.13 (m, 1H, CHH), 2.14-2.23 (m, 1H, CHH), 2.23-2.32 (m, 2H, CHH, CHH), 3.50 (s, 3H, CH₃), 4.34-4.63 (m, 2H, CH₂), 4.67-4.80 (m, 3H, CH₂, NCH), 5.25 (s, 4H, CH₂), 7.19 (s, 1H, NHH), 7.24-7.34 (m, 2H, Ar), 7.41-7.54 (m, 5H, Ar), 7.58 (br. s., 1H, NHH).

Step 2: To the CH₂Cl₂ solution of methyl 5-amino-4-(4-(4-(bromomethyl)benzyloxy)-1-oxoisoindolin-2-yl)-5-oxopentanoate (36.5 g, 77 mmol), was added morpholine (14.72 ml, 169 mmol) at room temperature. The mixture was stirred at room temperature for 1 hour. The resulting suspension was filtered, and the filtrate was concentrated on rota-yap. The resulting oil was dissolved in 350 mL of EtOAc and washed with water (50 mL×3). The organic layer was concentrated on rota-yap to give 4-carbamoyl-4-[4-(4-morpholin-4-ylmethyl-benzyloxy)-1-oxo-1,3-dihydro-isoindol-2-yl]-butyric acid methyl ester as a foamy solid (39 g, 100% yield): ¹H NMR (DMSO-d₆) 2.00-2.12 (m, 1H, CHH), 2.14-2.22 (m, 1H, CHH), 2.22-2.29 (m, 2H, CHH, CHH), 2.30-2.39 (m, 4H, CH₂, CH₂), 3.46 (s, 2H, CH₂), 3.50 (s, 3H, CH₃), 3.53-3.63 (m, 4H, CH₂, CH₂), 4.28-4.59 (m, 2H, CH₂), 4.73 (dd, J=4.7, 10.2 Hz, 1H, NCH), 5.22 (s, 2H, CH₂), 7.14-7.23 (m, 1H, NHH), 7.26-7.39 (m, 4H, Ar), 7.41-7.51 (m, 3H, Ar), 7.58 (s, 1H, NHH).

Step 3: To the THF solution of methyl 5-amino-4-(4-(4-(morpholinomethyl)benzyloxy)-1-oxoisoindolin-2-yl)-5-oxopentanoate (40 g, 83 mmol), was added potassium 2-methylpropan-2-olate (9.80 g, 87 mmol) portion wise at 0° C. The mixture was stirred at this temperature for 30 minutes. To the reaction mixture, was added 45 mL of 1N HCl solution, followed by 200 mL of saturated NaHCO₃ solution. The mixture was diluted with 500 mL of EtOAc at 0° C., stirred for 5 minutes and separated. The organic layer was washed with water (50 mL×3) and brine (100 mL), and concentrated on rota-yap to give a white solid, which was stirred in diethyl ether (300 mL) to give a suspension. The suspension was filtered to give 3-[4-(4-morpholin-4-ylmethyl-benzyloxy)-1-oxo-1,3-dihydro-isoindol-2-yl]-piperidine-2,6-dione as white solid (28.5 g, 72% yield): HPLC: Waters Symmetry C₁₈, 5 μm, 3.9×150 mm, 1 mL/min, 240 nm, gradient to 95/5 acetonitrile/0.1% H₃PO₄ in 5 min, t_{R}=4.78 min (98.5%); mp: 209-211° C.; ¹H NMR (DMSO-d₆) δ 1.86-2.09 (m, 1H, CHH), 2.29-2.38 (m, 4H, CH₂, CH₂), 2.44 (dd, J=4.3, 13.0 Hz, 1H, CHH), 2.53-2.64 (m, 1H, CHH), 2.82-2.99 (m, 1H, CHH), 3.46 (s, 2H, CH₂), 3.52-3.61 (m, 4H, CH₂, CH₂), 4.18-4.51 (m, 2H, CH₂), 5.11 (dd, J=5.0, 13.3 Hz, 1H, NCH), 5.22 (s, 2H, CH₂), 7.27-7.38 (m, 5H, Ar), 7.40-7.53 (m, 3H, Ar), 10.98 (s, 1H, NH); ¹³C NMR (DMSO-d₆) δ 22.36, 31.21, 45.09, 51.58, 53.14, 62.10, 66.17, 69.41, 114.97, 115.23, 127.64, 128.99, 129.81, 129.95, 133.31, 135.29, 137.68, 153.50, 168.01, 170.98, 172.83; LCMS: 465; Anal Calcd for C₂₅H₂₇N₃O₅+0.86 H₂O: C, 64.63; H, 6.22; N, 9.04. Found: C, 64.39; H, 6.11; N, 8.89; H₂O, 3.24.

### 5.4 Proteomic Screening for Proteins Up-Regulated or Down-Regulated in Response to Compound a Treatment in Focus and JHH4 Cells

HCC xenografts (excised xenograft tumors of HAK-1AJ and Focus) were treated with either with DMSO, or Compound A for either once (single) or multiple times. Cells were harvested at 24 or 72 hours time points, and proteins were fractionated using a basic pH reverse gradient method. Fractionated samples were labeled using a Tandem Mass Tag (TMT) method. Relative abundance was calculated.

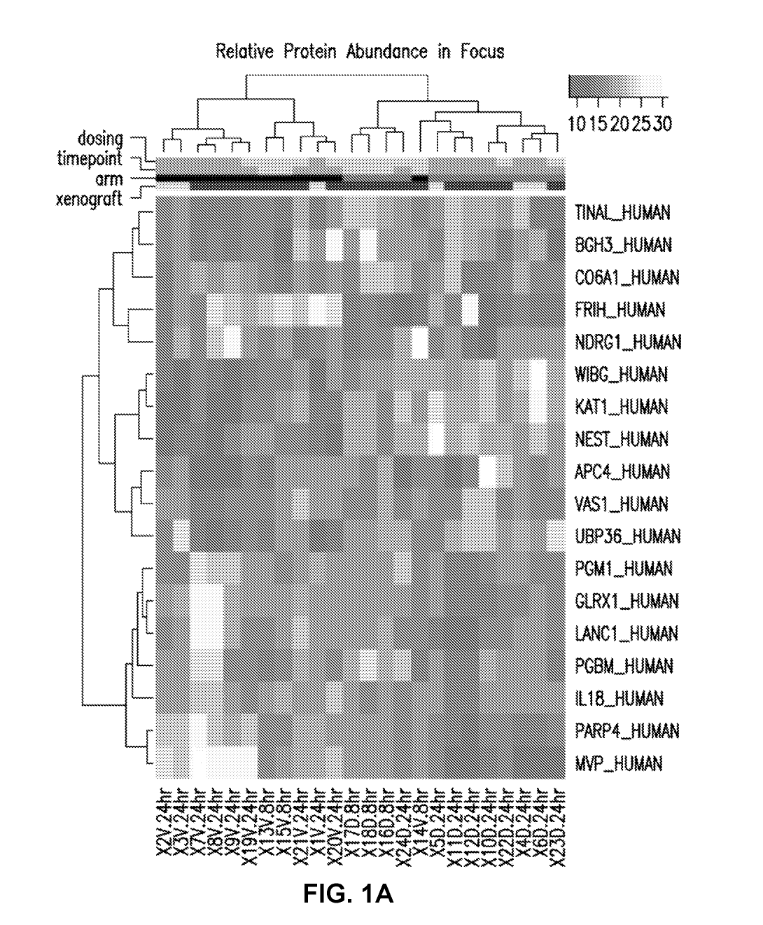

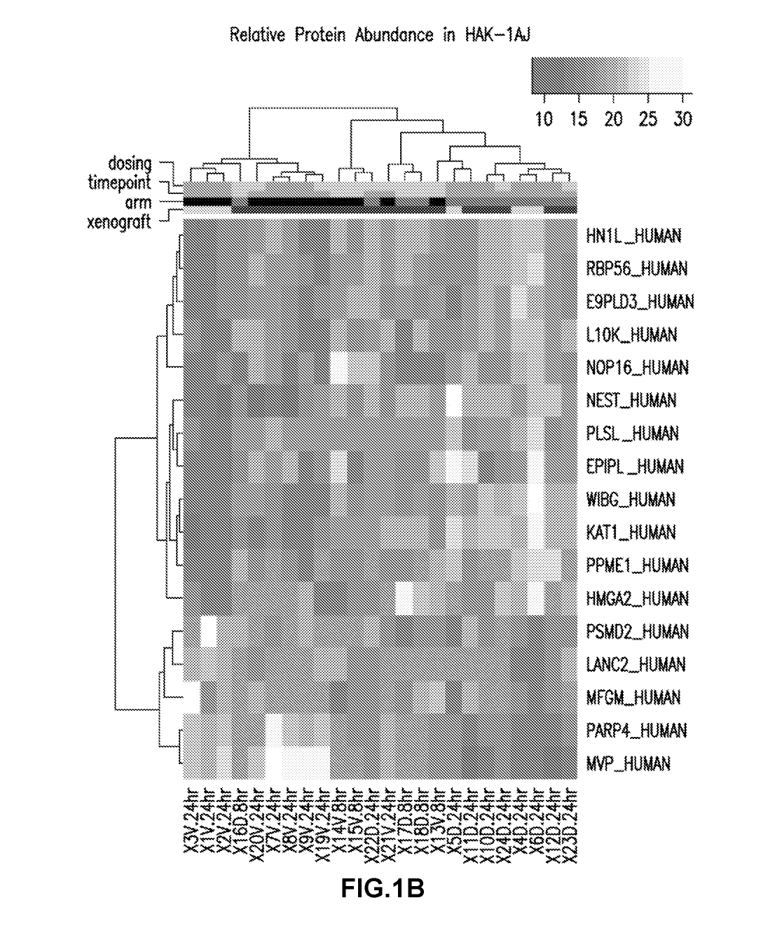

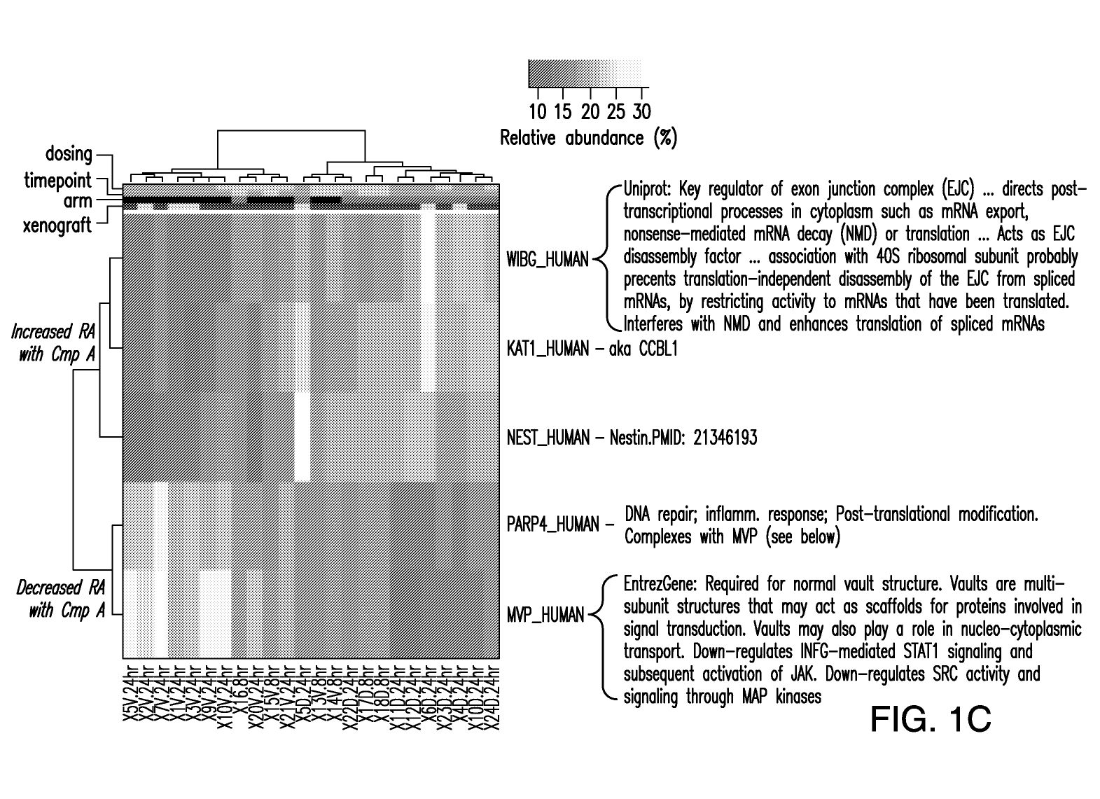

The results of the proteomics screening study are shown in FIG. 1A-C. FIG. 1A shows the relative abundance of the differentially abundant proteins after Compound A treatment compared with the DMSO treatment in Focus cells. FIG. 1B shows the relative abundance of the differentially abundant proteins after Compound A treatment compared with the DMSO treatment in HAK-1AJ cells. FIG. 1C shows proteins commonly differentially abundant at 24 hours after treatment with Compound A in both Focus and HAK-1AJ cells, e.g., WIBG, KAT1, Nestin, PARP4, and MVP.

Based on the proteomic screening study, certain proteins upregulated in response to Compound A treatment include Nestin, KAT1/CCBL1, and WIBG, and certain proteins downregulated in response to Compound A treatment include MVP, PARP4, ZFP91, and ZNF198.

Proteomic screening for proteins up-regulated or down-regulated in response to Compound A treatment was also performed using Focus and JHH4 cell lines. Representative proteins upregulated in response to Compound A treatment are shown in Table 3 and Table 4. Table 3 represents proteins upregulated by Compound A in the JHH4 cell line grown under 3-dimensional (3D) culture conditions using basement membrance extracellular matrix. Table 4 represents proteins upregulated by Compound A in FOCUS cell line under 3D conditions. Representative proteins downregulated in response to Compound A treatment are shown in Table 5 and Table 6. Table 5 represents proteins downregulated by Compound A in JHH4 cell line under 3D conditions. Table 6 represents proteins downregulated by Compound A in FOCUS cell line under 3D conditions.

Thus, these proteins in Tables 3-6 can be used as biomarkers in the various methods provided herein.

Representative proteins identified in the proteomic studies were confirmed in western blot analysis, such as those shown in FIG. 8. As shown, the protein levels of MST1R (RON), RARP4, MVP, ZFP91, IKBKE, PDE6D reduced in response to Compound A treatment; and protein levels of CK1alpha, LGALS2, KAT1, GSPT1 as well as CRBN increased in response to Compound A treatment.

TABLE 3
Upregulated Proteins in Response to Compound A Treatment (Set 1)

| | | | | |
|---|---|---|---|---|
| SEPT2 | A2M | A2mp | ACSM2B | ADAM22 |
| AFP | CD74 | HMBOX1 | NCOA3 | SERF2 |
| AGMAT | CDH16 | HYPK | NR2C1 | Serpina10 |
| AGT | CEACAM1 | ITFG2 | PAH | Serpinf1 |
| Ahsg | CLN3 | Itgb1 | PDZK1 | SLC25A51 |
| AHSG | COX5A | ITIH1 | Peg10 | SLC29A3 |
| ALB | CPVL | ITIH4 | PLEKHO2 | SPAG7 |
| Amacr | CRBN | KANSL3 | Postn | Suc1g2 |
| AMACR | CRYM | KCTD14 | POSTN | SULT1A1 |
| ANPEP | CTSL | KLC4 | PPP1R13L | SULT1A3 |
| Anxa5 | CTU1 | KPTN | Prdx3 | Tollip |
| ANXA9 | DAPK2 | Lamp2 | PRODH2 | Top2a |
| APBB2 | DLG5 | LGALS2 | Ptbp3 | Trap1 |
| APCS | DUSP23 | LGALS9 | RBKS | TSPAN31 |
| AQP1 | ELF3 | LIG3 | RGS14 | TTC13 |
| ATHL1 | Eps15 | LMBRD1 | RHBDD2 | Upp1 |
| Atp5c1 | ERBB3 | LOC100996516 | RNF126 | USP30 |
| C11orf52 | FAM83H | LPL | RNF7 | Vcam1 |
| C3 | Fkbp9 | LYN | Rp19 | VIL1 |
| C4A | FLRT3 | MAP2K7 | Rplp1 | Vim |
| CASR | FUK | MLLT6 | Rplp2 | ZNF770 |
| CCBL1 | GC | Mogs | RSBN1L | |
| CCDC28A | GPD1 | Mrps22 | SAT2 | |
| Cct3 | GPR39 | MYRF | SEPHS1 | |

TABLE 4
Upregulated Proteins in Response to Compound A Treatment (Set 2)

| | | | | |
|---|---|---|---|---|
| MARCH7 | CCAR2 | Eif2s1 | GPBP1L1 | LARP1B |
| SEPT2 | CCBL1 | ENTPD5 | Gtpbp4 | LGALS9 |
| Abcc1 | CCDC38 | EPDR1 | HABP4 | LMBRD1 |
| ABCG1 | CCDC71L | Eps8l2 | HIRIP3 | Lta4h |
| ABCG2 | CD33 | Epx | HK1 | LYRM1 |
| Adam10 | CD40 | FAM111A | HMGXB3 | MAT1A |
| AFF3 | CEACAM1 | FAM120B | HSP90AA4P | MBIP |
| Akap9 | CHKB | FAM206A | IGDCC4 | MBP |
| AP1AR | CHURC1 | FAM83H | IL36B | METTL21A |
| APOL1 | CLK4 | Fasn | IMMT | MLLT1 |
| ATP11A | Col15a1 | FASTKD3 | INA | MPV17L2 |
| BANP | CPN1 | FCGRT | KCTD18 | MTIF3 |
| BLZF1 | CSRP2BP | FLII | KIDINS220 | MTRF1L |
| C10orf118 | CXorf67 | FNBP1L | KIF16B | MYO5B |
| C4b | DGAT1 | Gak | KLC4 | N4BP2 |
| C6orf57 | DHRS2 | Gatad2a | KLHL24 | NABP2 |
| CAST | DLG5 | Gm906 | LAMTOR5 | NAP1L1 |
| Ndufa10 | POLK | SGCB | TEX9 | Vps37c |
| Ndufa2 | PORCN | SIRT7 | TGFBRAP1 | VPS54 |
| Ndufs2 | PPAPDC2 | SLC50A1 | TMC6 | VRK3 |
| NFRKB | Ppp2r4 | SMCR8 | TMEM120A | XRRA1 |
| NSMCE2 | RBPMS2 | SPAG1 | TMEM179B | YPEL5 |
| NTHL1 | RNF4 | SPECC1 | TMEM50A | ZBTB1 |
| Nup37 | SAMD1 | SUV39H1 | TOPORS | ZBTB40 |
| OVOS1 | SATB2 | Sypl1 | TOR4A | ZDHHC3 |
| PDCD2 | SELK | TAPBPL | TRAPPC9 | ZHX1 |
| Pgd | SERBP1 | Tars | Tspan9 | ZNF292 |
| PLA2G4A | Serpina10 | Tarsl2 | TSPYL1 | ZNF830 |
| PLEC | Serpinc1 | TCF3 | TUBB4A | |
| PLXNA3 | Serpinf2 | FEC | TXNDC11 | |
| POLG | Serping1 | TERF1 | UGCG | |

TABLE 5
Downregulated Proteins in Response to Compound A Treatment (Set 1)

| | | | | |
|---|---|---|---|---|
| ABHD6 | DEGS1 | KIF20B | PDE6D | SLC4A7 |
| ACVR1 | DHX40 | KIF22 | PHC3 | SMAD3 |
| AGR2 | EHD2 | KLC2 | PHLDA1 | SPANXA1 |
| AHNAK2 | EPHA2 | KRT9 | PLA2G4A | SQSTM1 |
| AKAP12 | EREG | LDLR | PLCD4 | TIMM8A |
| ANLN | ETHE1 | LPXN | PPIL4 | TK1 |
| AP5S1 | FAM160A1 | MAFF | PRC1 | TMEM56 |
| ARL4C | FAM172A | MELK | Prkca | TMSB10 |
| ARL6IP1 | FOSL1 | MET | PRKCDBP | TNFRSF12A |
| ARPIN | GHDC | MGAT4B | PTGES2 | TRIM44 |
| ASH1L | GJA1 | MGLL | PTGS1 | TRIM65 |
| AXL | GLRX | MMP7 | RUNX2 | TSC22D1 |
| C4orf3 | GLS | MST1R | SCD | UBE2C |
| CANX | GNG2 | MT2A | SDSL | UBE2E2 |
| CD44 | GNG5 | MVP | SEL1L3 | UPK1B |
| CD46 | GRB10 | MYOF | SEMA3A | VASP |
| CD59 | GRPEL2 | MYOF | SERPINE1 | VPS13B |
| CDC45 | Iap | NDRG1 | SIRPA | WIZ |
| CENPK | Igf2r | NNMT | SKA1 | YBX3 |
| CEP55 | IGFBP1 | NTN4 | SLC14A1 | ZFP91 |
| COL6A3 | IGFBP3 | OTUB2 | SLC25A29 | ZMYM2 |
| CPA4 | IKBKE | PALMD | SLC2A1 | |
| CTNNAL1 | JAG1 | PANK1 | SLC34A2 | |
| CYP27A1 | KIAA0100 | PARP4 | SLC34A2 | |
| CYR61 | KIAA1462 | PBK | SLC38A2 | |

TABLE 6
Downregulated Proteins in Response to Compound A Treatment (Set 2)

| | | | | |
|---|---|---|---|---|
| ACSL6 | FAM84B | MGAT4B | PTDSS1 | SLC25A3 |
| AHSG | FAM98A | MGST1 | RAB28 | SLC25A6 |
| ALB | FER | MGST2 | RAB4B | SLC52A2 |
| ARPP19 | FKBP2 | MRPL9 | RAB8B | SNRPF |
| ATP5EP2 | FUNDC2 | MRPS18C | RNF166 | SPANXA1 |
| ATP5I | GK5 | MRPS21 | ROBO1 | SPPL2B |
| ATP5J | GLRX | Mt2 | RPL13A | STIM2 |
| AZGP1 | Gm14139 | MT2A | Rp118 | STK11 |
| BCKDK | GOLT1B | MT-ATP8 | RPL18A | SYTL4 |
| BLOC1S3 | GPATCH3 | MVP | RPL19 | TAF15 |
| BTF3 | Hbb-b2 | MYO1F | RPL22 | TBC1D12 |
| C2orf76 | HIST1H1C | NDUFB1 | RPL28 | TFAP4 |
| CABLES2 | Hist1h1e | NME7 | RPL35 | TMBIM6 |
| CAPN15 | HIST1H1E | NMES1 | RPL36AL | TMCO1 |
| CCDC88C | HIST1H2AH | NOMO2 | RPL37 | TMED9 |
| CDH1 | HIST1H2BC | NT5C2 | Rpl7 | TMEM189 |
| CETN2 | HMGB2 | Nt5dc2 | RPL8 | TMEM222 |
| CLTC | HMGN3 | NT5E | RPS13 | TPM1 |
| COG5 | HSP90B1 | NUDT3 | RPS15 | TWSG1 |
| COX17 | HSPB11 | NUDT5 | RPS17L | UBE3B |
| COX7B | IKBKE | OASL | RPS19 | VAPA |
| Cpsf1 | JAGN1 | PARP4 | RPS25 | VHL |
| CSRP2 | Kxd1 | Parp4 | RPS29 | VNN1 |
| CXXC1 | LAMA1 | PCK1 | RTN4IP1 | ZDHHC20 |
| CYC1 | LPXN | PDE6D | S100A11 | ZEB1 |
| DAP3 | MAFG | PFN2 | SDHAF2 | ZFP91 |
| DNAJC19 | MAGEA10 | PGPEP1 | SDSL | ZMYM2 |
| DNM2 | MED11 | PIGK | Sec13 | |
| DPY30 | MED18 | PLOD1 | SERF2 | |
| EHMT2 | METTL5 | Prdx2 | SIK2 | |
| FAM162A | MFSD1 | PTBP2 | SLC20A2 | |

### 5.5 Proteomic Screening Shows Compound a Reduces the Level of ZFP91 Protein in Focus and JHH4 Cell Lines

Both HCC cells, Focus and JHH4 in 3D setting, were cultured continuously for 15 days treated with either DMSO or Compound A, or were first cultured for about 7 days prior to treatment with either DMSO or Compound A for 24 hours or 72 hours. Cells were harvested and proteins were prepared from the cells for proteomics analysis. The growth of the cells on control plates was also analyzed. In particular, cells were plated in media containing either DMSO or Compound A. Cells were cultured for certain time, and then were harvested onto filter plates. After the plates have dried, scintillation fluid was added to the plates and read on a Top-count reader.

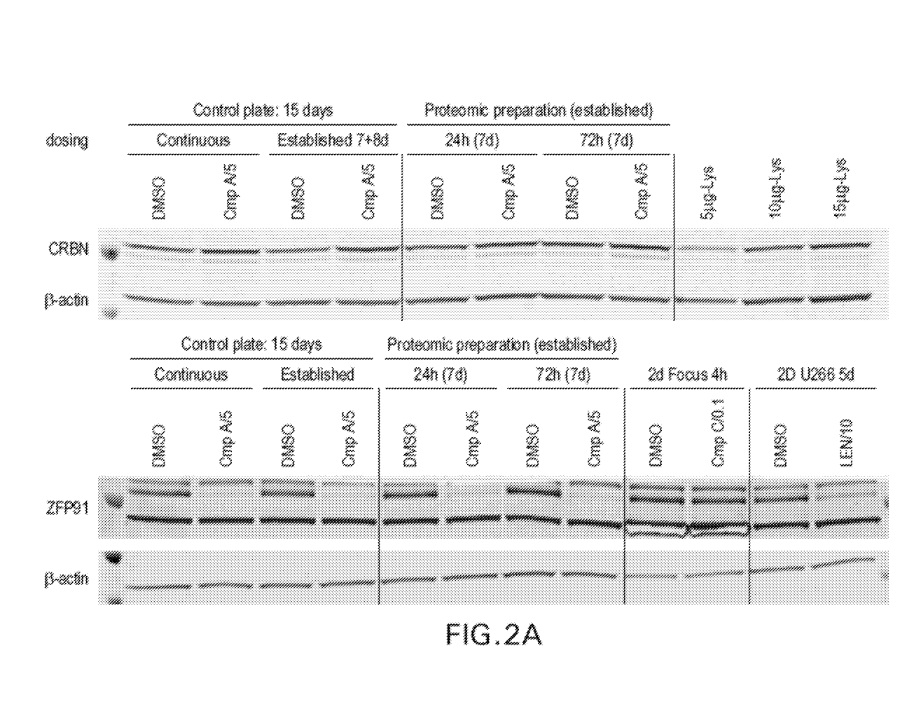

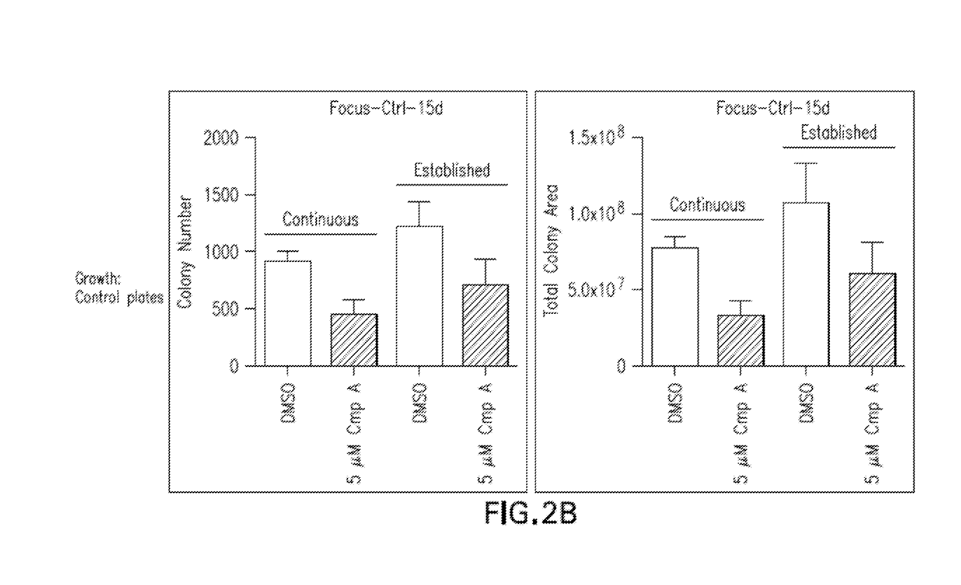

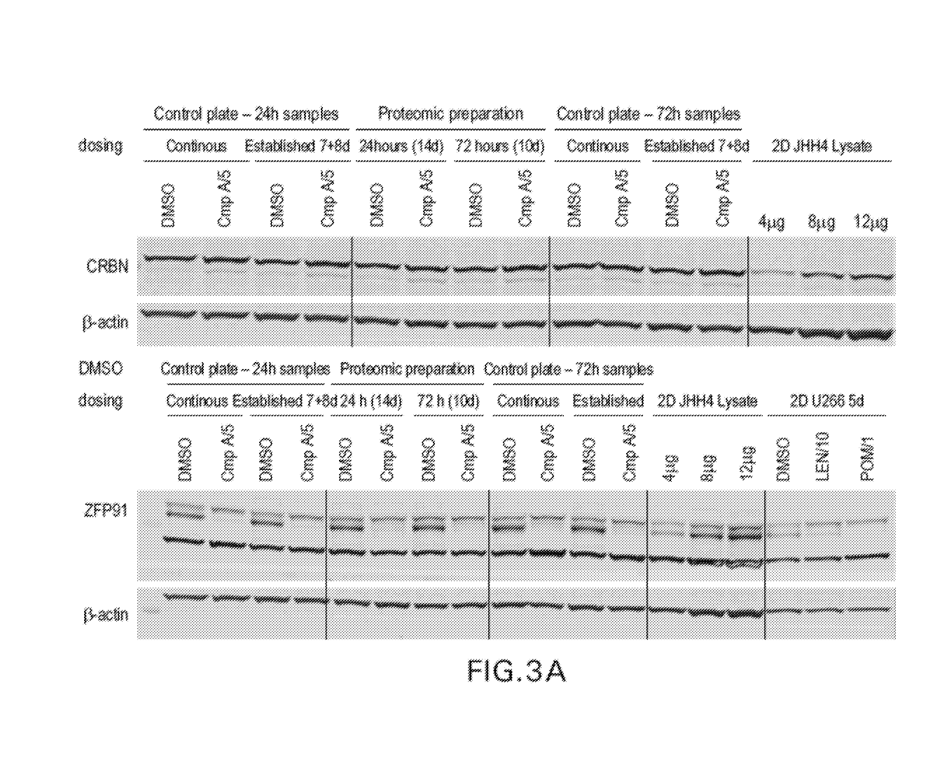

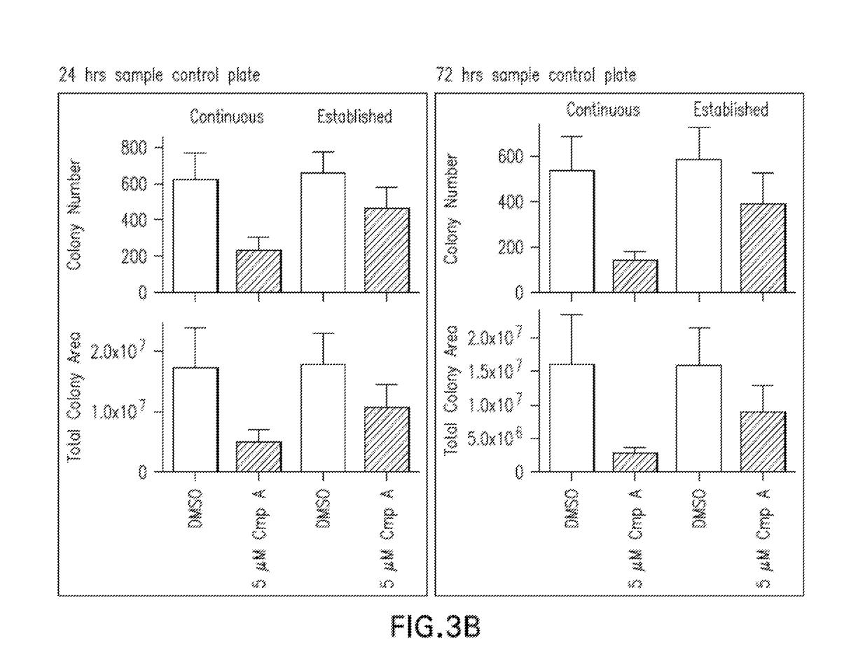

FIG. 2A shows the level of CRBN and ZFP91 in control plates and proteomic preparations (established) in 3D Focus cells treated with Compound A for various times. FIG. 2B shows the colony number and total colony area of control plates. FIG. 3A shows the level of CRBN and ZFP91 in control plate and proteomic preparation (established) in 3D JHH4 cells treated with Compound A for various times. FIG. 3B shows the colony number and total colony area of control plates. As shown, Compound A treatment reduced the colony number and the total colony area in both cell lines. The levels of CRBN and ZFP91 in control plate and proteomic preparation were analyzed using beta-actin as a control. As shown, in both HCC cell lines, Focus and JHH4, treatment with Compound A increased the level of CRBN protein, and reduced the level of ZFP91 protein.

### 5.6 Western Analysis Shows the Level of ZFP91 Reduces in Response to Compound A or Compound B Treatment in Focus and JHH4 Cell Lines

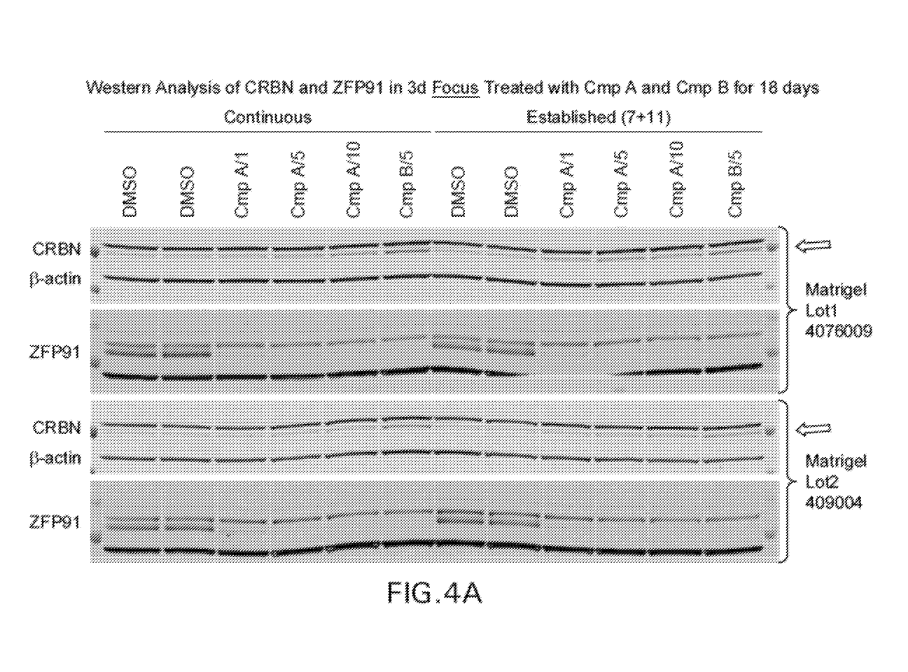

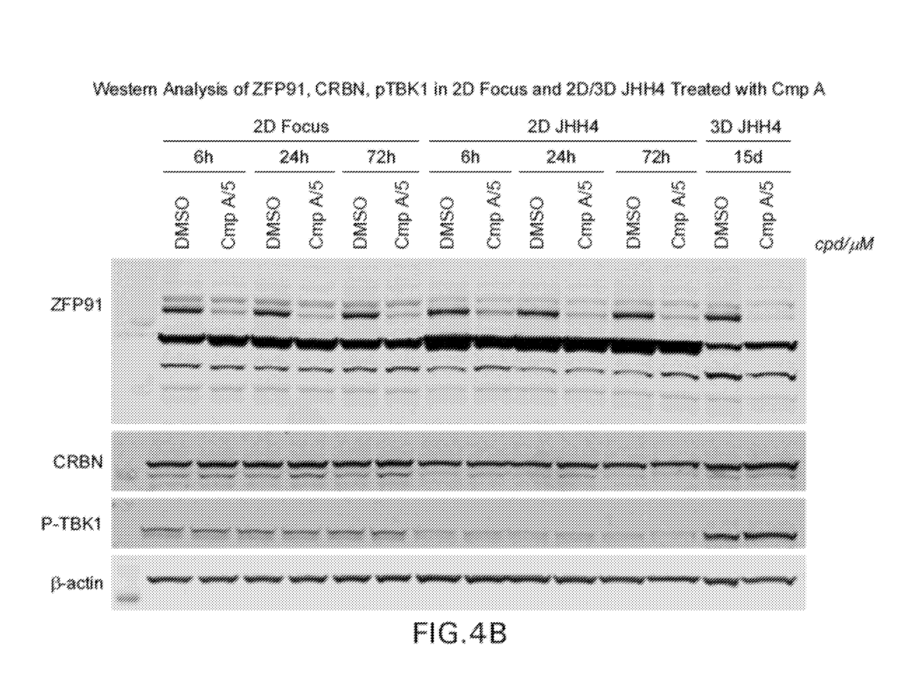

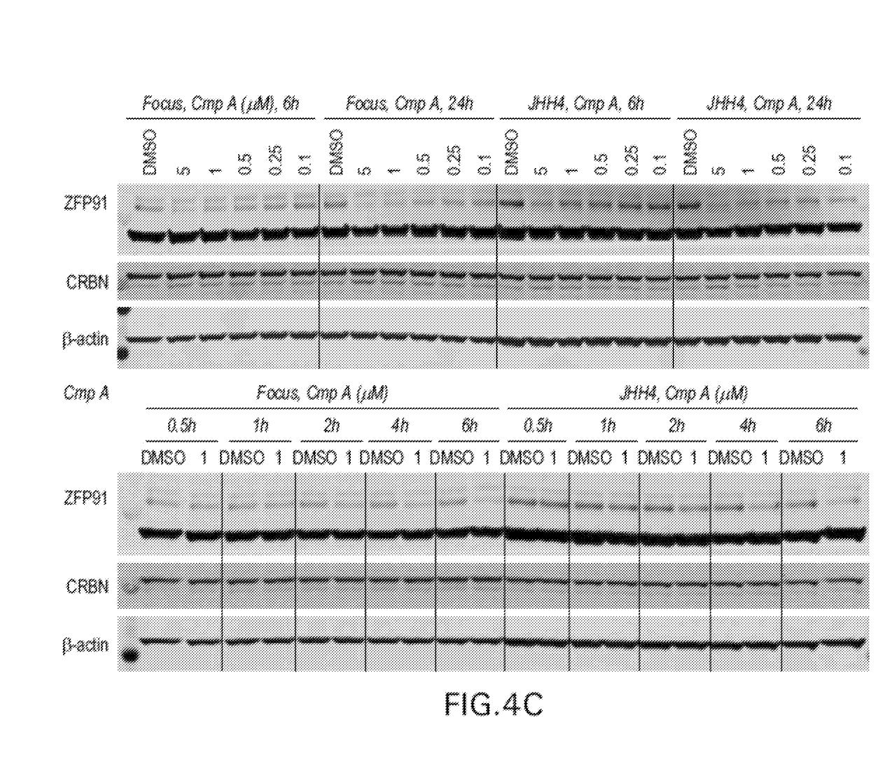

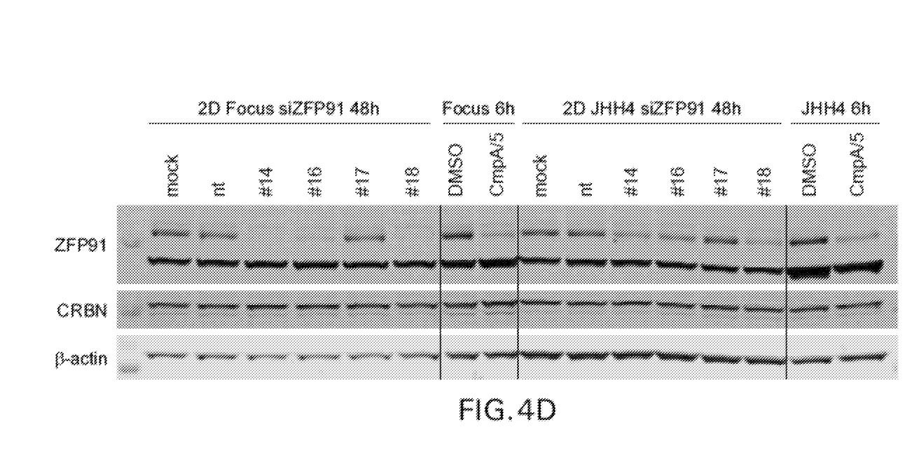

Focus cells were cultured for 18 days either continuously for 18 days in the medium containing DMSO, Compound A, or Compound B, or cultured for 7 days prior to treating with DMSO, Compound A, or Compound B for 11 days. Then cells were harvested and were treated in 200 μl cold cell lysis buffer (1% Triton X-100 from Cell Signaling supplemented with protease inhibitors and phophatase inhibitors from Roche) to generate cell lysate. Proteins from cell lysate were separated by 4-12% nupage gels (Invitrogen) and transferred to nitrocellulose membranes. Immunoblots were then probed with antibodies recognizing CRBN, ZFP91 (LSBio) and β-actin (Li-Cor). Signals were detected with a Li-Cor Odyssey Imager. The results of the western blot were shown in FIG. 4A. As shown, the level of ZFP91 reduced in response to treatment with Compound A or Compound B.

HCC cells from both Focus and JHH4 cell lines in 2D setting treated with Compound A for 6 hours, 24 hours, and 72 hours, and JHH4 cells in 3D setting treated with Compound A for 15 days were also analyzed by western blot. Cells were harvested and were treated in 200 μl cold cell lysis buffer (1% Triton X-100 from Cell Signaling supplemented with protease inhibitors and phophatase inhibitors from Roche) to generate cell lysate. Proteins from cell lysate were separated by 4-12% Nupage gels (Invitrogen) and transferred to nitrocellulose membranes. Immunoblots were then probed with antibodies recognizing CRBN, ZFP91 (LSBio), P-TBK1, and β-actin (Li-Cor). Signals were detected with a Li-Cor Odyssey Imager. The results were shown in FIG. 4B. As shown, the level of ZFP91 reduced in response to treatment with Compound A for 6 hours, 24 hours, or 72 hours in both cell lines in 2D setting. The level of ZFP91 also reduced in response to treatment with Compound A for 15 days in JHH4 in 3D setting. The level of CRBN increased in response to treatment with Compound A for 6 hours, 24 hours, or 72 hours in both cell lines in 2D setting. The level of CRBN also increased in response to treatment with Compound A for 15 days in JHH4 in 3D setting. It was also noted that P-TBK1 was upregulated in 3D setting upon Compound A treatment. ZFP91 degradation induced by various doses of Compound A for various time periods was also tested in both Focus and JHH4 cells, as shown in FIG. 4C. ZFP91 degradation in response to Compound A treatment is also confirmed using siRNAs targeting to ZFP91, as shown in FIG. 4D.

### 5.7 ZFP91 is Down-Regulated in Response to Treatment Compounds in a CRBN Dependent Pathway in Focus and JHH4 Cells

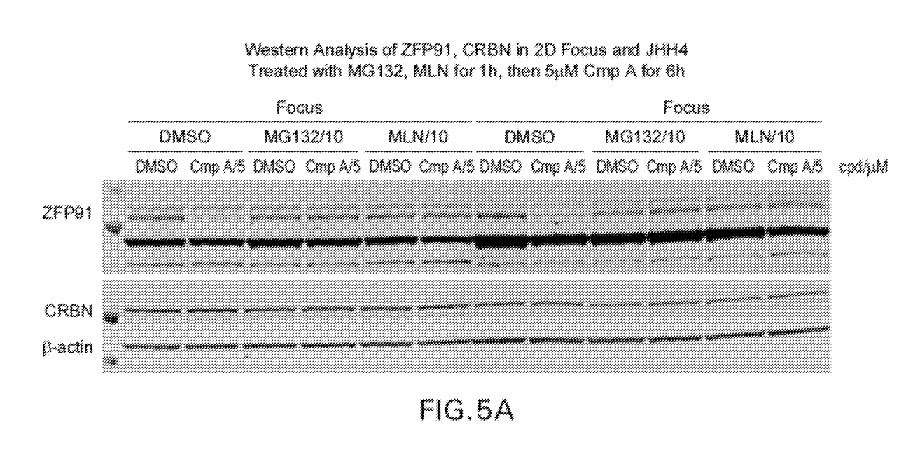

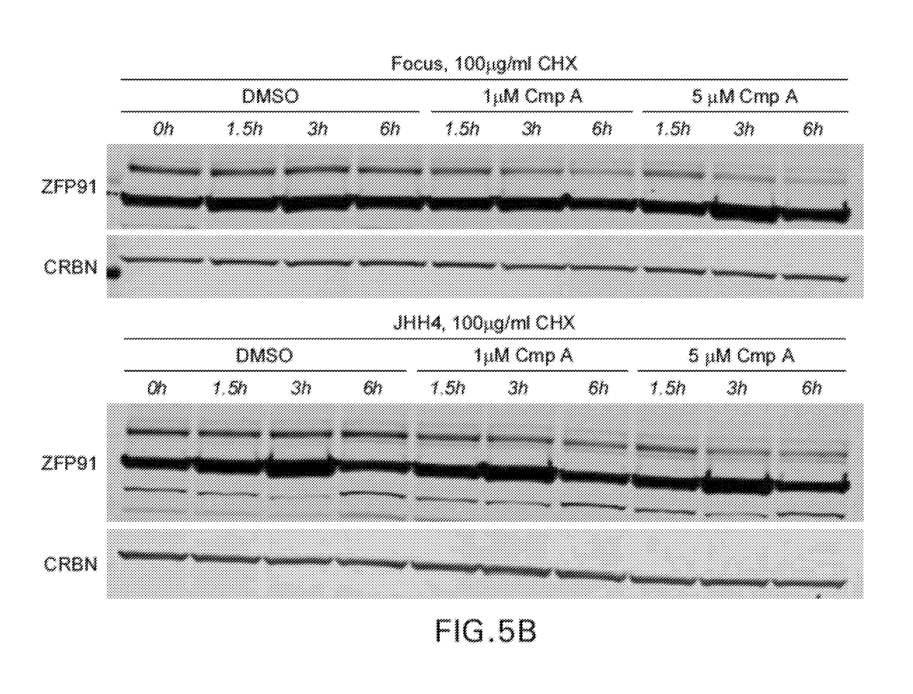

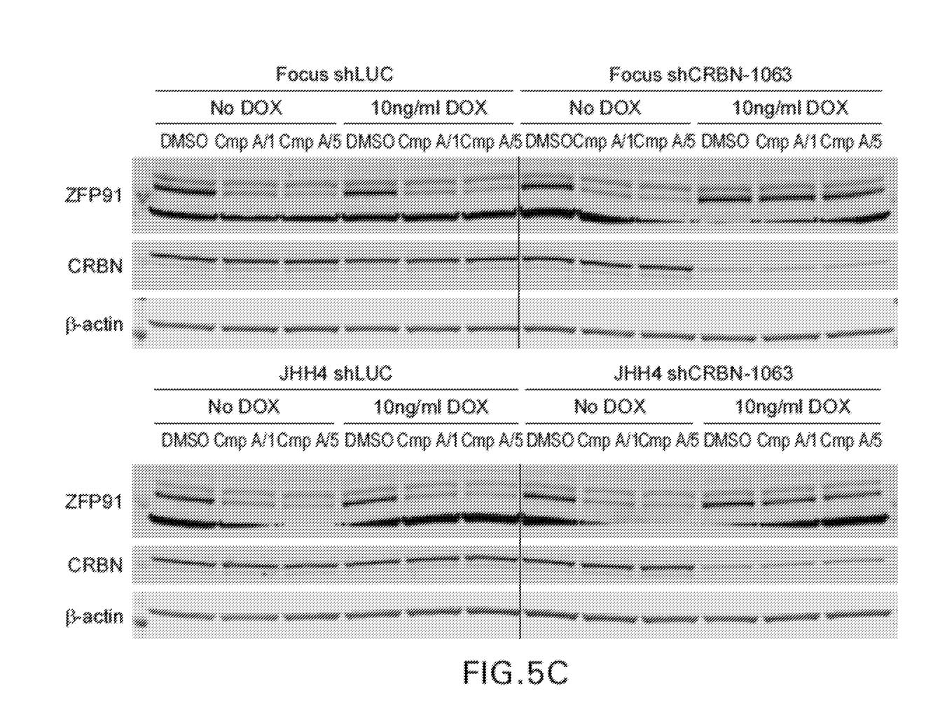

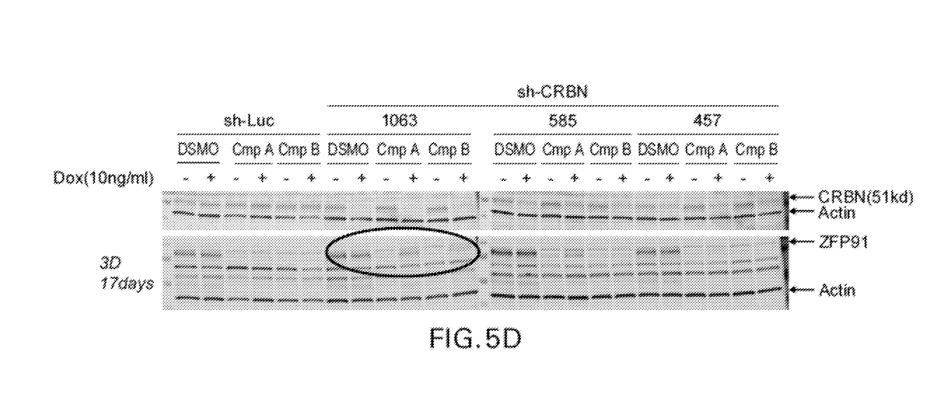

As shown in FIG. 5A, Western Analysis shows MG132 and MLN4924 block the reduction of the level of ZFP91 in response to Compound A treatment in HCC cell lines. HCC cells from both Focus and JHH4 cell lines in 2D setting were first treated with 10 μM MG132 or MLN4924 for 1 hour, and then were treated with 5 μM DMSO or 5 μM Compound A for 6 hours. Cells were harvested and were treated in cell lysis buffer to generate cell lysate. Proteins from cell lysate were separated by gel electrophoresis and transferred to nitrocellulose membranes. Immunoblots were then probed with antibodies recognizing CRBN, ZFP91 (LSBio), and β-actin (Li-Cor). The results were shown in FIG. 5A, and as shown, both 10 MG132 or MLN4924 inhibited ZFP91 degradation. MG132 activates c-Jun N-terminal kinase (JNK1), which initiates apoptosis. MG132 also inhibits NF-κB activation with an IC50 of 3 μM and prevents β-secretase cleavage. MLN4924 is a NAE1 inhibitor that blocks the activity of Cullin Ring Ligases (CRLs), such as CRL4CRBN. Both MG132 and MLN4924 inhibit CRBN activities, and thus these results indicate that ZFP91 is down-regulated in response to treatment compounds in a CRBN dependent pathway.

As shown in FIG. 5B, the level of ZFP91 decreased in response to Compound A treatment in Focus and JHH4 cells treated with a protein synthesis inhibitor-100 ug/ml cycloheximide (CHX). This result indicates that Compound A accelerates ZFP91 turnover through proteasome-mediated degradation.

As shown in FIG. 5C, knocking down of CRBN in Focus and JHH4 cells reversed the effect of Compound A on ZFP91 degradation. Similarly, as shown in FIG. 5D, knocking down of CRBN also reversed the effect of Compound B on ZFP91 protein level. Again, these results indicate that ZFP91 is down-regulated in response to treatment compounds in a CRBN dependent pathway.

### 5.7 ZFP91 is Down-Regulated in Response to Treatment Compounds in a CRBN Dependent Pathway in Hep3B Cell Lines

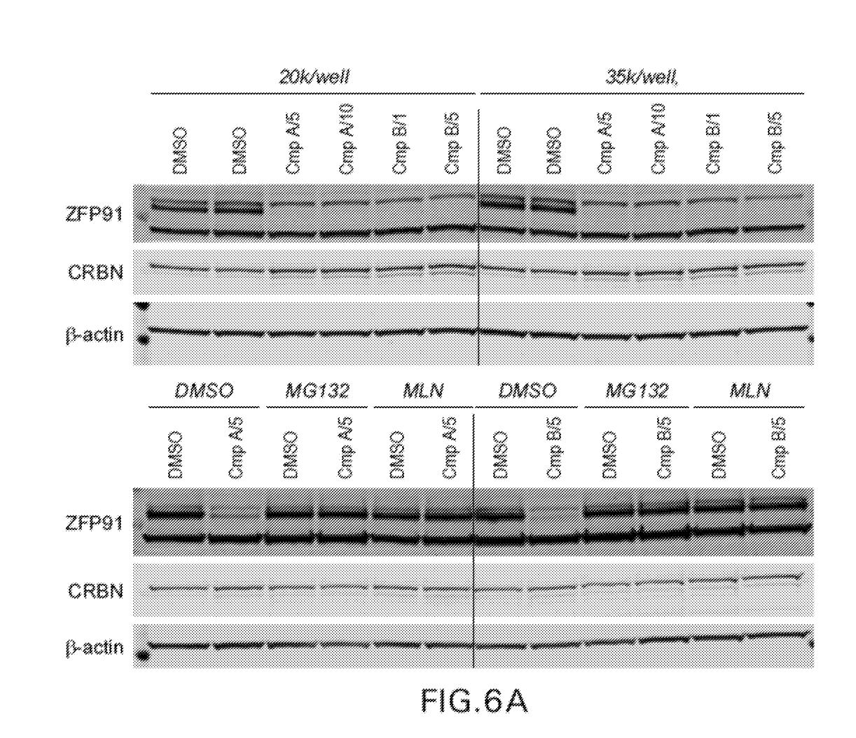

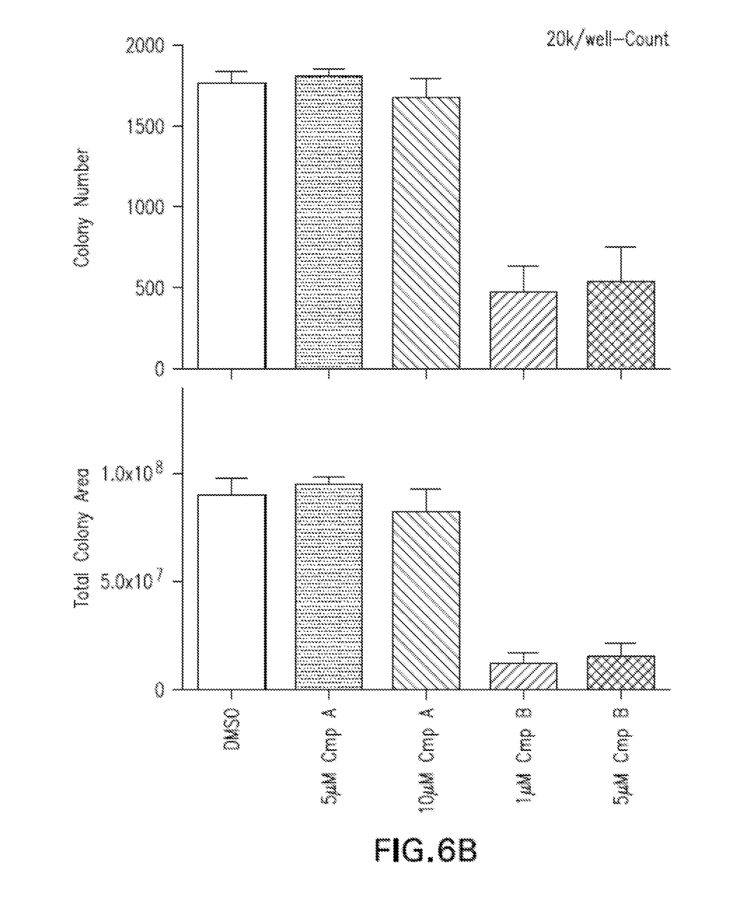

The change of ZFP91 level in response to Compound A or Compound B treatments was also evaluated in human hepatoma Hep3B cell lines. Hep3B cells were treated with Compound A or Compound B, and cells were harvested and treated in lysis buffer to generate cell lysate. Proteins from cell lysate were separated by electrophoresis gel and transferred to nitrocellulose membranes. Immunoblots were then probed with antibodies recognizing CRBN, ZFP91, and β-actin. Signals were detected with a Li-Cor Odyssey Imager. The results were shown in the upper panel of FIG. 6A. As shown, ZFP91 is down-regulated in response to treatment with Compound A or Compound B. Similarly to Focus and JHH4 cells, MG132 and MLN4924 blocked the reduction of the level of ZFP91 in response to Compound A or Compound B treatment, as shown in the lower panel of FIG. 6A. It was noted that Hep3B cells were sensitive to Compound B, but not to Compound A, as shown in FIG. 6B. Thus, ZFP91 may be degraded in response treatment compounds regardless of growth response of the cells to the treatment compounds.

### 5.8 ZFP91 is Down-Regulated in Response to Treatment Compounds in a CRBN Dependent Pathway in Giloblastoma Cell Lines

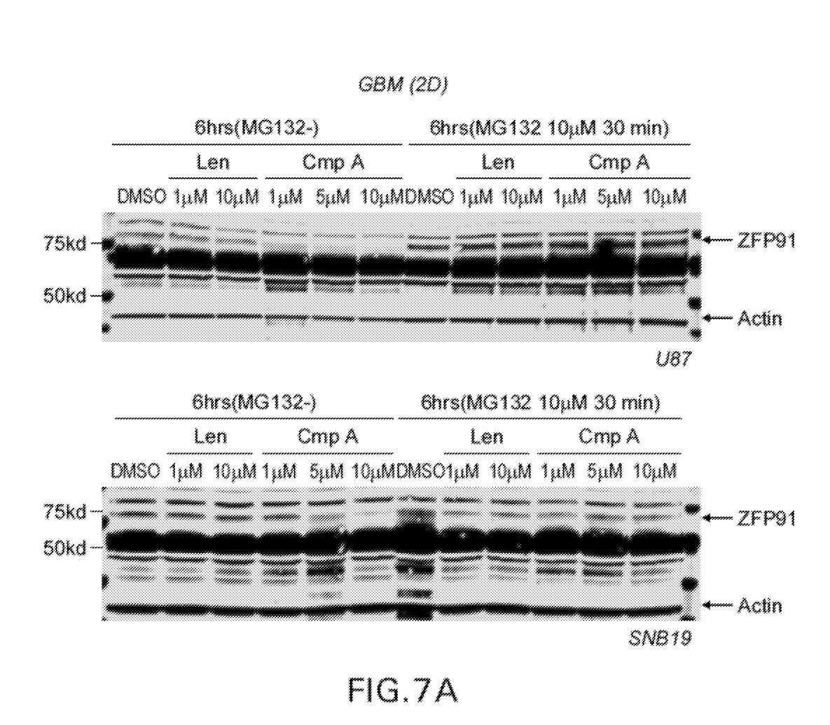

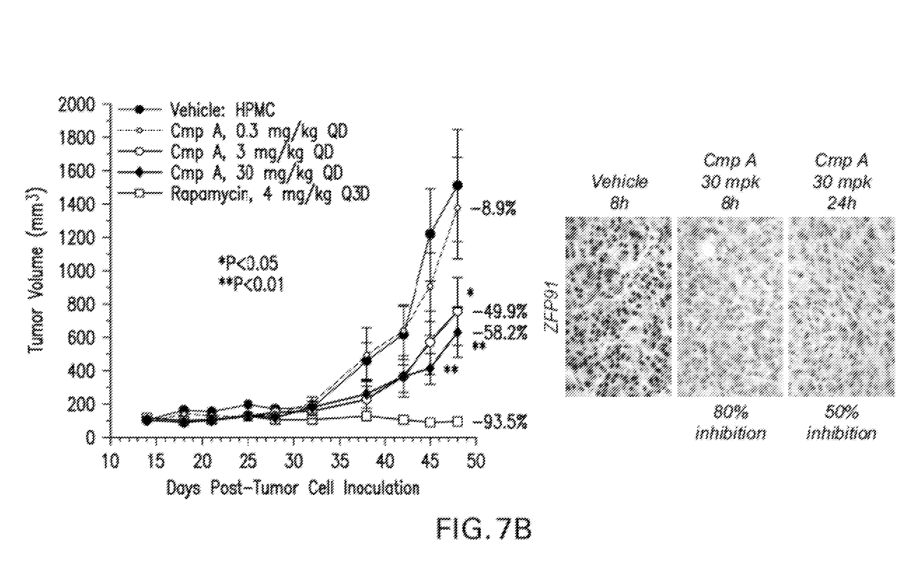

The change of ZFP91 level in response to treatment compounds was evaluated in other solid tumor cell lines, e.g., glioblastoma (GBM) cell lines. U87 human primary glioblastoma cells and SNB 19 giloma cells were treated with Compound A or lenalidomide, and cells were harvested and treated in lysis buffer to generate cell lysate. Proteins from cell lysate were separated by electrophoresis gel and transferred to nitrocellulose membranes. Immunoblots were then probed with antibodies recognizing ZFP91 and β-actin. Signals were detected with a Li-Cor Odyssey Imager. The results were shown in FIG. 7A. As shown, ZFP91 is down-regulated in response to treatment with Compound A or lenalidomide. As shown in the left panel of FIG. 7B, Compound A significantly inhibits U87 tumor growth at 3 and 30 mg/kg qd. Immunostaining assay showed that Compound A degraded ZFP91 in U87 xenograft tissue as shown in the right panel of FIG. 7B. Similarly to HCC cells, MG132 and MLN4924 blocked the reduction of the level of ZFP91 in response to treatment compounds in U87 and SNB19 cells, as shown in of FIG. 7A, indicating that the treatment compounds reduced the level of ZFP91 in a CRBN dependent pathway.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

What is claimed is:

1. A method of identifying a subject having a solid tumor who is likely to be responsive to a treatment compound, comprising:
(a) administering the treatment compound to a subject having a solid tumor;
(b) obtaining a sample from the subject;
(c) determining the level of a biomarker in the sample from the subject, wherein the biomarker is selected from the group consisting of biomarkers identified in Tables 1 to 6, and combinations thereof; and
(d) diagnosing the subject as being likely to be responsive to the treatment compound if the level of the biomarker in the sample of the subject changes as compared to a reference level of the biomarker.

2. A method of identifying a subject having a solid tumor who is likely to be responsive to a treatment compound, comprising:
(a) administering the treatment compound to a subject having a solid tumor;
(b) obtaining a sample from the subject;
(c) determining the level of a biomarker in the sample from the subject, wherein the biomarker is selected from the group consisting of biomarkers identified in Table 1, Table 3, and Table 4, and combinations thereof; and
(d) diagnosing the subject as being likely to be responsive to the treatment compound if the level of the biomarker in the sample of the subject is higher than a reference level of the biomarker.

3. A method of identifying a subject having a solid tumor who is likely to be responsive to a treatment compound, comprising:
(a) administering the treatment compound to a subject having a solid tumor;
(b) obtaining a sample from the subject;
(c) determining the level of a biomarker in the sample from the subject, wherein the biomarker is selected from the group consisting of biomarkers identified in Table 2, Table 5, and Table 6, and combinations thereof; and
(d) diagnosing the subject as being likely to be responsive to the treatment compound if the level of the biomarker in the sample of the subject is lower than a reference level of the biomarker.

4. A method of treating a solid tumor, comprising:
(a) obtaining a sample from the subject;
(b) determining the level of a biomarker in the sample from the subject, wherein the biomarker is selected from the group consisting of biomarkers identified in Tables 1-6, and combinations thereof;
(c) diagnosing the subject as being likely to be responsive to a treatment compound if the level of the biomarker in the sample of the subject changes as compared to a reference level of the biomarker; and
(d) administering a therapeutically effective amount of the treatment compound to the subject diagnosed to be likely to be responsive to the treatment compound.

5. A method of treating a solid tumor, comprising:
(a) obtaining a sample from the subject;
(b) determining the level of a biomarker in the sample from the subject, wherein the biomarker is selected from the group consisting of biomarkers identified in Table 1, Table 3, and Table 4, and combinations thereof;
(c) diagnosing the subject as being likely to be responsive to a treatment compound if the level of the biomarker in the sample of the subject is higher as than a reference level of the biomarker; and
(d) administering a therapeutically effective amount of the treatment compound to the subject diagnosed to be likely to be responsive to the treatment compound.

6. A method of treating a solid tumor, comprising:
(a) obtaining a sample from the subject;
(b) determining the level of a biomarker in the sample from the subject, wherein the biomarker is selected from the group consisting of biomarkers identified in Table 2, Table 5, and Table 6, and combinations thereof;
(c) diagnosing the subject as being likely to be responsive to a treatment compound if the level of the biomarker in the sample of the subject is lower as than a reference level of the biomarker; and
(d) administering a therapeutically effective amount of the treatment compound to the subject diagnosed to be likely to be responsive to the treatment compound.

7. A method of predicting the responsiveness of a subject having or suspected of having a solid tumor to a treatment compound, comprising:
(a) administering the treatment compound to a subject having a solid tumor;
(b) obtaining a sample from the subject;
(c) determining the level of a biomarker in the sample from the subject, wherein the biomarker is selected from the group consisting of biomarkers identified in Tables 1 to 6, and combinations thereof;
(d) diagnosing the subject as being likely to be responsive to a treatment of a solid tumor with the treatment compound if the level of the biomarker in the sample changes as compared to the level of the biomarker obtained from a reference sample.

8. A method of predicting the responsiveness of a subject having or suspected of having a solid tumor to a treatment compound, comprising:
(a) administering the treatment compound to a subject having a solid tumor;
(b) obtaining a sample from the subject;
(c) determining the level of a biomarker in the sample from the subject, wherein the biomarker is selected from the group consisting of biomarkers identified in Table 1, Table 3, and Table 4, and combinations thereof;
(d) diagnosing the subject as being likely to be responsive to a treatment of a solid tumor with the treatment compound if the level of the biomarker in the sample is higher than the level of the biomarker obtained from a reference sample.

9. A method of predicting the responsiveness of a subject having or suspected of having a solid tumor to a treatment compound, comprising:
(a) administering the treatment compound to a subject having a solid tumor;
(b) obtaining a sample from the subject;
(c) determining the level of a biomarker in the sample from the subject, wherein the biomarker is selected from the group consisting of biomarkers identified in Table 2, Table 5, and Table 6, and combinations thereof;
(d) diagnosing the subject as being likely to be responsive to a treatment of the solid tumor with the treatment compound if the level of the biomarker in the sample is less than the level of the biomarker obtained from a reference sample.

10. A method of monitoring the efficacy of a treatment of a solid tumor in a subject with a treatment compound, comprising:
(a) administering the treatment compound to a subject having a solid tumor;
(b) obtaining a sample from the subject;
(c) determining the level of a biomarker in the sample from the subject, wherein the biomarker is selected from the group consisting of biomarkers identified in Tables 1 to 6, and combinations thereof;
(d) comparing the level of the biomarker in the sample with the level of the biomarker obtained from a reference sample, wherein a change in the level as compared to the reference is indicative of the efficacy of the treatment compound in treating the solid tumor in the subject.

11. A method of monitoring the efficacy of a treatment of a solid tumor in a subject with a treatment compound, comprising:
(a) administering the treatment compound to a subject having a solid tumor;
(b) obtaining a sample from the subject;
(c) determining the level of a biomarker in the sample from the subject, wherein the biomarker is selected from the group consisting of biomarkers identified in Table 1, Table 3, and Table 4, and combinations thereof;
(d) comparing the level of the biomarker in the sample with the level of the biomarker obtained from a reference sample, wherein an increased level as compared to the reference is indicative of the efficacy of the treatment compound in treating the solid tumor in the subject.

12. A method of monitoring the efficacy of a treatment of a solid tumor in a subject with a treatment compound, comprising:
(a) administering the treatment compound to a subject having a solid tumor;
(b) obtaining a sample from the subject;
(c) determining the level of a biomarker in the sample from the subject, wherein the biomarker is selected from the group consisting of biomarkers identified in Table 2, Table 5, and Table 6, and combinations thereof;
(d) comparing the level of the biomarker in the sample with the level of the biomarker obtained from a reference sample, wherein a decreased level as compared to the reference is indicative of the efficacy of the treatment compound in treating the solid tumor in the subject.

13. The method of any one of claims 1, 4, 7, and 10, wherein the biomarker is selected from a group consisting of Nestin, KAT1/CCBL1, WIBG, MVP, PARP4, ZFP91, and ZNF198.

15. The method of any one of claims 2, 5, 8, and 11, wherein the biomarker is selected from a group consisting of Nestin, KAT1/CCBL1, and WIBG.

16. The method of claim 15, wherein the biomarker is Nestin.

17. The method of claim 15, wherein the biomarker is KAT1/CCBL1.

18. The method of claim 15, wherein the biomarker is WIBG.

19. The method of any one of claims 2, 5, 8, and 11, wherein the biomarker is CRBN.

20. The method of any one of claims 3, 6, 9, and 12, wherein the biomarker is selected from a group consisting of MVP, PARP4, ZFP91, and ZNF198.

21. The method of claim 20, wherein the biomarker is ZFP91.

22. The method of claim 20, wherein the biomarker is MVP.

23. The method of claim 20, wherein the biomarker is PARP4.

24. The method of claim 20, wherein the biomarker is ZNF198.

25. The method of any one of claims 1 to 15 and 20, wherein the level of only one biomarker is determined.

26. The method of any one of claims 1 to 15 and 20, wherein the levels of two, three, four, five or more biomarkers are determined.

27. The method of any one of claims 1 to 26, wherein the reference is prepared by using a control sample obtained from the subject prior to administration of the treatment compound to the subject; and wherein the control sample is from the same source as the sample.

28. The method of any one of claims 1 to 26, wherein the reference is prepared by using a control sample obtained from a healthy subject not having the solid tumor; and wherein the control sample is from the same source as the sample.

29. The method of any one of claims 1 to 28, wherein the levels of one or more of the biomarkers are measured by determining the mRNA levels of the biomarkers.

30. The method of any one of claims 1 to 28, wherein the levels of one or more of the biomarkers are measured by determining the cDNA levels of the biomarkers.

31. The method of any one of claims 1 to 28, wherein the levels of one or more of the biomarkers are measured by determining the protein levels of the biomarkers.

32. The method of claim 31, comprising contacting proteins within the sample with a first antibody that immunospecifically binds to the biomarker protein.

33. The method of claim 32, further comprising
(i) contacting the biomarker protein bound to the first antibody with a second antibody with a detectable label, wherein the second antibody immunospecifically binds to the biomarker protein, and wherein the second antibody immunospecifically binds to a different epitope on the biomarker protein than the first antibody;
(ii) detecting the presence of second antibody bound to the proteins; and
(iii) determining the amount of the biomarker protein based on the amount of detectable label in the second antibody.

34. The method of claim 32, further comprising:
(i) contacting the biomarker protein bound to the first antibody with a second antibody with a detectable label, wherein the second antibody immunospecifically binds to the first antibody;
(ii) detecting the presence of second antibody bound to the proteins; and
(iii) determining the amount of the biomarker protein based on the amount of detectable label in the second antibody.

35. The method of any one of claims 1 to 34, wherein the treatment compound is thalidomide, lenalidomide, pomalidomide, Compound A, or Compound B, or a stereoisomer thereof, or a pharmaceutically acceptable salt, solvate, hydrate, co-crystal, clathrate, or a polymorph thereof.

36. The method of claim 35, wherein the treatment compound is thalidomide, or a stereoisomer thereof, or a pharmaceutically acceptable salt, solvate, hydrate, co-crystal, clathrate, or a polymorph thereof.

37. The method of claim 35, wherein the treatment compound is lenalidomide, or a stereoisomer thereof, or a pharmaceutically acceptable salt, solvate, hydrate, co-crystal, clathrate, or a polymorph thereof.

38. The method of claim 35, wherein the treatment compound is pomalidomide, or a stereoisomer thereof, or a pharmaceutically acceptable salt, solvate, hydrate, co-crystal, clathrate, or a polymorph thereof.

39. The method of claim 35, wherein the treatment compound is Compound A, or a stereoisomer thereof, or a pharmaceutically acceptable salt, solvate, hydrate, co-crystal, clathrate, or a polymorph thereof.

40. The method of claim 35, wherein the treatment compound is Compound B, or a stereoisomer thereof, or a pharmaceutically acceptable salt, solvate, hydrate, co-crystal, clathrate, or a polymorph thereof.

41. The method of any one of claims 1-40, wherein the solid tumor is selected from a group consisting of sarcomas, carcinomas (epithelial tumors), melanomas, and glioblastomas.

42. The method of any one of claims 1-40, wherein the solid tumor is a brain cancer.

43. The method of claim 42, wherein the solid tumor is a glioblastoma (GBM).

44. The method of any one of claims 1-40, wherein the solid tumor is a liver cancer.

45. The method of claim 44, wherein the solid tumor is a hepatocellular cancer (HCC).
